(19) United States
(12) Patent Application Publication
(10) Pub. No.: US 2022/0280655 A1
(43) Pub. Date: Sep. 8, 2022

(54) New Adeno-Associated Virus (AAV) Variants and Uses Thereof for Gene Therapy

(71) Applicants: INSERM (INSTITUT NATIONAL DE LA SANTÉ ET DE LA RECHERCHE MÉDICALE), Paris (FR); Assistance Publique-Hôpitaux de Paris, Paris (FR); Genethon, Evry-Courcouronnes (FR); Sorbonne Université, Paris (FR); Universite de Paris, Paris (FR); Université d'Evry-Val-d'Essonne, Evry (FR); Universite Paris XIII Paris-Nord Villetaneuse, Villetaneuse (FR)

(72) Inventors: Jessica Zucman-Rossi, Paris (FR); Jean-Charles Nault, Paris (FR); Tiziana La Bella, Paris (FR); Giuseppe Ronzitti, Evry (FR); Sandrine Imbeaud, Paris (FR); Patrice Vidal, Evry (FR)

(73) Assignees: Institut National de la Santé et de la Recherche Médicale, Paris (FR); Assistance Publique-Hôpitaux de Paris, Paris (FR); Genethon, Evry-Courcouronnes (FR); Sorbonne Université, Paris (FR); Universite de Paris, Paris (FR); Université d'Evry-Val-d'Essonne, Evry (FR); Universite Paris XIII Paris-Nord Villetaneuse, Villetaneuse (FR)

(21) Appl. No.: 17/605,524

(22) PCT Filed: Apr. 23, 2020
(86) PCT No.: PCT/EP2020/061380

§ 371 Date: Oct. 21, 2021

(30) Foreign Application Priority Data
Apr. 23, 2019 (EP) 19305521.7

(51) Int. Cl. A61K 48/00; C12N 7/00; C12N 15/86

(52) U.S. Cl. CPC A61K 48/005; C12N 7/00; C12N 15/86; C12N 2750/14122; C12N 2750/14123; C12N 2750/14142

(57) ABSTRACT

Adeno-associated virus (AAV) is a defective mono-stranded DNA virus, endemic in human population (35-80%). Recurrent clonal AAV2 insertions are associated with the pathogenesis of rare human hepatocellular carcinoma (HCC) developed on normal liver. The aimed of the inventors was to characterize the natural history of AAV infection in the liver. Viral DNA was thus quantified in tumor and non-tumor liver tissues of 1461 patients. Presence of episomal form and viral mRNA expression were analyzed using a DNAse/TaqMan based assay and quantitative RT-PCR. In silico analyses using viral capture data explored viral variants and new clonal insertions. AAV DNA was detected in 21% of the patients equally distributed in 2 major viral subtypes: one similar to AAV2, the other hybrid between AAV2 and AAV13 sequences. Thus the inventors provided an integrated analysis of the wild type AAV infection in the liver with the identification of viral genotypes, molecular forms, helper virus relationship and viral integrations. These findings are important to understand wild type AAV biology and particularly relevant considering the large usage of AAV vector in liver-targeted gene therapy. Thus, the present invention relates to new adeno-associated virus (AAV) variants and uses thereof for gene therapy.

### FIELD OF THE INVENTION

The present invention relates to new adeno-associated virus (AAV) variants and uses thereof for gene therapy.

### BACKGROUND OF THE INVENTION

Adeno-associated virus (AAV) is a small non-enveloped DNA virus composed by an icosahedral capsid that contains a 4.7 kb linear single-stranded genome.^{1, 2} AAV genome codes for non-structural proteins (Rep78, 68, 52 and 40), capsid proteins (VP1, VP2, VP3) and the assembly-activating protein (AAP).^{3, 4} At the extremities, inverse tandem repeats (ITR) are important for the integration in host genomes.^{5, 6} AAV is a defective virus requiring the presence of a helper virus for an active infection, otherwise it establishes a latent infection through integration into host genome or persistence as circular episomal form.⁷⁻⁹ AAV seroprevalence showed that the infection is endemic in human populations (35-80%) starting during childhood.¹⁰⁻¹² Twelve distinct serotypes and more than 100 natural variants have been identified, among which AAV2 is the most frequent type in human.¹³⁻¹⁶

This small virus has attracted the attention of the gene therapy field because of the lack of identifiable associated disease and the remarkable ability of recombinant AAV (rAAV) vectors to transduce dividing and non-dividing cells with high efficiency, long-term transgene expression, low immunogenicity and specific tissue tropism.¹⁷ Although AAV discovery was performed in 1965, many questions regarding AAV infection remain unanswered.^{2, 18} It is well known that the vector predominantly persist in the nucleus as episomal form with sustained RNA expression raising question about the potential presence of episomal AAV in wild type infection.⁸ Several helper viruses have been identified but their precise association with wild type liver AAV infection remains unclear. Few data on the specific AAV genotypes in the population and on the frequency of AAV persistence after first infection are available.¹⁹

### SUMMARY OF THE INVENTION

As defined by the claims, the present invention relates to new adeno-associated virus (AAV) variants and uses thereof for gene therapy.

### DETAILED DESCRIPTION OF THE INVENTION

Adeno-associated virus (AAV) is a defective mono-stranded DNA virus, endemic in human population (35-80%). Recurrent clonal AAV2 insertions are associated with the pathogenesis of rare human hepatocellular carcinoma (HCC) developed in normal liver. The aim of the inventors was to characterize the natural history of AAV infection in the liver. Viral DNA was thus quantified in tumor and non-tumor liver tissues of 1461 patients. Presence of episomal form and viral mRNA expression were analyzed using a DNAse/TaqMan based assay and quantitative RT-PCR. In silico analyses of viral capture data identified viral variants and new clonal insertions. AAV DNA was detected in 21% of the patients equally distributed in 2 major viral subtypes: one similar to AAV2, the other hybrid between AAV2 and AAV13 sequences. Thus, the inventors provided an integrated analysis of the wild type AAV infection in the liver with the identification of viral genotypes, molecular forms, helper virus relationship and viral integrations. These findings are important to understand wild type AAV biology. The data are particularly relevant considering the therapeutic potential of this new AAV variants in liver-targeted gene therapy.

### Main Definitions

As used herein, the term “polynucleotide” or “nucleic acid” refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugars or phosphate groups. Alternatively, the backbone of the polynucleotide can comprise a polymer of synthetic subunits such as phosphoramidates and thus can be an oligodeoxynucleoside phosphoramidate (P—NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

As used herein, the terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.”

As used herein the term “wild type” or “native” is a term of the art understood by skilled persons and means the typical form of an organism, strain, gene or characteristic as it occurs in nature as distinguished from mutant forms.

As used herein, the term “mutation” has its general meaning in the art and refers to a substitution, deletion or insertion. In particular, the term “substitution” means that a specific amino acid residue at a specific position is removed and another amino acid residue is inserted into the same position.

As used herein, the term “variant” refers to a first composition (e.g., a first molecule), that is related to a second composition (e.g., a second molecule, also termed a “parent” molecule). The variant molecule can be derived from, isolated from, based on or homologous to the parent molecule. A variant molecule can have entire sequence identity with the original parent molecule, or alternatively, can have less than 100% sequence identity with the parent molecule. For example, a variant of a sequence can be a second sequence that is at least 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100% identical in sequence compared to the original sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

As used herein, the term “adeno-associated virus” or “AAV” has its general meaning in the art and refers to a Dependoparvovirus within the Parvoviridae genus of viruses. The term can refer to an AAV derived from a naturally occurring “wild-type” virus, or an AAV derived from a rAAV genome packaged into a capsid derived from capsid proteins encoded by a naturally occurring cap gene. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV, porcine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc. The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession Numbers NC-002077 (AAV-1), AF063497 (AAV-1), NC-001401 (AAV-2), AF043303 (AAV-2), NC-001729 (AAV-3), NC-001829 (AAV-4), U89790 (AAV-4), NC-006152 (AAV-5), AF513851 (AAV-7), AF513852 (AAV-8), and NC-006261 (AAV-8); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99:11854; Moris et al. (2004) Virology 33:375-383; international patent publications WO 00/28061, WO 99/61601, WO 98/11244; and U.S. Pat. No. 6,156,303. The complete genome of AAV2 is represented by the reference sequence as set forth in SEQ ID NO:1.

| SEQ ID NO: 1 | |
|---|---|
| >NCBI Reference Sequence: NC_001401.2 | |
| 1 | ttggccactc cctctctgcg cgctcgctcg ctcactgagg ccgggcgacc aaaggtcgcc |
| 61 | cgacgcccgg gctttgcccg ggcggcctca gtgagcgagc gagcgcgcag agagggagtg |
| 121 | gccaactcca tcactagggg ttcctggagg ggtggagtcg tgacgtgaat tacgtcatag |
| 181 | ggttagggag gtcctgtatt agaggtcacg tgagtgtttt gcgacatttt gcgacaccat |
| 241 | gtggtcacgc tgggtattta agcccgagtg agcacgcagg gtctccattt tgaagcggga |
| 301 | ggtttgaacg cgcagccgcc atgccggggt tttacgagat tgtgattaag gtccccagcg |
| 361 | accttgacga gcatctgccc ggcatttctg acagctttgt gaactgggtg gccgagaagg |
| 421 | aatgggagtt gccgccagat tctgacatgg atctgaatct gattgagcag gcacccctga |
| 481 | ccgtggccga gaagctgcag cgcgactttc tgacggaatg gcgccgtgtg agtaaggccc |
| 541 | cggaggccct tttctttgtg caatttgaga agggagagag ctacttccac atgcacgtgc |
| 601 | tcgtggaaac caccggggtg aaatccatgg ttttgggacg tttcctgagt cagattcgcg |
| 661 | aaaaactgat tcagagaatt taccgcggga tcgagccgac tttgccaaac tggttcgcgg |
| 721 | tcacaaagac cagaaatggc gccggaggcg ggaacaaggt ggtggatgag tgctacatcc |
| 781 | ccaattactt gctccccaaa acccagcctg agctccagtg ggcgtggact aatatggaac |
| 841 | agtatttaag cgcctgtttg aatctcacgg agcgtaaacg gttggtggcg cagcatctga |
| 901 | cgcacgtgtc gcagacgcag gagcagaaca aagagaatca gaatcccaat tctgatgcgc |
| 961 | cggtgatcag atcaaaaact tcagccaggt acatggagct ggtcgggtgg ctcgtggaca |
| 1021 | aggggattac ctcggagaag cagtggatcc aggaggacca ggcctcatac atctccttca |
| 1081 | atgcggcctc caactcgcgg tcccaaatca aggctgcctt ggacaatgcg ggaaagatta |
| 1141 | tgagcctgac taaaaccgcc cccgactacc tggtgggcca gcagcccgtg gaggacattt |
| 1201 | ccagcaatcg gatttataaa attttggaac taaacgggta cgatccccaa tatgcggctt |
| 1261 | ccgtctttct gggatgggcc acgaaaaagt tcggcaagag gaacaccatc tggctgtttg |
| 1321 | ggcctgcaac taccgggaag accaacatcg cggaggccat agcccacact gtgcccttct |
| 1381 | acgggtgcgt aaactggacc aatgagaact ttcccttcaa cgactgtgtc gacaagatgg |
| 1441 | tgatctggtg ggaggagggg aagatgaccg ccaaggtcgt ggagtcggcc aaagccattc |
| 1501 | tcggaggaag caaggtgcgc gtggaccaga aatgcaagtc ctcggcccag atagacccga |
| 1561 | ctcccgtgat cgtcacctcc aacaccaaca tgtgcgccgt gattgacggg aactcaacga |
| 1621 | ccttcgaaca ccagcagccg ttgcaagacc ggatgttcaa atttgaactc acccgccgtc |
| 1681 | tggatcatga ctttgggaag gtcaccaagc aggaagtcaa agactttttc cggtgggcaa |
| 1741 | aggatcacgt ggttgaggtg gagcatgaat tctacgtcaa aaagggtgga gccaagaaaa |
| 1801 | gacccgcccc cagtgacgca gatataagtg agcccaaacg ggtgcgcgag tcagttgcgc |
| 1861 | agccatcgac gtcagacgcg gaagcttcga tcaactacgc agacaggtac caaaacaaat |
| 1921 | gttctcgtca cgtgggcatg aatctgatgc tgtttccctg cagacaatgc gagagaatga |
| 1981 | atcagaattc aaatatctgc ttcactcacg gacagaaaga ctgtttagag tgctttcccg |
| 2041 | tgtcagaatc tcaacccgtt tctgtcgtca aaaaggcgta tcagaaactg tgctacattc |
| 2101 | atcatatcat gggaaaggtg ccagacgctt gcactgcctg cgatctggtc aatgtggatt |
| 2161 | tggatgactg catctttgaa caataaatga tttaaatcag gtatggctgc cgatggttat |
| 2221 | cttccagatt ggctcgagga cactctctct gaaggaataa gacagtggtg gaagctcaaa |
| 2281 | cctggcccac caccaccaaa gcccgcagag cggcataagg acgacagcag gggtcttgtg |
| 2341 | cttcctgggt acaagtacct cggacccttc aacggactcg acaagggaga gccggtcaac |
| 2401 | gaggcagacg ccgcggccct cgagcacgac aaagcctacg accggcagct cgacagcgga |
| 2461 | gacaacccgt acctcaagta caaccacgcc gacgcggagt ttcaggagcg ccttaaagaa |
| 2521 | gatacgtctt ttgggggcaa cctcggacga gcagtcttcc aggcgaaaaa gagggttctt |
| 2581 | gaacctctgg gcctggttga ggaacctgtt aagacggctc cgggaaaaaa gaggccggta |
| 2641 | gagcactctc ctgtggagcc agactcctcc tcgggaaccg gaaaggcggg ccagcagcct |
| 2701 | gcaagaaaaa gattgaattt tggtcagact ggagacgcag actcagtacc tgacccccag |
| 2761 | cctctcggac agccaccagc agccccctct ggtctgggaa ctaatacgat ggctacaggc |
| 2821 | agtggcgcac caatggcaga caataacgag ggcgccgacg gagtgggtaa ttcctcggga |
| 2881 | aattggcatt gcgattccac atggatgggc gacagagtca tcaccaccag cacccgaacc |
| 2941 | tgggccctgc ccacctacaa caaccacctc tacaaacaaa tttccagcca atcaggagcc |
| 3001 | tcgaacgaca atcactactt tggctacagc accccttggg ggtattttga cttcaacaga |
| 3061 | ttccactgcc acttttcacc acgtgactgg caaagactca tcaacaacaa ctggggattc |
| 3121 | cgacccaaga gactcaactt caagctcttt aacattcaag tcaaagaggt cacgcagaat |
| 3181 | gacggtacga cgacgattgc caataacctt accagcacgg ttcaggtgtt tactgactcg |
| 3241 | gagtaccagc tcccgtacgt cctcggctcg gcgcatcaag gatgcctccc gccgttccca |
| 3301 | gcagacgtct tcatggtgcc acagtatgga tacctcaccc tgaacaacgg gagtcaggca |
| 3361 | gtaggacgct cttcatttta ctgcctggag tactttcctt ctcagatgct gcgtaccgga |
| 3421 | aacaacttta ccttcagcta cacttttgag gacgttcctt tccacagcag ctacgctcac |
| 3481 | agccagagtc tggaccgtct catgaatcct ctcatcgacc agtacctgta ttacttgagc |
| 3541 | agaacaaaca ctccaagtgg aaccaccacg cagtcaaggc ttcagttttc tcaggccgga |
| 3601 | gcgagtgaca ttcgggacca gtctaggaac tggcttcctg gaccctgtta ccgccagcag |
| 3661 | cgagtatcaa agacatctgc ggataacaac aacagtgaat actcgtggac tggagctacc |
| 3721 | aagtaccacc tcaatggcag agactctctg gtgaatccgg gcccggccat ggcaagccac |
| 3781 | aaggacgatg aagaaaagtt ttttcctcag agcggggttc tcatctttgg gaagcaaggc |
| 3841 | tcagagaaaa caaatgtgga cattgaaaag gtcatgatta cagacgaaga ggaaatcagg |
| 3901 | acaaccaatc ccgtggctac ggagcagtat ggttctgtat ctaccaacct ccagagaggc |
| 3961 | aacagacaag cagctaccgc agatgtcaac acacaaggcg ttcttccagg catggtctgg |
| 4021 | caggacagag atgtgtacct tcaggggccc atctgggcaa agattccaca cacggacgga |
| 4081 | cattttcacc cctctcccct catgggtgga ttcggactta aacaccctcc tccacagatt |
| 4141 | ctcatcaaga acaccccggt acctgcgaat ccttcgacca ccttcagtgc ggcaaagttt |
| 4201 | gcttccttca tcacacagta ctccacggga caggtcagcg tggagatcga gtgggagctg |
| 4261 | cagaaggaaa acagcaaacg ctggaatccc gaaattcagt acacttccaa ctacaacaag |
| 4321 | tctgttaatg tggactttac tgtggacact aatggcgtgt attcagagcc tcgccccatt |
| 4381 | ggcaccagat acctgactcg taatctgtaa ttgcttgtta atcaataaac cgtttaattc |
| 4441 | gtttcagttg aactttggtc tctgcgtatt tctttcttat ctagtttcca tggctacgta |
| 4501 | gataagtagc atggcgggtt aatcattaac tacaaggaac ccctagtgat ggagttggcc |
| 4561 | actccctctc tgcgcgctcg ctcgctcact gaggccgggc gaccaaaggt cgcccgacgc |
| 4621 | ccgggctttg cccgggcggc ctcagtgagc gagcgagcgc gcagagaggg agtggccaa |

As used herein, the term “cap gene” refers to the polynucleotide that encode capsid proteins that form, or contribute to the formation of, the capsid, or protein shell, of the virus. In the case of AAV2, the capsid protein may be VP1, VP2, or VP3. For other parvoviruses, the names and numbers of the capsid proteins can differ. The VP1 capsid protein of AAV2 is represented by the reference amino acid sequence as set forth in SEQ ID NO:2. The VP2 capsid protein of AAV2 is represented by the amino acid sequence that ranges from the amino acid residue at position 138 to the last amino acid residue in SEQ ID NO:2. The VP3 capsid protein of AAV2 is represented by the amino acid sequence that ranges from the amino acid residue at position 203 to the last amino acid residue in SEQ ID NO:2.

SED ID NO: 2
>sp|P03135|CAPSD_AAV2S Capsid protein VP1
OS = Adeno-associated virus 2 (isolate Srivastava/
1982) OX = 648242 GN = VP1 PE = 1 SV = 2
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

As used herein, the term “rep gene” refers to the nucleic acid sequences that encode the non-structural proteins (rep78, rep68, rep52 and rep40) required for the replication and production of virus.

As used herein, the term “packaging” refers to a series of intracellular events that result in the assembly and encapsidation of an AAV virus.

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC. “Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans. For example, a plasmid or other expression vector comprising nucleotide sequences encoding one or more adenoviral proteins is transfected into a producer cell along with nucleotidic sequences enconding for the AAV rep and cap genes and a transgene expression cassette flanked by two ITRs to produce a rAAV

As used herein, the term “virus viral particle” is one that comprises a competently assembled viral capsid and is capable of delivering a polynucleotide component into a cell for which the viral species is tropic. The term does not necessarily imply any replication capacity of the virus. Assays for counting infectious viral particles are described elsewhere in this disclosure and in the art. Viral infectivity can be expressed as the ratio of infectious viral particles to total viral particles. Methods of determining the ratio of infectious viral particle to total viral particle are known in the art. See, e.g., Grainger et al. (2005) Mol. Ther. 11:S337 (describing a TCID50 infectious titer assay); and Zolotukhin et al. (1999) Gene Ther. 6:973. See also the Examples.

As used herein, the term “transgene” refers to a polynucleotide that is introduced into a cell and is capable of being transcribed into RNA and optionally, translated and/or expressed under appropriate conditions. In aspects, it confers a desired property to a cell into which it is introduced, or otherwise leads to a desired therapeutic outcome. In another aspect, it may be transcribed into a molecule that mediates RNA interference, such as miRNA, siRNA, or shRNA.

As used herein, the term “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

As used herein, the term “operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

As used herein, the term “heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV.

As used herein, the term “isolated” when referred to a plasmid, nucleic acid, vector, virus, virion, host cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this disclosure are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

The term “Pharmaceutically” or “pharmaceutically acceptable” refer to molecular entities and compositions that do not produce an adverse, allergic or other untoward reaction when administered to a mammal, especially a human, as appropriate.

By a “therapeutically effective amount” is meant a sufficient amount of the rAAV of the present invention to treat the disease at a reasonable benefit/risk ratio. It will be understood that the total daily usage of the rAAV of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. Thus, the doses of vectors may be adapted depending on the disease condition, the subject (for example, according to his weight, metabolism, etc.), the treatment schedule, etc. A preferred effective dose within the context of this invention is a dose allowing an optimal transduction of the cone photoreceptors. Typically, from 10⁸ to 10¹⁰ viral genomes (vg) are administered per dose in mice. Typically, the doses of AAV to be administered in humans may range from 10⁹ to 10¹² vg.

Variant AAV2 Capsid Protein:

An object of the present invention relates to a variant VP1 capsid protein that consists of the amino acid sequence as set forth in SEQ ID NO:2 comprising at least one mutation of the table 1.

An object of the present invention relates to a variant VP2 capsid protein that consists of the amino acid sequence that ranges from the amino acid at position 138 to the last amino acid in SEQ ID NO:2 comprising at least one mutation of the table 1.

An object of the present invention relates to a variant VP3 capsid protein that consists of the amino acid sequence that ranges from the amino acid at position 203 to the last amino acid in SEQ ID NO:2 comprising at least one mutation of the table 1.

TABLE 1
List of the mutations present in the capsid sequences

| Nucleotide Change | | |
|---|---|---|
| (coordinates on NC_001401) | Protein Change | Samples with variant |
| 2236G > C | 12E > Q | #2112 |
| 2237A > G | 12E > G | #1010 |
| 2238G > A | | #2112 |
| 2241C > G | 13D > E | #1010 |
| 2241C > A | 13D > E | #2112 |
| 2243_2244delinsAC | 14T > N | #1024 #2112 |
| 2244T > C | | #1010 |
| 2246T > C | 15L > P | #985 |
| 2247C > A | | #2112 |
| 2248_2249delinsAG | | #1024 |
| 2249C > T | 16S > F | #2112 |
| 2251G > C | 17E > Q | #1010 |
| 2251_2253delinsTTC | 17E > F | #2112 |
| 2252A > C | 17E > A | #985 |
| 2255_2256delinsAT | 18G > D | #2112 |
| 2259A > T | | #1024 |
| 2260A > C | | #1024 #2112 |
| 2262A > C | | #1024 |
| 2262A > T | 20R > S | #985 |
| 2263C > G | 21Q > E | #1024 |
| 2272_2275delinsGCTT | 24K > A | #1024 |
| 2277C > G | | #1024 |
| 2277C > T | | #442 |
| 2286_2287delinsAG | 29P > A | #1024 |
| 2289A > C | | #1024 |
| 2292A > T | | #1024 |
| 2292A > G | | #668 |
| 2294C > A | 31P > Q | #1024 |
| 2295A > G | | #M258 |
| 2298A > C | | #1024 |
| 2302C > T | 34P > S | #1602 |
| 2302_2313delinsGCAAATCAACAA | 34_37delinsANQQ | #1024 |
| 2316T > C | | #3013 #3511 #777 |
| 2317A > C | 39K > Q | #1024 #1273 #129 #1534 |
| | | #2102 #2112 |
| 2319G > A | | #1024 #767 |
| 2323G > A | 41D > N | #1024 #2112 #2806 #3086 |
| 2326_2329delinsGCTC | 42S > A | #1024 |
| 2326A > G | 42S > G | #1260 |
| 2327G > A | 42S > N | #1449 #668 |
| 2328C > T | | #3013 |
| 2346T > G | | #1024 |
| 2349G > T | | #1024 |
| 2355G > A | | #1024 |
| 2361C > T | | #1024 #2206 |
| 2367C > T | | #508 |
| 2368_2369delinsGG | 56F > G | #1024 |
| 2385G > A | | #1602 #1704 #2102 #3013 |
| | | #777 |
| 2388A > G | | #1024 #1343 #1449 #2040 |
| | | #3765 #387 #471 #473 |
| 2391G > A | | #1010 |
| 2402AG > CA | 67E > A | #1024 |
| 2412C > G | | #1024 |
| 2419C > A | 73L > I | #508 |
| 2424G > A | | #1010 #2497 #2557 #2806 |
| | | #3013 |
| 2433A > G | | #1010 #1017 #1020 #1024 |
| | | #1158 #1260 #1273 #1286 |
| | | #1343 #1350 #1449 #1534 |
| | | #1570 #1591 #1602 #163 |
| | | #1704 #1919 #2040 #2087 |
| | | #2102 #2107 #2112 #2128 |
| | | #2141 #217 #2206 #2208 |
| | | #2320 #2497 #2557 #2731 |
| | | #2806 #3013 #3086 #3142 |
| | | #3511 #367 #371 #3765 |
| | | #387 #442 #471 #473 #508 |
| | | #668 #685 #704 #714 #767 |
| | | #777 #790 #877 #976 #985 |
| | | #M258 |
| 2436C > A | | #1024 |
| 2443_2444delinsAA | 81R > K | #1024 |
| 2448G > A | | #767 |
| 2452_2456delinsGAGGC | 84DS > KA | #1024 |
| 2454C > A | 84D > E | #2102 |
| 2461G > C | 87D > H | #1010 |
| 2465A > T | 88N > I | #1010 |
| 2466C > T | | #1449 |
| 2478G > A | | #471 |
| 2499G > A | | #767 |
| 2502T > C | | #1704 |
| 2508G > T | 102E > D | #1273 |
| 2511C > T | | #1020 #1024 #129 #1350 |
| | | #1449 #1591 #2040 #2112 |
| | | #217 #2208 #2320 #2806 |
| | | #3013 #3086 #3142 #3511 |
| | | #3765 #387 #471 #508 #685 |
| | | #777 |
| 2538C > A | | #1343 |
| 2546G > C | 115G > A | #1704 |
| 2548C > A | | #1704 |
| 2556C > T | | #1704 |
| 2565G > A | | #1704 #714 #767 #877 |
| | | #M258 |
| 2565G > C | | #2557 |
| 2580T > C | | #1024 |
| 2584C > A | 128P > T | #2320 |
| 2604A > G | | #1343 #2112 #2141 #2557 |
| | | #2806 #3086 #3765 #471 |
| | | #714 #777 #M258 |
| 2606C > A | 135P > H | #2320 |
| 2606C > G | 135P > R | #2731 |
| 2613G > A | | #1273 #1343 #1350 #1449 |
| | | #2112 #2141 #387 #685 |
| | | #714 #777 |
| 2622G > T | | #667 #767 |
| 2625A > T | | #2320 |
| 2635C > G | 145P > A | #2320 |
| 2637G > A | | #2731 #508 |
| 2643G > A | | #471 |
| 2642_2705delins_56nt | 147_168delins_22aa | #2731 |
| 2646_2647delinsTG | 149S > A | #2320 |
| 2646C > T | | #2497 #3013 |
| 2651C > A | 150P > H | #2320 |
| 2654T > C | 151V > A | #1024 #1260 #129 #1534 |
| | | #1591 #1704 #2112 #2128 |
| | | #2206 #2208 #2557 #3013 |
| | | #3142 #3511 #3765 #387 |
| | | #471 #473 #508 #685 #714 |
| | | #767 #877 #M258 |
| 2655G > T | | #2208 |
| 2658G > A | | #2040 #508 |
| 2664C > T | | #1024 #1704 #2208 #3511 |
| | | #387 |
| 2670C > T | | #1017 |
| 2673G > C | | #1024 #1449 #1704 #2208 |
| | | #3511 #387 |
| 2678C > T | 159T > I | #714 |
| 2679C > T | | #1024 #1449 #1704 #2208 |
| | | #3511 #387 |
| 2679C > A | | #2087 |
| 2685G > A | | #1010 #1017 #1020 #1158 |
| | | #1260 #1273 #1286 #129 |
| | | #1343 #1350 #1534 #1570 |
| | | #1591 #1602 #163 #2040 |
| | | #2087 #2107 #2112 #217 |
| | | #2206 #2557 #2731 #2806 |
| | | #3086 #367 #371 #3765 |
| | | #442 #471 #473 #667 #668 |
| | | #685 #704 #714 #767 #777 |
| | | #790 #M258 |
| 2686G > T | 162A > S | #1024 #1449 #1704 #2208 |
| | | #3511 #387 |
| 2692C > A | 164Q > K | #2112 |
| 2692_2694delinsAAC | 164Q > N | #1534 #1704 #1919 #2087 |
| | | #2102 #2128 #2141 #2206 |
| | | #2557 #3142 #3765 #387 |
| | | #471 #508 #790 #877 |
| | | #M258 |
| 2694G > A | | #1024 #1449 #2208 #2806 |
| | | #3086 #3511 |
| 2703A > G | | #1343 #2112 #2806 #3013 |
| | | #3086 #777 |
| 2705G > A | 168R > K | #2806 #3086 |
| 2709A > G | | #1273 #1343 #1704 #1919 |
| | | #2102 #2557 #3013 #3511 |
| | | #387 #442 #508 #777 |
| 2713T > C | | #1017 #1158 #1260 #129 |
| | | #1350 #1602 #2040 #2206 |
| | | #2806 #3086 #3142 #3765 |
| 2716_2724delAATTTTGGT | 172_175delNFG | #1273 |
| 2718T > G | 172N > K | #2806 |
| 2721T > C | | #1010 #1017 #1158 #1260 |
| | | #129 #1343 #1350 #1449 |
| | | #1534 #1602 #1919 #2040 |
| | | #2087 #2102 #2128 #2141 |
| | | #2206 #2557 #3013 #3142 |
| | | #387 #442 #471 #473 #508 |
| | | #667 #668 #685 #714 #767 |
| | | #777 #877 #985 #M258 |
| 2724T > C | | #1010 |
| 2734_2742delinsCCTTATGCT | 178DA > PY | #1055 |
| 2737G > T | 179A > S | #1602 |
| 2742C > T | | #2731 |
| 2744_2751delinsGGCATTGC | 181_183delinsWHC | #2731 |
| 2745A > C | | #1010 #1017 #1020 #1024 |
| | | #1055 #1158 #1260 #1273 |
| | | #1286 #129 #1343 #1350 |
| | | #1449 #1534 #1570 #1591 |
| | | #163 #1704 #1919 #2040 |
| | | #2087 #2102 #2107 #2112 |
| | | #2128 #2141 #217 #2206 |
| | | #2208 #2320 #2557 #2806 |
| | | #3013 #3086 #3142 #3511 |
| | | #367 #371 #3765 #387 #442 |
| | | #471 #473 #508 #667 #668 |
| | | #685 #704 #714 #767 #777 |
| | | #790 #877 #976 #985 |
| | | #M258 |
| 2746_2776delins_31nt | 182_192delins_SSRDMRKVFSA | #1602 |
| 2751T > A | | #2208 |
| 2757C > T | | #2208 |
| 2760G > A | | #2102 #2128 #2208 |
| 2764C > A | 188L > I | #3142 |
| 2770C > A | 190Q > K | #1158 |
| 2770C > G | 190Q > E | #2208 #M258 |
| 2772G > A | | #2208 #442 #790 #M258 |
| 2788T > A | 196S > T | #129 #1602 #2208 #3142 |
| | | #508 #M258 |
| 2790T > A | | #129 #1602 #2208 #3142 |
| | | #508 #M258 |
| 2791G > A | 197G > S | #129 #1602 #2208 #3142 |
| | | #508 #M258 |
| 2794C > T | | #1286 #129 #1570 #1591 |
| | | #1602 #163 #1704 #2107 |
| | | #217 #2208 #2731 #3142 |
| | | #367 #508 #685 #767 |
| | | #M258 |
| 2800A > T | 200T > S | #1020 #1024 #1158 #1286 |
| | | #129 #1350 #1570 #1591 |
| | | #1602 #163 #1704 #2107 |
| | | #217 #2208 #2320 #2731 |
| | | #2806 #3086 #3142 #367 |
| | | #442 #508 #668 #685 #704 |
| | | #714 #767 #790 #976 |
| | | #M258 |
| 2804A > C | 201N > T | #1010 #1020 #1024 #1158 |
| | | #1286 #129 #1350 #1570 |
| | | #1591 #1602 #163 #1704 |
| | | #2040 #2107 #217 #2208 |
| | | #2320 #2731 #2806 #3086 |
| | | #3142 #367 #442 #508 #668 |
| | | #685 #704 #714 #767 #790 |
| | | #976 #M258 |
| 2808G > A | | #1020 #1024 #1158 #1286 |
| | | #129 #1350 #1570 #1591 |
| | | #1602 #163 #1704 #2107 |
| | | #217 #2208 #2320 #2731 |
| | | #2806 #3086 #3142 #367 |
| | | #442 #508 #668 #685 #704 |
| | | #714 #767 #790 #976 |
| | | #M258 |
| 2815A > T | 205T > S | #1017 #1602 #2087 #2206 |
| | | #2208 #2320 #M258 |
| 2821A > G | 207S > G | #1602 #2208 #M258 |
| 2831_2851delins_21nt | 210_217delinsLLLLESLS | #2731 |
| 2833A > G | 211M > V | #1010 #1286 #1570 #1591 |
| | | #1602 #163 #1704 #2107 |
| | | #2112 #217 #2208 #367 |
| | | #714 #767 #790 #976 |
| | | #M258 |
| 2837C > A | 212A > E | #667 |
| 2853C > T | | #1010 #1020 #1024 #1286 |
| | | #129 #1350 #1570 #1591 |
| | | #1602 #163 #1704 #2107 |
| | | #2112 #217 #2208 #2320 |
| | | #2731 #2806 #3086 #3142 |
| | | #367 #442 #508 #667 #668 |
| | | #685 #704 #714 #767 #790 |
| | | #976 #M258 |
| 2859C > T | | #1010 #1020 #1024 #1158 |
| | | #1286 #129 #1350 #1570 |
| | | #1591 #1602 #163 #1704 |
| | | #2107 #2112 #217 #2208 |
| | | #2320 #2731 #2806 #3086 |
| | | #3142 #367 #442 #508 #667 |
| | | #668 #685 #704 #714 #767 |
| | | #790 #976 #M258 |
| 2877G > A | | #1010 #1020 #1024 #1158 |
| | | #1286 #129 #1350 #1570 |
| | | #1591 #1602 #163 #2107 |
| | | #2112 #217 #2208 #2320 |
| | | #2731 #3086 #3142 #367 |
| | | #442 #667 #668 #685 #704 |
| | | #714 #767 #790 #976 |
| 2879_2880insG | N227Kfs277 | #M258 |
| 2899_2900delinsCA | 233T > Q | #1010 #1020 #1024 #1158 |
| | | #1286 #129 #1350 #1570 |
| | | #1591 #1602 #163 #1704 |
| | | #2107 #2112 #217 #2208 |
| | | #2320 #2731 #3086 #3142 |
| | | #367 #442 #667 #668 #685 |
| | | #704 #714 #767 #790 #976 |
| | | #985 |
| 2905A > C | 235M > L | #1010 #1020 #1024 #1158 |
| | | #1286 #129 #1350 #1570 |
| | | #1602 #163 #1704 #2102 |
| | | #2107 #2112 #217 #2208 |
| | | #2320 #2731 #3086 #3142 |
| | | #367 #442 #667 #668 #685 |
| | | #704 #714 #767 #790 #976 |
| | | #985 |
| 2905A > T | 235M > L | #1591 |
| 2911G > A | 237D > N | #685 |
| 2922C > T | | #3142 |
| 2937A > C | | #1055 #1260 #1273 #1343 |
| | | #1534 #1919 #2102 #3511 |
| | | #371 #473 #777 #877 |
| 2946C > T | | #1055 #1260 #1273 #1286 |
| | | #1343 #1449 #1534 #1919 |
| | | #2040 #2087 #2102 #2128 |
| | | #3013 #3511 #371 #3765 |
| | | #387 #471 #473 #508 #777 |
| | | #790 #877 #976 |
| 2964C > T | | #1024 |
| 2967C > T | | #1017 #1055 #1260 #1273 |
| | | #1343 #1449 #1534 #1919 |
| | | #2040 #2087 #2128 #2206 |
| | | #3013 #3511 #371 #3765 |
| | | #387 #471 #473 #777 #877 |
| 2970C > G | | #1055 #1260 #1273 #1343 |
| | | #1449 #1534 #1919 #2040 |
| | | #3013 #3511 #371 #3765 |
| | | #471 #473 #777 #877 |
| 2970C > T | | #129 |
| 2976_2997delins_22nt | 258_265delins_NFFQIKIN | #1010 |
| 2976A > G | | #1017 #1020 #1024 #1055 |
| | | #1158 #1260 #1273 #1286 |
| | | #129 #1343 #1350 #1449 |
| | | #1534 #1570 #1591 #1602 |
| | | #163 #1704 #1919 #2040 |
| | | #2087 #2102 #2107 #2112 |
| | | #2128 #217 #2206 #2208 |
| | | #2320 #2497 #2557 #2731 |
| | | #3013 #3086 #3142 #3511 |
| | | #367 #3765 #387 #442 #471 |
| | | #473 #508 #667 #668 #685 |
| | | #704 #714 #767 #777 #790 |
| | | #877 #976 #985 |
| 2979A > G | | #1017 #1055 #1260 #1273 |
| | | #1343 #1449 #1534 #1919 |
| | | #2040 #2087 #2128 #2206 |
| | | #2557 #3013 #3142 #3511 |
| | | #371 #3765 #387 #471 #473 |
| | | #508 #777 #877 |
| 2982T > C | | #1020 #1024 #1158 #1286 |
| | | #129 #1350 #1570 #1591 |
| | | #1602 #163 #1704 #2102 |
| | | #2107 #2112 #217 #2208 |
| | | #2320 #2731 #3086 #3142 |
| | | #367 #442 #508 #668 #704 |
| | | #714 #790 #976 #985 |
| 2982T > A | | #1273 #1534 |
| 2984C > G | 261S > C | #667 |
| 2991A > G | | #1055 #1260 #1273 #1534 |
| | | #1919 #2128 #2557 #3511 |
| | | #387 |
| 2994A > T | | #1055 #1260 #1273 #1534 |
| | | #1919 #2128 #2557 #3511 |
| | | #387 |
| 3000C > T | | #1017 #1024 #163 #2206 |
| | | #2320 #3142 #508 #790 |
| | | #985 |
| 3000C > A | | #1343 #3013 #777 |
| 3001TCG > AGC | | #1055 #1260 #1273 #1286 |
| | | #1534 #1602 #1919 #2208 |
| | | #2557 #3511 #371 #473 |
| | | #976 |
| 3003G > A | | #1017 #1020 #1158 #129 |
| | | #1350 #1449 #1570 #1591 |
| | | #163 #2040 #2087 #2107 |
| | | #2112 #2128 #217 #2206 |
| | | #2320 #2497 #2731 #3086 |
| | | #3142 #367 #3765 #387 |
| | | #442 #471 #667 #668 #685 |
| | | #704 #714 #790 #877 #985 |
| 3006C > T | | #985 |
| 3007_3012delGACAAT | 269_270delDN | #767 |
| 3012T > C | | #1017 #1020 #1024 #1055 |
| | | #1158 #1260 #1273 #1286 |
| | | #129 #1343 #1350 #1449 |
| | | #1534 #1570 #1591 #1602 |
| | | #163 #1704 #1919 #2040 |
| | | #2087 #2107 #2112 #2128 |
| | | #2141 #217 #2206 #2208 |
| | | #2320 #2497 #2557 #2731 |
| | | #3013 #3086 #3142 #3511 |
| | | #367 #371 #3765 #387 #442 |
| | | #471 #473 #508 #667 #668 |
| | | #685 #704 #714 #777 #790 |
| | | #877 #976 #985 |
| 3018C > T | | #790 |
| 3019_3020insC | N273Kfs277 | #M258 #M258 |
| 3025_3026insC | N255Kfs277 | #M258 #M258 |
| 3028A > T | | #M258 |
| 3032C > A | | #M258 |
| 3035_3536delinsGG | 278P > R | #667 |
| 3036T > C | | #1017 #1020 #1055 #1158 |
| | | #1260 #1273 #1286 #129 |
| | | #1343 #1350 #1534 #163 |
| | | #1919 #2102 #2112 #2141 |
| | | #217 #2206 #2320 #2557 |
| | | #2806 #3142 #3511 #367 |
| | | #371 #387 #442 #473 #508 |
| | | #668 #704 #714 #767 #790 |
| | | #877 #976 #985 #M258 |
| 3043T > G | 281Y > D | #667 |
| 3072C > T | | #1343 |
| 3075T > C | | #1017 #1343 #1704 #2206 |
| | | #3013 #3511 #777 |
| 3078A > G | | #1017 #1055 #1343 #1704 |
| | | #2206 |
| 3078A > C | | #3013 #3511 #777 |
| 3084T > C | | #1055 #1704 |
| 3087C > T | | #1020 #1704 #367 #767 |
| 3093A > G | | #1017 #1055 #2206 |
| 3111C > T | | #1017 |
| 3123A > T | | #1343 #1449 #1602 #2040 |
| | | #2102 #2208 #3142 #3765 |
| | | #387 #777 |
| 3123A > G | | #1055 #1273 #1286 #1602 |
| | | #1704 #2087 #2102 #2141 |
| | | #2208 #3142 #3511 #387 |
| | | #442 #473 #667 #685 #777 |
| | | #877 #976 #M258 |
| 3126C > A | | #1273 #1534 #2557 #471 |
| | | #473 |
| 3129G > A | | #1055 #1273 #1534 #2087 |
| | | #2102 #2208 #2320 #2497 |
| | | #2557 #2806 #3086 #3142 |
| | | #387 #473 #667 #685 #714 |
| | | #777 #M258 |
| 3131G > A | 310R > K | #163 #985 |
| 3137A > G | 312N > S | #3511 |
| 3147C > G | | #1017 #1055 #2102 #2208 |
| | | #387 |
| 3147C > T | | #2087 |
| 3147C > A | | #371 |
| 3151_3152delinsCT | 317N > L | #1010 |
| 3165A > G | | #1055 #1273 #1343 #1350 |
| | | #1449 #1534 #2320 #2557 |
| | | #3013 #3142 #3511 #387 |
| | | #471 #473 #777 #877 |
| 3191C > A | 330T > K | #217 |
| 3192G > A | | #1273 #1286 #1534 |
| 3195_3198delinsCCCC | 332I > P | #217 |
| 3198T > C | | #3765 |
| 3236A > G | 345D > G | #985 |
| 3242A > G | 347E > G | #985 |
| 3247_3248delinsGG | 349Q > G | #985 |
| 3250C > A | 350L > I | #M258 |
| 3254C > G | 351P > R | #985 |
| 3261C > T | | #3765 |
| 3264C > T | | #2087 |
| 3264C > G | | #473 |
| 3267C > T | | #1017 |
| 3276T > C | | #1020 #217 #367 #668 |
| 3287T > C | 362L > P | #2208 |
| 3297C > T | | #1024 #1158 #1343 #1570 |
| | | #1591 #163 #1919 #2107 |
| | | #2320 #2731 #3142 #508 |
| | | #714 #777 #790 #985 |
| 3303A > G | | #1010 #1020 #1024 #1158 |
| | | #1260 #1343 #1570 #1591 |
| | | #1602 #163 #1919 #2107 |
| | | #2112 #217 #2320 #2731 |
| | | #2806 #3086 #3142 #367 |
| | | #508 #667 #668 #704 #714 |
| | | #767 #777 #790 #985 |
| 3309C > T | | #2497 #442 |
| 3312C > T | | #685 |
| 3318G > C | | #1010 #1020 #1158 #1260 |
| | | #1286 #1343 #1350 #1570 |
| | | #1602 #163 #1704 #2107 |
| | | #2141 #217 #2320 #2497 |
| | | #2806 #3086 #3142 #367 |
| | | #442 #508 #668 #685 #777 |
| | | #790 #976 #985 |
| 3318G > T | | #667 #767 |
| 3333C > T | | #976 |
| 3342G > A | | #1024 #1449 #3142 #471 |
| 3351G > A | | #2128 |
| 3357G > A | | #1449 #2040 #3765 #471 |
| | | #714 |
| 3360A > G | | #1010 #1017 #1020 #1024 |
| | | #1055 #1158 #1260 #129 |
| | | #1343 #1350 #1570 #1591 |
| | | #1602 #163 #1704 #1919 |
| | | #2102 #2107 #2112 #217 |
| | | #2208 #2320 #2497 #2806 |
| | | #3013 #3086 #3142 #3511 |
| | | #367 #442 #473 #508 #667 |
| | | #668 #685 #714 #767 #777 |
| | | #790 #976 #M258 |
| 3363A > C | | #1055 #2102 |
| 3375A > C | | #1020 #1024 #1055 #1158 |
| | | #129 #1343 #1350 #1449 |
| | | #1570 #1591 #1602 #163 |
| | | #1704 #1919 #2102 #2107 |
| | | #217 #2208 #2320 #3086 |
| | | #3142 #367 #371 #442 #473 |
| | | #508 #667 #668 #685 #767 |
| | | #777 #790 #976 #985 |
| | | #M258 |
| 3385C > T | | #442 #M258 |
| 3387G > A | | #387 |
| 3390G > A | | #3765 |
| 3405G > A | | #2087 #2102 #2128 #2320 |
| | | #508 |
| 3411G > T | | #1020 #1055 #1260 #1273 |
| | | #1449 #1534 #1919 #2040 |
| | | #2102 #2112 #2208 #2557 |
| | | #3765 #471 #473 #704 #877 |
| 3411G > A | | #1704 |
| 3417C > T | | #1010 #1020 #1158 #1286 |
| | | #1350 #1570 #1591 #1602 |
| | | #163 #2107 #2112 #2320 |
| | | #2497 #2731 #3086 #3142 |
| | | #3511 #367 #442 #508 #668 |
| | | #685 #704 #790 #976 #985 |
| | | #M258 |
| 3417C > A | | #1024 #1055 |
| 3426C > T | | #1704 |
| 3429T > C | | #1704 #M258 |
| 3430ACC > CAG | 410T > Q | #2497 #2731 #1602 #1010 |
| | | #985 #1591 #508 #2112 |
| | | #1350 #668 #367 #1020 |
| | | #1158 #2107 #1570 #704 |
| | | #714 #3086 #3142 #1024 |
| | | #790 #976 #1286 #163 #685 |
| | | #442 #2320 |
| 3430ACC > CAA | 410T > Q | #M258 |
| 3435C > T | | #1602 #790 #976 |
| 3441C > T | | #1449 #2040 #2141 #3765 |
| | | #471 |
| 3444T > C | | #1010 #1017 #1020 #1055 |
| | | #1158 #1260 #1286 #1343 |
| | | #1350 #1449 #1534 #1570 |
| | | #1591 #163 #1919 #2040 |
| | | #2087 #2102 #2107 #2112 |
| | | #2141 #2206 #2208 #2320 |
| | | #2497 #2557 #2731 #3013 |
| | | #3086 #3142 #3511 #367 |
| | | #371 #3765 #387 #442 #471 |
| | | #473 #667 #668 #685 #704 |
| | | #714 #777 #790 #877 #976 |
| | | #985 #M258 |
| 3444T > A | | #2128 |
| 3450G > A | | #1010 #1020 #1024 #1055 |
| | | #1158 #1260 #1286 #1350 |
| | | #1534 #1570 #1602 #163 |
| | | #1704 #1919 #2102 #2107 |
| | | #2112 #2128 #217 #2208 |
| | | #2320 #2497 #2557 #2731 |
| | | #3086 #3142 #367 #442 |
| | | #473 #508 #667 #668 #685 |
| | | #704 #714 #790 #877 #976 |
| | | #985 #M258 |
| 3453C > T | | #2141 |
| 3456T > G | | #1024 #1534 #2128 #2557 |
| | | #508 #877 |
| 3459T > G | | #2141 |
| 3463_3465delinsACA | 421H > T | #2141 |
| 3471C > A | 423S > R | #2141 |
| 3474C > T | | #3142 |
| 3483C > T | | #1260 #1534 #1919 #2102 |
| | | #2208 #2557 #473 #877 |
| 3486G > A | | #1260 #1534 #1919 #2102 |
| | | #2208 #2557 #473 #877 |
| 3490C > T | | #1017 #1273 #1343 #1449 |
| | | #1919 #2040 #2087 #2128 |
| | | #2141 #2206 #2497 #3013 |
| | | #3511 #371 #3765 #387 |
| | | #471 #508 #777 |
| 3495C > T | | #1010 #1020 #1024 #1158 |
| | | #1286 #129 #1350 #1570 |
| | | #1591 #1602 #163 #1704 |
| | | #2107 #2112 #217 #2320 |
| | | #2731 #3086 #3142 #367 |
| | | #442 #508 #667 #668 #685 |
| | | #704 #714 #767 #790 #976 |
| | | #985 #M258 |
| 3496C > A | | #2112 |
| 3498T > G | | #1010 #1020 #1024 #1158 |
| | | #1286 #1343 #1350 #1570 |
| | | #1591 #1602 #163 #1704 |
| | | #2107 #2112 #217 #2320 |
| | | #2731 #3086 #3142 #367 |
| | | #442 #471 #508 #667 #668 |
| | | #685 #704 #714 #767 #777 |
| | | #790 #976 #985 #M258 |
| 3498T > C | | #3765 |
| 3501C > G | | #1158 #1286 #129 #1343 |
| | | #1350 #1570 #1591 #1602 |
| | | #163 #1704 #2107 #2112 |
| | | #217 #2320 #2731 #3086 |
| | | #3142 #367 #442 #508 #667 |
| | | #668 #685 #704 #714 #767 |
| | | #777 #790 #976 #985 |
| | | #M258 |
| 3501C > A | | #1024 |
| 3513C > G | | #1010 #1020 #1024 #1158 |
| | | #1286 #1343 #1350 #1570 |
| | | #1591 #1602 #163 #2107 |
| | | #2112 #217 #2320 #2731 |
| | | #3086 #3142 #367 #442 |
| | | #508 #667 #668 #685 #704 |
| | | #714 #767 #777 #790 #976 |
| | | #985 #M258 |
| 3516C > T | | #1591 #163 #2112 #2320 |
| | | #2806 #3086 #704 #790 |
| | | #985 |
| 3525C > T | | #1343 #777 |
| 3528G > A | | #1260 |
| 3534C > T | | #1010 #1020 #1024 #1158 |
| | | #1286 #129 #1350 #1570 |
| | | #1591 #1602 #163 #1704 |
| | | #2107 #2112 #217 #2320 |
| | | #2731 #2806 #3086 #3142 |
| | | #367 #442 #508 #668 #685 |
| | | #704 #714 #767 #790 #976 |
| | | #985 #M258 |
| 3535T > C | | #1010 #1020 #1024 #1158 |
| | | #1286 #129 #1350 #1570 |
| | | #1591 #1602 #163 #1704 |
| | | #2107 #2112 #217 #2320 |
| | | #2731 #2806 #3086 #3142 |
| | | #367 #442 #508 #668 #685 |
| | | #704 #714 #767 #790 #976 |
| | | #985 #M258 |
| 3537G > A | | #1024 #1260 #129 #508 |
| | | #685 #790 |
| 3539G > A | 446S > N | #1010 #1020 #1024 #1158 |
| | | #1286 #129 #1350 #1570 |
| | | #1591 #1602 #163 #1704 |
| | | #2107 #2112 #217 #2320 |
| | | #2731 #2806 #3086 #3142 |
| | | #367 #442 #508 #668 #685 |
| | | #704 #714 #767 #790 #976 |
| | | #985 #M258 |
| 3541A > G | 447R > G | #2102 |
| 3542G > A | 447R > K | #1010 #1020 #1158 #1286 |
| | | #1343 #1350 #1570 #1591 |
| | | #1602 #163 #1704 #2107 |
| | | #2112 #217 #2320 #2731 |
| | | #2806 #3086 #3142 #367 |
| | | #442 #668 #685 #704 #714 |
| | | #767 #777 #790 #976 #985 |
| | | #M258 |
| 3543A > G | | #1024 #1158 #1350 #1591 |
| | | #1602 #163 #1704 #2320 |
| | | #2806 #3086 #3142 #442 |
| | | #508 #668 #685 #714 #790 |
| | | #976 #985 #M258 |
| 3547_3549delinsCAA | 449N > Q | #129 #2731 #1602 #1010 |
| | | #985 #667 #2806 #M258 |
| | | #217 #767 #1591 #508 |
| | | #1704 #2112 #668 #367 |
| | | #1020 #1158 #2107 #1570 |
| | | #704 #714 #3086 #3142 |
| | | #1024 #790 #976 #1286 |
| | | #163 #685 #442 #2320 |
| 3547_3549delinsCAG | 449N > Q | #1350 |
| 3550A > T | 450T > S | #1010 #1020 #1158 #1286 |
| | | #1350 #1591 #163 #2107 |
| | | #2112 #217 #2320 #2731 |
| | | #2806 #3086 #3142 #367 |
| | | #442 #668 #685 #704 #714 |
| | | #767 #790 #976 #985 |
| | | #M258 |
| 3550A > G | 450T > A | #1343 #1570 #777 |
| 3552T > A | | #1010 #1020 #1024 #1158 |
| | | #1286 #129 #1350 #1570 |
| | | #1591 #1602 #163 #1704 |
| | | #2107 #2112 #217 #2320 |
| | | #2731 #2806 #3086 #3142 |
| | | #367 #442 #508 #668 #685 |
| | | #704 #714 #790 #976 #985 |
| | | #M258 |
| 3552T > G | | #1055 #1260 #2206 |
| 3552_3554delTCC | 451delP | #767 |
| 3553_3555delinsAAT | 451P > N | #2731 #1602 #1010 #985 |
| | | #2806 #217 #1591 #1704 |
| | | #2112 #1350 #668 #367 |
| | | #1020 #1158 #2107 #1570 |
| | | #704 #714 #3086 #3142 |
| | | #790 #976 #1286 #163 #685 |
| | | #442 #2320 |
| 3553_3555delinsGCC | 451P > A | #M258 #508 #1024 |
| 3555A > G | | #1260 #1055 |
| 3555A > C | | #129 |
| 3558T > C | | #1017 #1055 #1260 #1273 |
| | | #1343 #1449 #1534 #1919 |
| | | #2040 #2087 #2102 #2128 |
| | | #2206 #2208 #2497 #2557 |
| | | #3013 #3511 #371 #3765 |
| | | #387 #471 #473 #777 #877 |
| 3560G > A | 453G > E | #2806 |
| 3564C > T | | #1010 #1020 #1158 #1286 |
| | | #129 #1350 #1570 #1591 |
| | | #1602 #163 #1704 #2107 |
| | | #2112 #217 #2320 #3086 |
| | | #3142 #367 #668 #685 #704 |
| | | #714 #767 #790 #976 #985 |
| | | #M258 |
| 3564_3566delCAC | 454T_del | #1024 #508 |
| 3564C > A | | #2806 #442 |
| 3565_3567delinsCTT | 455T > L | #1602 #1010 #985 #767 |
| | | #1591 #1704 #2112 #1350 |
| | | #668 #367 #1020 #1158 |
| | | #2107 #1570 #704 #714 |
| | | #3086 #790 #976 #1286 |
| | | #163 #685 #442 #2320 |
| 3565_3567delinsGTT | 455T > V | #M258 #3142 |
| 3565A > C | 455T > P | #129 |
| 3566C > A | 455T > N | #217 |
| 3567C > T | | #1017 #1273 #877 #2557 |
| | | #1919 #1260 #2208 #2806 |
| 3567_3620delins_54nt | 456_473delins_18aa | #2806 |
| 3568_3569delinsCA | 456T > Q | #1602 #1010 #985 #M258 |
| | | #767 #1591 #508 #1704 |
| | | #2112 #1350 #668 #367 |
| | | #1020 #1158 #2107 #1570 |
| | | #704 #714 #3086 #3142 |
| | | #1024 #976 #1286 #163 |
| | | #685 #442 #2320 |
| 3568_3570delinsCAA | 456T > Q | #790 |
| 3571_3572delinsAT | 457Q > M | #1343 #777 #3511 #3013 |
| | | #2731 |
| 3576A > T | | #777 #129 #1602 #1010 |
| | | #667 #M258 #767 #1591 |
| | | #508 #1704 #2112 #1350 |
| | | #668 #367 #1020 #1158 |
| | | #2107 #1570 #704 #714 |
| | | #3086 #3142 #1024 #790 |
| | | #976 #1286 #163 #685 #442 |
| | | #2320 |
| 3576A > C | | #2102 #371 #1017 #473 |
| | | #1273 #2087 #2206 #877 |
| | | #1534 #2557 #1919 #1260 |
| | | #3765 #471 #387 #2040 |
| | | #1449 #2128 #1343 #3511 |
| | | #3013 #2208 #1055 #2806 |
| 3577A > C | | #129 #1602 #1010 #667 |
| | | #M258 #767 #1591 #508 |
| | | #1704 #2112 #1350 #668 |
| | | #367 #1020 #1158 #2107 |
| | | #1570 #704 #714 #3086 |
| | | #3142 #1024 #790 #976 |
| | | #1286 #163 #685 #442 |
| | | #2320 |
| 3579G > A | | #2206 |
| 3580C > T | | #767 |
| 3582T > A | | #1010 #1020 #1024 #1158 |
| | | #1286 #129 #1350 #1570 |
| | | #1591 #1602 #163 #1704 |
| | | #2107 #2112 #2320 #2731 |
| | | #3086 #3142 #367 #442 |
| | | #508 #667 #668 #685 #704 |
| | | #714 #767 #790 #976 |
| | | #M258 |
| 3584A > T | 461Q > L | #1010 #1020 #1024 #1158 |
| | | #1286 #129 #1350 #1570 |
| | | #1591 #1602 #163 #1704 |
| | | #2107 #2112 #2320 #2731 |
| | | #3086 #3142 #367 #442 |
| | | #508 #667 #668 #685 #704 |
| | | #714 #767 #790 #976 |
| | | #M258 |
| 3589_3590delinsAG | | #2731 #1010 #667 #767 |
| | | #1591 #1704 #2112 #1350 |
| | | #668 #367 #1020 #1158 |
| | | #2107 #1570 #704 #714 |
| | | #790 #1286 #163 #2320 |
| 3589_3591delinsAGC | | #129 #1602 #M258 #508 |
| | | #3086 #3142 #1024 #976 |
| | | #685 #442 |
| 3594G > A | | #1010 #1020 #1024 #1158 |
| | | #1286 #129 #1350 #1449 |
| | | #1570 #1591 #1602 #163 |
| | | #1704 #2107 #2112 #2320 |
| | | #2731 #3086 #3142 #367 |
| | | #442 #508 #667 #668 #685 |
| | | #704 #714 #767 #790 #976 |
| | | #M258 |
| 3597C > T | | #1010 #1020 #1024 #1158 |
| | | #1286 #129 #1350 #1570 |
| | | #1591 #1602 #163 #1704 |
| | | #2107 #2112 #2320 #2731 |
| | | #2806 #3086 #3142 #367 |
| | | #442 #508 #667 #668 #685 |
| | | #704 #714 #767 #790 #976 |
| | | #M258 |
| 3597C > A | | #1343 |
| 3601_3603delinsCCC | 467A > P | #129 #2731 #1010 #667 |
| | | #M258 #767 #508 #1704 |
| | | #2112 #1350 #668 #367 |
| | | #1020 #1158 #2107 #1570 |
| | | #704 #714 #3086 #3142 |
| | | #1024 #790 #976 #1286 |
| | | #163 #685 #442 #2320 |
| 3601_3603delinsCCA | 467A > P | #1602 |
| 3603G > A | | #2102 #2206 #1919 #1260 |
| | | #3765 #471 #387 #1449 |
| | | #2128 #1343 #777 |
| 3605_3606delinsCC | 468S > T | #2731 #1602 #1010 #667 |
| | | #M258 #767 #508 #1704 |
| | | #2112 #1350 #668 #367 |
| | | #1020 #1158 #2107 #1570 |
| | | #704 #714 #3086 #3142 |
| | | #1024 #790 #976 #1286 |
| | | #163 #685 #442 #2320 |
| 3605_3606delinsCA | 468S > T | #129 #2806 |
| 3607_3608delinsAG | 469D > S | #129 #2731 #1010 #667 |
| | | #2806 #M258 #767 #508 |
| | | #1704 #2112 #1350 #668 |
| | | #367 #1020 #1158 #2107 |
| | | #1570 #704 #714 #3142 |
| | | #1024 #790 #976 #1286 |
| | | #163 #685 #442 #2320 |
| 3607G > A | 469D > N | #1602 #3086 |
| 3612T > G | 470I > M | #1010 #1020 #1024 #1158 |
| | | #1286 #129 #1350 #1570 |
| | | #1602 #163 #1704 #2107 |
| | | #2112 #2320 #2731 #3086 |
| | | #3142 #367 #442 #508 #667 |
| | | #668 #685 #704 #714 #767 |
| | | #790 #976 #M258 |
| 3613_3615delinsTCT | 471R > S | #129 #2731 #1602 #1010 |
| | | #667 #M258 #767 #508 |
| | | #1704 #2112 #1350 #668 |
| | | #367 #1020 #1158 #2107 |
| | | #1570 #704 #714 #3086 |
| | | #3142 #1024 #790 #976 |
| | | #1286 #163 #685 #442 |
| | | #2320 |
| 3616_3618delinsCTT | 472D > L | #129 #2731 #1602 #1010 |
| | | #667 #M258 #767 #508 |
| | | #1704 #2112 #1350 #668 |
| | | #367 #1020 #1158 #2107 |
| | | #1570 #704 #714 #3086 |
| | | #3142 #1024 #790 #976 |
| | | #1286 #163 #685 #442 |
| | | #2320 |
| 3618C > T | | #2141 |
| 3621G > A | 473Q > H | #1010 #1020 #1024 #1158 |
| | | #1286 #129 #1350 #1570 |
| | | #1602 #163 #1704 #2107 |
| | | #2112 #2320 #2806 #3086 |
| | | #3142 #367 #442 #508 #667 |
| | | #668 #685 #704 #714 #767 |
| | | #790 #976 #M258 |
| 3621G > C | | #2731 |
| 3622T > G | 474S > A | #1010 #1020 #1024 #1158 |
| | | #1286 #129 #1350 #1570 |
| | | #1602 #163 #1704 #2107 |
| | | #2112 #2320 #2731 #2806 |
| | | #3086 #3142 #367 #442 |
| | | #508 #667 #668 #685 #704 |
| | | #714 #767 #790 #976 |
| | | #M258 |
| 3624T > C | | #2806 |
| 3626_3627delins > AA | 475R > K | #129 #2731 #1602 #1010 |
| | | #667 #M258 #508 #1704 |
| | | #2112 #1350 #668 #367 |
| | | #1020 #1158 #2107 #1570 |
| | | #704 #714 #3086 #3142 |
| | | #1024 #790 #976 #1286 |
| | | #163 #685 #442 #2320 |
| 3627G > A | | #1343 #777 #2806 |
| 3628_3629delinsGT | 476N > V | #2806 |
| 3630C > T | | #442 |
| 3636T > G | | #1010 #1024 #1158 #1286 |
| | | #129 #1350 #1570 #1602 |
| | | #163 #1704 #2107 #2112 |
| | | #217 #2320 #2731 #2806 |
| | | #3086 #3142 #367 #442 |
| | | #508 #667 #668 #685 #704 |
| | | #714 #790 #976 #M258 |
| 3636T > A | | #1020 |
| 3645C > T | | #1020 #1024 #1158 #1286 |
| | | #1350 #1570 #1602 #163 |
| | | #1704 #2107 #2112 #217 |
| | | #2320 #2731 #2806 #3086 |
| | | #3142 #367 #442 #508 #667 |
| | | #668 #685 #704 #714 #790 |
| | | #976 #M258 |
| 3645C > G | | #129 |
| 3648T > C | | #1020 #1024 #1158 #1286 |
| | | #1350 #1570 #1602 #163 |
| | | #1704 #2107 #2112 #217 |
| | | #2320 #2731 #2806 #3086 |
| | | #3142 #367 #442 #508 #667 |
| | | #668 #685 #704 #714 #790 |
| | | #976 #M258 |
| 3651C > T | | #129 #2141 |
| 3652C > A | | #1020 #1024 #1158 #1286 |
| | | #1350 #1570 #1602 #163 |
| | | #1704 #2107 #2112 #217 |
| | | #2320 #2731 #2806 #3086 |
| | | #3142 #367 #442 #508 #667 |
| | | #668 #685 #704 #714 #790 |
| | | #976 #M258 |
| 3654C > A | | #1020 #1024 #1158 #1286 |
| | | #1350 #1570 #1602 #163 |
| | | #1704 #2107 #2112 #217 |
| | | #2320 #2731 #2806 #3086 |
| | | #3142 #367 #442 #508 #667 |
| | | #668 #685 #704 #714 #790 |
| | | #976 #M258 |
| 3657G > A | | #1024 |
| 3660G > A | | #371 |
| 3663A > T | | #1020 #1024 #1158 #1286 |
| | | #129 #1350 #1570 #1602 |
| | | #163 #1704 #2107 #2112 |
| | | #217 #2320 #2731 #2806 |
| | | #3086 #3142 #367 #442 |
| | | #508 #667 #668 #685 #704 |
| | | #714 #790 #976 #M258 |
| 3664_3666delinsTTG | 488V > L | #129 #3086 #1024 |
| 3664_3666delinsCTG | 488V > L | #2731 #1602 #667 #M258 |
| | | #217 #508 #1704 #2112 |
| | | #1350 #668 #367 #1020 |
| | | #1158 #2107 #1570 #704 |
| | | #714 #3142 #790 #976 |
| | | #1286 #163 #685 #442 |
| | | #2320 |
| 3664G > T | 488V > L | #2806 |
| 3672G > A | | #1017 #1350 |
| 3673_3675delinsCAG | 491T > Q | #129 #2731 #1602 #667 |
| | | #2806 #M258 #217 #508 |
| | | #1704 #2112 #1350 #668 |
| | | #367 #1020 #1158 #2107 |
| | | #1570 #704 #714 #3086 |
| | | #3142 #1024 #790 #976 |
| | | #1286 #163 #685 #442 |
| | | #2320 |
| 3676T > G | 492S > A | #1020 #1024 #1055 #1158 |
| | | #1273 #1286 #129 #1343 |
| | | #1350 #1449 #1534 #1570 |
| | | #1602 #163 #1704 #1919 |
| | | #2040 #2087 #2102 #2107 |
| | | #2112 #217 #2208 #2320 |
| | | #2497 #2557 #2731 #2806 |
| | | #3013 #3086 #3142 #3511 |
| | | #367 #442 #473 #508 #667 |
| | | #668 #685 #704 #714 #777 |
| | | #790 #877 #976 #M258 |
| 3676T > C | 492S > P | #3765 #387 #471 |
| 3678T > A | | #1020 #1024 #1158 #1286 |
| | | #129 #1350 #1570 #1602 |
| | | #163 #1704 #2107 #2112 |
| | | #217 #2320 #2731 #2806 |
| | | #3086 #3142 #367 #442 |
| | | #508 #667 #668 #685 #704 |
| | | #714 #790 #976 #M258 |
| 3679_3681delinsAAC | 493A > N | #129 #2731 #1602 #667 |
| | | #2806 #M258 #217 #508 |
| | | #1704 #2112 #1350 #668 |
| | | #367 #1020 #1158 #2107 |
| | | #1570 #704 #714 #3086 |
| | | #1024 #790 #976 #1286 |
| | | #163 #685 #442 |
| 3679_3681delinsAGC | 493A > S | #3142 |
| 3679G > A | 493A > T | #2320 |
| 3681G > A | | #2128 |
| 3684T > C | | #1020 #1024 #1158 #1273 |
| | | #1286 #129 #1343 #1350 |
| | | #1570 #1602 #163 #1704 |
| | | #2087 #2102 #2107 #2112 |
| | | #217 #2497 #2557 #2731 |
| | | #2806 #3013 #3086 #3142 |
| | | #3511 #367 #442 #508 #667 |
| | | #668 #685 #704 #714 #777 |
| | | #790 #877 #976 |
| 3684T > A | 494D > E | #M258 |
| 3687C > A | 495N > K | #767 |
| 3689A > G | 496N > S | #2102 #2497 |
| 3690C > T | | #2806 #3086 |
| 3693C > T | | #1024 #2128 |
| 3693delC | N497Kfs511 | #2112 |
| 3696T > C | | #1020 #1024 #1158 #1286 |
| | | #129 #1350 #1570 #1591 |
| | | #1602 #163 #1704 #2107 |
| | | #2112 #217 #2731 #2806 |
| | | #3086 #3142 #367 #442 |
| | | #508 #667 #668 #685 #704 |
| | | #714 #790 #976 #M258 |
| 3697_3699delinsAAC | 499E > N | #129 #2731 #1602 #667 |
| | | #2806 #M258 #217 #1591 |
| | | #508 #1350 #668 #367 |
| | | #1020 #1158 #2107 #1570 |
| | | #704 #714 #3086 #3142 |
| | | #1024 #790 #976 #1286 |
| | | #163 #685 #442 |
| 3697_3699delinsAAT | 499E > N | #1704 |
| 3699A > T | 499E > D | #1343 #777 #3511 #3013 |
| 3699A > G | | #2497 #2102 #2087 |
| 3701_3702delinsTT | 500Y > F | #129 #2731 #1602 #667 |
| | | #2806 #M258 #217 #1591 |
| | | #1704 #1350 #668 #367 |
| | | #1020 #1158 #2107 #1570 |
| | | #704 #714 #3086 #3142 |
| | | #1024 #790 #976 #1286 |
| | | #163 #685 #442 #2320 |
| 3702C > T | | #371 #1534 |
| 3703_3705delinsCCC | 501S > P | #129 #2731 #1602 #667 |
| | | #2806 #M258 #217 #1591 |
| | | #1704 #1350 #668 #367 |
| | | #1020 #1158 #2107 #1570 |
| | | #704 #714 #3086 #3142 |
| | | #1024 #790 #976 #1286 |
| | | #163 #685 #442 #2320 |
| 3711T > A | | #217 |
| 3713_3714delinsCG | 504G > A | #667 #M258 #767 #1591 |
| | | #1704 #1350 #668 #367 |
| | | #1158 #704 #714 #3142 |
| | | #1024 #790 #976 #1286 |
| | | #163 #685 #442 #2320 |
| 3713G > C | 504G > A | #2731 #1602 #2806 #1020 |
| | | #2107 #1570 #3086 |
| 3714A > T | | #2497 |
| 3720C > A | | #1020 #1024 #1158 #1286 |
| | | #1350 #1570 #1591 #1602 |
| | | #163 #1704 #2107 #2112 |
| | | #2320 #2731 #2806 #3086 |
| | | #3142 #367 #442 #667 #668 |
| | | #685 #704 #714 #767 #790 |
| | | #976 #M258 |
| 3723G > A | | #371 |
| 3726C > T | | #1020 #1158 #1286 #1350 |
| | | #1570 #1591 #1602 #163 |
| | | #1704 #2107 #2320 #2731 |
| | | #2806 #3086 #3142 #367 |
| | | #442 #667 #668 #685 #704 |
| | | #714 #767 #790 #976 |
| | | #M258 |
| 3728A > C | | |
| 3729C > T | | #1020 #1158 #1286 #1350 |
| | | #1570 #1591 #163 #1704 |
| | | #2107 #2112 #2320 #2731 |
| | | #2806 #3086 #3142 #367 |
| | | #442 #667 #668 #685 #704 |
| | | #714 #767 #790 #976 |
| | | #M258 |
| 3732C > A | | #1020 #1158 #1286 #1350 |
| | | #1570 #1591 #163 #1704 |
| | | #2107 #2112 #2731 #2806 |
| | | #3086 #3142 #367 #667 |
| | | #668 #685 #704 #714 #767 |
| | | #790 #976 |
| 3733_3734insT | | #2112 |
| 3734_3756delins_26nt | 511_518delinsGYKVSTKW | #2112 |
| 3738C > A | | #1343 #2087 #2102 #2497 |
| | | #777 |
| 3738C > T | | #387 #508 |
| 3739_3741delinsCGG | | #3511 #3013 #667 #2806 |
| | | #M258 #767 #1591 #1704 |
| | | #1350 #668 #367 #1020 |
| | | #1158 #2107 #1570 #704 |
| | | #714 #3086 #3142 #790 |
| | | #976 #1286 #163 #685 #442 |
| | | #2320 |
| 3741A > T | 513R > S | #2731 |
| 3747T > G | | #1020 #1158 #1286 #1350 |
| | | #1570 #1591 #163 #2107 |
| | | #2320 #2731 #2806 #3013 |
| | | #3086 #3142 #367 #442 |
| | | #667 #668 #685 #704 #714 |
| | | #767 #790 #976 #M258 |
| 3747T > A | | |
| 3748C > T | | #1020 #1055 #1158 #1286 |
| | | #129 #1350 #1570 #1591 |
| | | #163 #2107 #2112 #2320 |
| | | #2731 #2806 #3013 #3086 |
| | | #3142 #367 #442 #508 #667 |
| | | #668 #685 #704 #714 #767 |
| | | #790 #976 #M258 |
| 3751_3753delinsATT | 517V > I | #2731 |
| 3753G > T | | #3013 #667 #2806 #M258 |
| | | #767 #1591 #1350 #668 |
| | | #367 #1020 #1158 #2107 |
| | | #1570 #704 #714 #3086 |
| | | #3142 #790 #976 #1286 |
| | | #163 #685 #442 #2320 |
| | | #1449 |
| 3753G > A | | #1449 |
| 3756T > C | | #129 #508 |
| 3759G > A | | #1020 #1158 #1286 #1350 |
| | | #1570 #1591 #163 #1704 |
| | | #1919 #2107 #2320 #2731 |
| | | #2806 #3013 #3086 #3142 |
| | | #3511 #367 #442 #667 #685 |
| | | #704 #714 #767 #790 #976 |
| | | #M258 |
| 3762C > A | | #1020 #1158 #1286 #1570 |
| | | #1591 #163 #1704 #2107 |
| | | #2112 #2320 #2731 #2806 |
| | | #3013 #3086 #3142 #3511 |
| | | #367 #442 #667 #685 #704 |
| | | #714 #767 #790 #976 |
| | | #M258 |
| 3762C > G | | #1350 |
| 3763_3765delinsACA | 521P > T | #1343 #777 |
| 3765G > A | | #3511 #3013 #2731 #667 |
| | | #2806 #M258 #217 #767 |
| | | #1591 #1704 #2112 #1350 |
| | | #367 #1020 #1158 #2107 |
| | | #1570 #704 #714 #3086 |
| | | #3142 #790 #976 #1286 |
| | | #163 #685 #442 #2320 |
| 3765G > T | | #508 #1024 |
| 3768C > T | | #1020 #1024 #1158 #1286 |
| | | #1350 #1570 #1591 #163 |
| | | #1704 #2107 #2112 #217 |
| | | #2320 #2731 #2806 #3086 |
| | | #3142 #367 #442 #508 #667 |
| | | #668 #685 #704 #714 #767 |
| | | #790 #976 #M258 |
| 3774A > C | | #1017 #1020 #1024 #1055 |
| | | #1158 #1260 #1273 #1286 |
| | | #129 #1343 #1350 #1449 |
| | | #1534 #1570 #1591 #163 |
| | | #1704 #1919 #2040 #2087 |
| | | #2102 #2107 #2112 #2128 |
| | | #2141 #217 #2206 #2208 |
| | | #2320 #2497 #2557 #2731 |
| | | #2806 #3013 #3086 #3142 |
| | | #3511 #367 #371 #3765 |
| | | #387 #442 #471 #473 #508 |
| | | #667 #668 #685 #704 #714 |
| | | #767 #777 #790 #877 #976 |
| | | #M258 |
| 3777C > T | | #1020 #1024 #1704 #2141 |
| | | #2806 #3086 #3142 #M258 |
| 3780C > T | | #2806 #3086 |
| 3783G > A | | #1024 #1158 #1286 #1350 |
| | | #1570 #1591 #163 #2107 |
| | | #2112 #2141 #217 #2320 |
| | | #2731 #2806 #3013 #3086 |
| | | #3511 #367 #442 #508 #668 |
| | | #685 #704 #714 #767 #790 |
| | | #976 |
| 3786C > T | | #1020 #1704 #3142 #442 |
| | | #714 #M258 |
| 3789T > C | | #1020 #1704 #3142 #714 |
| | | #M258 |
| 3795A > G | | #1449 |
| 3795_3796insGAA | 531_533insE | #1570 |
| 3800T > A | 533F > Y | #1343 #3013 #3511 #777 |
| 3804T > C | | #1020 #1024 #1158 #1286 |
| | | #129 #1350 #1449 #1570 |
| | | #1591 #163 #1704 #2107 |
| | | #2112 #217 #2320 #2806 |
| | | #3086 #3142 #367 #442 |
| | | #508 #668 #685 #704 #714 |
| | | #767 #790 #976 #M258 |
| 3807T > C | | #1020 #1024 #1158 #1286 |
| | | #129 #1350 #1570 #1591 |
| | | #163 #1704 #2107 #2112 |
| | | #217 #2320 #3086 #3142 |
| | | #367 #442 #508 #668 #685 |
| | | #704 #714 #767 #790 #976 |
| | | #M258 |
| 3808_3809delinsAT | 536Q > M | #129 #M258 #217 #767 |
| | | #1591 #508 #1704 #2112 |
| | | #1350 #668 #367 #1020 |
| | | #1158 #2107 #1570 #704 |
| | | #714 #3086 #3142 #1024 |
| | | #790 #976 #1286 #163 #685 |
| | | #442 #2320 |
| 3810G > A | | #1055 #1449 #1534 #2040 |
| | | #2087 #2206 #2208 #2557 |
| | | #371 #3765 #471 #877 |
| 3810G > C | 536Q > H | #2497 |
| 3811_3813delinsCAT | 537S > H | #129 #M258 #217 #1591 |
| | | #508 #1704 #2112 #1350 |
| | | #668 #367 #1020 #1158 |
| | | #2107 #1570 #704 #714 |
| | | #3086 #3142 #1024 #790 |
| | | #976 #1286 #163 #685 #442 |
| | | #2320 |
| 3811_3813delinsCCT | 537S > P | #767 |
| 3816G > A | | #1020 #1024 #1158 #1286 |
| | | #129 #1350 #1570 #1591 |
| | | #163 #1704 #2107 #2112 |
| | | #217 #2320 #3086 #3142 |
| | | #367 #442 #508 #668 #685 |
| | | #704 #714 #767 #790 #976 |
| | | #M258 |
| 3817_3819delinsACC | 539V > T | #129 #M258 #217 #767 |
| | | #1591 #508 #2112 #1350 |
| | | #668 #367 #1020 #1158 |
| | | #2107 #1570 #704 #714 |
| | | #3086 #3142 #1024 #790 |
| | | #976 #1286 #163 #685 #442 |
| | | #2320 |
| 3817_3819delinsACA | 539V > T | #1704 |
| 3820C > T | | #1704 |
| 3822C > G | | #1020 #1024 #1158 #1286 |
| | | #1350 #1570 #1591 #163 |
| | | #1704 #2107 #2112 #217 |
| | | #2320 #3086 #3142 #367 |
| | | #442 #508 #668 #685 #704 |
| | | #714 #767 #790 #976 |
| | | #M258 |
| 3822C > A | | #129 |
| 3825C > A | | #1020 #1024 #1158 #1260 |
| | | #1286 #1350 #1570 #1591 |
| | | #163 #1704 #2107 #2112 |
| | | #217 #2320 #3086 #3142 |
| | | #367 #442 #508 #668 #685 |
| | | #704 #714 #767 #790 #976 |
| | | #M258 |
| 3828T > C | | #1343 #3013 #777 |
| 3831G > A | | #371 #1017 #473 #1273 |
| | | #2206 #2128 #1343 #777 |
| | | #3511 #3013 |
| 3831G > T | | #M258 #217 #767 #1591 |
| | | #508 #1704 #2112 #1350 |
| | | #668 #367 #1020 #1158 |
| | | #2107 #1570 #704 #714 |
| | | #3086 #3142 #1024 #790 |
| | | #976 #1286 #163 #685 #442 |
| | | #2320 |
| 3834G > A | | #1017 #1020 #1024 #1158 |
| | | #1273 #1286 #1343 #1350 |
| | | #1570 #1591 #163 #1704 |
| | | #2107 #2112 #217 #2206 |
| | | #2320 #2806 #3013 #3086 |
| | | #3142 #3511 #367 #371 |
| | | #442 #473 #508 #668 #685 |
| | | #704 #714 #767 #777 #790 |
| | | #976 #M258 |
| 3835C > G | 545Q > E | #1024 #3142 #508 #767 |
| 3839G > A | 546G > D | #1017 #1273 #1343 #3013 |
| | | #3511 #473 #777 |
| 3840C > A | | #M258 #217 #767 #1591 |
| | | #508 #1704 #2112 #1350 |
| | | #668 #367 #1020 #1158 |
| | | #2107 #1570 #704 #714 |
| | | #3086 #3142 #1024 #790 |
| | | #976 #1286 #163 #685 #442 |
| | | #2320 |
| 3840C > T | | #2128 #1343 |
| 3841T > A | 547S > T | #1020 #1024 #1158 #1286 |
| | | #1350 #1570 #1591 #163 |
| | | #1704 #2107 #2112 #217 |
| | | #2320 #3086 #3142 #367 |
| | | #442 #508 #668 #685 #704 |
| | | #714 #767 #790 #976 |
| | | #M258 |
| 3843A > G | | #1273 #1343 #3013 #3511 |
| | | #777 |
| 3843A > T | | #2320 #442 #M258 |
| 3844_3846delinsAAT | 548E > N | #217 #767 #1591 #508 |
| | | #1704 #2112 #1350 #668 |
| | | #367 #1020 #1158 #2107 |
| | | #1570 #704 #714 #3086 |
| | | #1024 #790 #976 #1286 |
| | | #163 #685 |
| 3845_3846delinsGA | 548E > G | #1017 #473 #1273 #1343 |
| | | #777 #3511 #3013 #M258 |
| | | #442 |
| 3844_3846delinsAAA | 548E > K | #2320 |
| 3844_3846delinsACT | 548E > T | #3142 |
| 3847A_3849delinsGCT | 549K > A | #217 #767 #1591 #508 |
| | | #2112 #1350 #668 #367 |
| | | #1158 #2107 #1570 #704 |
| | | #714 #1024 #790 #976 |
| | | #1286 #163 |
| 3847A_3849delinsTCT | 549K > S | #3142 |
| 3847A_3849delinsTGC | 549K > C | #2320 |
| 3848A > G | 549K > R | #M258 #442 |
| 3847_3849delinsGCC | 549K > A | #1704 #1020 #3086 #685 |
| 3850_3852delinsGAC | 550T > D | #M258 #442 |
| 3851_3852delinsAC | 550T > N | #217 #767 #1591 #1704 |
| | | #2112 #1350 #668 #367 |
| | | #1020 #1158 #2107 #1570 |
| | | #704 #714 #3086 #3142 |
| | | #790 #976 #1286 #163 #685 |
| | | #2320 |
| 3852A > T | | #1017 #473 #1273 #1343 |
| | | #777 #3511 #3013 |
| 3852A > C | | #129 #2806 #508 #1024 |
| 3853_3855delinsGAC | 551N > D | #217 #1591 #1704 #2112 |
| | | #1350 #668 #367 #1020 |
| | | #1158 #2107 #1570 #704 |
| | | #714 #3086 #790 #976 |
| | | #1286 #163 #685 #2320 |
| 3855T > C | | #129 #M258 #767 #508 |
| | | #3142 #1024 #442 |
| 3857T > C | 552V > A | #129 #M258 #217 #767 |
| | | #1591 #508 #1704 #2112 |
| | | #1350 #668 #367 #1020 |
| | | #1158 #2107 #1570 #704 |
| | | #714 #3086 #3142 #1024 |
| | | #790 #976 #1286 #163 #685 |
| | | #442 #2320 |
| 3858G > A | | #217 #767 #367 |
| 3859_3864del | 553_554delVD | #2128 |
| 3861C > T | | #M258 #217 #1591 #508 |
| | | #1704 #2112 #1350 #668 |
| | | #367 #1020 #1158 #2107 |
| | | #1570 #704 #714 #3086 |
| | | #3142 #1024 #790 #976 |
| | | #1286 #163 #685 #442 |
| | | #2320 |
| 3861C > A | 553D > E | #129 #767 |
| 3862_3864delinsTTG | 554I > L | #129 #M258 #217 #767 |
| | | #1591 #508 #1704 #2112 |
| | | #1350 #668 #367 #1020 |
| | | #1158 #2107 #1570 #704 |
| | | #714 #3086 #3142 #1024 |
| | | #790 #976 #1286 #163 #685 |
| | | #442 #2320 |
| 3867A > C | 555E > D | #1024 #1158 #1286 #1350 |
| | | #1570 #1591 #163 #2107 |
| | | #2112 #217 #2320 #3086 |
| | | #367 #442 #508 #668 #685 |
| | | #704 #714 #790 #976 |
| | | #M258 |
| 3868_3870delinsCAT | 556K > H | #3142 |
| 3870G > T | 556K > N | #1020 #1024 #1158 #1286 |
| | | #129 #1350 #1570 #1591 |
| | | #163 #1704 #2107 #2112 |
| | | #217 #2320 #3086 #3142 |
| | | #367 #442 #508 #668 #685 |
| | | #704 #714 #767 #790 #976 |
| | | #M258 |
| 3873C > T | | #1286 #1591 #1704 #2320 |
| | | #3511 |
| 3885C > T | | #1024 #1158 #1286 #129 |
| | | #1350 #1570 #1591 #163 |
| | | #1704 #2107 #2112 #217 |
| | | #2320 #3086 #3142 #367 |
| | | #442 #668 #685 #704 #714 |
| | | #767 #790 #976 #M258 |
| 3887A > G | 562E > G | #2141 |
| 3891G > A | | #1010 #1020 #1024 #1158 |
| | | #1286 #1350 #1570 #1591 |
| | | #163 #1704 #2107 #2112 |
| | | #217 #2320 #3086 #3142 |
| | | #367 #387 #442 #668 #685 |
| | | #704 #714 #767 #790 #976 |
| | | #M258 |
| 3898_3900delinsCGC | | #1010 #M258 #217 #767 |
| | | #1591 #2112 #1350 #668 |
| | | #367 #1158 #2107 #1570 |
| | | #704 #3086 #1024 #790 |
| | | #976 #1286 #163 #685 #442 |
| | | #2320 |
| 3903A > C | | #1010 #1017 #1020 #1024 |
| | | #1055 #1158 #1260 #1273 |
| | | #1286 #129 #1343 #1350 |
| | | #1449 #1534 #1570 #1591 |
| | | #163 #1704 #1919 #2040 |
| | | #2087 #2102 #2107 #2112 |
| | | #2128 #2141 #217 #2206 |
| | | #2208 #2320 #2497 #2557 |
| | | #2806 #3013 #3086 #3142 |
| | | #3511 #367 #371 #3765 |
| | | #387 #442 #471 #473 #668 |
| | | #685 #704 #714 #767 #777 |
| | | #790 #877 #976 #M258 |
| 3906C > A | | #1024 #3086 #508 |
| 3906C > T | | #3142 |
| 3912C > T | | #1024 #3086 #3142 #508 |
| 3912C > A | | #976 |
| 3915G > A | | #1024 #1158 #3086 #508 |
| 3915G > C | | #2141 |
| 3918T > C | | #2141 |
| 3921G > A | | #129 #371 |
| 3930T > C | | #1010 #1158 #1286 #1350 |
| | | #1570 #1591 #163 #1704 |
| | | #2107 #2112 #217 #2320 |
| | | #2806 #3086 #3142 #367 |
| | | #442 #508 #667 #668 #685 |
| | | #704 #714 #767 #790 #976 |
| | | #985 #M258 |
| 3933T > A | | #1010 #1024 #1158 #1286 |
| | | #1350 #1570 #1591 #163 |
| | | #1704 #2107 #2112 #217 |
| | | #2320 #2731 #2806 #3086 |
| | | #3142 #367 #442 #508 #667 |
| | | #668 #685 #704 #714 #767 |
| | | #790 #976 #985 #M258 |
| 3933T > G | | #1020 |
| 3934T > G | 578S > A | #2497 #1449 #1020 |
| 3935C > A | 578S > Y | #1010 #985 #667 #2806 |
| | | #M258 #217 #767 #1591 |
| | | #508 #1704 #2112 #1350 |
| | | #668 #367 #1158 #2107 |
| | | #1570 #704 #714 #3086 |
| | | #1024 #790 #976 #1286 |
| | | #163 #685 #442 #2320 |
| 3934_3935delinsAA | 578S > N | #3142 |
| 3939A > G | | #1010 #985 #667 #2806 |
| | | #M258 #217 #767 #1591 |
| | | #508 #1704 #2112 #1350 |
| | | #668 #367 #1020 #1158 |
| | | #2107 #1570 #704 #714 |
| | | #3086 #3142 #1024 #790 |
| | | #976 #1286 #685 #442 |
| | | #2320 |
| 3939A > C | | #163 |
| 3942T > A | | #1010 #985 #667 #2806 |
| | | #M258 #217 #767 #1591 |
| | | #508 #1704 #2112 #1350 |
| | | #668 #367 #1020 #1158 |
| | | #2107 #1570 #704 #714 |
| | | #3086 #3142 #1024 #790 |
| | | #976 #1286 #163 #685 #442 |
| | | #2320 |
| 3944_3945delinsAT | 581T > N | #1010 #985 #667 #2806 |
| | | #M258 #217 #767 #1591 |
| | | #508 #1704 #2112 #1350 |
| | | #668 #367 #1020 #1158 |
| | | #2107 #1570 #704 #714 |
| | | #3086 #3142 #1024 #790 |
| | | #976 #1286 #163 #685 #442 |
| | | #2320 |
| 3948C > T | | #1010 #985 #667 #2806 |
| | | #M258 #217 #767 #1591 |
| | | #508 #1704 #2112 #1350 |
| | | #668 #367 #1020 #1158 |
| | | #2107 #1570 #704 #714 |
| | | #3086 #3142 #1024 #790 |
| | | #976 #1286 #163 #685 #442 |
| | | #2320 |
| 3949_3951_delinsTTG | | #1010 #985 #667 #2806 |
| | | #M258 #217 #767 #1591 |
| | | #508 #1704 #2112 #1350 |
| | | #668 #367 #1020 #1158 |
| | | #2107 #1570 #704 #714 |
| | | #3086 #3142 #1024 #790 |
| | | #976 #1286 #163 #685 #442 |
| | | #2320 |
| 3951C > T | | #3013 |
| 3954G > A | | #1343 #777 #3013 #1010 |
| | | #985 #667 #2806 #M258 |
| | | #217 #767 #1591 #508 |
| | | #1704 #2112 #668 #367 |
| | | #1020 #1158 #2107 #1570 |
| | | #704 #714 #3086 #3142 |
| | | #1024 #790 #976 #1286 |
| | | #163 #685 #442 #2320 |
| 3956_3957delinsAC | 585R > N | #1010 #985 #667 #2806 |
| | | #M258 #217 #767 #1591 |
| | | #508 #1704 #2112 #668 |
| | | #367 #1020 #1158 #2107 |
| | | #1570 #704 #714 #3086 |
| | | #3142 #1024 #790 #976 |
| | | #1286 #163 #685 #442 |
| | | #2320 |
| 3957A > C | 585R > S | #2497 #2102 #371 #1017 |
| | | #473 #1273 #2087 #2206 |
| | | #877 #1534 #2557 #1919 |
| | | #1260 #3765 #471 #387 |
| | | #2040 #1449 #2128 #1343 |
| | | #777 #3511 #3013 #2208 |
| | | #1055 #1350 |
| 3958_3960delinsTCA | 586G > S | #1010 #985 #667 #2806 |
| | | #M258 #217 #767 #1591 |
| | | #508 #1704 #2112 #668 |
| | | #367 #1020 #1158 #2107 |
| | | #1570 #704 #714 #3086 |
| | | #3142 #1024 #790 #976 |
| | | #1286 #163 #685 #442 |
| | | #2320 |
| 3963C > T | | #1010 #985 #667 #2806 |
| | | #217 #767 #1591 #2112 |
| | | #668 #367 #1158 #2107 |
| | | #1570 #704 #714 #3086 |
| | | #3142 #1024 #790 #976 |
| | | #163 #685 #2320 |
| 3965_3966delinsCT | 588R > T | #985 #667 #2806 #M258 |
| | | #217 #767 #1591 #508 |
| | | #1704 #2112 #668 #367 |
| | | #1020 #1158 #2107 #1570 |
| | | #704 #714 #3086 #3142 |
| | | #1024 #790 #976 #1286 |
| | | #163 #685 #442 #2320 |
| 3959G > C | 588R > T | #2497 #2102 #371 #1017 |
| | | #473 #1273 #2087 #2206 |
| | | #877 #1534 #2557 #1919 |
| | | #1260 #3765 #471 #387 |
| | | #2040 #1449 #2128 #2141 |
| | | #1343 #777 #3511 #3013 |
| | | #2208 #1055 #1350 |
| 3967_3969delinsGGT | 589Q > G | #985 #667 #2806 #217 #767 |
| | | #1591 #1704 #2112 #668 |
| | | #367 #1020 #1158 #2107 |
| | | #1570 #704 #714 #3086 |
| | | #3142 #790 #976 #1286 |
| | | #163 #685 #442 #2320 |
| 3968A > G | 589Q > R | #2731 |
| 3967_3969delinsGCT | 589Q > A | #M258 #508 #1024 |
| 3970G > C | 590A > P | #1020 #1158 #1286 #1570 |
| | | #1591 #163 #1704 #2107 |
| | | #2112 #217 #2320 #2806 |
| | | #3086 #3142 #367 #442 |
| | | #667 #668 #685 #704 #714 |
| | | #767 #790 #976 #985 |
| | | #M258 |
| 3972A > G | | #M258 |
| 3973G > A | 591A > T | #985 #667 #2806 #217 #767 |
| | | #1591 #508 #1704 #2112 |
| | | #668 #367 #1020 #1158 |
| | | #2107 #1570 #704 #714 |
| | | #3086 #3142 #1024 #790 |
| | | #976 #1286 #163 #685 #442 |
| 3973_3974delinsAG | 591A > S | #M258 |
| 3978C > T | | #1020 #1024 #1158 #1286 |
| | | #1350 #1570 #1591 #163 |
| | | #1704 #2107 #2112 #217 |
| | | #2320 #2806 #3086 #3142 |
| | | #367 #442 #508 #667 #668 |
| | | #685 #704 #714 #767 #790 |
| | | #976 #985 #M258 |
| 3979G > T | 593A > S | #1017 #1055 #1260 #1273 |
| | | #1343 #1449 #1534 #1919 |
| | | #2087 #2102 #2206 #2208 |
| | | #2497 #3013 #3511 #473 |
| | | #777 |
| 3980C > G | 593A > G | #985 #667 #2806 #M258 |
| | | #217 #767 #1591 #1704 |
| | | #2112 #1350 #668 #367 |
| | | #1020 #1158 #2107 #1570 |
| | | #704 #714 #3086 #790 #976 |
| | | #1286 #163 #685 #442 |
| | | #2320 |
| 3980C > A | 593A > E | #508 #3142 #1024 |
| 3982_3983delinsAC | 594D > T | #985 #667 #2806 #M258 |
| | | #217 #767 #1591 #508 |
| | | #1704 #2112 #1350 #668 |
| | | #367 #1020 #1158 #2107 |
| | | #1570 #704 #714 #3086 |
| | | #1024 #790 #976 #1286 |
| | | #163 #685 #442 #2320 |
| 3982G > A | 594D > N | #3142 |
| 3987C > A | | #163 |
| 3990C > T | | #1020 #1024 #1158 #1286 |
| | | #1350 #1570 #1591 #163 |
| | | #1704 #2107 #2112 #217 |
| | | #2320 #2806 #3086 #3142 |
| | | #367 #442 #508 #667 #668 |
| | | #685 #704 #714 #767 #790 |
| | | #976 #985 #M258 |
| 3991A > G | 597T > A | #1017 #473 #1273 #1260 |
| 3991_3993delinsCAC | 597T > H | #985 #667 #2806 #M258 |
| | | #217 #767 #1591 #508 |
| | | #1704 #2112 #1350 #668 |
| | | #367 #1020 #1158 #2107 |
| | | #1570 #704 #714 #3086 |
| | | #1024 #790 #976 #1286 |
| | | #163 #685 #442 #2320 |
| 3992_3993delinsAC | 597T > N | #3142 |
| 3999C > T | | #1017 #1273 #2087 #1260 |
| 3999C > A | | #985 #667 #2806 #M258 |
| | | #217 #767 #1591 #508 |
| | | #1704 #2112 #1350 #668 |
| | | #367 #1020 #1158 #2107 |
| | | #1570 #704 #714 #3086 |
| | | #3142 #1024 #790 #976 |
| | | #1286 #163 #685 #442 |
| | | #2320 |
| 4000G > A | 600V > I | #2497 #1449 |
| 4001_4002delinsCG | 600V > A | #985 #667 #2806 #M258 |
| | | #217 #767 #1591 #508 |
| | | #1704 #2112 #1350 #668 |
| | | #367 #1020 #1158 #2107 |
| | | #1570 #704 #714 #3086 |
| | | #3142 #1024 #790 #976 |
| | | #1286 #163 #685 #442 |
| | | #2320 |
| 4003_4005delinsTTA | | #985 #667 #2806 #M258 |
| | | #217 #767 #1591 #508 |
| | | #1704 #2112 #1350 #668 |
| | | #367 #1020 #1158 #2107 |
| | | #1570 #704 #714 #3086 |
| | | #3142 #1024 #790 #976 |
| | | #1286 #163 #685 #442 |
| | | #2320 |
| 4006C > T | 602P > S | #777 |
| 4008A > T | | #1020 #1024 #1158 #1286 |
| | | #1350 #1570 #1591 #163 |
| | | #1704 #2107 #2112 #217 |
| | | #2320 #2806 #3013 #3086 |
| | | #3142 #3511 #367 #371 |
| | | #442 #508 #667 #668 #685 |
| | | #704 #714 #767 #790 #976 |
| | | #985 #M258 |
| 4011C > T | | #1020 #1024 #1158 #1286 |
| | | #1350 #1570 #1591 #163 |
| | | #1704 #2107 #2112 #217 |
| | | #2320 #2806 #3086 #3142 |
| | | #367 #442 #508 #667 #668 |
| | | #685 #704 #714 #767 #790 |
| | | #976 #985 #M258 |
| 4017C > G | | #1020 #1024 #1158 #1286 |
| | | #1350 #1570 #1591 #163 |
| | | #1704 #2107 #2112 #217 |
| | | #2320 #2806 #3086 #3142 |
| | | #367 #442 #508 #667 #668 |
| | | #685 #704 #714 #767 #790 |
| | | #976 #985 #M258 |
| 4017C > A | | #2557 |
| 4026C > T | | #1020 #1024 #1158 #1286 |
| | | #1350 #1570 #1591 #163 |
| | | #1704 #2107 #2112 #217 |
| | | #2320 #2806 #3086 #3142 |
| | | #367 #442 #667 #668 #685 |
| | | #704 #714 #767 #790 #976 |
| | | #985 #M258 |
| 4027A > C | | #1020 #1158 #1286 #1350 |
| | | #1570 #1591 #163 #2107 |
| | | #2112 #217 #2320 #2806 |
| | | #3086 #367 #442 #508 #667 |
| | | #668 #685 #704 #714 #767 |
| | | #790 #976 #985 |
| 4032T > C | | #1020 #1024 #1055 #1158 |
| | | #1260 #1273 #1286 #1343 |
| | | #1350 #1449 #1570 #1591 |
| | | #163 #1704 #1919 #2040 |
| | | #2087 #2102 #2107 #2112 |
| | | #2128 #2141 #217 #2206 |
| | | #2208 #2320 #2497 #2557 |
| | | #2806 #3013 #3086 #3142 |
| | | #3511 #367 #371 #3765 |
| | | #387 #442 #471 #473 #508 |
| | | #667 #668 #685 #704 #714 |
| | | #767 #777 #790 #877 #976 |
| | | #985 #M258 |
| 4035G > A | | #1704 |
| 4041T > G | | #1017 #1020 #1024 #1055 |
| | | #1158 #1260 #1273 #1286 |
| | | #1343 #1350 #1449 #1534 |
| | | #1570 #1591 #163 #1919 |
| | | #2087 #2102 #2107 #2112 |
| | | #2128 #2141 #217 #2206 |
| | | #2208 #2320 #2497 #2557 |
| | | #2806 #3013 #3086 #3142 |
| | | #3511 #367 #371 #3765 |
| | | #387 #442 #471 #473 #667 |
| | | #668 #685 #704 #714 #767 |
| | | #777 #790 #877 #976 #985 |
| | | #M258 |
| 4041T > A | | #1704 #508 |
| 4047G > A | | #1020 #1024 #1158 #1286 |
| | | #1343 #1350 #1449 #1570 |
| | | #1591 #163 #2107 #2112 |
| | | #217 #2208 #2320 #2497 |
| | | #2806 #3013 #3086 #3142 |
| | | #3511 #367 #442 #508 #667 |
| | | #668 #685 #704 #714 #767 |
| | | #790 #976 #985 #M258 |
| 4047G > T | | #1704 |
| 4050C > T | | #1534 #2102 #2141 #2557 |
| | | #387 #877 |
| 4053C > T | | #1020 #1024 #1158 #1286 |
| | | #1350 #1449 #1570 #1591 |
| | | #163 #1704 #2107 #2112 |
| | | #217 #2208 #2320 #3142 |
| | | #367 #442 #471 #508 #667 |
| | | #668 #685 #704 #714 #767 |
| | | #790 #976 #985 #M258 |
| 4053C > A | | #2806 #3086 |
| 4059A > C | | #1020 #1024 #1158 #1286 |
| | | #1350 #1570 #1591 #163 |
| | | #1704 #2107 #2112 #217 |
| | | #2208 #2320 #2806 #3086 |
| | | #3142 #367 #442 #508 #667 |
| | | #668 #685 #704 #714 #767 |
| | | #790 #976 #985 #M258 |
| 4062G > A | | #1024 #471 |
| 4068A > T | | #1020 #1024 #1158 #1286 |
| | | #1350 #1570 #1591 #163 |
| | | #1704 #2107 #2112 #217 |
| | | #2208 #2320 #2806 #3086 |
| | | #367 #442 #508 #667 #668 |
| | | #685 #704 #714 #767 #790 |
| | | #976 #985 #M258 |
| 4068A > C | | #1260 #2128 #777 |
| 4074G > C | | #1020 #1024 #1158 #1350 |
| | | #1570 #1591 #163 #1704 |
| | | #2107 #2112 #217 #2208 |
| | | #2320 #2806 #3086 #367 |
| | | #442 #508 #667 #668 #685 |
| | | #704 #714 #767 #790 #976 |
| | | #985 #M258 |
| 4074G > A | | #2206 |
| 4077C > T | | #1020 #1024 #1158 #1350 |
| | | #1570 #1591 #163 #1704 |
| | | #2107 #2112 #2128 #217 |
| | | #2208 #2320 #2806 #3086 |
| | | #367 #3765 #442 #471 #508 |
| | | #667 #668 #685 #704 #714 |
| | | #767 #777 #790 #976 #985 |
| | | #M258 |
| 4083T > C | | #1020 #1024 #1158 #1260 |
| | | #1350 #1570 #1591 #163 |
| | | #1704 #2107 #2112 #2128 |
| | | #217 #2208 #2320 #2497 |
| | | #2806 #3013 #3086 #3142 |
| | | #3511 #367 #3765 #442 |
| | | #471 #508 #667 #668 #685 |
| | | #704 #714 #767 #777 #790 |
| | | #976 #985 #M258 |
| 4089C > T | | #1020 #1024 #1158 #1286 |
| | | #1350 #1570 #1591 #163 |
| | | #1704 #2107 #2112 #217 |
| | | #2208 #2320 #2806 #3086 |
| | | #3142 #367 #442 #508 #667 |
| | | #668 #685 #704 #767 #790 |
| | | #976 #985 #M258 |
| 4092C > T | | #1020 #1024 #1158 #1260 |
| | | #1286 #1343 #1350 #1570 |
| | | #1591 #163 #1704 #2107 |
| | | #2112 #2128 #217 #2208 |
| | | #2320 #2497 #2806 #3013 |
| | | #3086 #3142 #3511 #367 |
| | | #3765 #442 #471 #508 #667 |
| | | #668 #685 #704 #714 #767 |
| | | #777 #790 #976 #985 |
| | | #M258 |
| 4095T > C | | #777 |
| 4098C > A | | #1020 #1158 #1286 #1350 |
| | | #1591 #163 #2112 #217 |
| | | #2320 #367 #442 #668 #685 |
| | | #714 #767 #790 #976 #985 |
| 4098C > G | | #1024 #1570 #1704 #2107 |
| | | #2208 #2806 #3086 #3142 |
| | | #704 #777 #M258 |
| 4098C > T | | #1260 #1343 #2128 #3765 |
| | | #471 |
| 4101C > G | | #1020 #1024 #1158 #1286 |
| | | #1350 #1570 #1591 #163 |
| | | #1704 #2107 #2112 #217 |
| | | #2208 #2320 #2806 #3086 |
| | | #367 #442 #668 #685 #704 |
| | | #714 #767 #790 #976 #985 |
| | | #M258 |
| 4101C > T | | #1260 #1343 #2128 #3142 |
| | | #3765 #471 |
| 4107T > C | | #1017 #1055 #1260 #1273 |
| | | #1534 #1919 #2040 #2087 |
| | | #2141 #2206 #2557 #3013 |
| | | #3511 #371 #387 #508 #877 |
| 4107T > A | | #1020 #1024 #1158 #1286 |
| | | #1343 #1350 #1570 #1591 |
| | | #163 #1704 #2107 #2112 |
| | | #2128 #217 #2208 #2320 |
| | | #2806 #3086 #3142 #367 |
| | | #3765 #442 #471 #668 #685 |
| | | #704 #714 #767 #777 #790 |
| | | #976 #985 #M258 |
| 4110A > T | | #1020 #1024 #1158 #1286 |
| | | #1350 #1570 #1591 #163 |
| | | #1704 #2107 #2112 #217 |
| | | #2208 #2320 #2806 #3086 |
| | | #3142 #367 #442 #668 #685 |
| | | #704 #714 #767 #790 #976 |
| | | #985 #M258 |
| 4113C > T | | #1020 #1024 #1055 #1158 |
| | | #1286 #1343 #1350 #1534 |
| | | #1570 #1591 #163 #1704 |
| | | #2087 #2107 #2112 #2128 |
| | | #2141 #217 #2206 #2208 |
| | | #2320 #2557 #2806 #3013 |
| | | #3086 #3142 #3511 #367 |
| | | #3765 #442 #471 #668 #685 |
| | | #704 #714 #767 #777 #790 |
| | | #877 #976 #985 #M258 |
| 4116A > G | | #1020 #1158 #1286 #1350 |
| | | #1570 #1591 #2107 #2112 |
| | | #217 #367 #668 #685 #767 |
| | | #790 |
| 4117C > T | | #2087 #2206 |
| 4119T > G | | #1017 #1055 #1343 #1919 |
| | | #2087 #3013 #3511 #877 |
| 4119T > C | | #1020 #1024 #1158 #1286 |
| | | #1350 #1570 #1591 #163 |
| | | #1704 #2107 #2112 #217 |
| | | #2208 #2320 #3086 #3142 |
| | | #367 #442 #508 #668 #685 |
| | | #704 #714 #767 #790 #976 |
| | | #985 #M258 |
| 4119T > A | | #2206 |
| 4122A > G | | #1055 #1343 #1919 #2087 |
| | | #2206 #3013 #3511 #877 |
| 4123C > A | 641H > N | #508 |
| 4128T > G | | #1020 #1158 #1286 #1350 |
| | | #1570 #1591 #163 #1704 |
| | | #2107 #2112 #217 #367 |
| | | #668 #685 #714 #790 #985 |
| 4128T > A | | #1024 #2208 #2320 #3086 |
| | | #3142 #442 #508 #704 #767 |
| | | #976 #M258 |
| 4128T > C | | #1343 |
| 4131T > C | | #1260 #1343 #2087 #2128 |
| | | #2141 #2206 #3013 #3511 |
| | | #3765 #471 #473 #777 |
| 4134A > T | | #1020 #1024 #1158 #1286 |
| | | #1350 #1570 #1591 #163 |
| | | #1704 #2107 #2112 #217 |
| | | #2208 #2320 #3086 #3142 |
| | | #367 #442 #508 #668 #685 |
| | | #704 #714 #767 #790 #976 |
| | | #985 #M258 |
| 4134A > G | | #1260 #1343 #2040 #2087 |
| | | #2128 #2141 #2206 #3013 |
| | | #3511 #3765 #387 #471 |
| | | #473 #777 |
| 4137G > A | | #1017 #1273 #2040 #2206 |
| | | #3142 #387 |
| 4140T > C | | #1020 #1024 #1158 #1286 |
| | | #1350 #1570 #1591 #163 |
| | | #1704 #2107 #2112 #217 |
| | | #2208 #2320 #2806 #3086 |
| | | #3142 #367 #442 #508 #667 |
| | | #668 #685 #704 #714 #767 |
| | | #790 #976 #985 #M258 |
| 4141_4143delinsATG | 647L > M | #2208 #985 #667 #2806 |
| | | #M258 #217 #767 #1591 |
| | | #508 #1704 #2112 #1350 |
| | | #668 #367 #1020 #1158 |
| | | #2107 #1570 #704 #714 |
| | | #3086 #3142 #1024 #790 |
| | | #976 #1286 #163 #685 #442 |
| | | #2320 |
| 4146C > T | | #1055 #2206 #442 |
| 4149G > A | | #1020 #1024 #1158 #1286 |
| | | #1350 #1570 #1591 #163 |
| | | #1704 #2102 #2107 #2112 |
| | | #217 #2208 #2320 #2806 |
| | | #3086 #3142 #367 #3765 |
| | | #442 #471 #508 #667 #668 |
| | | #685 #704 #714 #767 #790 |
| | | #976 #985 #M258 |
| 4151A > G | 650N > S | #667 |
| 4152C > T | | #1055 #1343 #2206 #3013 |
| | | #3511 #777 |
| 4153A > T | 651T > S | #667 |
| 4155C > T | | #1020 #1024 #1158 #1286 |
| | | #1350 #1570 #1591 #163 |
| | | #1704 #2107 #2112 #217 |
| | | #2208 #2320 #2806 #3086 |
| | | #3142 #367 #442 #508 #667 |
| | | #668 #685 #704 #714 #767 |
| | | #790 #976 #985 #M258 |
| 4158G > C | | #1020 #1024 #1158 #1286 |
| | | #1350 #1591 #163 #1704 |
| | | #2112 #217 #2208 #2320 |
| | | #2806 #3086 #3142 #367 |
| | | #442 #508 #667 #668 #685 |
| | | #714 #767 #777 #790 #976 |
| | | #985 #M258 |
| 4158G > A | | #1055 #1570 #2107 #704 |
| 4158G > T | | #1273 |
| 4161A > T | | #1010 #1020 #1024 #1158 |
| | | #1286 #1350 #1570 #1591 |
| | | #163 #1704 #2107 #2112 |
| | | #217 #2208 #2320 #2806 |
| | | #3086 #3142 #367 #442 |
| | | #508 #667 #668 #685 #704 |
| | | #714 #767 #790 #976 #985 |
| | | #M258 |
| 4164T > A | | #1010 #1020 #1024 #1158 |
| | | #1286 #1350 #1591 #163 |
| | | #1704 #2107 #2112 #217 |
| | | #2208 #2320 #2806 #3086 |
| | | #3142 #367 #442 #508 #667 |
| | | #668 #685 #704 #714 #767 |
| | | #790 #976 #985 #M258 |
| 4165_4209delins_46nt | 655_669delins_15aa | #1570 |
| 4167G > C | | #1010 #1020 #1024 #1158 |
| | | #1286 #1350 #1591 #163 |
| | | #1704 #2107 #2112 #217 |
| | | #2208 #2320 #2806 #3086 |
| | | #3142 #367 #442 #508 #667 |
| | | #668 #685 #704 #714 #767 |
| | | #790 #976 #985 #M258 |
| 4167G > A | | #1534 #2206 #2557 |
| 4174T > C | 658S > P | #1010 #1020 #1024 #1158 |
| | | #1286 #1350 #1570 #1591 |
| | | #163 #1704 #2107 #2112 |
| | | #217 #2208 #2320 #2806 |
| | | #3086 #3142 #367 #442 |
| | | #508 #667 #668 #685 #704 |
| | | #714 #767 #790 #976 #985 |
| | | #M258 |
| 4176G > T | | #1010 #1020 #1055 #1158 |
| | | #1286 #1343 #1570 #1591 |
| | | #217 #2206 #3013 #3086 |
| | | #3142 #367 #667 #767 #790 |
| | | #877 #976 #M258 |
| 4176G > A | | #1017 #471 #685 |
| 4176G > C | | #1024 #1350 #163 #1704 |
| | | #2107 #2112 #2208 #2320 |
| | | #442 #508 #668 #704 #714 |
| | | #985 |
| 4178C > G | | |
| 4179C > A | | #1010 #1020 #1024 #1158 |
| | | #1286 #1350 #1591 #163 |
| | | #2107 #2112 #217 #2208 |
| | | #2320 #2806 #3086 #3142 |
| | | #367 #442 #508 #667 #668 |
| | | #685 #704 #714 #767 #790 |
| | | #976 #985 #M258 |
| 4179C > G | | #1704 |
| 4181C > A | 660T > N | #1010 #1020 #1024 #1158 |
| | | #1286 #1350 #1591 #163 |
| | | #1704 #2107 #2112 #217 |
| | | #2208 #2320 #2806 #3086 |
| | | #3142 #367 #442 #508 #667 |
| | | #668 #685 #704 #714 #767 |
| | | #790 #976 #985 #M258 |
| 4182C > T | | #2206 #3765 #714 #877 |
| 4185C > T | | #1024 #1570 #2128 #2320 |
| 4188T > C | | #1055 #1260 #1343 #2206 |
| | | #3013 |
| 4189_4191delinsTCT | 663A > S | #2208 #1010 #985 #667 |
| | | #2806 #217 #1591 #508 |
| | | #1704 #2112 #1350 #668 |
| | | #367 #1020 #1158 #2107 |
| | | #704 #714 #3086 #790 #976 |
| | | #1286 #163 #685 #442 |
| | | #2320 |
| 4191G > A | | #2087 #3765 #471 |
| 4192G > T | 664A > S | #1602 #1570 |
| 4194A > C | | #1010 #1020 #1158 #1286 |
| | | #1350 #1591 #1602 #163 |
| | | #1704 #2107 #2112 #217 |
| | | #2208 #2320 #2806 #3086 |
| | | #367 #442 #508 #667 #668 |
| | | #685 #704 #714 #790 #976 |
| | | #985 |
| 4197G > A | | #767 #877 #M258 |
| 4206C > T | | #1010 #1020 #1024 #1158 |
| | | #1286 #1350 #1534 #1591 |
| | | #1602 #163 #1704 #2107 |
| | | #2112 #217 #2206 #2320 |
| | | #2557 #2806 #3086 #3142 |
| | | #367 #442 #508 #667 #668 |
| | | #685 #704 #714 #790 #877 |
| | | #976 #985 |
| 4209C > T | | #2141 #473 #767 #985 |
| | | #M258 |
| 4212C > T | | #1273 #2208 #371 #3765 |
| | | #471 |
| 4221C > T | | #1024 #1055 #1158 #1260 |
| | | #1286 #129 #1350 #1570 |
| | | #1591 #1602 #163 #1704 |
| | | #1919 #2040 #2087 #2107 |
| | | #2112 #2128 #2208 #2320 |
| | | #2806 #3086 #3765 #387 |
| | | #442 #508 #668 #685 #767 |
| | | #777 #976 #985 #M258 |
| 4230A > G | | #1010 #1017 #1020 #1024 |
| | | #1055 #1158 #1260 #1273 |
| | | #1286 #1343 #1350 #1449 |
| | | #1534 #1570 #1602 #163 |
| | | #1704 #1919 #2087 #2107 |
| | | #2112 #2128 #2141 #217 |
| | | #2206 #2208 #2320 #2557 |
| | | #2806 #3013 #3142 #3511 |
| | | #367 #371 #3765 #387 #442 |
| | | #471 #473 #508 #667 #668 |
| | | #704 #714 #767 #777 #790 |
| | | #877 #976 #985 |
| 4233G > A | | #1024 #1570 #2107 #2320 |
| 4239C > T | | #2128 #2557 |
| 4239C > A | 679S > R | #2806 |
| 4242G > A | | #1260 |
| 4245G > A | | #1260 #714 #767 |
| 4248C > T | | #2557 #685 |
| 4248C > A | | #3511 |
| 4251G > A | | #1055 #371 |
| 4258C > T | | #2040 #387 |
| 4260G > A | | #1286 #1534 |
| 4263G > A | | #1055 |
| 4266G > A | | #1273 #1343 #1449 #3142 |
| | | #371 #668 #777 |
| 4269A > G | | #1020 #1024 #1055 #1158 |
| | | #1260 #1273 #1286 #129 |
| | | #1343 #1350 #1449 #1534 |
| | | #1570 #1591 #1602 #163 |
| | | #1704 #1919 #2040 #2087 |
| | | #2107 #2112 #2128 #2141 |
| | | #217 #2206 #2208 #2320 |
| | | #2557 #3013 #3086 #3142 |
| | | #3511 #367 #371 #3765 |
| | | #387 #442 #471 #473 #508 |
| | | #667 #668 #685 #704 #714 |
| | | #767 #777 #790 #877 #976 |
| | | #985 #M258 |
| 4278A > G | | #2128 #2208 |
| 4287T > C | | #1024 #129 #1343 #1449 |
| | | #1570 #2087 #2102 #2107 |
| | | #2128 #2208 #2320 #3013 |
| | | #3086 #3142 #3511 #3765 |
| | | #387 #442 #668 #704 #777 |
| | | #790 #M258 |
| 4290C > T | | #2208 #387 |
| 4293A > G | | #1010 #1020 #1024 #1260 |
| | | #1273 #129 #1343 #1449 |
| | | #1534 #1570 #1602 #163 |
| | | #1704 #1919 #2040 #2087 |
| | | #2102 #2107 #2112 #2128 |
| | | #2141 #2206 #2208 #2320 |
| | | #2557 #2806 #3013 #3086 |
| | | #3142 #3511 #367 #371 |
| | | #3765 #387 #442 #471 #473 |
| | | #508 #667 #668 #685 #704 |
| | | #767 #777 #790 #877 #976 |
| | | #985 #M258 |
| 4296T > C | | #1024 #1260 #129 #1343 |
| | | #1449 #1570 #1704 #1919 |
| | | #2040 #2087 #2102 #2107 |
| | | #2112 #2128 #2208 #2320 |
| | | #2497 #2557 #2806 #3013 |
| | | #3086 #3142 #3511 #3765 |
| | | #387 #442 #508 #668 #704 |
| | | #767 #777 #M258 |
| 4299G > A | | #2128 #2206 #471 |
| 4302C > T | | #1158 #1286 #3511 #714 |
| 4308C > A | | #1055 #367 #667 #877 |
| 4308C > T | | #3086 |
| 4311C > T | | #1449 #3142 |
| 4314C > T | | #1260 #1919 #2087 #2208 |
| | | #668 |
| 4320G > A | | #1010 #1017 #1020 #1024 |
| | | #1055 #1158 #1260 #1273 |
| | | #1286 #129 #1449 #1534 |
| | | #1570 #1591 #1602 #163 |
| | | #2040 #2102 #2107 #2128 |
| | | #217 #2206 #2208 #3013 |
| | | #3086 #3142 #3511 #371 |
| | | #3765 #442 #471 #473 #685 |
| | | #714 #790 #976 |
| 4331T > C | 710V > A | #2557 |
| 4336T > C | 712F > L | #2557 |
| 4344G > A | | #1273 |
| 4356C > T | | #1010 #1020 #1024 #1055 |
| | | #1158 #1260 #1273 #1286 |
| | | #129 #1343 #1449 #1534 |
| | | #1570 #1591 #1602 #163 |
| | | #1919 #2102 #2107 #2112 |
| | | #2141 #217 #2206 #2208 |
| | | #2497 #2557 #2806 #3086 |
| | | #3511 #371 #3765 #387 |
| | | #442 #471 #508 #667 #668 |
| | | #685 #704 #714 #777 #790 |
| | | #976 #M258 |
| 4369C > G | 723P > A | #129 |
| 4400G > T | 733R > L | #2102 |
| 4401T > C | | #163 |

An object of the present invention relates to a variant VP1 capsid protein that consists of the amino acid sequence as set forth in SEQ ID NO:2 comprising at least one mutation selected from the group consisting of V151A, Q164N; T200S; N201T; M211V; T233Q; M235L; T410Q; S446N; R447K; N449Q; T450S; P451N; T455L; T456Q; Q461L; A467P; S468T; D469S; I470M; R471S; D472L; S474A; R475K; V488L; T491Q; S492A; A493N; E499N; Y500F; S501P; G504A; Q536M; S537H; V539T; S547T; E548N; K549A; T550N; N551D; V552A; I554L; E555D; K556N; S578Y; T581N; R585N; R585S; G586S; R588T; Q589G; A590P; A591T; A593S; A593G; D594T; T597H; V600A; L647M; S658P; T660N; and A663S.

In some embodiments, the variant VP1 capsid protein of the present invention has an amino acid sequence selected from the group consisting of SEQ ID NO: 3 to SEQ ID NO:61.

A further object of the present invention relates to a variant VP1 capsid protein of the present invention has an amino acid sequence selected from the group consisting of SEQ ID NO:26 (i.e. capsid #3013), SEQ ID NO:9 (i.e. capsid #2087), SEQ ID NO: 20 (i.e. capsid #1449), SEQ ID NO: 10 (i.e. capsid #2206), SEQ ID NO: 12 (i.e. capsid #1534), SEQ ID NO: 56 (i.e. capsid #163), SEQ ID NO: 23 (i.e. capsid #1343), SEQ ID NO: 53 (i.e. capsid #790), SEQ ID NO: 6 (i.e. capsid #1017), SEQ ID NO: 11 (i.e. capsid #877), SEQ ID NO:8 (i.e. capsid #1273), SEQ ID NO: 3 (i.e. capsid #2497), SEQ ID NO: 60 (i.e. capsid #1055), SEQ ID NO:14 (i.e. capsid #1919), SEQ ID NO: 4, SEQ ID NO:5, SEQ ID NO:7, SEQ ID NO:13, SEQ ID NO:15 to SEQ ID NO:19, SEQ ID NO:21, SEQ ID NO:22, SEQ ID NO:24, SEQ ID NO:25, SEQ ID NO:27 to SEQ ID NO:55, SEQ ID NO:57 to SEQ ID NO:59, SEQ ID NO:61.

SEQ ID NO: 3
>#2497_AAV.FL.linear_-_major_coat_protein_VP1_translation
CHC2497N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLSRTNTPSGTTTQSRLQFSQAGASDIRDQSRNWLPG
PCYRQQRVSKTAADNSNSEYSWTGATKYHLNGRDSLVNPGPAMASHKDDEEKFFPHSGVLIFGKQGSEKTNVDIEKVMIT
DEEEIRTTNPVATEQYGTVSTNLQSGNTQAATSDVNTQGILPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 4
>#2102_AAV.FL.linear_-_major_coat_protein_VP1_translation
CHC2102N_Adeno-Associated virus isolates in liver tumor, linear form
MAADGYLPDWLEDTLSEGIRQWWKLKPGPPPPKPAERHQDDSRGLVLPGYKYLGPFNGLDKGEPVNEADAAALEHDKAYD
RQLESGDNPYLKYNHADAEFQERLKEDTSFGGNLGRAVFQAKKRVLEPLGLVEEPVKTAPGKKRPVEHSPVEPDSSSGTG
KAGNQPARKRLNFGQTGDADSVPDPQPLGQPPAAPSGLGTNTMATGSGAPMADNNEGADGVGNSSGNWHCDSTWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSGTNTPSGTTTQSRLQFSQAGASDIRDQSRNWLPG
PCYRQQRVSKTAADNSNSEYSWTGATKYHLNGRDSLVNPGPAMASHKDDEEKFFPQSGVLIFGKQGSEKTNVDIEKVMIT
DEEEIRTTNPVATEQYGSVSTNLQSGNTQAATSDVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTLNL*
SEQ ID NO: 5
>#371_AAV.FL.linear_-_major_coat_protein_VP1_translation
CHC371N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTSADNNNSEYSWTGATKYHLNGRDSLVNPGPAMASHKDDEEKFFPQSGVLIFGKQGSEKTNVDIEKVMIT
DEEEIRTTNPVATEQYGSVSTNLQSGNTQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 6
>#1017_AAV.FL.linear_-_major_coat_protein_VP1_translation
CHC1017N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGTNTMASGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTSADNNNSEYSWTGATKYHLNGRDSLVNPGPAMASHKDDEEKFFPQSGVLIFGKQDSGKTNVDIEKVMIT
DEEEIRTTNPVATEQYGAVSTNLQSGNTQAATSDVNAQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 7
>#473_AAV.FL.linear_-_major_coat_protein_VP1_translation
CHC473N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGQQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTAADNNNSEYSWTGATKYHLNGRDSLVNPGPAMASHKDDEEKFFPQSGVLIFGKQDSGKTNVDIEKVMIT
DEEEIRTTNPVATEQYGAVSTNLQSGNTQAATSDVNAQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 8
>#1273_AAV.FL.linear_-_major_coat_protein_VP1_translation
CHC1273N_Adeno-Associated virus isolates in liver tumor, linear form
MAADGYLPDWLEDTLSEGIRQWWKLKPGPPPPKPAERHQDDSRGLVLPGYKYLGPFNGLDKGEPVNEADAAALEHDKAYD
RQLDSGDNPYLKYNHADAEFQDRLKEDTSFGGNLGRAVFQAKKRVLEPLGLVEEPVKTAPGKKRPVEHSPVEPDSSSGTG
KAGQQPARKRLQTGDADSVPDPQPLGQPPAAPSGLGTNTMATGSGAPMADNNEGADGVGNSSGNWHCDSTWMGDRVITTS
TRTWALPTYNNHLYKQISSQSGASNDNHYFGYSTPWGYFDFNRFHCHFSPRDWQRLINNNWGFRPKRLNFKLFNIQVKEV
TQNDGTTTIANNLTSTVQVFTDSEYQLPYVLGSAHQGCLPPFPADVFMVPQYGYLTLNNGSQAVGRSSFYCLEYFPSQML
RTGNNFTFSYTFEDVPFHSSYAHSQSLDRLMNPLIDQYLYYLSRTNTPSGTTTQSRLQFSQAGASDIRDQSRNWLPGPCY
RQQRVSKTAADNNNSEYSWTGATKYHLNGRDSLVNPGPAMASHKDDEEKFFPQSGVLIFGKQDSGKTNVDIEKVMITDEE
EIRTTNPVATEQYGAVSTNLQSGNTQAATSDVNAQGVLPGMVWQDRDVYLQGPIWAKIPHTDGHFHPSPLMGGFGLKHPP
PQILIKNTPVPANPSTTFSAAKFASFITQYSTGQVSVEIEWELQKENSKRWNPEIQYTSNYNKSVNVDFTVDTNGVYSEP
RPIGTRYLTRNL*
SEQ ID NO: 9
>#2087_AAV.FL.linear_-_major_coat_protein_VP1_translation
CHC2087N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGNQPARKRLNFGQTGDADSVPDPQPLGQPPAAPSGLGTNTMASGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTAADNNNSEYSWTGATKYHLNGRDSLVNPGPAMASHKDDEEKFFPQSGVLIFGKQGSEKTNVDIEKVMIT
DEEEIRTTNPVATEQYGAVSTNLQSGNTQAATSDVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 10
>#2206_AAV.FL.linear_-_major_coat_protein_VP1_translation
CHC2206N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGNQPARKRLNFGQTGDADSVPDPQPLGQPPAAPSGLGTNTMASGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTSADNNNSEYSWTGATKYHLNGRDSLVNPGPAMASHKDDEEKFFPQSGVLIFGKQGSEKTNVDIEKVMIT
DEEEIRTTNPVATEQYGAVSTNLQSGNTQAATSDVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 11
>#877_AAV.FL.linear_-_major_coat_protein_VP1_translation
CHC877N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGNQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTAADNNNSEYSWTGATKYHLNGRDSLVNPGPAMASHKDDEEKFFPQSGVLIFGKQGSEKTNVDIEKVMIT
DEEEIRTTNPVATEQYGSVSTNLQSGNTQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 12
>#1534_AAV.FL.linear_-_major_coat_protein_VP1_translation
CHC1534N_Adeno-Associated virus isolates in liver tumor, linear form
MAADGYLPDWLEDTLSEGIRQWWKLKPGPPPPKPAERHQDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGNQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTAADNNNSEYSWTGATKYHLNGRDSLVNPGPAMASHKDDEEKFFPQSGVLIFGKQGSEKTNVDIEKVMIT
DEEEIRTTNPVATEQYGSVSTNLQSGNTQAATSDVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 13
>#2557_AAV.FL.linear_-_major_coat_protein_VP1_translation
CHC2557N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGNQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTAADNNNSEYSWTGATKYHLNGRDSLVNPGPAMASHKDDEEKFFPQSGVLIFGKQGSEKTNVDIEKVMIT
DEEEIRTTNPVATEQYGSVSTNLQSGNTQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNADLTVDTNGVY
SEPRPIGTRYLTRNL*
SEQ ID NO: 14
>#1919_AAV.FL.linear_-_major_coat_protein_VP1_translation
CHC1919N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGNQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTAADNNNSEYSWTGATKYHLNGRDSLVNPGPAMASHKDDEEKFFPQSGVLIFGKQGSEKTNVDIEKVMIT
DEEEIRTTNPVATEQYGAVSTNLQSGNTQAATSDVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 15
>#1260_AAV.FL.linear_-_major_coat_protein_VP1_translation
CHC1260N_Adeno-Associated virus isolates in liver tumor, linear form
MAADGYLPDWLEDTLSEGIRQWWKLKPGPPPPKPAERHKDDGRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGQQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTSADNNNSEYSWTGATKYHLNGRDSLVNPGPAMASHKDDEEKFFPQSGVLIFGKQGSEKTNVDIEKVMIT
DEEEIRTTNPVATEQYGAVSTNLQSGNTQAATSDVNAQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 16
>#3765_AAV.FL.linear_-_major_coat_protein_VP1_translation
CHC3765N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGNQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTPADNNNSEYSWTGATKYHLNGRDSLVNPGPAMASHKDDEEKFFPQSGVLIFGKQGSEKTNVDIEKVMIT
DEEEIRTTNPVATEQYGSVSTNLQSGNTQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 17
>#471_AAV.FL.linear_-_major_coat_protein_VP1_translation
CHC471N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGNQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTPADNNNSEYSWTGATKYHLNGRDSLVNPGPAMASHKDDEEKFFPQSGVLIFGKQGSEKTNVDIEKVMIT
DEEEIRTTNPVATEQYGSVSTNLQSGNTQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 18
>#387_AAV.FL.linear_-_major_coat_protein_VP1_translation
CHC387N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KSGNQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTPADNNNSEYSWTGATKYHLNGRDSLVNPGPAMASHKDDEEKFFPQSGVLIFGKQGSEKTNVDIEKVMIT
DEEEIRTTNPVATEQYGSVSTNLQSGNTQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 19
>#2040_AAV.FL.linear_-_major_coat_protein_VP1_translation
CHC2040N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGTTTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTAADNNNSEYSWTGATKYHLNGRDSLVNPGPAMASHKDDEEKFFPQSGVLIFGKQGSEKTNVDIEKVMIT
DEEEIRTTNPVATEQYGSVSTNLQSGNTQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 20
>#1449_AAV.FL.linear_-_major_coat_protein_VP1_translation
CHC1449N_Adeno-Associated virus isolates in liver tumor, linear form
MAADGYLPDWLEDTLSEGIRQWWKLKPGPPPPKPAERHKDDNRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KSGQQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTPADNNNSEYSWTGATKYHLNGRDSLVNPGPAMASHKDDEEKFFPQSGVLIFGKQGSEKTNVDIEKVMIT
DEEEIRTTNPVATEQYGTVSTNLQSGNTQAATSDVNTQGILPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 21
>#2128_AAV.FL.linear_-_major_coat_protein_VP1_translation
CHC2128N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGNQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTSADNNNSEYSWTGATKYHLNGRDSLVNPGPAMASHKDDEEKFFPQSGVLIFGKQGSEKTNVEKVMITDE
EEIRTTNPVATEQYGAVSTNLQSGNTQAATADVNTQGVLPGMVWQDRDVYLQGPIWAKIPHTDGHFHPSPLMGGFGLKHP
PPQILIKNTPVPANPSTTFSAAKFASFITQYSTGQVSVEIEWELQKENSKRWNPEIQYTSNYNKSVNVDFTVDTNGVYSE
PRPIGTRYLTRNL*
SEQ ID NO: 22
>#2141_AAV.FL.linear_-_major_coat_protein_VP1_translation
CHC2141N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGNQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFTSRYAHSQSLDRLMNPLIDQYLYYLSRTNTPSGTTTQSRLQFSQAGASDIRDQSRNWLPG
PCYRQQRVSKTSADNNNSEYSWTGATKYHLNGRDSLVNPGPAMASHKDDEEKFFPQSGVLIFGKQGSEKTNVDIEKVMIT
DGEEIRTTNPVATEQYGSVSTNLQRGNTQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 23
>#1343_AAV.FL.linear_-_major_coat_protein_VP1_translation
CHC1343N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSKTNAPSGTTTMSRLQFSQAGASDIRDQSRNWLPG
PCYRQQRVSKTAADNNNSDYSWTGATKYHLNGRDSLVNPGTAMASHKDDEEKYFPQSGVLIFGKQDSGKTNVDIEKVMIT
DEEEIRTTNPVATEQYGSVSTNLQSGNTQAATSDVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 24
>#777_AAV.FL.linear_-_major_coat_protein_VP1_translation
CHC777N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSKTNAPSGTTTMSRLQFSQAGASDIRDQSRNWLPG
PCYRQQRVSKTAADNNNSDYSWTGATKYHLNGRDSLVNPGTAMASHKDDEEKYFPQSGVLIFGKQDSGKTNVDIEKVMIT
DEEEIRTTNPVATEQYGSVSTNLQSGNTQAATSDVNTQGVLSGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 25
>#3511_AAV.FL.linear_-_major_coat_protein_VP1_translation
CHC3511N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KSGQQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLSFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTMSRLQFSQAGASDIRDQSRNWLPG
PCYRQQRVSKTAADNNNSDYSWTGATKYHLNGRDSLVNPGPAMASHKDDEEKYFPQSGVLIFGKQDSGKTNVDIEKVMIT
DEEEIRTTNPVATEQYGSVSTNLQSGNTQAATSDVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 26
>#3013_AAV.FL.linear_-_major_coat_protein_VP1_translation
CHC3013N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGQQPARKRLNFGQTGDADSVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTMSRLQFSQAGASDIRDQSRNWLPG
PCYRQQRVSKTAADNNNSDYSWTGATKYHLNGRDSLVNPGPAMASHKDDEEKYFPQSGVLIFGKQDSGKTNVDIEKVMIT
DEEEIRTTNPVATEQYGSVSTNLQSGNTQAATSDVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 27
>#2208_AAV.FL.linear_-_major_coat_protein_VP1_translation
CHC2208N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KSGQQPARKRLNFGQTGDADSVPDPQPLGEPPAAPTSLGSTTMASGGGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCPPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTAADNNNSEYSWTGATKYHLNGRDSLVNPGPAMASHKDDEEKFFPQSGVLIFGKQGSEKTNVDIEKVMIT
DEEEIRTTNPVATEQYGSVSTNLQSGNTQAATSDVNTQGVLPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 28
>#129_AAV.FL.linear_-_major_coat_protein_VP1_translation
CHC129N_Adeno-Associated virus isolates in liver tumor, linear form
MAADGYLPDWLEDTLSEGIRQWWKLKPGPPPPKPAERHQDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGQQPARKRLNFGQTGDADSVPDPQPLGQPPAAPTSLGSTTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLNRTQTXSGTPTQSRLLFSQAGPTSMSLQAKNWLPG
PCYRQQRLSKQANDNNNSNFPWTGATKYHLNGRDSLVNPGPAMASHKDDEEKFFPMHGTLIFGKQGSEKTNAELENVMIT
DEEEIRTTNPVATEQYGSVSTNLQRGNRQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEARPIGTRYLTRNL*
SEQ ID NO: 29
>#2731_AAV.FL.linear_-_major_coat_protein_VP1_translation
CHC2731N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEERVKTAPGKKRPVAVAHQWQTITRVPM
EWVIPQEKRLNFGQTGDADWHCDPQPLGQPPAAPSGLGSTTMATGSGALLLLESLSADGVGNSSGNWHCDSQWLGDRVIT
TSTRTWALPTYNNHLYKQISSQSGASNDNHYFGYSTPWGYFDFNRFHCHFSPRDWQRLINNNWGFRPKRLNFKLFNIQVK
EVTQNDGTTTIANNLTSTVQVFTDSEYQLPYVLGSAHQGCLPPFPADVFMVPQYGYLTLNNGSQAVGRSSFYCLEYFPSQ
MLRTGNNFQFSYTFEDVPFHSSYAHSQSLDRLMNPLIDQYLYYLNKTQSNSGTTTMSRLLFSQAGPTSMSLHAKNWLPGP
CYRQQRLSKQANDNNNSNFPWTAATKYHLNGSDSLINPGPAMASHKDDEEKFFPQSGVLIFGKQGSEKTNVDIEKVMITD
EEEIRTTNPVATEQYGSVSTNLQRGNRRAATADVNTQGVLPGMVWQDRDVYLQGPIWAKIPHTDGHFHPSPLMGGFGLKH
PPPQILIKNTPVPANPSTTFSAAKFASFITQYSTGQVSVEIEWELQKENSKRWNPEIQYTSNYNKSVNVDFTVDTNGVYS
EPRPIGTRYLTRNL*
SEQ ID NO: 30
>#1602_AAV.FL.linear_-_major_coat_protein_VP1_translation
CHC1620N_Adeno-Associated virus isolates in liver tumor, linear form
MAADGYLPDWLEDTLSEGIRQWWKLKPGPPPPKSAERHKDDSRGLVLPGYKYLGPFXGLDKGEPVNEADAAALEHDKAYD
RQLDSGDNPYLKYNHADAEFQERLKEDTSFGGNLGRAVFQAKKRVLXPLGXVEEPVKTAPGKKRPVEHSPVEPDSSSGTG
KAGQQPARKRLNFGQTGDSDSSSRDMRKVESAAAPTSLGSTTMASGGGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQTNSGTLQQSRLLFSQAGPTNMSLQAKNWLPG
PCYRQQRLSKQANDNNNSNFPWTAATKYHLNGRDSLVNPGPAMASHKDDEEKFFPQSGVLIFGKQGSEKTNVDIEKVMIT
DEEEIRTTNPVATEQYGSVSTNLQRGNRQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKEXSKRWNPEIQYTSNYNKSVNVDFTVDTNGVY
SEPRPIGTRYLTRNL*
SEQ ID NO: 31
>#1010_AAV.FL.linear_-_major_coat_protein_VP1_translation
CHC1010N_Adeno-Associated virus isolates in liver tumor, linear form
MAADGYLPDWLGETLSQGIRQWWKLKPGPPPPKPAERHKXDSRGLVLPGYKYLGPFNGLDKGEPVNEADAAALEHDKAYD
RQLDSGHIPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGTTTMATGSGAPVADNNEGADGVGNSSGNWHCDSQWLGDRVI
TTSTRTWALPTYNNHLYNFFQIKINASNDNHYFGYSTPWGYFDFNRFHCHFSPRDWQRLINNNWGFRPKRLNFKLFLIQV
KEVTQNDGTTTIANNLTSTVQVFTXSEYQLPYVLGSAHQGCXPPFPADVFMVPQYGYLTLNNGSQAXGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRVSKTSADNNNSEYSWTGATKYHLNGRDSLVNPGPAMASHKDDEEKFFPQSGVLIFGKQGSEKTNVDIEKVMIT
DEEEIRTTNPVATEQYGYVSNNLQNSNRQAATADVNTQGVLPGMVWQDRDVYLQGPIWAKIPHTDGHFHPSPLMGGFGLK
HPPPQILIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 32
>#985_AAV.FL.linear_-_major_coat_protein_VP1_translation
CHC985N_Adeno-Associated virus isolates in liver tumor, linear form
MAADGYLPDWLEDTPSAGISQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGTNTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKKLNFKLFNIQV
KEVTQNDGTTTIANNLTSTVQVFTGSGYGLRYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLQFSQAGASDIRDQSRNWLPG
PCYRQQRVSKTSADNNNSEYSWTGATKYHLNGRDSLVNPGPAMASHKDDEEKFFPQSGVLIFGKQGSEKTNVDIEKVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 33
>#667_AAV.FL.linear_-_major_coat_protein_VP1_translation
CHC667N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGTNTMATGSGAPMEDNNEGADGVGNSSGNWHCDSQWLGDRVI
TTSTRTWALPTYNNHLYKQICSQSGASNDNHYFGYSTRWGDFDFNRFHCHFSPRDWQRLINNNWGFRPKRLNFKLFNIQV
KEVTQNDGTXTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLLFSQAGPTSMSLQAKNWLPG
PCYRQQRLSKQANDNNNSNFPWTAATKYHLNGRDSLVNPGPAMASHKDDEEKFFPQSGVLIFGKQGSEKTNVDIEKVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKSSXVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 34
>#2806_AAV.FL.linear_-_major_coat_protein_VP1_translation
CHC2806N_Adeno-Associated virus isolates in liver tumor, linear form
MAADGYLPDWLEDTLSEGIRQWWKLKPGPPPPKPAERHKDNSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPAKKRLKFGQTGDADSVPDPQPLGQPPAAPSGLGSTTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLNKTQSNSETTFSSATPLKTFLSTAATLTARVWLPG
PCYRQQRLSKQANDNNNSNFPWTAATKYHLNGRDSLVNPGPAMASHKDDEEKFFPQSGVLIFGKQGSEKTNVDIEKVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVRVEIEWELQKENSKRWNPEIQYTSNYNKXVNVDFTVDTNGVY
SEXRPIXTRXLTRNL*
SEQ ID NO: 35
>#217_AAV.FL.linear_-_major_coat_protein_VP1_translation
CHC217N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTKTPANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLNKTQSNSGTNTQSRLQFSQAGASDIRDQSRNWLPG
PCYRQQRLSKQANDNNNSNFPWTGATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 36
>#767_AAV.FL.linear_-_major_coat_protein_VP1_translation
CHC767N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGQQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNHYFGYSTPWGYFDFNRFHCHFSPRDWQRLINNNWGFRPKRLNFKLFNIQVKE
VTQNDGTTTIANNLTSTVQVFTDSEYQLPYVLGSAHQGCLPPFPADVFMVPQYGYLTLNNGSQAVGRSSFYCLEYFPSQM
LRTGNNFTFSYTFEDVPFHSSYAHSQSLDRLMNPLIDQYLYYLNKTQSSGTLQQSRLLFSQAGPTSMSLQARNWLPGPCY
RQQRVSKTSADKNNSEYSWTAATKYHLNGRDSLVNPGPAMASHKDDEEKFFPMPGTLIFGKEGTNANNAELENVMITDEE
EIRTTNPVATEQYGYVSNNLQNSNTGPTTGTVNHQGALPGMVWQDRDVYLQGPIWAKIPHTDGHFHPSPLMGGFGLKHPP
PQIMIKNTPVPANPPTNFSAAKFASFITQYSTGQVSVEIEWELQKENSKRWNPEIQYTSNYNKSVNVDFTVDTNGVYSEP
RPIGTRYLTRNL*
SEQ ID NO: 37
>#1591_AAV.FL.linear_-_major_coat_protein_VP1_translation
CHC1591N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGQQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGASDIRDQSRNWLPG
PCYRQQRVSKTSADNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 38
>#508_AAV.FL.linear_-_major_coat_protein_VP1_translation
CHC508N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAAIEHDKAYD
RQLDSGDNPYLKYNHADAEFQERLKEDTSFGGNLGRAVFQAKKRVLEXXGLVEEPVKTAPGKKRPVAHSPAEPDSSSGTG
KAGNQPARKRLNFGQTGDADSVPDPQPLGQPPAAPTSLGSTTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNRTQTASGTQQSRLLFSQAGPTSMSLQAKNWLPGP
CYRQQRLSKQANDNNNSNYSWTGATKYHLNGRDSLVXPGPAMASHXDDEXKFFPMHGTLIFGKEGTNATNADLDNVMITD
EEEIRTTNPVATEQYGYVSNNLQNSNTAATTETVNHQGALPGMVWQDRDVYLQGPIWAKIPHTDGHFHPSPLMGGFGLKN
PPPQIMIKNTPVPANPPTNFSSAKFASFITQYSTGQVSVEIEWELQKENSKRWNPEIQYTSNYNKSVNVDFTVDTNGVYS
EPRPIGTRYLTRNL*
SEQ ID NO: 39
>#1704_AAV.FL.linear_-_major_coat_protein_VP1_translation
CHC1704N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLARAVFQAKKRVLEPLGLVEEPVKTAPGKKRPVEHSPAEPDSSSGTG
KSGNQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLNKTQTNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLENVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 40
>#2112_AAV.FL.linear_-_major_coat_protein_VP1_translation
CHC2112N_Adeno-Associated virus isolates in liver tumor, linear form
MAADGYLPDWLQENLFFDIRQWWKLKPGPPPPKPAERHQDNSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGXQPAXKRLNFGQTGXADSVPDPQPLGQPPAAPSGLGTNTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNKAGKRQQQQQLSLDCGYKVSTKWPGPAMASHKDDEEKFFPMHGTLIFGKQGTNANDADLDNVMI
TDEEEIRTTNPVATEQYGYVSNNLQNSNTGPTTGTVNHQGALPGMVWQDRDVYLQGPIWAKIPHTDGHFHPSPLMGGFGL
KHPPPQIMIKNTPVPANPPTNFSSAKFASFITQYSTGQVSVEIEWELQKENSKRWNPEIQYTSNYNKSVNVDFTVDTNGV
YSEPRPIGTRYLTRNL*
SEQ ID NO: 41
>#1350_AAV.FL.linear_-_major_coat_protein_VP1_translation
CHC1350N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQSGNTQAATGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 42
>#668_AAV.FL.linear_-_major_coat_protein_VP1_translation
CHC668N_Adeno-Associated virus isolates in liver tumor, linear form
MAADGYLPDWLEDTLSEGIRQWWKLKPGPPPPKPAERHKDDNRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 43
>#367_AAV.FL.linear_-_major_coat_protein_VP1_translation
CHC367N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 44
>#1020_AAV.FL.linear_-_major_coat_protein_VP1_translation
CHC1020N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLENVMIT
DEEEIRTTNPVATEQYGTVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 45
>#1158_AAV.FL.linear_-_major_coat_protein_VP1_translation
CHC1158N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGKPPAAPSGLGSTTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSSKFASFITQYSTGQVSVEIEWELQKENSKRWNPEIQYTSNYNKSVNVDFTVDTNGVY
SEPRPIGTRYLTRNL*
SEQ ID NO: 46
>#2107_AAV.FL.linear_-_major_coat_protein_VP1_translation
CHC2107N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 47
>#1570_AAV.FL.linear_-_major_coat_protein_VP1_translation
CHC1570N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQANSGTLQQSRLLFSQAGPTSMSLQAKNWLPG
PCYRQQRLSKQANDNNNSNFPWTAATKYHLNGRDSLVNPGPAMASHKDDEEEKFFPMHGTLIFGKQGTNANDADLDNVMI
TDEEEIRTTNPVATEQYXYVSNNLQNSNTGPTTGTVNHQGALPGMVWQDRDVYLQGPIWAKIPHTDGHFHPSPLMGGFGL
KHPPPQIMIKNTPVPSPLMGGFGLKHPPPQITQYSTGQVSVEIEWELQKENSKRWNPEIQYTSNYNKSVNVDFTVDTNGV
YSEPRPIGTRYLTRNL*
SEQ ID NO: 48
>#704_AAV.FL.linear_-_major_coat_protein_VP1_translation
CHC704N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 49
>#714_AAV.FL.linear_-_major_coat_protein_VP1_translation
CHC714N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGIG
KAGQQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 50
>#3086_AAV.FL.linear_-_major_coat_protein_VP1_translation
CHC3086N_Adeno-Associated virus isolates in liver tumor, linear form
MAADGYLPDWLEDTLSEGIRQWWKLKPGPPPPKPAERHKDNSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPAKKRLNFGQTGDADSVPDPQPLGQPPAAPSGLGSTTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTNMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 51
>#3142_AAV.FL.linear_-_major_coat_protein_VP1_translation
CHC2143N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGNQPARKRLNFGQTGDADSVPDPQPIGQPPAAPTSLGSTTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTVQQSRLLFSQAGPTSMSLQAKNWLPG
PCYRQQRLSKQASDNNNSNFPWTAATKYHLNGRDSLVNPGPAMASHKDDEEKFFPMHGTLIFGKEGTTSNNADLEHVMIT
DEEEIRTTNPVATEQYGNVSNNLQNSNTGPTTENVNNQGALPGMVWQDRDVYLQGPIWAKIPHTDGHFHPSPLMGGFGLK
HPPPQIMIKNTPVPANPPTNFSAAKFASFITQYSTGQVSVEIEWELQKENSKRWNPEIQYTSNYNKSVNVDFTVDTNGVY
SEPRPIGTRYLTRNL*
SEQ ID NO: 52
>#1024_AAV.FL.linear_-_major_coat_protein_VP1_translation
CHC1024N_Adeno-Associated virus isolates in liver tumor, linear form
MAADGYLPDWLEDNLSEGIREWWALKPGAPQPKANQQHQDNARGLVLPGYKYLGPGNGLDKGEPVNAADAAALEHDKAYD
KQLKAGDNPYLKYNHADAEFQERLKEDTSFGGNLGRAVFQAKKRVLEPLGLVEEPVKTAPGKKRPVEHSPAEPDSSSGTG
KSGQQPARKRLNFGQTGDADSVPDPQPLGQPPAAPSGLGSTTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNRTQTASGTQQSRLLFSQAGPTSMSLQAKNWLPGP
CYRQQRLSKQANDNNNSNFPWTAATKYHLNGRDSLVNPGPAMASHKDDEEKFFPMHGTLIFGKEGTNATNADLDNVMITD
EEEIRTTNPVATEQYGYVSNNLQNSNTAATTETVNHQGALPGMVWQDRDVYLQGPIWAKIPHTDGHFHPSPLMGGFGLKH
PPPQIMIKNTPVPANPPTNFSAAKFASFITQYSTGQVSVEIEWELQKENSKRWNPEIQYTSNYNKSVNVDFTVDTNGVYS
EPRPIGTRYLTRNL*
SEQ ID NO: 53
>#790_AAV.FL.linear_-_major_coat_protein_VP1_translation
CHC790N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGNQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 54
>#976_AAV.FL.linear_-_major_coat_protein_VP1_translation
CHC976N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 55
>#1286_AAV.FL.linear_-_major_coat_protein_VP1_translation
CHC1286N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO: 56
>#163_AAV.FL.linear_-_major_coat_protein_VP1_translation
CHC163N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPVADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKKLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO : 57
>#685_AAV.FL.linear_-_major_coat_protein_VP1_translation
CHC685N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGQQPARKRLNFGQTGDADSVPDPQPLGQPPAAPSGLGSTTMATGSGAPMADNNEGADGVGNSSGNWHCDSQWLGNRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTNANDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO : 58
>#442_AAV.FL.linear_-_major_coat_protein_VP1_translation
CHC442N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGDADSVPDPQPLGQPPAAPSGLGSTTMATGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQANDNNNSNFPWTAATKYHLNGRDSLVNPGPAMASHKDDEEKFFPMHGTLIFGKQGTGRDNADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO : 59
>#2320_AAV.FL.linear_-_major_coat_protein_VP1_translation
CHC2320N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLETLGLVEEHVKTAPGKKRAVEHAHVEPDSSSGTG
KAGQQPARKRLNFGQTGDADSVPDPQPLGQPPAAPXGLGSTTMASGSGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNKTQSNSGTLQQSRLLFSQAGPTSMSLQAKNWLPG
PCYRQQRLSKQATDNNNSEFPWTAATKYHLNGRDSLVNPGPAMASHKDDEEKFFPMHGTLIFGKQGTKCNDADLDNVMIT
DEEEIRTTNPVATEQYGYVSNNLQNSNTGPTTGTVNHQGALPGMVWQDRDVYLQGPIWAKIPHTDGHFHPSPLMGGFGLK
HPPPQIMIKNTPVPANPPTNFSSAKFASFITQYSTGQVSVEIEWELQKENSKRWNPEIQYTSNYNKSVNVDFTVDTNGVY
SEPRPIGTRYLTRNL*
SEQ ID NO : 60
>#1055_AAV.linear_-_major_coat_protein_VP1_translation
CHC1055N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPVEPDSSSGTG
KAGQQPARKRLNFGQTGPYASVPDPQPLGQPPAAPSGLGTNTMATGSGAPMADNNEGADGVGNSSGNWHCDSTWMGDRVI
TTSTRTWALPTYNNHLYKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNTPSGTTTQSRLQFSQAGASDIRDQSRNWLPG
PCYRQQRVSKTAADNNNSEYSWTGATKYHLNGRDSLVNPGPAMASHKDDEEKFFPQSGVLIFGKQGSEKTNVDIEKVMIT
DEEEIRTTNPVATEQYGAVSTNLQSGNTQAATSDVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDTNGVY
SEPRPIGTRYLTRNL*
SEQ ID NO: 61
>#M258_AAV.linear_-_major_coat_protein_VP1_translation
BCM258N_Adeno-Associated virus isolates in liver tumor, linear form
MAADGYLPDWLEDTLSEGIRQWWKLKPGPPPPKPAERHKDDSRGLVLPGYKYLGPFNGLDKGEPVNEADAAALEHDKAYD
RQLDSGDNPYLKYNHADAEFQERLKEDTSFGGNLGRAVFQAKKRVLEPLGLVEEPVKTAPGKKRPVEHSPAEPDSSSGTG
KAGNQPARKRLNFGQTGDADSVPDPQPLGEPPAAPTSLGSTTMASGGGAPVADNNEGADGVGNSSGKLALRFPMAGRQSH
YHQHPNLGPAHLQQPSVQANFQPIRSQQRQPLILAHCNPWGYFDFNRFHCHFSPRDWQRLINNNWGFRPKRLNFKLFNIQ
VKEVTQNDGTTTIANNLTSTVQVFTDSEYQIPYVLGSAHQGCLPPFPADVFMVPQYGYLTLNNGSQAVGRSSFYCLEYFP
SQMLRTGNNFQFSYTFEDVPFHSSYAHSQSLDRLMNPLIDQYLYYLNKTQSASGTVQQSRLLFSQAGPTSMSLQAKNWLP
GPCYRQQRLSKQANENNNSNFPWTAATKYHLNGRDSLVNPGPAMASHKDDEEKFFPMHGTLIFGKQGTGRDNADLDNVMI
TDEEEIRTTNPVATEQYGYVSNNLQNSNTAPSTGTVNHQGALPGMVWQDRDVYLQGPIWAKIPHTDGHFHPSPLMGGFGL
KHPPPQIMIKNTPVPANPPTNFSAAKFASFITQYSTGQVSVEIEWELQKENSKRWNPEIQYTSNYNKSVNVDFTVDTNGV
YSEPRPIGTRYLTRNL*
SEQ ID NO: 121
>consensus_AAV2
MAADGYLPDW LEDTLSEGIR QWWKLKPGPP PPKPAERHkD DSRGLVLPGY KYLGPFNGLD
KGEPVNEADA AALEHDKAYD RQLDSGDNPY LKYNHADAEF QeRLKEDTSF GGNLGRAVFQ
AKKRVLEPLG LVEEPVKTAP GKKRPVEHSP vEPDSSSGTG KaGqQPARKR LnfgQTGDAD
SVPDPQPLGq PPAAPsgLGt nTMAtGsGAP mADNNEGADG VGNSSGNWHC DStWmGDRVI
TTSTRTWALP TYNNHLYKQI SSQSGASNDN HYFGYSTPWG YFDFNRFHCH FSPRDWQRLI
NNNWGFRPKR LNFKLFNIQV KEVTQNDGTT TIANNLTSTV QVFTDSEYQL PYVLGSAHQG
ClPPFPADVF MVPQYGYLTL NNGSQAVGRS SFYCLEYFPS QMLRTGNNFT FSYTFEDVPF
HSSYAHSQSL DRLMNPLIDQ YLYYLSrTNT PSGTTTqSRL QFSQAGASDI RDQSRNWLPG
PCYRQQRVSK TaADNnNSeY SWTGATKYHL NGRDSLVNPG PAMASHKDDE EKfFPQSGVL
IFGKQgSeKT NVDIEKVMIT DEEEIRTTNP VATEQYGsVS TNLQSGNTQA ATsDVNtQGV
LPGMVWQDRD VYLQGPIWAK IPHTDGHFHP SPLMGGFGLK HPPPQIlIKN TPVPANPsTt
FSaAKFASFI TQYSTGQVSV EIEWELQKEN SKRWNPEIQY TSNYNKSVNV DFTVDTNGVY
SEPRPIGTRY LTRNL

A further object of the present invention relates to a variant VP2 capsid protein that consists of the amino acid sequence that ranges from the amino acid at position 138 to the last amino acid in SEQ ID NO:2 comprising at least one mutation selected from the group consisting of V151A, Q164N; T200S; N201T; M211V; T233Q; M235L; T410Q; S446N; R447K; N449Q; T450S; P451N; T455L; T456Q; Q461L; A467P; S468T; D469S; I470M; R471S; D472L; S474A; R475K; V488L; T491Q; S492A; A493N; E499N; Y500F; S501P; G504A; Q536M; S537H; V539T; S547T; E548N; K549A; T550N; N551D; V552A; I554L; E555D; K556N; S578Y; T581N; R585N; R585S; G586S; R588T; Q589G; A590P; A591T; A593S; A593G; D594T; T597H; V600A; L647M; S658P; T660N; and A663S

In some embodiments, the variant VP2 capsid protein of the present invention has an amino acid sequence that ranges from the amino acid at position 138 to the last amino acid in SEQ ID NO:3 to SEQ ID NO:61.

A further object of the present invention relates to a variant VP2 capsid protein of the present invention has an amino acid sequence that ranges from the amino acid at position 138 to the last amino acid in SEQ ID NO:26 (i.e. capsid #3013), SEQ ID NO:9 (i.e. capsid #2087), SEQ ID NO: 20 (i.e. capsid #1449), SEQ ID NO: 10 (i.e. capsid #2206), SEQ ID NO: 12 (i.e. capsid #1534), SEQ ID NO: 56 (i.e. capsid #163), SEQ ID NO: 23 (i.e. capsid #1343), SEQ ID NO: 53 (i.e. capsid #790), SEQ ID NO: 6 (i.e. capsid #1017), SEQ ID NO: 11 (i.e. capsid #877), SEQ ID NO:8 (i.e. capsid #1273), SEQ ID NO: 3 (i.e. capsid #2497), SEQ ID NO: 60 (i.e. capsid #1055), SEQ ID NO:14 (i.e. capsid #1919), SEQ ID NO: 4, SEQ ID NO:5, SEQ ID NO:7, SEQ ID NO:13, SEQ ID NO:15 to SEQ ID NO:19, SEQ ID NO:21, SEQ ID NO:22, SEQ ID NO:24, SEQ ID NO:25, SEQ ID NO:27 to SEQ ID NO:55, SEQ ID NO:57 to SEQ ID NO:59, SEQ ID NO:61.

A further object of the present invention relates to a variant VP3 capsid protein that consists of the amino acid sequence that ranges from the amino acid at position 203 to the last amino acid in SEQ ID NO:2 comprising at least one mutation selected from the group consisting of M211V; T233Q; M235L; T410Q; S446N; R447K; N449Q; T450S; P451N; T455L; T456Q; Q461L; A467P; S468T; D469S; I470M; R471S; D472L; S474A; R475K; V488L; T491Q; S492A; A493N; E499N; Y500F; S501P; G504A; Q536M; S537H; V539T; S547T; E548N; K549A; T550N; N551D; V552A; I554L; E555D; K556N; S578Y; T581N; R585N; R585S; G586S; R588T; Q589G; A590P; A591T; A593S; A593G; D594T; T597H; V600A; L647M; S658P; T660N; and A663S

In some embodiments, the variant VP3 capsid protein of the present invention has an amino acid sequence that ranges from the amino acid at position 203 to the last amino acid in SEQ ID NO:3 to SEQ ID NO:61.

A further object of the present invention relates to a variant VP3 capsid protein of the present invention has an amino acid sequence that ranges from the amino acid at position 203 to the last amino acid in SEQ ID NO:26 (i.e. capsid #3013), SEQ ID NO:9 (i.e. capsid #2087), SEQ ID NO: 20 (i.e. capsid #1449), SEQ ID NO: 10 (i.e. capsid #2206), SEQ ID NO: 12 (i.e. capsid #1534), SEQ ID NO: 56 (i.e. capsid #163), SEQ ID NO: 23 (i.e. capsid #1343), SEQ ID NO: 53 (i.e. capsid #790), SEQ ID NO: 6 (i.e. capsid #1017), SEQ ID NO: 11 (i.e. capsid #877), SEQ ID NO:8 (i.e. capsid #1273), SEQ ID NO: 3 (i.e. capsid #2497), SEQ ID NO: 60 (i.e. capsid #1055), SEQ ID NO:14 (i.e. capsid #1919), SEQ ID NO: 4, SEQ ID NO:5, SEQ ID NO:7, SEQ ID NO:13, SEQ ID NO:15 to SEQ ID NO:19, SEQ ID NO:21, SEQ ID NO:22, SEQ ID NO:24, SEQ ID NO:25, SEQ ID NO:27 to SEQ ID NO:55, SEQ ID NO:57 to SEQ ID NO:59, SEQ ID NO:61.

Polynucleotides that Encodes for the Variant Capsid Proteins:

A further object of the present invention relates to a polynucleotide that encodes for a variant capsid protein of the present invention.

In some embodiments, the polynucleotide of the present invention that encodes for a variant VP1 capsid of the present invention consists of the nucleic sequence that ranges from the nucleotide at position 2203 to the nucleotide at position 4410 in SEQ ID NO:1 and that comprises at least one mutation selected from Table 1.

In some embodiments, the polynucleotide of the present invention that encodes for a variant VP1 capsid of the present invention consists of the nucleic sequence that ranges from the nucleotide at position 2203 to the nucleotide at position 4410 in SEQ ID NO:1 and that comprises at least one mutation selected from the group consisting of 2433A>G; 2511C>T; 2654T>C; 2685G>A; 2692CAG>AAC; 2721T>C; 2745A>C; 2794C>T; 2800A>T; 2804A>C; 2808G->A; 2833A>G; 2853C>T; 2859C>T; 2877G>A; 2899AC>CA; 2905A>C; 2946C>T; 2967C>T; 2970C>G; 2976A>G; 2979A>G; 2982T>C; 3003G>A; 3012T>C; 3036T>C; 3123A>G; 3129G>A; 3165A>G; 3297C>T; 3303A>G; 3318G>C; 3360A>G; 3375A>C; 3411G>T; 3417C>T; 3430ACC>CAG; 3444T>C; 3450G>A; 3490C>T; 3495C>T; 3498T>G; 3501C>G; 3513C>G; 3534C>T; 3535T>C; 3539G>A; 3542GA>AG; 3547AAC>CAA; 3550ACT>TCA; 3553CCA>AAT; 3558T>C; 3564C>T; 3565ACC>CTT; 3568AC>CA; 3576A>C; 3576A>T; 3577A>C; 3582T>A; 3584A>T; 3589TC>AG; 3594G>A; 3597C>T3601GCG>CCC 3605GT>CC 3607GA>AG 3612T>G 3613CGG>TCT 3616GAC>CTT 3621G>A 3622T>G 3626GG>AA 3636T>G 3645C>T 3648T>C 3652CGC>AGA 3663A>T 3664GTA>CTG 3672ACA>CAG 3676TCT>GCA 3679GCG>AAC 3684T>C 3696T>C 3697GAA>AAC 3701AC>TT 3703TCG>CCC 3713GA>CG 3720C>A 3726C>T 3729C>T 3732C>A 3739AGA>CGG 3747T>G 3748C>T 3753G>T 3759G>A 3762C>A 3765G>A 3768C>T 3774A>C 3783G>A 3804T>C 3807T>C 3808CA>AT 3811AGC>CAT 3816G>A 3817GTT>ACC 3822C>G 3825C>A 3831G>A 3834G>A 3840C>A 3841T>A 3844GAG>AAT 3847AAA>GCT 3851CA>AC 3853AAT>GAC 3857T>C 3861C>T 3862ATT>TTG 3867A>C 3870>T 3885C>T 3891G>A 3898AGG>CGC 3903A>C 3930T>C 3933T>A 3935C>A 3939A>G 3942T>A 3944CC>AT 3948C>T 3949CTC>TTG 3954G>A 3956GA>AC 3957A>C 3958GGC>TCA 3963C>T 3965G>C 3967CAA>GGT 3970G>C 3973G>A 3978C>T 3979G>T 3980C>G 3982GA>AC 3990C>T 3991ACA>CAC 3999C>A 4001TT>CG 4003CTT>TTA 4008A>T 4011C>T 4017C>G 4026C>T 4027A>C 4032T>C 4041T>G 4047G>A 4053C>T 4059A>C 4068A>T 4074G>C 4077C>T 4083T>C 4089C>T 4092C>T 4098C>A 4101C>G 4107T>C 4110A>T 4113C>T 4116A>G 4119T>G 4128T>G 4134A>G 4140T>C 4141CTC>ATG 4149G>A 4155C>T 4158G>C 4161A>T 4164T>A 4167G>C 4174TCG>CCC 4179C>A 4181C>A 4189GCG>TCT 4194A>C 4206C>T 4221C>T 4230A>G 4269A>G 4287T>C 4293A>G 4296T>C 4320G>A and 4356C>T.

In some embodiments, the polynucleotide of the present invention that encodes for a variant VP1 capsid of the present invention consists the nucleic sequence that ranges from the nucleotide at position 2203 to the nucleotide at position 4410 in SEQ ID NO: 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; or 118.

In some embodiments, the polynucleotide of the present invention that encodes for a variant VP1 capsid of the present invention consists the nucleic sequence that ranges from the nucleotide at position 2203 to the nucleotide at position 4410 in SEQ ID NO: 85 (i.e. capsid #3013), SEQ ID NO: 68 (i.e. capsid #2087), SEQ ID NO: 79 (i.e. capsid #1449), SEQ ID NO: 69 (i.e. capsid #2206), SEQ ID NO: 71 (i.e. capsid #1534), SEQ ID NO: 115 (i.e. capsid #163), SEQ ID NO: 82 (i.e. capsid #1343), SEQ ID NO: 112 (i.e. capsid #790), SEQ ID NO:65 (i.e. capsid #1017), SEQ ID NO: 70 (i.e. capsid #877), SEQ ID NO: 67 (i.e. capsid #1273), SEQ ID NO: 62 (i.e. capsid #2497); SEQ ID NO: 119 (i.e. capsid #1055); SEQ ID NO: 73 (i.e. capsid #1919), SEQ ID NO:63, SEQ ID NO:64; SEQ ID NO:66, SEQ ID NO: 72, SEQ ID NO: 74, SEQ ID NO: 75, SEQ ID NO: 76, SEQ ID NO: 77, SEQ ID NO: 78, SEQ ID NO:80, SEQ ID NO:81, SEQ ID NO:83, SEQ ID NO:84, SEQ ID NO:86, SEQ ID NO:87; SEQ ID NO:88; SEQ ID NO:89; SEQ ID NO:90; SEQ ID NO: 91; SEQ ID NO:92; SEQ ID NO:93; SEQ ID NO:94; SEQ ID NO:95; SEQ ID NO:96; SEQ ID NO:97; SEQ ID NO:98, SEQ ID NO: 99; SEQ ID NO:100; SEQ ID NO:101; SEQ ID NO:102; SEQ ID NO:103, SEQ ID NO:104; SEQ ID NO:105, SEQ ID NO: 106; SEQ ID NO:107, SEQ ID NO:108, SEQ ID NO:109, SEQ ID NO: 110; SEQ ID NO:111, SEQ ID NO:113; SEQ ID NO:114; SEQ ID NO:116, SEQ ID NO: 117; SEQ ID NO:118 or SEQ ID NO:120.

In some embodiments, the polynucleotide of the present invention that encodes for a variant VP2 capsid of the present invention consists of the nucleic sequence that ranges from the nucleotide at position 2614 to the nucleotide at position 4410 in SEQ ID NO:1 and that comprises at least one mutation selected from Table 1.

In some embodiments, the polynucleotide of the present invention that encodes for a variant VP2 capsid of the present invention consists of the nucleic sequence that ranges from the nucleotide at position 2614 to the nucleotide at position 4410 in SEQ ID NO:1 and that comprises at least one mutation selected from the group consisting of 2654T>C; 2685G>A; 2692CAG>AAC; 2721T>C; 2745A>C; 2794C>T; 2800A>T; 2804A>C; 2808G->A; 2833A>G; 2853C>T; 2859C>T; 2877G>A; 2899AC>CA; 2905A>C; 2946C>T; 2967C>T; 2970C>G; 2976A>G; 2979A>G; 2982T>C; 3003G>A; 3012T>C; 3036T>C; 3123A>G; 3129G>A; 3165A>G; 3297C>T; 3303A>G; 3318G>C; 3360A>G; 3375A>C; 3411G>T; 3417C>T; 3430ACC>CAG; 3444T>C; 3450G>A; 3490C>T; 3495C>T; 3498T>G; 3501C>G; 3513C>G; 3534C>T; 3535T>C; 3539G>A; 3542GA>AG; 3547AAC>CAA; 3550ACT>TCA; 3553CCA>AAT; 3558T>C; 3564C>T; 3565ACC>CTT; 3568AC>CA; 3576A>C; 3576A>T; 3577A>C; 3582T>A; 3584A>T; 3589TC>AG; 3594G>A; 3597C>T3601GCG>CCC 3605GT>CC 3607GA>AG 3612T>G 3613CGG>TCT 3616GAC>CTT 3621G>A 3622T>G 3626GG>AA 3636T>G 3645C>T 3648T>C 3652CGC>AGA 3663A>T 3664GTA>CTG 3672ACA>CAG 3676TCT>GCA 3679GCG>AAC 3684T>C 3696T>C 3697GAA>AAC 3701AC>TT 3703TCG>CCC 3713GA>CG 3720C>A 3726C>T 3729C>T 3732C>A 3739AGA>CGG 3747T>G 3748C>T 3753G>T 3759G>A 3762C>A 3765G>A 3768C>T 3774A>C 3783G>A 3804T>C 3807T>C 3808CA>AT 3811AGC>CAT 3816G>A 3817GTT>ACC 3822C>G 3825C>A 3831G>A 3834G>A 3840C>A 3841T>A 3844GAG>AAT 3847AAA>GCT 3851CA>AC 3853AAT>GAC 3857T>C 3861C>T 3862ATT>TTG 3867A>C 3870>T 3885C>T 3891G>A 3898AGG>CGC 3903A>C 3930T>C 3933T>A 3935C>A 3939A>G 3942T>A 3944CC>AT 3948C>T 3949CTC>TTG 3954G>A 3956GA>AC 3957A>C 3958GGC>TCA 3963C>T 3965G>C 3967CAA>GGT 3970G>C 3973G>A 3978C>T 3979G>T 3980C>G 3982GA>AC 3990C>T 3991ACA>CAC 3999C>A 4001TT>CG 4003CTT>TTA 4008A>T 4011C>T 4017C>G 4026C>T 4027A>C 4032T>C 4041T>G 4047G>A 4053C>T 4059A>C 4068A>T 4074G>C 4077C>T 4083T>C 4089C>T 4092C>T 4098C>A 4101C>G 4107T>C 4110A>T 4113C>T 4116A>G 4119T>G 4128T>G 4134A>G 4140T>C 4141CTC>ATG 4149G>A 4155C>T 4158G>C 4161A>T 4164T>A 4167G>C 4174TCG>CCC 4179C>A 4181C>A 4189GCG>TCT 4194A>C 4206C>T 4221C>T 4230A>G 4269A>G 4287T>C 4293A>G 4296T>C 4320G>A and 4356C>T.

In some embodiments, the polynucleotide of the present invention that encodes for a variant VP2 capsid of the present invention consists of the nucleic sequence that ranges from the nucleotide at position 2614 to the nucleotide at position 4410 in SEQ ID NO: 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; or 118.

In some embodiments, the polynucleotide of the present invention that encodes for a variant VP2 capsid of the present invention consists of the nucleic sequence that ranges from the nucleotide at position 2614 to the nucleotide at position 4410 in SEQ ID NO: 85 (i.e. capsid #3013), SEQ ID NO: 68 (i.e. capsid #2087), SEQ ID NO: 79 (i.e. capsid #1449), SEQ ID NO: 69 (i.e. capsid #2206), SEQ ID NO: 71 (i.e. capsid #1534), SEQ ID NO: 115 (i.e. capsid #163), SEQ ID NO: 82 (i.e. capsid #1343), SEQ ID NO: 112 (i.e. capsid #790), SEQ ID NO:65 (i.e. capsid #1017), SEQ ID NO: 70 (i.e. capsid #877), SEQ ID NO: 67 (i.e. capsid #1273), SEQ ID NO: 62 (i.e. capsid #2497); SEQ ID NO: 119 (i.e. capsid #1055); SEQ ID NO: 73 (i.e. capsid #1919), SEQ ID NO:63, SEQ ID NO:64; SEQ ID NO:66, SEQ ID NO: 72, SEQ ID NO: 74, SEQ ID NO: 75, SEQ ID NO: 76, SEQ ID NO: 77, SEQ ID NO: 78, SEQ ID NO:80, SEQ ID NO:81, SEQ ID NO:83, SEQ ID NO:84, SEQ ID NO:86, SEQ ID NO:87; SEQ ID NO:88; SEQ ID NO:89; SEQ ID NO:90; SEQ ID NO: 91; SEQ ID NO:92; SEQ ID NO:93; SEQ ID NO:94; SEQ ID NO:95; SEQ ID NO:96; SEQ ID NO:97; SEQ ID NO:98, SEQ ID NO: 99; SEQ ID NO:100; SEQ ID NO:101; SEQ ID NO:102; SEQ ID NO:103, SEQ ID NO:104; SEQ ID NO:105, SEQ ID NO: 106; SEQ ID NO:107, SEQ ID NO:108, SEQ ID NO:109, SEQ ID NO: 110; SEQ ID NO:111, SEQ ID NO:113; SEQ ID NO:114; SEQ ID NO:116, SEQ ID NO: 117; SEQ ID NO:118 or SEQ ID NO:120

In some embodiments, the polynucleotide of the present invention that encodes for a variant VP3 capsid of the present invention consists of the nucleic sequence that ranges from the nucleotide at position 2809 to the nucleotide at position 4410 in SEQ ID NO:1 and that comprises at least one mutation selected from Table 1.

In some embodiments, the polynucleotide of the present invention that encodes for a variant VP3 capsid of the present invention consists of the nucleic sequence that ranges from the nucleotide at position 2809 to the nucleotide at position 4410 in SEQ ID NO:1 and that comprises at least one mutation selected from the group consisting of 2833A>G; 2853C>T; 2859C>T; 2877G>A; 2899AC>CA; 2905A>C; 2946C>T; 2967C>T; 2970C>G; 2976A>G; 2979A>G; 2982T>C; 3003G>A; 3012T>C; 3036T>C; 3123A>G; 3129G>A; 3165A>G; 3297C>T; 3303A>G; 3318G>C; 3360A>G; 3375A>C; 3411G>T; 3417C>T; 3430ACC>CAG; 3444T>C; 3450G>A; 3490C>T; 3495C>T; 3498T>G; 3501C>G; 3513C>G; 3534C>T; 3535T>C; 3539G>A; 3542GA>AG; 3547AAC>CAA; 3550ACT>TCA; 3553CCA>AAT; 3558T>C; 3564C>T; 3565ACC>CTT; 3568AC>CA; 3576A>C; 3576A>T; 3577A>C; 3582T>A; 3584A>T; 3589TC>AG; 3594G>A; 3597C>T3601GCG>CCC 3605GT>CC 3607GA>AG 3612T>G 3613CGG>TCT 3616GAC>CTT 3621G>A 3622T>G 3626GG>AA 3636T>G 3645C>T 3648T>C 3652CGC>AGA 3663A>T 3664GTA>CTG 3672ACA>CAG 3676TCT>GCA 3679GCG>AAC 3684T>C 3696T>C 3697GAA>AAC 3701AC>TT 3703TCG>CCC 3713GA>CG 3720C>A 3726C>T 3729C>T 3732C>A 3739AGA>CGG 3747T>G 3748C>T 3753G>T 3759G>A 3762C>A 3765G>A 3768C>T 3774A>C 3783G>A 3804T>C 3807T>C 3808CA>AT 3811AGC>CAT 3816G>A 3817GTT>ACC 3822C>G 3825C>A 3831G>A 3834G>A 3840C>A 3841T>A 3844GAG>AAT 3847AAA>GCT 3851CA>AC 3853AAT>GAC 3857T>C 3861C>T 3862ATT>TTG 3867A>C 3870>T 3885C>T 3891G>A 3898AGG>CGC 3903A>C 3930T>C 3933T>A 3935C>A 3939A>G 3942T>A 3944CC>AT 3948C>T 3949CTC>TTG 3954G>A 3956GA>AC 3957A>C 3958GGC>TCA 3963C>T 3965G>C 3967CAA>GGT 3970G>C 3973G>A 3978C>T 3979G>T 3980C>G 3982GA>AC 3990C>T 3991ACA>CAC 3999C>A 4001TT>CG 4003CTT>TTA 4008A>T 4011C>T 4017C>G 4026C>T 4027A>C 4032T>C 4041T>G 4047G>A 4053C>T 4059A>C 4068A>T 4074G>C 4077C>T 4083T>C 4089C>T 4092C>T 4098C>A 4101C>G 4107T>C 4110A>T 4113C>T 4116A>G 4119T>G 4128T>G 4134A>G 4140T>C 4141CTC>ATG 4149G>A 4155C>T 4158G>C 4161A>T 4164T>A 4167G>C 4174TCG>CCC 4179C>A 4181C>A 4189GCG>TCT 4194A>C 4206C>T 4221C>T 4230A>G 4269A>G 4287T>C 4293A>G 4296T>C 4320G>A and 4356C>T.

In some embodiments, the polynucleotide of the present invention that encodes for a variant VP3 capsid of the present invention consists of the nucleic sequence that ranges from the nucleotide at position 2809 to the nucleotide at position 4410 in SEQ ID NO: 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; or 118.

In some embodiments, the polynucleotide of the present invention that encodes for a variant VP3 capsid of the present invention consists of the nucleic sequence that ranges from the nucleotide at position 2809 to the nucleotide at position 4410 in SEQ ID NO: 112, SEQ ID NO: 115, SEQ ID NO: 101, SEQ ID NO:105, SEQ ID NO: 111, SEQ ID NO:98, SEQ ID NO:107, SEQ ID NO:109, SEQ ID NO:108, SEQ ID NO:103, SEQ ID NO:116, SEQ ID NO: 62; SEQ ID NO:63; SEQ ID NO:64; SEQ ID NO:65; SEQ ID NO:66; SEQ ID NO:67; SEQ ID NO:68; SEQ ID NO:69; SEQ ID NO:70; SEQ ID NO:71; SEQ ID NO:72; SEQ ID NO:73; SEQ ID NO:74; SEQ ID NO:75; SEQ ID NO:76; SEQ ID NO:77; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:86; SEQ ID NO:87; SEQ ID NO:88; SEQ ID NO:89; SEQ ID NO:90; SEQ ID NO: 91; SEQ ID NO:92; SEQ ID NO:93; SEQ ID NO:94; SEQ ID NO:95; SEQ ID NO:96; SEQ ID NO:97; SEQ ID NO: 99; SEQ ID NO:100; SEQ ID NO:101; SEQ ID NO:102; SEQ ID NO:104; SEQ ID NO: 106; SEQ ID NO: 110; SEQ ID NO:113; SEQ ID NO:114; SEQ ID NO: 117; SEQ ID NO:118, SEQ ID NO:119 or SEQ ID NO:120 SEQ ID NO: 85 (i.e. capsid #3013), SEQ ID NO: 68 (i.e. capsid #2087), SEQ ID NO: 79 (i.e. capsid #1449), SEQ ID NO: 69 (i.e. capsid #2206), SEQ ID NO: 71 (i.e. capsid #1534), SEQ ID NO: 115 (i.e. capsid #163), SEQ ID NO: 82 (i.e. capsid #1343), SEQ ID NO: 112 (i.e. capsid #790), SEQ ID NO:65 (i.e. capsid #1017), SEQ ID NO: 70 (i.e. capsid #877), SEQ ID NO: 67 (i.e. capsid #1273), SEQ ID NO: 62 (i.e. capsid #2497); SEQ ID NO: 119 (i.e. capsid #1055); SEQ ID NO: 73 (i.e. capsid #1919), SEQ ID NO:63, SEQ ID NO:64; SEQ ID NO:66, SEQ ID NO: 72, SEQ ID NO: 74, SEQ ID NO: 75, SEQ ID NO: 76, SEQ ID NO: 77, SEQ ID NO: 78, SEQ ID NO:80, SEQ ID NO:81, SEQ ID NO:83, SEQ ID NO:84, SEQ ID NO:86, SEQ ID NO:87; SEQ ID NO:88; SEQ ID NO:89; SEQ ID NO:90; SEQ ID NO: 91; SEQ ID NO:92; SEQ ID NO:93; SEQ ID NO:94; SEQ ID NO:95; SEQ ID NO:96; SEQ ID NO:97; SEQ ID NO:98, SEQ ID NO: 99; SEQ ID NO:100; SEQ ID NO:101; SEQ ID NO:102; SEQ ID NO:103, SEQ ID NO:104; SEQ ID NO:105, SEQ ID NO: 106; SEQ ID NO:107, SEQ ID NO:108, SEQ ID NO:109, SEQ ID NO: 110; SEQ ID NO:111, SEQ ID NO:113; SEQ ID NO:114; SEQ ID NO:116, SEQ ID NO: 117; SEQ ID NO:118 or SEQ ID NO:120.

SEQ ID NO: 62
>#2497_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAATGGCGCCGTGTGAGTAAGGCCC
CGGAGGCCCTTTTCTTTGTGCAATTTGAGAAGGGAGAGAGCTACTTCCACATGCACGTGC
TCGTGGAAACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTASCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCAAATTCTGACGSRC
CGGTGATCAGATCAAAAACCTCAGCCAGGTACATGGAGCTGGTCGGGTGGCTCGTGGAYA
AGGGGATTACCTCGGAGAAGCAGTGGATCCAGGAGGAYCAGGCTTCATACATCTCCTTCA
ATGCGGCCTCCAACTCGCGGTCCCAAATCAAGGCTGCCTTGGACAATGCGGGAAAGATCA
TGAGCCTCACTAAAACTGCCCCCGACTACCTGGTGGGCCAGCAGCCCGTGGAGGACATTT
CCAGCAATCGGATTTATAAAATTTTGGAACTAAACGGGTACGACCCCCAATACGCGGCTT
CCGTCTTTCTGGGATGGGCCACGAAAAAGTTCGGCAAGAGAAACACCATCTGGCTGTTTG
GGCCTGCAACTACCGGGAAGACCAACATCGCGGAGGCCATAGCCCACACGGTGCCCTTCT
ACGGGTGCGTAAACTGGACCAATGAGAACTTTCCCTTCAATGACTGCGTCGACAAGATGG
TGATCTGGTGGGAGGAGGGGAGGATGACCGCCAAGGTCGTGGAGTCGACCAAAGCCATTC
TTGGCGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACAAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTYAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAARGGAGAGCCGGTCAAC
GAGGCAGACGCCGCGGCCCTCGAACACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCATTCTCCTGTGGAGCCAGACTCCTCCTCGGGAACCGGAAAGGCGGGCCAGCAGCCT
GCAAGAAAAAGATTGAATTTTGGTCAGACTGGAGACGCAGACTCAGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGAACC
TGGGCCCTGCCCACCTACAACAACCACCTCTACAAGCAAATTTCCAGCCAATCAGGAGCC
TCAAACGACAACCACTACTTTGGCTACAGCACCCCTTGGGGGTATTTTGACTTCAACAGA
TTCCACTGCCACTTTTCACCACGTGACTGGCAAAGACTCATCAACAACAACTGGGGATTC
CGACCCAAAAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTTTTCATGGTCCCACAGTATGGATACCTCACCCTGAACAACGGGAGTCAGGCG
GTAGGACGCTCTTCMTTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTTTGGACCGTCTCATGAATCCTCTCATCGACCAGTACCTGTATTACTTGAGC
AGAACAAACACTCCAAGCGGAACCACCACGCAGTCAAGGCTTCAGTTTTCTCAGGCCGGA
GCGAGTGACATTCGGGACCAGTCTAGGAACTGGCTTCCTGGACCCTGTTACCGCCAGCAG
CGAGTATCAAAGACAGCTGCGGACAACAGCAACAGTGAGTACTCGTGGACTGGTGCTACC
AAGTACCACCTCAATGGAAGAGACTCTCTGGTGAATCCGGGCCCGGCCATGGCCAGCCAC
AAGGACGATGAAGAAAAGTTTTTTCCTCACAGCGGGGTTCTCATCTTTGGGAAGCAAGGC
TCAGAGAAAACAAATGTGGACATTGAAAAGGTCATGATTACAGACGAAGAGGAAATCAGG
ACCACCAATCCCGTGGCTACGGAGCAGTATGGTACTGTATCTACCAACCTCCAGAGCGGC
AACACACAAGCAGCTACCTCAGATGTCAACACACAAGGCATTCTTCCAGGCATGGTCTGG
CAGGACAGAGACGTGTACCTGCAGGGACCCATCTGGGCAAAGATTCCACACACGGACGGA
CACTTTCACCCTTCTCCCCTCATGGGTGGATTCGGACTTAAACACCCTCCTCCACAGATT
CTCATCAAGAACACCCCGGTACCTGCGAATCCTTCGACCACCTTCAGTGCGGCAAAGTTT
GCTTCCTTCATCACACAGTACTCCACGGGACAGGTCAGCGTGGAGATCGAGTGGGAGCTG
CAGAAGGAAAACAGCAAACGCTGGAATCCCGAAATCCAGTACACTTCCAACTACAACAAR
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTATTTCTTTCTTATCTAGTTTCCATGGCTACGTA
GATAAGTAGCATGGCGGGTTAATCATTAACTACAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 63
>#2102_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCTCGCCCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACATCAT
GTGGTCACGCTTGGGTATTTAAGCCCGAGTGAGCACGCAGGGTCTCCATTTTGAAGCGGG
AGGTTTGAACGTGCAGCCGCCATGCCGGGGTTTTACGAGATTGTGATTAAGGTCCCCAGC
GACCTTGACGAGCATCTGCCCGGCATTTCTGACAGCTTTGTGAACTGGGTGGCCGAGAAG
GAATGGGAGTTGCCGCCAGATTCTGACATGGATCTGAATCTGATTGAGCAGGCACCCCTG
ACCGTGGCCGAGAAGCTGCAGCGCGACTTTCTGACGGAGTGGCGCCGTGTGAGTAAGGCC
CCGGAGGCCCTCTTCTTTGTGCAATTTGAGAAGGGAGAGAGCTACTTCCACATGCACGTG
CTTGTGGAGACCACAGGGGTGAAATCCATGGTTTTGGGACGTTTCCTGAGTCAGATTCGC
GAAAAACTGATTCAGAGAATTTACCGCGGGATCGAGCCGACTTTGCCAAACTGGTTCGCG
GTCACAAAGACCCGAAATGGCGCCGGAGGCGGGAACAAGGTGGTGGATGAGTGCTACATC
CCCAATTACCTGCTCCCCAAAACCCAGCCTGAGCTTCAGTGGGCGTGGACTAATATGGAA
CAGTATTTAAGCGCCTGTTTGAATCTCGCGGAGCGTAAACGGTTGGTGGCGCAGCATCTG
ACGCACGTGTCGCAGACGCAGGAGCAGAACAAAGAGAATCAGAATCCCAATTCTGACGCG
CCGGTGATTAGATCAAAAACCTCAGCCAGGTACATGGAGCTGGTCGGGTGGCTCGTGGAT
AAGGGGATTACGTCGGAGAAGCAGTGGATCCAGGAGGACCAGGCTTCATACATCTCCTTC
AATGCGGCCTCCAACTCGCGGTCCCAAATCAAGGCTGCTCTGGACAATGCGGGAAAGATC
ATGAGCCTCACTAAAACCGCCCCMGACTACCTRGTGGGCCAGCAGCCCGTGGAGGACATT
TCCAGCAATCGGATTTATAAAATCTTGGAACTGAACGGGTACGATCCCCAATACGCGGCT
TCCGTCTTTCTGGGATGGGCCACGAAAAAGTTCGGCAAGAGGAACACCATCTGGCTGTTT
GGGCCTGCAACTACCGGGAAGACCAACATCGCGGAGGCCATAGCCCACACTGTGCCCTTC
TACGGGTGCGTAAACTGGACCAATGAGAACTTTCCCTTCAACGACTGTGTCGACAAGATG
GTGATCTGGTGGGAGGAGGGAAAGATGACCGCCAAGGTCGTGGAGTCGGCCAAAGCCATT
CTCGGCGGAAGCAAGGTGCGCGTGGACCAGAAATGCAAGTCCTCGGCCCAGATAGACCCG
ACTCCCGTGATCGTCACCTCCAACACCAACATGTGCGCCGTGATTGACGGGAACTCAACG
ACCTTCGAACACCAGCAGCCGTTGCAAGACCGGATGTTCAAATTTGAACTCACCCGCCGT
CTGGATCATGACTTTGGGAAGGTCACCAAGCAGGAAGTCAAAGACTTTTTCCGGTGGGCA
AAGGATCACGTGGTTGAGGTGGAGCATGAATTCTACGTCAAAAAGGGTGGAGCCAAGAAA
AGRCCCGCCCCCAGTGACGCAGATATAAGTGAGCCCAAACGGGCGCGCGAGTCAGTTGCG
CAGCCATCGACGTCAGACGCGGAAGCTTCGATCAACTACGCGGACAGGTACCAAAACAAA
TGTTCTCGTCACGTGGGCATGAATCTGATGCTGTTTCCCTGCAGACAATGCGAGAGAATG
AATCAGAATTCAAATATCTGCTTCACTCACGGACAGAAAGACTGTTTAGAGTGCTTTCCC
GTGTCAGAATCTCAACCCGTTTCTGTCGTCAAAAAGGCGTATCAGAAACTTTGCTACATT
CATCATATCATGGGAAAGGTGCCAGACGCTTGCACTGCCTGCGATCTGGTCAATGTGGAT
TTGGATGACTGCATCTCTGAACAATAAATGATTTAAATCAGGTATGGCTGCCGATGGTTA
TCTTCCAGATTGGCTCGAGGACACTCTCTCTGAAGGAATAAGACAGTGGTGGAAGCTCAA
ACCTGGCCCACCACCACCAAAGCCCGCAGAGCGGCATCAGGACGACAGCAGGGGTCTTGT
GCTTCCTGGGTACAAGTACCTCGGACCCTTCAACGGACTCGACAAAGGAGAGCCGGTCAA
CGAGGCAGACGCCGCGGCCCTCGAGCACGACAAGGCCTACGACCGGCAGCTCGAAAGCGG
AGACAACCCGTACCTCAAGTACAACCACGCCGACGCGGAGTTTCAGGAGCGCCTTAAAGA
AGATACGTCTTTTGGGGGCAACCTCGGACGAGCAGTCTTCCAGGCGAAAAAGAGGGTTCT
TGAACCTCTGGGCCTGGTTGAGGAACCTGTTAAGACGGCTCCGGGAAAAAAGAGGCCGGT
AGAGCACTCTCCTGTGGAGCCAGACTCCTCCTCGGGAACCGGAAAGGCGGGCAACCAGCC
TGCAAGAAAGAGATTGAATTTCGGTCAGACTGGAGACGCAGACTCCGTACCTGACCCCCA
ACCTCTCGGACAGCCACCAGCAGCCCCCTCTGGTCTGGGAACTAATACGATGGCTACAGG
CAGTGGCGCACCAATGGCAGACAATAACGAGGGCGCCGACGGAGTGGGTAATTCCTCGGG
AAATTGGCATTGCGATTCCACATGGCTGGGCGACAGAGTCATCACCACCAGCACCCGCAC
CTGGGCTCTGCCCACCTACAACAACCACCTCTACAAGCAAATCTCCAGCCAATCAGGAGC
CTCGAACGACAATCACTACTTTGGCTACAGCACCCCCTGGGGGTATTTTGACTTCAACAG
ATTCCACTGCCACTTTTCACCACGTGACTGGCAAAGACTCATCAACAACAATTGGGGATT
CCGGCCCAAAAGACTCAACTTCAAGCTGTTTAACATTCAAGTCAAAGAGGTCACGCAGAA
TGACGGTACGACGACGATTGCCAATAACCTTACCAGCACGGTTCAGGTGTTTACTGACTC
GGAGTACCAGCTCCCGTACGTCCTCGGCTCGGCGCATCAAGGATGCCTCCCGCCGTTCCC
AGCAGACGTCTTCATGGTGCCACAGTATGGATACCTCACCCTGAACAACGGGAGTCAGGC
GGTCGGACGCTCTTCCTTTTACTGCCTGGAGTACTTTCCTTCTCAAATGCTTCGTACCGG
AAACAACTTTACCTTCAGCTACACCTTTGAAGACGTTCCTTTCCACAGCAGCTACGCTCA
CAGTCAAAGTCTGGACCGTCTCATGAATCCTCTCATCGACCAGTACCTGTATTACTTGAG
CGGAACAAACACTCCAAGCGGAACCACCACGCAGTCCAGGCTTCAGTTTTCTCAGGCCGG
AGCAAGTGACATTCGGGACCAGTCTAGGAACTGGCTTCCTGGACCCTGTTACCGCCAGCA
GCGAGTATCAAAGACAGCTGCGGACAACAGCAACAGTGAGTACTCGTGGACTGGAGCTAC
CAAGTACCACCTCAATGGAAGAGACTCTCTGGTGAATCCGGGCCCGGCCATGGCCAGCCA
CAAGGACGATGAAGAAAAGTTTTTTCCTCAGAGCGGGGTTCTCATCTTTGGGAAGCAAGG
CTCAGAGAAAACAAATGTGGACATTGAAAAGGTCATGATTACAGACGAAGAGGAAATCAG
GACCACCAATCCCGTGGCTACGGAGCAGTATGGTTCTGTATCTACCAACCTCCAGAGCGG
CAACACACAAGCAGCTACCTCAGATGTCAACACACAAGGCGTTCTTCCAGGCATGGTCTG
GCAGGACAGAGACGTGTACCTGCAGGGGCCTATCTGGGCAAAGATTCCACACACGGACGG
ACATTTTCACCCCTCTCCCCTCATGGGTGGATTCGGACTTAAACACCCTCCTCCACAGAT
TCTCATCAAAAACACCCCGGTACCTGCGAATCCTTCGACCACCTTCAGTGCGGCAAAGTT
TGCTTCCTTCATCACACAGTACTCCACGGGACAGGTCAGCGTGGAGATCGAGTGGGAGCT
GCAGAAGGAAAACAGCAAACGCTGGAACCCCGAGATCCAGTACACTTCCAACTACAACAA
ATCTGTTAATGTGGACTTTACTGTGGACACTAATGGTGTGTATTCAGAGCCTCGCCCCAT
TGGCACCAGATACCTGACTCTTAATCTGTAATTGCTTGTTAATCAATAAACCGTTTAATT
CGTTTCAGTTGAACTTTGGTCTCTGCGTACTTCTTTCTTATCTAGTTTCCATGGCTACGT
AGATAAGTAGCATGGCGGGTTAATCATTAACTACAAGGAACCCCTAGTGATGGAGTTGGC
CACTCCCTCTCTGCGCGCTCGCTCGCTCACTGAGGCCGGGCGACCAAAGGTCGCCCGACG
CCCGGGCTTTGCCCGGGCGGCCTCAGTGAGCGAGCGAGCGCGCAGAGAGGGAGTGGCCAA
SEQ ID NO: 64
>#371_AAV.FL.linear [organism = Adeno-associated virus][mol_type = genomic DNA]
[isolation_source = liver tissue][topology = linear]
TTGGCCACTCCCTCTCTGCGCGCTCGCTCGCTCACTGAGGCCGGGCGACCAAAGGTCGAC
CGACGCCCGGGCTTTGGTCGCCCGGCCTCAGTGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCGGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGTGTGAGTAAGGCCC
CGGAGGCCCTCTTCTTTGTGCAATTTGAGAAGGGAGAGAGCTACTTCCACATGCACGTGC
TGGTGGAGACCACTGGGGTTAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACCTCGGAGAAGCAGTGGATCCAGGAGGACCAGGCCTCATACATCTCCTTCA
ATGCGGCCTCCAACTCGCGGTCCCAGATAAAGGCTGCTCTGGACAATGCGGGAAAGATCA
TGAGCCTGACTAAAACGGCCCCCGACTACCTGGTGGGCCAGCAGCCCGTGGAGGACATTT
CCAGCAATCGGATTTATAAAATCTTGGAACTGAACGGGTACGATCCCCAATATGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCTTTCAACGACTGTGTCGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAATCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCTAAAAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAAAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGCACC
TGGGCTCTGCCCACCTACAACAACCATCTGTACAAACAGATTTCCAGCCAATCAGGAGCC
AGCAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTATTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTGCCACAGTATGGATACCTCACCCTGAACAACGGGAGTCAGGCA
GTAGGACGCTCTTCCTTTTACTGCCTGGAGTACTTTCCTTCTCAGATGCTGCGTACCGGA
AACAACTTTACCTTCAGCTACACCTTTGAGGACGTTCCTTTCCACAGCAGCTACGCTCAC
AGCCAGAGTTTGGACCGTCTCATGAATCCTCTCATCGACCAGTACCTGTATTACTTGAGC
AGAACAAACACTCCAAGCGGAACCACCACGCAGTCCAGGCTTCAGTTTTCTCAGGCCGGA
GCGAGTGACATTCGGGACCAGTCTAGGAACTGGCTTCCTGGACCCTGTTACCGCCAGCAA
CGAGTATCAAAGACATCTGCGGATAACAACAACAGTGAATATTCGTGGACTGGAGCTACC
AAATACCACCTCAATGGCAGAGACTCTCTGGTGAATCCGGGCCCGGCCATGGCCAGCCAC
AAGGACGATGAAGAAAAGTTTTTTCCTCAAAGCGGGGTTCTCATCTTTGGAAAACAAGGC
TCAGAGAAAACAAATGTGGACATTGAAAAGGTCATGATTACAGACGAAGAGGAAATCAGG
ACCACCAATCCCGTGGCTACAGAGCAGTATGGTTCTGTATCTACCAACCTCCAGAGCGGC
AACACACAAGCAGCTACCGCAGATGTCAACACACAAGGCGTTCTTCCTGGCATGGTCTGG
CAGGACAGAGACGTGTACCTGCAGGGGCCCATCTGGGCAAAGATTCCACACACGGACGGA
CATTTTCACCCCTCTCCCCTCATGGGCGGATTCGGACTTAAACACCCTCCTCCACAGATT
CTCATCAAGAACACCCCGGTACCTGCGAATCCTTCGACCACCTTCAGTGCGGCAAAGTTT
GCTTCCTTCATTACACAGTACTCCACGGGGCAGGTCAGCGTGGAGATCGAATGGGAGCTG
CAGAAAGAGAACAGCAAACGCTGGAATCCCGAGATTCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTCTTTTATTATCTAGTTTCCATGGCTACGT
AGATAAGTAGCATGGCGGGTTAATCATTAACTCTAAGGAACCCCTAGTGATGGAGTTGGC
CACTCCCTCTCTGCGCGCTCGCTCGCTCACTGAGGCCGGGCGACCAAAGGTCGCCCGACG
CCCGGGCTTTGCCCGGGCGGCCTCAGTGAGCGAGCGAGCGCGCAGAGAGGGAGTGGCCAA
SEQ ID NO: 65
>#1017_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAATGGCGCCGTGTGAGTAAGGCCC
CGGAGGCCCTCTTCTTTGTGCAATTTGAGAAGGGAGAGAGCTACTTCCACATGCACGTGC
TCGTGGAGACTACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATTTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACGTCGGAGAAGCAGTGGATCCAGGAGGACCAGGCCTCATACATCTCCTTCA
ATGCGGCCTCCAACTCGCGGTCCCAAATCAAGGCTGCTCTGGACAATGCGGGAAAGATCA
TGAGCCTCACTAAAACCGCCCCCGACTACCTGGTGGGCCAGCAGCCCGTGGAGGACATTT
CCAGCAATCGGATTTATAAAATCTTGGAACTGAACGGGTACGATCCCCAATATGCGGCTT
CCGTCTTTCTGGGATGGGCCACAAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCTTTCAACGACTGTGTCGACAAGATGG
TGATCTGGTGGGAGGAGGGAAAGATGACCGCCAAGGTTGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCTTCGGGAACCGGAAAAGCGGGCCAGCAGCCT
GCAAGAAAAAGACTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTTCAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGAACC
TGGGCCCTGCCCACCTACAACAACCATCTCTACAAGCAGATTTCCAGCCAATCAGGAGCT
TCAAACGACAACCACTACTTTGGCTACAGCACCCCCTGGGGGTATTTTGACTTCAACAGA
TTCCACTGCCACTTCTCGCCACGTGACTGGCAGAGACTCATCAACAACAACTGGGGATTC
CGTCCCAAGAGACTCAACTTCAAGCTGTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGTTCGGCGCATCAAGGATGCCTCCCGCCGTTCCCA
GCAGACGTCTTCATGGTGCCACAGTATGGATACCTCACCCTGAACAACGGGAGTCAGGCG
GTAGGACGCTCTTCATTTTACTGCCTGGAGTACTTTCCTTCTCAGATGCTGCGTACCGGA
AACAACTTTACCTTCAGCTACACCTTTGAGGACGTTCCTTTCCACAGCAGCTACGCTCAC
AGCCAGAGTTTGGACCGTCTCATGAATCCTCTCATCGACCAGTACCTGTATTACTTGAGC
AGAACAAACACTCCAAGCGGAACCACTACGCAGTCCAGGCTTCAGTTTTCTCAGGCCGGA
GCGAGTGACATTCGGGACCAGTCTAGGAACTGGCTTCCTGGACCCTGTTACCGCCAGCAG
CGAGTATCAAAAACATCTGCGGATAACAACAACAGTGAATACTCGTGGACTGGAGCTACC
AAGTACCACCTCAATGGCAGAGACTCTCTGGTGAATCCGGGCCCGGCCATGGCCAGCCAC
AAGGACGATGAAGAAAAGTTTTTTCCTCAGAGCGGGGTTCTCATCTTTGGAAAACAAGAC
TCAGGAAAAACTAATGTGGACATTGAAAAGGTCATGATTACAGACGAAGAGGAAATCAGG
ACCACCAATCCCGTGGCTACGGAGCAGTATGGTGCTGTATCTACCAACCTCCAGAGCGGC
AACACACAAGCAGCTACCTCAGATGTCAACGCACAAGGTGTTCTTCCAGGCATGGTCTGG
CAGGACAGAGAYGTGTACCTGCAGGGGCCCATCTGGGCAAAGATTCCWCACACGGAYGGA
CAYTTTCACCCCTCTCCCCTCATGGGCGGATTCGGACTGAAACACCCTCCTCCACAAATT
CTCATCAAGAACACCCCGGTACCTGCGAATCCTTCAACCACCTTCAGTGCGGCAAAGTTT
GCTTCCTTCATYACACAGTACTCCACGGGGCAGGTCAGCGTGGAGATCGAGTGGGAGCTG
CAGAAGGAAAACAGCAAACGCTGGAATCCCGAAATTCAGTACACTTCCAACTACAACAAA
TCTGTTAATGTGGACTTTACTGTGGACACTAATGGCGTGTATTCAGAGCCTCGCCCCATT
GGCACCAGATACCTGACTCGTAATCTGTAATTGCTTGTTAATCAATAAACCGTGTAATTC
GTTTCAGTTGAACTTTGGTCTCTGCGTACTTATTTCTTATCTAGTTTCCATGGCTACGTA
GATAAGTAGCATGGCGGGTTAATCATTAACTCTAAGGAACCCCTAGTGATGGAGTTGGCC
ACTCCCTCTCTGCGCGCTCGCTCGCTCACTGAGGCCGCCCGGGCAAAGGTCGCCCGACGC
CCGGGCGTCGGGCGACCGGCCTCAGTGAGCGAGCGAGCGCGCAGAGAGGGAGTGGCCAA
SEQ ID NO: 66
>#473_AAV.FL.linear [organism = Adeno-associated virus][mol_type = genomic DNA]
[isolation_source = liver tissue][topology = linear]
TTGGCCACTCCCTCTCTGCGCGCGCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGATGGGTGGAGTCGTGACGTAAAGTACGTCATAG
GGTTAGGGAGGTCCTGTATTAGAGGTCACGTGAGTGTTTTGCGACATTTTGCGACACCAT
GTGGTTACGCTGGGTATTTAAGCCCGAGTGAGCACGCAGGGTCTCCATTTTGAAGCGGGA
GGTTTGAACGCGCAGTCGCCATGCCGGGGTTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAATGGCGCCGTGTGAGTAAGGCCC
CGGAGGCCCTCTTCTTTGTGCAATTTGAGAAGGGAGAAAGCTACTTCCACATGCACGTGC
TGGTGGAGACCACCGGGGTGAAATCAATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGACA
AGGGGATTACCTCGGAGAAGCAGTGGATCCAGGAGGACCAGGCTTCATACATCTCCTTCA
ATGCGGCCTCCAACTCGCGGTCCCAGATCAAGGCTGCTCTGGACAATGCGGGAAAGATCA
TGAGCCTGACTAAAACGGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGAAAGACCAACATCGCGGAGGCCATAGCCCACACTGTGCCCTTCT
ACGGGTGCGTAAACTGGACCAATGAGAACTTTCCCTTCAACGACTGTGTCGACAAGATGG
TGATCTGGTGGGAGGAGGGGAAGATGACCGCCAAGGTTGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACAAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTACCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGGGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGCGGAGCCAGACTCCTCCTCGGGAACCGGAAAAGCGGGCCAGCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGCACC
TGGGCTCTGCCCACCTACAACAACCATCTGTACAAGCAGATTTCCAGCCAATCAGGAGCC
AGCAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGGCCAAAAAGACTCAACTTCAAGCTCTTTAACATTCAAGTCAAGGAGGTCACGCAGAAT
GACGGTACGACGACGATTGCCAATAACCTTACCAGCACGGTTCAGGTGTTTACTGACTCG
GAGTACCAGCTCCCGTACGTCCTGGGCTCGGCGCATCAAGGATGCCTCCCGCCGTTCCCA
GCAGACGTCTTCATGGTGCCACAGTATGGATACCTCACCCTGAACAACGGGAGTCAGGCG
GTAGGACGCTCTTCCTTTTACTGCCTGGAGTACTTTCCTTCTCAGATGCTTCGTACCGGA
AACAACTTTACCTTCAGCTACACCTTTGAAGACGTTCCTTTCCACAGCAGCTACGCTCAC
AGTCAAAGTCTGGACCGTCTCATGAATCCTCTCATCGACCAGTACCTGTATTACTTGAGC
AGAACAAACACTCCAAGCGGAACCACCACGCAGTCCAGGCTTCAGTTTTCTCAGGCCGGA
GCGAGTGACATTCGGGACCAGTCTAGGAACTGGCTTCCTGGACCCTGTTACCGCCAGCAG
CGAGTATCAAAGACAGCTGCGGATAACAACAACAGTGAATACTCGTGGACTGGAGCTACC
AAGTACCACCTCAATGGCAGAGACTCTCTGGTGAATCCGGGCCCGGCCATGGCCAGCCAC
AAGGACGATGAAGAAAAGTTTTTTCCTCAGAGCGGGGTTCTCATCTTTGGAAAACAAGAC
TCAGGAAAAACTAATGTGGACATTGAAAAGGTCATGATTACAGACGAAGAGGAAATCAGG
ACCACCAATCCCGTGGCTACGGAGCAGTATGGTGCTGTATCTACCAACCTCCAGAGCGGC
AACACACAAGCAGCTACCTCAGATGTCAACGCACAAGGCGTTCTTCCAGGCATGGTCTGG
CAGGACAGAGACGTGTACCTGCAGGGGCCCATCTGGGCAAAGATTCCACACACGGACGGA
CATTTTCACCCCTCTCCCCTCATGGGTGGATTCGGACTTAAACACCCTCCCCCGCAGATT
CTCATCAAGAACACCCCGGTACCTGCGAATCCTTCGACCACCTTCAGTGCGGCAAAGTTT
GCTTCCTTTATCACACAGTACTCCACGGGGCAGGTCAGCGTGGAGATCGAGTGGGAGCTG
CAGAAGGAGAACAGCAAACGCTGGAATCCCGAGATTCAGTACACTTCCAACTACAACAAA
TCTGTTAATGTGGACTTTACTGTGGACACTAATGGCGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTATTTCTTATCTAGTTTCCATGGCTACGTA
GATAAGTAGCATGGCGGGTTAATCATTAACTCTAAGGAACCCCTAGTGATGGAGTTGGCC
ACTCCCTCTCTGCGCGCTCGCTCGCTCACTGAGGCCGCCCGGGCAAAGCCCGGGCGACGC
CCGGGCTTTGCCCGGGCGGCCTCAGTGAGCGAGCGAGCGCGCAGAGAGGGAGTGGCCAA
SEQ ID NO: 67
>#1273_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCACGTGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCAAATTCTGACGCGC
CGGTGATTAGATCAAAAACCTCAGCCAGGTATATGGAGCTGGTCGGGTGGCTCGTGGATA
AGGGGATTACCTCGGAGAAGCAGTGGATCCAGGAGGATCAGGCTTCATACATCTCCTTCA
ATGCGGCCTCTAACTCGCGATCCCAGATCAAGGCTGCTCTGGACAATGCGGGAAAGATCA
TGAGCCTGACTAAAACCGCCCCCGACTACCTGGTGGGCCAGCATCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACGGTGCCCTTCT
ACGGGTGCGTAAACTGGACCAATGAGAACTTTCCATTCAACGACTGTGTCGACAAGATGG
TGATCTGGTGGGAGGAGGGGAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAAAAAA
GACCCGCCCCCAGTGACGCAGATATAAGTGAGCCCAAACGGGTGCGCGAGTCAGTTGCGC
AGCCATCGACGTCAGACGCGGAAGCTTCAATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATTTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATCAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGATCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAAACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAGAGATTGCAGACTGGAGACGCAGACTCCGTACCTGACCCCCAGCCTCTCGGA
CAGCCACCAGCAGCCCCCTCTGGTCTGGGAACTAATACGATGGCTACAGGCAGTGGCGCA
CCAATGGCAGACAATAACGAGGGCGCCGACGGAGTGGGTAATTCCTCGGGAAATTGGCAT
TGCGATTCCACATGGATGGGCGACAGAGTCATCACCACCAGCACCCGCACCTGGGCTCTG
CCCACCTACAACAACCATCTGTACAAGCAGATATCCAGCCAGTCTGGAGCCAGCAACGAC
AACCACTACTTTGGCTACAGCACCCCCTGGGGGTATTTTGACTTCAACAGATTCCACTGC
CACTTTTCACCACGTGACTGGCAAAGACTCATCAACAACAACTGGGGATTCCGGCCAAAA
AGACTCAACTTCAAGCTCTTTAACATTCAAGTCAAGGAGGTCACGCAGAATGACGGTACG
ACAACGATTGCCAATAACCTTACCAGCACGGTTCAGGTGTTTACTGACTCGGAGTACCAG
CTCCCGTACGTCCTCGGCTCGGCGCATCAAGGATGCCTCCCGCCGTTCCCAGCAGACGTC
TTCATGGTGCCACAGTATGGATACCTCACCCTGAACAACGGGAGTCAGGCAGTAGGACGC
TCTTCATTTTACTGCCTGGAGTACTTTCCTTCTCAGATGCTTCGTACCGGAAACAACTTT
ACCTTCAGCTACACTTTTGAGGACGTTCCTTTCCACAGCAGCTACGCTCACAGCCAGAGT
TTGGACCGTCTCATGAATCCTCTCATCGACCAGTACCTGTATTACTTGAGCAGAACAAAC
ACTCCAAGCGGAACCACTACGCAGTCCAGGCTTCAGTTTTCTCAGGCCGGAGCGAGTGAC
ATTCGGGACCAGTCTAGGAACTGGCTTCCTGGACCCTGTTACCGCCAGCAGCGAGTATCA
AAGACAGCTGCGGACAACAACAACAGTGAATACTCGTGGACTGGAGCTACCAAGTACCAC
CTCAATGGCAGAGACTCTCTGGTGAATCCGGGCCCGGCCATGGCCAGCCACAAGGACGAT
GAAGAAAAGTTTTTTCCTCAGAGCGGGGTTCTCATCTTTGGAAAACAAGACTCGGGAAAA
ACTAATGTGGACATTGAAAAGGTCATGATTACAGACGAAGAGGAAATCAGGACCACCAAT
CCCGTGGCTACGGAGCAGTATGGTGCTGTATCTACCAACCTCCAGAGCGGCAACACACAA
GCAGCTACCTCAGATGTCAACGCACAAGGTGTTCTTCCAGGCATGGTCTGGCAGGACAGA
GACGTGTACCTGCAGGGGCCCATCTGGGCAAAGATTCCACACACGGACGGACATTTTCAC
CCCTCTCCCCTCATGGGCGGATTCGGACTTAAACACCCTCCTCCACAAATTCTCATCAAG
AACACCCCTGTACCTGCGAATCCTTCGACCACCTTCAGTGCGGCAAAGTTTGCTTCCTTC
ATTACACAGTACTCCACGGGGCAGGTCAGCGTGGAGATCGAGTGGGAGCTGCAGAAAGAG
AACAGCAAACGCTGGAATCCCGAGATTCAGTACACTTCCAACTACAACAAATCTGTTAAT
GTGGACTTTACTGTAGACACTAATGGTGTGTATTCAGAGCCTCGCCCCATTGGCACCAGA
TACCTGACTCGTAATCTGTAATTGCTTGTTAATCAATAAACCGTTTAATTCGTTTCAGTT
GAACTTTGGTCTCTGCGTACTTATTTCTTATCTGGTTTCCATGGCTACGTAGATAAGTAG
CATGGCGGGTTAATCATTAAATCTAAGGAACCCCTAGTGATGGAGTTGGCCACTCCCTCT
CTGCGCGCTCGCTCGCTCACTGAGGCCGGGCGACCAAAGGTCGCCCGACGCCCGGGCTTT
GCCCGGGCGGCCTCAGTGAGCGAGCGAGCGCGCAGAGAGGGAGTGGCCAA
SEQ ID NO: 68
>#2087_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACGCCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCACGTGC
TCGTGGAAACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCAGAAATGGCGCCGGAGGCGGGAACAAGGTGGTGGATGAGTGCTACATCC
CCAATTACCTGCTCCCCAAAACCCAACCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACGTCGGAGAAGCAGTGGATCCAGGAGGATCAGGCTTCATACATCTCCTTCA
ATGCGGCCTCCAACTCGCGGTCTCAAATCAAGGCTGCCTTGGACAATGCGGGAAAGATTA
TGAGCCTGACTAAAACCGCCCCCGACTACCTGGTGGGCCAGCAGCCCGTGGAGGACATTT
TCAGCAATCGGATTTATAAAATCTTGGAACTGAACGGGTACGATCCCCAATACGCTGCTT
CCGTCTTTCTGGGATGGGCCACAAAAAAGTTCGGCAAGAGGAACACCATCTGGCTGTTTG
GGCCTGCAACTACCGGGAAGACCAACATCGCGGAGGCCATAGCCCACACTGTACCCTTCT
ACGGGTGTGTAAACTGGACCAATGAGAACTTTCCCTTCAACGACTGCGTCGACAAGATGG
TGATCTGGTGGGAGGAGGGGAAGATGACCGCCAAGGTCGTGGAGTCGGCCAAAGCCATTC
TCGGCGGAAGCAAGGTGCGCGTGGACCAAAAATGCAAGTCCTCGGCCCAGATCGACCCGA
CTCCCGTGATCGTCACTTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTTTACGTCAAAAAGGGTGGAGCCAAAAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACAGGAAAAGCGGGCAACCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTTCAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGAACC
TGGGCTCTGCCCACCTACAACAACCATCTCTACAAGCAGATTTCCAGCCAATCAGGAGCC
TCAAACGACAACCACTACTTTGGCTACAGCACCCCTTGGGGGTATTTTGACTTCAACAGA
TTCCACTGCCACTTTTCACCACGTGACTGGCAAAGACTCATCAACAACAACTGGGGATTC
CGGCCCAAAAGACTCAACTTCAAGCTTTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTTGGCTCGGCGCATCAAGGATGCCTCCCGCCGTTCCCA
GCAGACGTCTTCATGGTGCCACAGTATGGATACCTCACCCTGAACAACGGGAGTCAGGCA
GTAGGACGCTCTTCATTTTACTGCCTGGAGTACTTTCCTTCTCAAATGCTGCGTACCGGA
AACAACTTTACCTTCAGCTACACCTTTGAGGACGTTCCTTTCCACAGCAGCTACGCTCAC
AGCCAGAGTTTGGACCGTCTCATGAATCCTCTCATCGACCAGTACCTGTATTACTTGAGC
AGAACAAACACTCCAAGCGGAACCACCACGCAGTCCAGGCTTCAGTTTTCTCAGGCCGGA
GCGAGTGACATTCGGGACCAGTCTAGGAACTGGCTTCCTGGACCCTGTTACCGCCAGCAG
CGAGTATCAAAGACAGCTGCGGACAACAACAACAGTGAGTACTCGTGGACTGGAGCTACC
AAGTACCACCTCAATGGAAGAGACTCTCTGGTGAATCCGGGCCCGGCCATGGCCAGCCAC
AAGGACGATGAAGAAAAGTTTTTTCCTCAAAGCGGGGTTCTCATCTTTGGGAAGCAAGGC
TCAGAGAAAACAAATGTGGACATTGAAAAGGTCATGATTACAGACGAAGAGGAAATCAGG
ACCACCAATCCCGTGGCTACGGAGCAGTATGGTGCTGTATCTACCAACCTCCAGAGCGGC
AACACACAAGCAGCTACCTCAGATGTCAACACACAAGGTGTTCTTCCAGGCATGGTCTGG
CAGGACAGAGACGTGTACCTGCAGGGGCCCATCTGGGCAAAGATTCCACACACGGACGGA
CATTTTCACCCCTCTCCCCTCATGGGCGGATTTGGATTGAAGCACCCTCCCCCGCAGATT
CTCATCAAGAACACCCCGGTACCTGCGAATCCTTCGACCACCTTCAGTGCAGCAAAGTTT
GCTTCCTTCATCACACAGTATTCCACGGGGCAGGTCAGCGTGGAGATCGAGTGGGAGCTG
CAGAAGGAGAACAGCAAACGCTGGAACCCCGAGATCCAGTACACTTCCAACTATAACAAG
TCTGTTAATGTGGACTTTACTGTGGACACTAATGGCGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTATTTTTTATCTAGTTTCCATGGCTACGTA
GATAAGTAGCATGGAGGGTTAATCATTATCTATAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 69
>#2206_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTTGGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATCTGCCCGGTATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCACGTGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTTGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAACTCGCGGTCCCAAATCAAGGCTGCTCTGGACAATGCGGGAAAAATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATATGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCCTTCAACGACTGCGTCGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACTTCCAACACCAACATGTGCGCCGTGATTGACGGGAACTCAACGA
CCTTCGAACACCAGCAGCCGTTGCAAGACCGGATGTGCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTTGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGCGGAGCCAGACTCCTCCTCGGGAACCGGAAAAGCGGGCAACCAGCCT
GCAAGAAAAAGACTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTTCAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGAACC
TGGGCCCTGCCCACCTACAACAACCATCTCTACAAGCAGATTTCCAGCCAATCAGGAGCT
TCAAACGACAACCACTACTTTGGCTACAGCACCCCCTGGGGGTATTTTGACTTCAACAGA
TTCCACTGCCACTTCTCGCCACGTGACTGGCAGAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTGCCACAGTATGGATACCTCACCCTGAACAACGGGAGTCAGGCA
GTAGGACGCTCTTCATTTTACTGCCTGGAGTACTTTCCTTCTCAGATGCTGCGTACCGGA
AACAACTTTACCTTCAGCTACACCTTTGAGGACGTTCCTTTCCACAGCAGCTACGCTCAC
AGCCAGAGTTTGGACCGTCTCATGAATCCTCTCATCGACCAGTACCTGTATTACTTGAGC
AGAACAAACACGCCAAGCGGAACCACCACGCAGTCCAGACTTCAGTTTTCTCAGGCCGGA
GCAAGTGACATTCGGGACCAGTCTAGGAACTGGCTTCCTGGACCCTGTTACCGCCAGCAG
CGAGTATCAAAGACATCTGCGGATAACAACAACAGTGAATACTCGTGGACTGGAGCTACC
AAGTACCACCTCAATGGCAGAGACTCTCTGGTGAATCCGGGCCCGGCCATGGCCAGCCAC
AAGGACGATGAAGAAAAGTTTTTTCCTCAAAGCGGGGTTCTCATCTTTGGAAAACAAGGC
TCAGAGAAAACAAATGTGGACATTGAAAAGGTCATGATTACAGACGAAGAGGAAATCAGG
ACCACCAATCCCGTGGCTACGGAGCAGTATGGTGCTGTATCTACCAACCTCCAGAGCGGC
AACACACAAGCAGCTACCTCAGATGTCAACACACAAGGCGTTCTTCCAGGCATGGTCTGG
CAGGACAGAGACGTGTACCTGCAGGGGCCCATCTGGGCAAAGATTCCACACACAGACGGA
CATTTTCACCCCTCTCCCCTCATGGGCGGATTTGGATTAAAGCACCCTCCCCCGCAAATT
CTCATTAAGAATACCCCGGTACCTGCAAATCCTTCTACCACTTTCAGCGCGGCAAAGTTT
GCTTCTTTCATCACACAGTACTCCACGGGGCAGGTCAGCGTGGAGATCGAGTGGGAGCTG
CAGAAGGAGAACAGCAAACGCTGGAATCCCGAGATTCAATACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGTGTACTTCTTTCTTATCTAGTTTCCATGGCTACGTA
GATAAGTACCATGGATGGTTAATCATTAACTATAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 70
>#877_AAV.FL.linear [organism = Adeno-associated virus][mol_type = genomic DNA]
[isolation_source = liver tissue][topology = linear]
TTGGCCACTCCCTCTCTGCGCGCTCGCTCGCTCACTGAGGCCGGGCGACCAAAGGTCGCC
CGACGTCCGACCTTTGGTCGCCCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGCCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATTTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCACGTGC
TGGTGGAGACTACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGACA
AGGGGATTACCTCTGAGAAGCAGTGGATCCAGGAGGACCAGGCCTCATACATCTCCTTCA
ATGCGGCCTCCAACTCGCGGTCCCAGATCAAGGCTGCTCTGGACAATGCGGGAAAGATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATATGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCAGAGGCCATAGCCCACACTGTGCCCTTCT
ACGGGTGCGTAAACTGGACCAATGAGAACTTTCCATTCAACGACTGTGTCGACAAGATGG
TGATCTGGTGGGAGGAGGGAAAGATGACCGCCAAGGTTGTGGAGTCGGCCAAAGCTATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTTGAGCACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCACATCATGGGAAAGGTGCCAGACGCTTGCACTGCCTGCAATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCAAAAAAGAGGGTTCTT
GAACCTCTGGGCCTGGTTGAGGAACCTGTTAAGACGGCTCCGGGAAAAAAGAGGCCGGTA
GAGCACTCTCCTGCGGAGCCAGACTCCTCCTCGGGAACCGGAAAGGCGGGCAACCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGCACC
TGGGCTCTGCCCACCTACAACAACCATCTGTACAAGCAGATTTCCAGCCAATCAGGAGCC
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGGCCCAAGAGACTCAACTTCAAGCTCTTTAACATTCAAGTCAAGGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTGCCACAGTATGGATACCTCACCCTGAACAACGGGAGTCAGGCA
GTAGGACGCTCTTCATTTTACTGCCTGGAGTACTTTCCTTCTCAGATGCTTCGTACCGGA
AACAACTTTACCTTCAGCTACACCTTTGAAGACGTGCCTTTCCACAGCAGCTACGCTCAC
AGTCAAAGTCTGGACCGTCTCATGAATCCTCTCATCGACCAGTACCTGTATTACTTGAGC
AGAACAAACACTCCAAGCGGAACCACTACGCAGTCCAGGCTTCAGTTTTCTCAGGCCGGA
GCGAGTGACATTCGGGACCAGTCTAGGAACTGGCTTCCTGGACCCTGTTACCGCCAGCAG
CGAGTATCAAAGACAGCTGCGGACAACAACAACAGTGAATACTCGTGGACTGGAGCTACC
AAGTACCACCTCAATGGCAGAGACTCTCTGGTGAATCCGGGCCCGGCCATGGCCAGCCAC
AAGGACGATGAAGAAAAGTTTTTTCCTCAAAGCGGGGTTCTCATCTTTGGGAAGCAAGGC
TCAGAGAAAACAAATGTGGACATTGAAAAGGTCATGATTACAGACGAAGAGGAAATCAGG
ACCACCAATCCCGTGGCTACGGAGCAGTATGGTTCTGTATCTACCAACCTCCAGAGCGGC
AACACACAAGCAGCTACCGCAGATGTCAACACACAAGGCGTTCTTCCAGGCATGGTCTGG
CAGGACAGAGACGTGTACCTGCAGGGGCCTATCTGGGCAAAGATTCCACACACGGACGGA
CATTTTCACCCCTCTCCCCTCATGGGCGGATTTGGACTGAAGCACCCTCCTCCACAGATT
CTCATCAAGAACACCCCGGTACCTGCGAATCCTTCTACCACTTTCAGTGCGGCAAAATTT
GCTTCTTTCATCACACAGTACTCCACGGGGCAGGTCAGCGTGGAGATCGAGTGGGAGCTG
CAGAAGGAGAACAGCAAACGCTGGAATCCCGAGATTCAGTACACTTCAAACTACAACAAG
TCTGTTAATGTGGACTTTACTGTGGACACTAATGGCGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTCTTTCTTATCTAGTTTCCATGGCTACGTA
GATAAGTACCATGGAGGGTTAATCATTAACACAAGGAACCCCTAGTGATGGAGTTGGCCA
CTCCCTCTCTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGAGAGGGAGTGGCCAA
SEQ ID NO: 71
>#1534_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGCCCGGGCAAAGCCCGGG
CGTCGGGCGACCTTTGGTCGCCCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTACGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAATGGCGCCGTGTGAGTAAGGCCC
CGGAGGCCCTCTTCTTTGTGCAATTTGAGAAGGGAGAAAGCTACTTCCACATGCACGTGC
TCGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGACA
AAGGGATTACCTCGGAGAAGCAGTGGATCCAGGAGGACCAGGCCTCATACATCTCCTTCA
ATGCGGCCTCCAACTCGCGGTCCCAGATCAAGGCTGCTCTGGACAATGCGGGAAAAATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTATTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCATTCAATGACTGCGTCGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGTGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATCAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGCGGAGCCAGACTCCTCCTCGGGAACCGGAAAAGCGGGCAACCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGCACC
TGGGCTCTGCCCACCTACAACAACCATCTGTACAAGCAGATATCCAGCCAGTCTGGAGCC
AGCAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCAAAAAGACTCAACTTCAAGCTCTTTAACATTCAAGTCAAGGAGGTCACGCAGAAT
GACGGTACGACAACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTGCCACAGTATGGATACCTCACCCTGAACAACGGGAGTCAGGCA
GTAGGACGCTCTTCATTTTACTGCCTGGAGTACTTTCCTTCTCAGATGCTTCGTACCGGA
AACAACTTTACCTTCAGCTACACCTTTGAAGACGTGCCTTTCCACAGCAGCTACGCTCAC
AGTCAAAGTCTGGACCGTCTCATGAATCCTCTCATCGACCAGTACCTGTATTACTTGAGC
AGAACAAACACTCCAAGCGGAACCACCACGCAGTCCAGGCTTCAGTTTTCTCAGGCCGGA
GCGAGTGACATTCGGGACCAGTCTAGGAACTGGCTTCCTGGACCCTGTTACCGCCAGCAG
CGAGTATCAAAGACAGCTGCGGATAACAACAACAGTGAATATTCGTGGACTGGAGCTACC
AAGTACCACCTCAATGGCAGAGACTCTCTGGTGAATCCGGGCCCGGCCATGGCCAGCCAC
AAGGACGATGAAGAAAAGTTTTTTCCTCAAAGCGGGGTTCTCATCTTTGGGAAGCAAGGC
TCAGAGAAAACAAATGTGGACATTGAAAAGGTCATGATTACAGACGAAGAGGAAATCAGG
ACCACCAATCCCGTGGCTACGGAGCAGTATGGTTCTGTATCTACCAACCTCCAGAGCGGC
AACACACAAGCAGCTACCTCAGATGTCAACACACAAGGCGTTCTTCCAGGCATGGTCTGG
CAGGACAGAGATGTGTACCTGCAGGGGCCTATCTGGGCAAAGATTCCACACACGGACGGA
CATTTTCACCCCTCTCCCCTCATGGGCGGATTTGGACTTAAACACCCTCCTCCACAGATT
CTCATCAAGAACACCCCGGTACCTGCAAATCCTTCGACCACCTTCAGTGCGGCAAAGTTT
GCTTCTTTCATCACACAGTACTCCACGGGGCAGGTCAGCGTGGAGATCGAGTGGGAGCTA
CAGAAGGAGAACAGCAAACGCTGGAATCCCGAGATTCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTAAACTTTGGTCTCCGCGTACTTCTTTTCTTATCTAGTTTCCATGGCTACGT
AGATAAGTACCATGGCGGGTTAATCATTAACTCTAAGGAACCCCTAGTGATGGAGTTGGC
CACTCCCTCTCTGCGCGCTCGCTCGCTCACTGAGGCCGGGCGACCAAAGGTCGCCCGACG
CCCGGGCTTTGCCCGGGCGGCCTCAGTGAGCGAGCGAGCGCGCAGACTGAAACGAATTAA
SEQ ID NO: 72
>#2557_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCSATTTTGAAGCGGGA
GGTTTGAACGCGCAGCCGCCATGCCGGGATTTTACGAGATTGTGATTAAGGTCCCCAGCG
ACCTTGACGAGCATCTACCCGGCATTTCTGACAGCTTTGTGAACTGGGTAGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGYGCCGCGTGAGTAAGGCCC
CGGAGGCCCTCTTCTTTGTGCAATTTGAGAAGGGAGAGAGCTACTTCCACATGCACGTGC
TGGTGGAGACAACCGGGGTGAAATCCATGGTTTTGGGACGTTTCCTGAGTCAGATTCGCG
AAAAACTGATTCAGAGAATCTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
GGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGRTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAACTCGAGGTCCCAAATCAAGGCTGCCTTGGACAATGCGGGGAAGATTA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATATGCGGCTT
CCGTCTTCTTGGGATGGGCTACGAAAAAGTTCGGCAAGAGGAACACCATCTGGCTGTTTG
GGCCTGCAACTACCGGGAAGACCAACATCGCGGAGGCCATAGCCCACACTGTTCCCTTCT
ACGGGTGCGTAAACTGGACCAATGAGAACTTTCCCTTCAACGACTGTGTCGACAAGATGG
TGATCTGGTGGGAGGAGGGGAAGATGACCGCCAAGGTCGTGGAGTCGGCCAAAGCCATTC
TCGGAGGAAGCAAAGTGCGTGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTCCCGTGGCCACCAAGCAGGAAGTCAAAGATTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATYC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAACACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCCAAAAAGAGGGTTCTT
GAACCTCTGGGCCTGGTTGAGGAGCCTGTTAAGACGGCTCCGGGAAAAAAGAGGCCAGTA
GAGCACTCTCCTGCGGAGCCAGACTCCTCCTCGGGAACCGGAAAAGCGGGCAACCAGCCT
GCAAGAAAGAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGAACC
TGGGCCCTGCCCACCTACAACAACCACCTCTACAAGCAGATTTCCAGCCAGTCTGGAGCC
AGCAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCAAAAAGACTCAACTTCAAGCTCTTTAACATTCAAGTCAAGGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTGCCACAGTATGGATACCTCACCCTGAACAACGGGAGTCAGGCA
GTAGGACGCTCTTCATTTTACTGCCTGGAGTACTTTCCTTCTCAGATGCTTCGTACCGGA
AACAACTTTACCTTCAGCTACACCTTTGAAGACGTGCCTTTCCACAGCAGCTACGCTCAC
AGTCAAAGTCTGGACCGTCTCATGAATCCTCTCATCGACCAGTACCTGTATTACTTGAGC
AGAACAAACACTCCAAGCGGAACCACTACGCAGTCCAGGCTTCAGTTTTCTCAGGCCGGA
GCGAGTGACATTCGGGACCAGTCTAGGAACTGGCTTCCTGGACCCTGTTACCGCCAGCAG
CGAGTATCAAAGACAGCTGCGGACAACAACAACAGTGAATACTCGTGGACTGGAGCTACC
AAGTACCACCTCAATGGCAGAGACTCTCTGGTGAATCCGGGCCCGGCCATGGCCAGCCAC
AAGGACGATGAAGAAAAGTTTTTTCCTCAAAGCGGGGTTCTCATCTTTGGGAAGCAAGGC
TCAGAGAAAACAAATGTGGACATTGAAAAGGTCATGATTACAGACGAAGAGGAAATCAGG
ACCACCAATCCCGTGGCTACGGAGCAGTATGGTTCTGTATCTACCAACCTCCAGAGCGGC
AACACACAAGCAGCTACCGCAGATGTCAACACACAAGGCGTTCTTCCAGGCATGGTATGG
CAGGACAGAGACGTGTACCTGCAGGGGCCTATCTGGGCAAAGATTCCACACACGGACGGA
CATTTTCACCCCTCTCCCCTCATGGGCGGATTTGGACTTAAACACCCTCCTCCACAGATT
CTCATCAAGAACACCCCGGTACCTGCAAATCCTTCGACCACCTTCAGTGCGGCAAAGTTT
GCTTCTTTCATCACACAGTACTCCACGGGGCAGGTCAGTGTGGAGATTGAGTGGGAGCTG
CAGAAGGAGAACAGCAAACGCTGGAATCCCGAGATCCAGTACACTTCCAACTACAACAAR
TCTGTTAATGCGGACCTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTGCTTCTTTCTTATCTAGTTTACATGGCTACGTA
GATAAGTAGCATGGCGGGTTAATCATTAACTACAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 73
>#1919_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGCCCTT
TCCCCCACACCCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATCAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGTGTGAGTAAGGCCC
CGGAGGCCCTCTTCTTTGTGCAATTTGAGAAGGGAGAGAGCTACTTCCACATGCACGTGC
TTGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTTGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTAATTAGATCAAAAACCTCAGCCAGGTACATGGAGCTGGTTGGGTGGCTCGTGGATA
AGGGGATTACCTCGGAGAAGCAGTGGATCCAGGAGGACCAGGCCTCGTACATCTCCTTCA
ATGCGGCCTCCAACTCGCGGTCGCAGATCAAGGCTGCTCTGGACAATGCGGGAAAGATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATATGCGGCTT
CCGTTTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAAAACTTTCCATTCAATGACTGTGTTGACAAGATGG
TGATCTGGTGGGAGGAGGGAAAGATGACCGCCAAGGTCGTGGAGTCGGCCAAAGCCATTC
TCGGCGGAAGCAAGGTGCGCGTGGACCAGAAATGCAAGTCCTCGGCCCAGATAGACCCGA
CTCCCGTGATCGTCACTTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCATATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTGCCAGATGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAGGCGGGCAACCAGCCT
GCAAGAAAGAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGCACC
TGGGCTCTGCCCACCTACAACAACCATCTGTACAAGCAGATTTCCAGCCAGTCTGGAGCC
AGCAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTGCCACAGTATGGATACCTCACCCTGAACAACGGGAGTCAGGCG
GTAGGACGCTCTTCCTTTTACTGCCTGGAGTACTTTCCTTCTCAGATGCTTCGTACCGGA
AACAACTTTACCTTCAGCTACACCTTTGAAGACGTTCCTTTCCACAGCAGCTACGCTCAC
AGTCAAAGTTTGGACCGTCTCATGAATCCTCTCATCGACCAGTACCTGTATTACTTGAGC
AGAACAAACACTCCAAGCGGAACCACTACGCAGTCCAGGCTTCAGTTTTCTCAGGCCGGA
GCAAGTGACATTCGGGACCAGTCTAGGAACTGGCTTCCTGGACCCTGTTACCGCCAGCAG
CGAGTATCAAAGACAGCTGCGGATAACAACAACAGTGAATACTCGTGGACTGGAGCTACC
AAGTACCACCTCAATGGCAGAGACTCTCTGGTGAATCCAGGCCCGGCCATGGCCAGCCAC
AAGGACGATGAAGAAAAGTTTTTTCCTCAGAGCGGGGTTCTCATCTTTGGGAAGCAAGGC
TCAGAGAAAACAAATGTGGACATTGAAAAGGTCATGATTACAGACGAAGAGGAAATCAGG
ACCACCAATCCCGTGGCTACGGAGCAGTATGGTGCTGTATCTACCAACCTCCAGAGCGGC
AACACACAAGCAGCTACCTCAGATGTCAACACACAAGGCGTTCTTCCAGGCATGGTCTGG
CAGGACAGAGACGTGTACCTGCAGGGGCCCATCTGGGCAAAGATTCCACACACGGACGGA
CATTTTCACCCCTCTCCCCTCATGGGCGGATTCGGACTGAAGCACCCTCCTCCACAGATT
CTCATCAAGAACACCCCGGTACCTGCGAATCCTTCGACCACCTTCAGTGCGGCAAAGTTT
GCTTCCTTCATCACACAGTATTCCACGGGGCAGGTCAGCGTGGAGATCGAGTGGGAGCTG
CAGAAGGAGAACAGCAAACGCTGGAATCCCGAGATCCAGTACACTTCCAACTATAACAAG
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTGTAATTC
GTTTCAGTTGAACTTTGGTCTCTGCGTACTTCTTTTCTTATCTAGTTTCCATGGCTACGT
AGATAAGTAGCATGGCGAGTTAATCATTAACTCTAAGGAACCCCTAGTGATGGAGTTGGC
CACTCCCTCTCTGCGCGCTCGCTCGCTCACTGAGGCCGCCCGGGCAAAGCCCGGGCGTCG
GGCGAGCTTTGCCCGGGCGGCCTCAGTGAGCGAGCGAGCGCGCAGAGAGGGAGTGGCCAA
SEQ ID NO: 74
>#1260_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAAAAGGGAGAGAGCTACTTCCACATGCACGTGC
TTGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAACCTGAACTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAAAATCAGAATCCCAATTCTGACGCGC
CGGTGATTAGATCAAAAACCTCAGCCAGGTACATGGAGCTGGTCGGGTGGCTCGTGGATA
AGGGGATTACGTCGGAGAAGCAGTGGATCCAGGAGGACCAGGCTTCATACATCTCCTTCA
ATGCGGCCTCCAACTCGCGGTCCCAGATCAAAGCTGCTCTGGACAATGCGGGAAAGATCA
TGAGCCTCACTAAAACTGCCCCCGACTACCTGGTGGGCCAGCAGCCCGTGGAGGACATTT
CCAGCAATCGGATTTATAAAATCTTGGAACTGAACGGGTACGATCCCCAATACGCTGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCTTTCAATGATTGCGTCGACAAGATGG
TGATCTGGTGGGAGGAGGGAAAGATGACCGCCAAGGTTGTGGAGTCGGCCAAAGCCATTC
TCGGAGGAAGCAAGGTGCGCGTGGACCAAAAGTGCAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTCGCCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGTGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAAAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTCAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACGGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGCGGAGCCAGACTCCTCCTCGGGAACCGGAAAAGCGGGCCAGCAGCCT
GCAAGAAAAAGACTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGCACC
TGGGCTCTGCCCACCTACAACAACCATCTGTACAAGCAGATTTCCAGCCAGTCTGGAGCC
AGCAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCGGACGTCTTCATGGTCCCACAGTATGGATACCTCACCCTGAACAACGGGAGTCAGGCG
GTAGGACGCTCTTCATTTTACTGCCTGGAGTACTTTCCTTCTCAGATGCTTCGTACCGGA
AACAACTTTACCTTCAGCTACACCTTTGAAGACGTTCCTTTCCACAGCAGCTACGCTCAC
AGTCAAAGTCTGGACCGTCTCATGAATCCTCTCATCGACCAGTACCTATATTACTTAAGC
AGAACAAACACGCCGAGCGGAACCACTACGCAGTCCAGGCTTCAGTTTTCTCAGGCCGGA
GCAAGTGACATTCGGGACCAGTCTAGGAACTGGCTTCCTGGACCCTGTTACCGCCAGCAG
CGAGTATCAAAGACATCTGCGGATAACAACAACAGTGAATACTCGTGGACTGGAGCTACC
AAGTACCACCTCAATGGCAGAGACTCTCTGGTGAATCCGGGCCCGGCCATGGCCAGCCAC
AAGGACGATGAAGAAAAGTTTTTTCCTCAGAGCGGGGTTCTCATATTTGGGAAGCAAGGC
TCAGAGAAAACAAATGTGGACATTGAAAAGGTCATGATTACAGACGAAGAGGAAATCAGG
ACCACCAATCCCGTGGCTACGGAGCAGTATGGTGCTGTATCTACCAACCTCCAGAGCGGC
AACACACAAGCAGCTACCTCAGATGTCAACGCACAAGGTGTTCTTCCAGGCATGGTCTGG
CAGGACAGAGACGTGTACCTGCAGGGGCCCATCTGGGCAAAGATTCCCCACACGGACGGA
CACTTTCACCCTTCTCCTCTTATGGGCGGATTCGGACTTAAACACCCTCCCCCGCAGATT
CTCATCAAGAACACCCCGGTACCTGCGAATCCTTCGACCACCTTCAGCGCGGCAAAGTTT
GCTTCCTTCATCACACAGTATTCCACGGGGCAGGTCAGCGTAGAAATCGAGTGGGAGCTG
CAGAAGGAGAACAGCAAACGCTGGAATCCCGAGATCCAGTACACTTCCAACTATAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTCTTTCTTATCTAGTTTCCATGGCTACGTA
GATAAGTAGCATGGCGGGTTAATCATTAACACAAGGAACCCCTAGTGATGGAGTTGGCCA
CTCCCTCTCTGCGCGCTCGCTCGCTCACTGAGGCCGCCCGGGCAAAGCCCGGGCGTCGGG
CGACCTTTGGTCGGGCGGCCTCAGTGAGCGAGCGAGCGCGCAGAGAGGGAGTGGCCAA
SEQ ID NO: 75
>#3765_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTAAATTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCAGGATTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGTGTGAGTAAGGCCC
CGGAGGCCCTCTTCTTTGTGCAATTTGAGAAGGGAGAGAGCTACTTCCACATGCACGTGC
TCGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATCAGATCAAAAACCTCAGCCAGGTACATGGAGCTGGTCGGGTGGCTCGTGGATA
AGGGGATTACCTCGGAGAAGCAGTGGATCCAGGAGGATCAGGCTTCATACATCTCCTTCA
ATGCGGCCTCCAACTCGCGGTCCCAAATCAAGGCTGCTCTGGACAATGCGGGAAAGATTA
TGAGCCTGACTAAAACTGCCCCCGACTACCTGGTGGGCCAGCAGCCCGTGGAGGACATTT
CCAGCAATCGGATTTATAAAATCTTGGAACTGAACGGGTACGATCCCCAATACGCTGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAAGCCATAGCCCACACTGTGCCCTTCT
ACGGGTGCGTAAACTGGACCAATGAGAACTTTCCATTCAACGACTGTGTCGACAAGATGG
TGATCTGGTGGGAGGAGGGGAAGATGACCGCCAAGGTTGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTCACCCGCCGTC
TGGATCATGACTTTGGGAAGGTCACCAAGCAGGAAGTGAAAGACTTTTTCCGGTGGGCAA
ATGATCACGTGGTCGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCAATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATTATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGGGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAGCCTGTTAAGACGGCTCCGGGAAAAAAGAGGCCGGTA
GAGCACTCTCCTGCGGAGCCAGACTCCTCCTCGGGAACCGGAAAAGCGGGCAACCAGCCT
GCAAGAAAAAGACTGAATTTTGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGAACC
TGGGCTCTGCCCACCTACAACAACCATCTGTACAAGCAGATTTCCAGCCAATCAGGAGCC
TCAAACGACAACCACTACTTTGGCTACAGCACCCCTTGGGGGTATTTTGACTTCAACAGA
TTCCACTGCCACTTTTCACCACGTGACTGGCAAAGACTCATCAACAACAATTGGGGATTC
CGACCCAAGAGACTCAACTTCAAGCTCTTTAACATTCAAGTCAAAGAGGTCACGCAGAAT
GACGGTACGACGACGATCGCCAATAACCTTACCAGCACGGTTCAGGTGTTTACTGACTCG
GAGTACCAGCTCCCGTACGTTCTCGGCTCGGCGCATCAAGGATGCCTCCCGCCGTTCCCA
GCAGACGTCTTCATGGTGCCACAGTATGGATACCTCACCCTGAACAACGGGAGTCAAGCA
GTAGGACGCTCTTCATTTTACTGCCTGGAATACTTTCCTTCTCAGATGCTTCGTACCGGA
AACAACTTTACCTTCAGCTATACCTTTGAGGACGTTCCTTTCCACAGCAGCTACGCTCAC
AGCCAGAGTTTGGACCGCCTCATGAATCCTCTCATCGACCAGTACCTGTATTACTTGAGC
AGAACAAACACTCCAAGCGGAACCACCACGCAGTCCAGGCTTCAGTTTTCTCAGGCCGGA
GCAAGTGACATTCGGGACCAGTCTAGGAACTGGCTTCCTGGACCCTGTTACCGCCAGCAG
CGAGTATCAAAGACACCTGCGGATAACAACAACAGTGAATACTCGTGGACTGGAGCTACC
AAGTACCACCTCAATGGCAGAGACTCTCTGGTGAATCCGGGCCCGGCCATGGCCAGCCAC
AAGGACGATGAAGAAAAGTTTTTTCCTCAAAGCGGGGTTCTCATCTTTGGGAAGCAAGGC
TCAGAGAAAACAAATGTGGACATTGAAAAGGTCATGATTACAGACGAAGAGGAAATCAGG
ACCACCAATCCCGTGGCTACGGAGCAGTATGGTTCTGTATCTACCAACCTCCAGAGCGGC
AACACACAAGCAGCTACCGCAGATGTCAACACACAAGGCGTTCTTCCAGGCATGGTCTGG
CAGGACAGAGACGTGTACCTGCAGGGGCCCATCTGGGCAAAGATTCCACACACGGATGGA
CACTTTCACCCTTCTCCTCTTATGGGAGGATTTGGACTTAAACACCCTCCCCCGCAGATT
CTCATCAAAAACACCCCGGTACCTGCGAATCCTTCGACCACTTTCAGTGCAGCAAAGTTT
GCTTCCTTCATTACACAGTATTCCACGGGGCAGGTCAGCGTGGAGATCGAGTGGGAGCTG
CAGAAGGAGAACAGCAAACGCTGGAACCCCGAGATCCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCCGCGTACTTCTTTCTTATCTAGTTTCCATGGCTACGTA
GATAAGTAGCATGGCGGGTTAATCATTAACTTTAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 76
>#471_AAV.FL.linear [organism = Adeno-associated virus][mol_type = genomic DNA]
[isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTATATTAGAGGTCACGTGAGTGTTTTGCGACATTTTGCGACATCAT
GTGGTCACGCTAGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTTCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACAGAATGGCGCCGTGTGAGTAAGGCCC
CGGAGGCCCTTTTCTTTGTGCAATTTGAGAAGGGAGAGAGCTACTTCCACATGCACGTGC
TGGTGGAAACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCAAATTCTGACGCGC
CGGTGATCAGATCAAAAACCTCAGCCAGGTACATGGAGCTGGTCGGGTGGCTCGTGGATA
AGGGGATTACCTCTGAGAAGCAGTGGATCCAGGAGGACCAGGCCTCCTACATCTCCTTCA
ATGCGGCCTCCAACTCGCGGTCACAGATCAAGGCTGCTCTGGACAATGCGGGAAAGATTA
TGAGCCTGACTAAAACGGCCCCCGACTACCTGGTGGGCCAGCAGCCCGTGGAGGACATTT
CCGGCAATCGGATTTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACAAACATCGCGGAGGCCATAGCCCACACGGTGCCCTTCT
ACGGGTGCGTAAACTGGACCAATGAGAACTTTCCTTTCAACGACTGTGTCGACAAGATGG
TGATCTGGTGGGAGGAGGGGAAGATGACCGCCAAGGTCGTGGAGTCGGCCAAAGCCATTC
TTGGCGGAAGCAAGGTGCGCGTGGACCAGAAATGCAAGTCCTCGGCCCAGATAGACCCGA
CTCCTGTGATTGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAACAGGAAGTCAAAGACTTTTTCCGGTGGGCAA
AGGATCACGTGGTTGAGGTGGAGCATGAATTCTACGTCAAAAAGGGTGGAGCTAAGAAAA
GGCCCGCCCCCAGTGACGCAGATATAAGTGAGCCCAAACGGGCGCGCGAGTCAGTTGCGC
AGCCATCTACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGGGAGCCGGTCAAC
GAGGCAGACGCCGCGGCCCTCGAGCACGACAAGGCCTACGACCGGCAGCTCGACAGCGGA
GACAACCCGTACCTCAAATACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAGCCTGTTAAGACGGCTCCGGGAAAAAAGAGGCCGGTA
GAACACTCTCCTGCGGAGCCAGACTCCTCCTCGGGAACCGGAAAAGCGGGCAACCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGAACC
TGGGCTCTGCCCACCTACAACAACCATCTGTACAAGCAGATTTCCAGCCAATCAGGAGCC
TCAAACGACAACCACTACTTTGGCTACAGCACCCCTTGGGGGTATTTTGACTTCAACAGA
TTCCACTGCCACTTTTCACCACGTGACTGGCAAAGACTCATCAACAACAACTGGGGATTC
CGACCAAAGAGACTCAACTTCAAGCTCTTTAACATTCAAGTCAAGGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTGCCACAGTATGGATACCTCACCCTAAACAACGGGAGTCAAGCA
GTAGGACGCTCTTCATTTTACTGCCTGGAGTACTTTCCTTCTCAGATGCTTCGTACCGGA
AACAACTTTACCTTCAGCTATACCTTTGAGGACGTTCCTTTCCACAGCAGCTACGCTCAC
AGCCAGAGTTTGGACCGCCTCATGAATCCTCTCATCGACCAGTACCTGTATTACTTGAGC
AGAACAAACACTCCAAGCGGAACCACCACGCAGTCCAGGCTTCAGTTTTCTCAGGCCGGA
GCAAGTGACATTCGGGACCAGTCTAGGAACTGGCTTCCTGGACCCTGTTACCGCCAGCAG
CGAGTATCAAAGACACCTGCGGATAACAACAACAGTGAATACTCGTGGACTGGAGCTACC
AAGTACCACCTCAATGGCAGAGACTCTCTGGTGAATCCGGGCCCGGCCATGGCCAGCCAC
AAGGACGATGAAGAAAAGTTTTTTCCTCAAAGCGGGGTTCTCATCTTTGGGAAGCAAGGC
TCAGAGAAAACAAATGTGGACATTGAAAAGGTCATGATTACAGACGAAGAGGAAATCAGG
ACCACCAATCCCGTGGCTACGGAGCAGTATGGTTCTGTATCTACCAACCTCCAGAGCGGC
AACACACAAGCAGCTACCGCAGATGTCAACACACAAGGCGTTCTTCCAGGCATGGTCTGG
CAGGACAGAGACGTGTACCTGCAGGGGCCCATTTGGGCAAAAATTCCACACACGGATGGA
CACTTTCACCCTTCTCCTCTTATGGGAGGATTTGGACTTAAACACCCTCCCCCGCAGATT
CTCATCAAAAACACCCCGGTACCTGCGAATCCTTCAACCACCTTCAGTGCAGCAAAGTTT
GCTTCCTTCATTACACAGTACTCCACGGGGCAGGTCAGCGTGGAGATCGAGTGGGAGCTG
CAGAAGGAGAACAGCAAACGCTGGAATCCCGAGATTCAATACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTGTAATTC
GTTTCAGTTGAACTTTGGTCTCTGCGTACTTCTTTCTTATCTAGTTTCCATGTCTACGTA
GATAAGTAGCATGGCGGGTTAATCATTAACACAAGGAACCCCTAGTGATGGAGTTGGCCA
CTCCCTCTCTGCGCGCTCGCTCGCTCACTGAGGCCGCCCGGGCAAAGCCCGGGCGTCGGG
CGACCTTTGGTCGGGCGGCCTCAGTGAGCGAGCGAGCGCGCAGAGAGGGAGTGGCCAA
SEQ ID NO: 77
>#387_AAV.FL.linear [organism = Adeno-associated virus][mol_type = genomic DNA]
[isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGCTTTGCGACATTTTGCGACACTAT
GTGGTTACGCAGGGTATTTAAGCCCGAGTGAGCACGCAGGGTCTCCATTTTGAAGCGGGA
GGTTTGAACGCGCAGCCGCCATGCCGGGGTTTTACGAGATCGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAATTGCAGCGCGACTTTCTGACGGAATGGCGCCGTGTGAGTAAGGCCC
CCGAGGCCCTTTTCTTTGTGCAATTTGAGAAGGGAGAGAGCTACTTCCACATGCACGTGC
TCGTGGAGACCACGGGGGTCAAATCCATGGTTTTGGGACGTTTCCTGAGTCAAATTCGCG
AAAAACTGATTCAGAGAATTTACCGCGGGATCGAACCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAACCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACCCAGGAGCAGAACAAAGAGAATCAGAATCCCAATTCTGACGCGC
CGGTGATCAGATCAAAAACATCAGCCAGGTACATGGAGCTGGTCGGGTGGCTCGTGGATA
AGGGGATTACCTCGGAGAAGCAGTGGATTCAGGAGGACCAGGCCTCATACATCTCCTTCA
ATGCGGCCTCCAACTCGCGGTCCCAGATCAAGGCTGCTCTGGACAATGCGGGAAAGATCA
TGAGCCTGACTAAAACGGCCCCCGACTACCTGGTGGGCCAGCAGCCCGTGGAGGACATTT
CCAGCAATCGGATTTATAAAATCTTGGAACTGAACGGGTACGATCCCCAATATGCGGCTT
CCGTCTTTCTGGGATGGGCCACGAAAAAGTTCGGCAAGAGGAACACCATCTGGCTGTTTG
GACCTGCAACTACCGGGAAGACCAACATCGCAGAGGCCATAGCCCACACGGTTCCCTTCT
ACGGGTGCGTAAACTGGACCAATGAGAACTTTCCTTTCAACGATTGCGTCGACAAGATGG
TGATCTGGTGGGAGGAGGGGAAGATGACCGCCAAGGTCGTGGAGTCGGCCAAAGCCATTC
TCGGCGGAAGCAAGGTGCGCGTGGACCAGAAATGCAAGTCCTCGGCCCAGATAGACCCGA
CTCCCGTGATAGTCACCTCCAACACCAACATGTGCGCCGTGATTGACGGGAACTCCACGA
CCTTCGAACACCAGCAGCCGTTGCAAGACCGGATGTTCAAATTTGAACTTACCCGCCGTC
TGGATCATGACTTTGGAAAGGTCACCAAGCAGGAAGTGAAAGACTTTTTCCGGTGGGCAC
AGGATAACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCAATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGGGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAAACGGCTCCGGGAAAAAAGAGGCCGGTA
GAGCACTCTCCTGCGGAGCCAGATTCCTCCTCCGGAACTGGAAAGTCGGGCAACCAGCCT
GCAAGAAAGAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGAACC
TGGGCTCTGCCCACCTACAACAACCATCTCTACAAGCAGATTTCCAGCCAGTCTGGAGCC
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAATTGGGGATTC
CGGCCCAAAAGACTCAACTTCAAGCTGTTTAACATTCAAGTCAAGGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTGCCACAGTATGGATACCTCACCCTGAACAACGGGAGTCAGGCA
GTAGGACGCTCTTCATTTTACTGCCTAGAGTACTTTCCTTCTCAGATGCTGCGTACCGGA
AACAACTTTACCTTCAGCTACACCTTTGAGGACGTTCCTTTCCACAGCAGCTACGCTCAC
AGCCAGAGTTTGGACCGTCTCATGAATCCTCTCATCGACCAGTACCTGTATTACTTGAGC
AGAACAAACACTCCAAGCGGAACCACCACGCAGTCCAGGCTTCAGTTTTCTCAGGCCGGA
GCAAGTGACATTCGGGACCAGTCTAGGAACTGGCTTCCTGGACCCTGTTACCGCCAGCAG
CGAGTATCAAAGACACCTGCGGATAACAACAACAGTGAATACTCGTGGACTGGAGCTACC
AAGTACCACCTCAATGGTAGAGACTCTCTGGTGAATCCGGGCCCGGCCATGGCCAGCCAC
AAGGACGATGAAGAAAAGTTTTTTCCTCAGAGCGGGGTTCTCATCTTTGGGAAGCAAGGC
TCAGAGAAAACAAATGTGGACATTGAAAAGGTCATGATTACAGACGAAGAAGAAATCAGG
ACCACCAATCCCGTGGCTACGGAGCAGTATGGTTCTGTATCTACCAACCTCCAGAGCGGC
AACACACAAGCAGCTACCGCAGATGTCAACACACAAGGCGTTCTTCCAGGCATGGTCTGG
CAGGACAGAGACGTGTACCTGCAGGGGCCTATCTGGGCAAAGATTCCACACACGGACGGA
CATTTTCACCCCTCTCCCCTCATGGGCGGATTCGGACTTAAACACCCTCCTCCGCAAATT
CTCATCAAGAACACCCCGGTACCTGCGAATCCTTCGACCACCTTCAGTGCGGCAAAGTTT
GCTTCCTTCATCACACAGTATTCCACGGGGCAGGTCAGCGTGGAGATCGAGTGGGAGTTG
CAGAAGGAGAACAGCAAACGCTGGAACCCTGAGATCCAGTACACTTCCAACTACAACAAG
TCTGTTAATGTGGACTTTACTGTGGACACTAATGGTGTGTATTCAGAGCCTCGCCCCATT
GGCACCAGATACCTGACTCGTAATCTGTAATTGTTTGTTAATCAATAAACCGTTTAATTC
GTTTCAGTTGAACTTTGGTCTCTGCGTACTTATTTCTTATCTAGTTGCCATGGCTACGTA
AATAAGTAGCATGGCGGGTTAATCATAAACTCTAAGGAACCCCTAGTGATGGAGTTGGCC
ACTCCCTCTCTGCGCGCTCGCTCGCTCACTGAGGCCGCCCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 78
>#2040_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCAGGGTATTTAAGCCCGAGTGAGCACGCAGGGTCTCCATTTTGAAGCGGGA
GGTTTGAACGCGCAGTCACCATGCCGGGATTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATTTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCACGTGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TTACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGATACATGGAGCTGGTCGGGTGGCTCGTGGATA
AGGGGATTACCTCGGAGAAGCAGTGGATCCAGGAGGACCAGGCCTCATACATCTCCTTCA
ATGCGGCCTCCAACTCGCGGTCCCAGATCAAGGCTGCTCTGGACAATGCGGGAAAGATCA
TGAGCCTCACTAAAACCGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTTGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCCTTCAACGACTGTGTCGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCGTGCAAAACCTGCGAGAGAATGA
ATCAGATTTCAAATATCTGTTTCACGCACGGTATCAAAGACTGTGGGGAGTGCTTTCCCG
TGTCAGAATCTCAACCCGTTTCTGTCGTCAAAAAGAAGACTTATCAGAAACTGTGTCCAA
TTCATCACATTTTGGGAAGAGCACCCGAGATTGCGTGTTCGGCCTGCGATCTGGCCAATG
TAGACTTGGATGACTGTGTTTCTGAACAATAAATGACTTAAACCAGGTATGGCTGCCGAT
GGTTATCTTCCAGATTGGCTCGAGGACACTCTCTCTGAAGGAATAAGACAGTGGTGGAAG
CTCAAACCTGGCCCACCACCACCAAAGCCCGCAGAGCGGCATAAGGACGACAGCAGGGGT
CTTGTGCTTCCTGGGTACAAGTACCTCGGACCCTTCAACGGACTCGACAAGGGGGAGCCG
GTCAACGAGGCAGACGCCGCGGCCCTCGAGCACGACAAGGCCTACGACCGGCAGCTCGAC
AGCGGAGACAACCCGTACCTCAAGTACAACCACGCCGACGCGGAGTTTCAGGAGCGTCTT
AAAGAAGATACGTCTTTTGGGGGCAACCTCGGACGAGCAGTCTTCCAGGCGAAAAAGAGG
GTTCTTGAACCTCTGGGCCTGGTTGAGGAACCTGTTAAGACGGCTCCGGGAAAAAAGAGG
CCGGTAGAGCACTCTCCTGTGGAACCAGACTCCTCCTCGGGAACCGGAAAAGCGGGCCAG
CAGCCTGCAAGAAAAAGACTGAATTTCGGTCAGACTGGAGACGCAGACTCCGTACCTGAC
CCCCAGCCTCTCGGACAGCCACCAGCAGCCCCCTCTGGTCTGGGAACTACTACGATGGCT
ACAGGCAGTGGCGCACCAATGGCAGACAATAACGAGGGCGCCGACGGAGTGGGTAATTCC
TCGGGAAATTGGCATTGCGATTCCACATGGATGGGCGACAGAGTCATCACCACCAGCACC
CGAACCTGGGCTCTGCCCACCTACAACAACCATCTGTACAAGCAGATTTCCAGCCAATCA
GGAGCCTCAAACGACAACCACTACTTTGGCTACAGCACCCCTTGGGGGTATTTTGACTTC
AACAGATTCCACTGCCACTTTTCACCACGTGACTGGCAAAGACTCATCAACAACAATTGG
GGATTCCGACCCAAGAGACTCAACTTCAAGCTCTTTAACATTCAAGTCAAAGAGGTCACG
CAGAATGACGGTACGACGACGATTGCCAATAACCTTACCAGCACGGTTCAGGTGTTTACT
GACTCGGAGTACCAGCTCCCGTACGTCCTCGGCTCGGCGCATCAAGGATGCCTCCCGCCG
TTCCCAGCAGACGTCTTCATGGTGCCACAGTATGGATACCTCACCCTGAACAACGGGAGT
CAAGCAGTAGGACGCTCTTCATTTTACTGCCTGGAGTACTTTCCTTCTCAGATGCTTCGT
ACCGGAAACAACTTTACCTTCAGCTATACCTTTGAGGACGTTCCTTTCCACAGCAGCTAC
GCTCACAGCCAGAGTTTGGACCGTCTCATGAATCCTCTCATCGACCAGTACCTGTATTAC
TTGAGCAGAACAAACACTCCAAGCGGAACCACCACGCAGTCCAGGCTTCAGTTTTCTCAG
GCCGGAGCGAGTGACATTCGGGACCAGTCTAGGAACTGGCTTCCTGGACCCTGTTACCGC
CAGCAGCGAGTATCAAAGACAGCTGCGGATAACAACAACAGTGAATACTCGTGGACTGGA
GCTACCAAGTACCACCTCAATGGCAGAGACTCTCTGGTGAATCCGGGCCCGGCCATGGCC
AGCCACAAGGACGATGAAGAAAAGTTTTTTCCTCAAAGCGGGGTTCTCATCTTTGGGAAG
CAAGGCTCAGAGAAAACAAATGTGGACATTGAAAAGGTCATGATTACAGACGAAGAGGAA
ATCAGGACCACCAATCCCGTGGCTACGGAGCAGTATGGTTCTGTATCTACCAACCTCCAG
AGCGGCAACACACAAGCAGCTACCGCAGATGTCAACACACAAGGCGTTCTTCCAGGCATG
GTCTGGCAGGACAGAGACGTGTACCTTCAGGGGCCCATCTGGGCAAAGATTCCACACACG
GACGGACATTTTCACCCCTCTCCCCTCATGGGCGGATTCGGACTTAAACACCCTCCTCCG
CAAATTCTCATCAAGAACACCCCGGTACCTGCGAATCCTTCGACCACCTTCAGTGCGGCA
AAGTTTGCTTCCTTCATCACACAGTATTCCACGGGACAGGTCAGCGTGGAGATCGAGTGG
GAGTTGCAGAAGGAGAACAGCAAACGCTGGAATCCCGAGATCCAGTACACTTCCAACTAC
AACAAATCTGTTAATGTGGACTTTACTGTGGACACTAATGGCGTGTATTCAGAGCCTCGC
CCCATTGGCACCAGATACCTGACTCGTAATCTGTAATTGCTTGTTAATCAATAAACCGTT
TAATTCGTTTCATTTGAACTTTGGTCTCTGCGTACTTCTTTTCTTATCTAGTTTCCATGG
CTACGTAAATAAGTACCATGGCGGGTTAATCATTAACTTTAAGGAACCCCTAGTGATGGA
GTTGGCCACTCCCTCTCTGCGCGCTCGCTCGCTCACTGAGGCCGCCCGGGCAAAGCCCGC
CCGACGCCCGGGCTTTGCCCGGGCGGCCTCAGTGAGCGAGCGAGCGCGCAGAGAGGGAGT
GGCCAA
SEQ ID NO: 79
>#1449_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGGAGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAAAAGCTGCAGCGCGACTTTCTGACAGAATGGCGCCGTGTGAGTAAGGCCC
CCGAGGCCCTTTTCTTTGTGCAATTTGAGAAGGGAGAAAGCTACTTCCACATGCACGTGC
TTGTGGAAACCACGGGGGTGAAGTCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACCCAGGAGCAGAACAAAGAGAATCAGAATCCCAATTCTGACGCGC
CGGTGATTAGATCAAAAACCTCAGCCAGATACATGGAGCTGGTCGGGTGGCTCGTGGATA
AGGGGATTACCTCCGAGAAGCAGTGGATCCAGGAGGACCAGGCTTCATACATCTCCTTCA
ATGCGGCCTCCAACTCGCGGTCCCAAATCAAGGCTGCTCTGGACAATGCGGGAAAGATCA
TGAGCCTGACTAAAACGGCCCCCGACTACCTGGTGGGCCAGCAGCCCGTGGAGGACATTT
CCGGCAATCGGATTTATAAAATCTTGGAACTGAACGGGTATGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACGGTGCCCTTCT
ACGGGTGCGTAAACTGGACCAACGAGAACTTTCCTTTCAATGATTGCGTCGACAAGATGG
TGATCTGGTGGGAGGAGGGGAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTTGAGCACCAGCAGCCGTTGCAAGACCGGATGTTCAAATTTGAACTTACCCGCCGTC
TGGATCATGACTTTGGGAAGGTCACAAAGCAGGAAGTCAAAGACTTTTTCCGGTGGGCAA
AGGATCACGTGGTTGAGGTGGAGCATGAATTCTACGTCAAAAAGGGTGGAGCTAAGAAAA
GGCCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCGTGCAAAACCTGCGAGAGAATGA
ATCAGATTTCAAATGTCTGTTTCACGCACGGTGTCAAAGACTGTGGGGAGTGCTTTCCCG
TGTCAGAATCTCAACCCGTTTCTGTCGTCAAAAAGAAGACTTATCAGAAACTGTGTCCAA
TTCATCACATTTTGGGAAGAGCACCCGAGATTGCGTGTTCGGCCTGCGATCTGGCCAATG
TGGATTTGGATGACTGTGTTTCTGAACAATAAATGACTTAAACCAGGTATGGCTGCCGAT
GGTTATCTTCCAGATTGGCTCGAGGACACTCTCTCTGAAGGAATAAGACAGTGGTGGAAG
CTCAAACCTGGCCCACCACCACCAAAGCCCGCAGAGCGGCATAAGGACGACAACAGGGGT
CTTGTGCTTCCTGGGTACAAGTACCTCGGACCCTTCAACGGACTCGACAAGGGGGAGCCG
GTCAACGAGGCAGACGCCGCGGCCCTCGAGCACGACAAGGCCTACGACCGGCAGCTCGAC
AGCGGAGACAATCCGTACCTCAAGTACAACCACGCCGACGCGGAGTTTCAGGAGCGTCTT
AAAGAAGATACGTCTTTTGGGGGCAACCTCGGACGAGCAGTCTTCCAGGCGAAAAAGAGG
GTTCTTGAACCTCTGGGCCTGGTTGAGGAACCTGTTAAAACGGCTCCGGGAAAAAAGAGG
CCGGTAGAGCACTCTCCTGTGGAGCCAGACTCCTCCTCCGGAACTGGAAAGTCGGGCCAA
CAGCCTGCAAGAAAAAGATTGAATTTCGGTCAGACTGGAGACGCAGACTCCGTACCTGAC
CCCCAGCCTCTCGGACAGCCACCAGCAGCCCCCTCTGGTCTGGGAACTAATACGATGGCT
ACAGGCAGTGGCGCACCAATGGCAGACAATAACGAGGGCGCCGACGGAGTGGGTAATTCC
TCGGGAAATTGGCATTGCGATTCCACATGGATGGGCGACAGAGTCATCACCACCAGCACC
CGAACCTGGGCTCTGCCCACCTACAACAACCATCTGTACAAGCAGATTTCCAGCCAATCA
GGAGCCTCAAACGACAACCACTACTTTGGCTACAGCACCCCTTGGGGGTATTTTGACTTC
AACAGATTCCACTGCCACTTTTCACCACGTGACTGGCAAAGACTCATCAACAACAATTGG
GGATTCCGACCCAAGAGACTCAACTTCAAGCTCTTTAACATTCAAGTCAAGGAGGTCACG
CAGAATGACGGTACGACGACGATTGCCAATAACCTTACCAGCACGGTTCAGGTGTTTACT
GACTCGGAGTACCAGCTCCCGTACGTCCTCGGCTCGGCGCATCAAGGATGCCTCCCGCCG
TTCCCAGCAGACGTCTTCATGGTGCCACAGTATGGATACCTCACCCTAAACAACGGGAGT
CAAGCAGTAGGACGCTCTTCCTTTTACTGCCTGGAGTACTTTCCTTCTCAGATGCTTCGT
ACCGGAAACAACTTTACCTTCAGCTATACCTTTGAGGACGTTCCTTTCCACAGCAGCTAC
GCTCACAGCCAGAGTTTGGACCGTCTCATGAATCCTCTCATCGACCAGTACCTGTATTAC
TTGAGCAGAACAAACACTCCAAGCGGAACCACCACGCAGTCCAGGCTTCAGTTTTCTCAA
GCCGGAGCAAGTGACATTCGGGACCAGTCTAGGAACTGGCTTCCTGGACCCTGTTACCGC
CAGCAGCGAGTATCAAAGACACCTGCGGATAACAACAACAGTGAATACTCGTGGACTGGA
GCTACCAAGTACCACCTCAATGGCAGAGACTCTCTGGTAAATCCGGGCCCGGCCATGGCC
AGCCACAAGGACGATGAAGAGAAGTTTTTCCCTCAAAGCGGGGTTCTCATCTTTGGGAAG
CAAGGCTCAGAGAAAACAAATGTGGACATTGAAAAGGTCATGATTACAGACGAAGAGGAA
ATCAGGACCACCAATCCCGTGGCTACGGAGCAGTATGGTACTGTATCTACCAACCTCCAG
AGCGGCAACACACAAGCAGCTACCTCAGATGTCAACACACAAGGCATTCTTCCAGGCATG
GTCTGGCAGGACAGAGACGTGTACCTGCAGGGACCCATTTGGGCAAAGATTCCACACACG
GACGGACATTTTCACCCCTCTCCCCTCATGGGTGGATTCGGACTTAAACACCCTCCTCCA
CAGATTCTCATCAAGAACACCCCGGTACCTGCGAATCCTTCGACCACCTTCAGTGCGGCA
AAGTTTGCTTCCTTCATCACACAGTACTCCACGGGGCAGGTCAGCGTGGAGATCGAGTGG
GAGCTGCAGAAAGAGAACAGCAAACGCTGGAACCCCGAGATCCAGTACACTTCCAATTAC
AACAAATCTGTTAATGTGGACTTTACTGTGGACACTAATGGTGTGTATTCAGAGCCTCGC
CCCATTGGCACCAGATACCTGACTCGTAATCTGTAATTGCTTGTTAATCAATAAACCGTT
TAATTCGTCTCAGTTGAACTTTGGTCTCTGCGTACTTCTTTCTTATCTAGTTTCCATGGC
TACGTAGATAAGTAGCATGGCGGGTTAATCATTAACTACAAGGAACCCCTAGTGATGGAG
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAA
SEQ ID NO: 80
>#2128_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCAGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCACGTGC
TGGTGGAAACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCAGAAATGGCGCCGGAGGCGGGAACAAGGTGGTGGATGAGTGCTACATCC
CCAATTACCTGCTCCCCAAAACCCAGCCTGAACTCCAGTGGACTAATATGGAACAGTATT
TAAGCGCCTGTTTGAATCTCGCGGAGCGTAAACGGTTGGTGGCGCAGCATCTGACGCACG
TGTCGCAGACGCAGGAGCAGAACAAAGAGAATCAGAATCCCAATTCTGACGCGCCGGTGA
TTAGATCAAAAACCTCAGCCAGGTACATGGAGCTGGTCGGGTGGCTCGTGGATAAGGGGA
TTACGTCGGAGAAGCAGTGGATCCAGGAGGACCAGGCCTCATACATCTCCTTCAATGCGG
CCTCCAACTCGCGGTCCCAAATCAAGGCTGCTCTGGACAATGCGGGAAAGATCATGAGCC
TGACTAAAACCGCCCCCGACTACCTGGTGGGCCAGCAGCCCGTGGAGGACATTTCCAGCA
ATCGGATTTATAAAATCTTGGAACTGAACGGGTACGATCCCCAATACGCGGCTTCCGTCT
TTCTGGGATGGGCCACGAAAAAGTTCGGCAAGAGGAACACCATCTGGCTGTTTGGGCCTG
CAACTACCGGGAAGACCAACATCGCGGAGGCCATAGCCCACACGGTGCCCTTCTACGGGT
GCGTAAACTGGACCAATGAGAACTTTCCTTTCAACGACTGTGTCGACAAGATGGTGATCT
GGTGGGAGGAGGGAAAGATGACCGCCAAGGTCGTGGAGTCGGCCAAAGCCATTCTCGGAG
GAAGCAAGGTGCGCGTGGACCAGAAATGCAAGTCCTCGGCCCAGATAGACCCGACTCCCG
TGATCGTCACCTCCAACACCAACATGTGCGCCGTGATTGACGGGAACTCAACGACCTTCG
AACACCAGCAGCCGTTGCAAGACCGGATGTTCAAATTTGAACTCACCCGCCGCCTGGATC
ATGACTTTGGGAAGGTCACCAAGCAGGAAGTCAAAGACTTTTTCCGGTGGGCAAAGGATC
ACGTGGTTGAGGTGGAGCATGAATTCTACGTCAAAAAGGGTGGAGCCAAGAAAAGGCCCG
CCCCCAGTGACGCAGATATAAGTGAGCCCAAACGGGCGCGCGAGTCAGTTGCGCAGCCAT
CGACGTCAGACGCGGAAGCTTCAATCAACTACGCGGACAGGTACCAAAACAAATGTTCTC
GTCACGTGGGCATGAATCTGATGCTGTTTCCCTGCAGACAATGCGAGAGAATGAATCAAA
ATTCAAATATCTGCTTCACTCACGGACAGAAAGACTGTTTAGAGTGCTTTCCCGTGTCAG
AATCTCAACCCGTTTCTGTCGTCAAAAAGGCGTATCAGAAACTGTGCTACATTCATCATA
TCATGGGAAAGGTGCCAGACGCTTGCACTGCCTGCGATCTGGTCAATGTGGATTTGGATG
ACTGCATCTCTGAACAATAAATGATTTAAATCAGGTATGGCTGCCGATGGTTATCTTCCA
GATTGGCTCGAGGACACTCTCTCTGAAGGAATAAGACAGTGGTGGAAGCTCAAACCTGGC
CCACCACCACCAAAGCCCGCAGAGCGGCATAAGGACGACAGCAGGGGTCTTGTGCTTCCT
GGGTACAAGTACCTCGGACCCTTCAACGGACTCGACAAGGGAGAGCCGGTCAACGAGGCA
GACGCCGCGGCCCTCGAGCACGACAAGGCCTACGACCGGCAGCTCGACAGCGGAGACAAC
CCGTACCTCAAGTACAACCACGCCGACGCGGAGTTTCAGGAGCGCCTTAAAGAAGATACG
TCTTTTGGGGGCAACCTCGGACGAGCAGTCTTCCAGGCGAAAAAGAGGGTTCTTGAACCT
CTGGGCCTGGTTGAGGAACCTGTTAAGACGGCTCCGGGAAAAAAGAGGCCGGTAGAGCAC
TCTCCTGCGGAGCCAGACTCCTCCTCGGGAACCGGAAAGGCGGGCAACCAGCCTGCAAGA
AAAAGATTGAATTTCGGTCAGACTGGAGACGCAGACTCCGTACCTGACCCCCAACCTCTC
GGACAGCCACCAGCAGCCCCCTCTGGTCTGGGAACTAATACGATGGCTACAGGCAGTGGC
GCACCAATGGCAGACAATAACGAGGGCGCCGACGGAGTGGGTAATTCCTCGGGAAATTGG
CATTGCGATTCCACATGGATGGGCGACAGAGTCATCACCACCAGCACCCGAACCTGGGCT
CTGCCCACCTACAACAACCATCTCTACAAGCAGATTTCCAGCCAGTCTGGAGCCTCAAAC
GACAACCACTACTTTGGCTACAGCACCCCTTGGGGGTATTTTGACTTCAACAGATTCCAC
TGCCACTTTTCACCACGTGACTGGCAAAGACTCATCAACAACAACTGGGGATTCCGACCC
AAGAGACTCAACTTCAAGCTCTTTAACATTCAAGTCAAAGAGGTCACGCAGAATGACGGT
ACGACGACGATTGCCAATAACCTTACCAGCACGGTTCAGGTGTTTACTGACTCGGAGTAC
CAGCTCCCGTACGTCCTCGGCTCGGCGCATCAAGGATGCCTCCCGCCGTTCCCAGCAGAC
GTCTTCATGGTGCCACAGTATGGATACCTCACCCTGAACAACGGAAGTCAGGCAGTAGGA
CGCTCTTCATTTTACTGCCTGGAGTACTTTCCTTCTCAAATGCTGCGTACCGGAAACAAC
TTTACCTTCAGCTACACATTTGAAGACGTGCCTTTCCACAGCAGCTACGCTCACAGCCAG
AGTTTGGACCGTCTCATGAATCCTCTCATCGACCAGTACCTGTATTACTTGAGCAGAACA
AACACTCCAAGCGGAACCACCACGCAGTCCAGGCTTCAGTTTTCTCAGGCCGGAGCAAGT
GACATTCGGGACCAGTCTAGGAACTGGCTTCCTGGACCCTGTTACCGCCAGCAGCGAGTA
TCAAAGACATCTGCAGATAACAACAATAGTGAATACTCGTGGACTGGAGCTACCAAGTAC
CACCTCAATGGCAGAGACTCTCTGGTGAATCCGGGCCCGGCCATGGCCAGCCACAAGGAC
GATGAAGAAAAGTTTTTTCCTCAGAGCGGGGTTCTCATCTTTGGAAAGCAAGGTTCAGAG
AAAACAAATGTGGAAAAGGTCATGATTACAGACGAAGAGGAAATCAGGACCACCAATCCC
GTGGCTACGGAGCAGTATGGTGCTGTATCTACCAACCTCCAGAGCGGCAACACACAAGCA
GCTACCGCAGATGTCAACACACAAGGCGTTCTTCCAGGCATGGTCTGGCAGGACAGAGAC
GTGTACCTGCAGGGGCCCATCTGGGCAAAGATTCCCCACACGGATGGACACTTTCACCCT
TCTCCTCTTATGGGAGGATTTGGACTTAAACACCCTCCCCCGCAGATTCTCATCAAGAAC
ACCCCGGTACCTGCGAATCCTTCGACCACCTTTAGTGCGGCAAAGTTTGCTTCCTTCATC
ACACAGTATTCCACGGGGCAGGTCAGTGTGGAGATCGAGTGGGAGCTGCAGAAGGAGAAC
AGCAAGCGCTGGAACCCCGAGATCCAATACACTTCCAACTACAACAAATCTGTTAATGTG
GACTTTACTGTGGACACTAATGGCGTGTATTCAGAGCCTCGCCCCATTGGCACCAGATAC
CTGACTCGTAATCTGTAATTGCTTGTTAATCAATAAACCGTTTAATTCGTTTCAATTGAA
CTTTGGTCTCTGCGTACTTCTTTCTTATATAGTTTCCATGGCTACGTAGATAAGTAGCAT
GGCGGGTTAATCATTAACACAAGGAACCCCTAGTGATGGAGTTGGCCACTCCCTCTCTGC
GCGCTCGCTCGCTCACTGAGGCCGGGCGACCAAAGGTCGCCCGACGCCCGGGCTTTGCCC
GGGCGGCCTCAGTGAGCGAGCGAGCGCGCAGAGAGGGAGTGGCCAA
SEQ ID NO: 81
>#2141_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGGGCAAAGTTTACGAGATTGTGATTAAGGTCCCCAGCG
ACCTTGACGAGCATCTGCCAGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTTTTCTTTGTGCAATTTGAGAAGGGAGAGAGCTACTTCCACATGCACGTGC
TGGTGGATACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAAGTTGTGGATGAGTGCTACATCC
CCGATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCTAATTCTGACGCGC
CGGTGATCAGATCAAAACCCTCAGCCAGGTACATGGAGCTGGTCGGGTGGCTCGTGGATA
AGGGGATTACGTCGGAGAAGCAGTGGATCCAGGAGGACCAGGCCTCCTACATCTCCTTCA
ATGCGGCCTCCACCTCGCGGTCCCAAATCAAGGCTGCCTTGGACAATGCGGGGAAGATCA
TGAGCCTGACTAAAACTGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTTGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCTTTCAACGACTGTGTCGACAAGATGG
TGATCTGGTGGGAGGAGGGGAAGATGACCGCCAAGGTCGTGGAGTCGGCCAAAGCCATTC
TCGGAGGAAGTAAGGTGCGCGTGGACCAGAAATGCAAGTCCTCGGCCCAGATAGACCCGA
CTCCTGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTATCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTACCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAGCCTGTTAAAACGGCTCCGGGAAAAAAGAGGCCGGTA
GAGCACTCTCCTGTGGAGCCAGACTCCTCCTCGGGAACCGGAAAGGCGGGCAACCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGAACC
TGGGCCCTGCCCACCTACAACAACCACCTCTACAAACAAATTTCCAGCCAATCAGGAGCC
TCGAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGGCCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTCCCACAGTATGGATACCTCACCCTGAACAACGGGAGTCAGGCA
GTAGGACGCTCTTCATTTTACTGCCTGGAGTACTTTCCTTCTCAGATGCTGCGTACCGGA
AACAACTTTACCTTCAGCTATACCTTTGAGGATGTTCCGTTCACAAGCAGATACGCTCAC
AGCCAGAGTTTGGACCGTCTCATGAATCCTCTCATCGACCAGTACCTGTATTACTTGAGC
AGAACAAACACTCCAAGTGGAACCACCACGCAGTCAAGGCTTCAGTTTTCTCAGGCCGGA
GCGAGTGACATTCGGGATCAGTCTAGGAACTGGCTTCCTGGACCCTGTTATCGCCAGCAG
CGAGTATCAAAGACATCTGCGGATAACAACAACAGTGAATACTCGTGGACTGGAGCTACC
AAGTACCACCTCAATGGCAGAGACTCTCTGGTGAATCCGGGCCCGGCCATGGCCAGTCAC
AAAGACGATGAAGAAAAGTTTTTTCCTCAGAGCGGGGTTCTCATCTTTGGGAAGCAAGGC
TCAGAGAAAACAAATGTGGACATTGAAAAGGTCATGATTACAGACGGAGAGGAAATCAGG
ACCACCAATCCCGTCGCCACGGAGCAGTATGGTTCTGTATCTACCAACCTCCAGAGAGGC
AACACACAAGCAGCTACCGCAGATGTCAACACACAAGGCGTTCTTCCAGGCATGGTCTGG
CAGGACAGAGACGTGTACCTGCAGGGGCCTATCTGGGCAAAGATTCCACACACGGACGGA
CATTTTCACCCCTCTCCCCTCATGGGCGGATTTGGACTTAAACACCCTCCCCCGCAGATT
CTCATCAAGAACACCCCGGTACCTGCGAATCCTTCGACCACCTTCAGTGCGGCAAAGTTT
GCTTCCTTTATCACACAGTACTCCACGGGGCAGGTCAGCGTGGAGATCGAGTGGGAGCTG
CAGAAGGAGAACAGCAAACGCTGGAATCCCGAGATTCAGTACACTTCCAACTACAACAAG
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTGTAATTC
GTTTCAGTTGAACTTTGGTCTCTGCGTACTTCTTTCTTATCTAGTTTCCATGGCTACGTA
GATAAGTACCATGGCGGGTTAATCATTAACTATAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 82
>#1343_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTGGCTCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GATTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATTTGAATCTGATTGAGCAGGCACCCCTGA
CCGTGGCCGAGAAGCTGCAGCGCGACTTTCTGACGGAATGGCGCCGCGTGAGTAAGGCCC
CGGAGGCCCTCTTCTTTGTGCAATTTGAAAAGGGAGAGAGCTACTTCCACATGCACGTGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAAAGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGATACATGGAGCTGGTCGGGTGGCTCGTGGATA
AGGGGATTACCTCGGAGAAGCAGTGGATCCAGGAGGACCAGGCCTCATACATCTCCTTCA
ATGCGGCCTCCAACTCGCGGTCCCAAATCAAGGCTGCTCTGGACAATGCGGGAAAGATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTATTTCTGGGATGGGCCACGAAAAAGTTCGGCAAGAGAAACACCATCTGGCTGTTTG
GGCCTGCAACTACCGGGAAGACCAACATCGCGGAGGCTATAGCCCACACTGTGCCCTTCT
ACGGGTGCGTAAACTGGACCAATGAGAACTTTCCTTTCAACGACTGTGTCGACAAGATGG
TGATCTGGTGGGAGGAGGGGAAGATGACCGCCAAGGTCGTGGAGTCGGCCAAAGCCATTC
TCGGAGGAAGCAAGGTGCGCGTGGACCAGAAATGCAAGTCCTCGGCCCAAATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GGCCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAAAATTCAAATATTTGTTTCACTCACGGACAGAAAGACTGTTTGGAGTGCTTTCCCG
TGTCAGAATCTCAACCCGTTTCTGTCGTCAAAAAGGCGTATCAGAAACTGTGCTATATTC
ATCATATCATGGGAAAGGTACCAGACGCTTGCACTGCTTGCGACCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGGGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGAAACCTCGGACGAGCAGTCTTCCAGGCGAAAAAGAGGGTTCTT
GAACCTCTGGGCCTGGTTGAGGAGCCTGTTAAAACGGCTCCGGGAAAAAAGAGGCCGGTA
GAGCACTCTCCTGTGGAGCCAGACTCCTCCTCGGGAACCGGAAAAGCGGGCCAGCAGCCT
GCGAGAAAGAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGCACC
TGGGCTCTGCCCACCTACAACAACCATCTGTACAAGCAGATTTCCAGCCAATCAGGAGCA
TCGAACGACAACCACTACTTTGGCTACAGCACCCCCTGGGGGTATTTTGACTTCAACAGA
TTCCACTGCCATTTCTCCCCACGTGACTGGCAAAGACTCATCAACAACAATTGGGGATTC
CGACCCAAGAGACTCAACTTCAAGCTCTTTAACATTCAAGTCAAGGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACCGGA
AACAACTTTACCTTCAGCTACACCTTTGAGGACGTTCCTTTCCACAGCAGCTACGCTCAC
AGCCAGAGTTTGGACCGGCTGATGAATCCTCTGATCGACCAGTATCTGTATTACTTGAGC
AAAACAAACGCTCCAAGCGGAACCACCACGATGTCCAGGCTTCAGTTTTCTCAGGCAGGA
GCAAGTGACATTCGGGACCAGTCTAGAAACTGGCTTCCTGGACCCTGTTACCGCCAGCAG
CGAGTATCAAAGACAGCTGCGGACAACAACAACAGTGATTACTCGTGGACTGGAGCTACC
AAGTACCACCTCAATGGAAGAGACTCTCTGGTGAATCCGGGCACAGCCATGGCCAGCCAC
AAGGACGATGAAGAAAAGTATTTTCCTCAGAGCGGGGTTCTCATCTTCGGAAAACAAGAT
TCGGGAAAAACTAATGTGGACATTGAAAAGGTCATGATTACAGACGAAGAGGAAATCAGG
ACCACCAATCCCGTGGCTACGGAGCAGTATGGTTCTGTATCTACCAACCTCCAAAGCGGC
AACACACAAGCAGCTACCTCAGATGTCAACACACAAGGCGTTCTTCCAGGCATGGTCTGG
CAGGACAGAGACGTGTACCTGCAGGGACCCATCTGGGCAAAGATTCCACACACGGACGGA
CATTTTCACCCTTCTCCTCTTATGGGAGGATTTGGACTGAAGCACCCCCCCCCGCAGATT
CTCATCAAGAATACCCCGGTACCTGCGAATCCTTCTACCACCTTCAGCGCGGCAAAGTTT
GCTTCCTTCATCACACAGTACTCCACGGGGCAGGTCAGCGTGGAGATCGAGTGGGAGCTG
CAGAAAGAGAACAGCAAACGCTGGAACCCCGAGATCCAGTACACTTCCAACTACAACAAG
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTCTTTCTTATCTAGTTTCCATGGCTACGTA
GATAAGTAGCCTGGCGGGTTAATCATTAACTACAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 83
>#777_AAV.FL.linear [organism = Adeno-associated virus][mol_type = genomic DNA]
[isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATATAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTAAACTGGGTGGCCGAGAAGG
AATGGGAGTTGCCGCCAGATTCTGACATGGATCTGAATCTGATTGAGCAGGCACCCCTGA
CCGTGGCCGAAAAGCTGCAGCGCGACTTTCTGACGGAGTGGCGCCGTGTGAGTAAGGCTC
CGGAAGCCCTTTTCTTTGTGCAATTTGAGAAAGGGGAGAGCTACTTCCACATGCACGTGC
TTGTGGAGACCACCGGGGTGAAATCCATGGTCTTGGGACGTTTCCTGAGTCAGATTCGCG
AAAAACTGATTCAGAGAATTTACCGCGGGATCGAGCCGACTTTGCCAAATTGGTTCGCGG
TCACAAAGACCCGAAATGGCGCCGGAGGCGGGAACAAGGTGGTGGATGAGTGCTATATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATCAGATCAAAAACCTCAGCCAGGTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAACTCGCGGTCCCAAATCAAGGCTGCTTTGGACAATGCGGGAAAGATCA
TGAGCCTCACTAAAACCGCCCCCGACTACCTGGTGGGCCAACAGCCCGTGGAGGACATTT
CCAGCAATCGGATTTATAAAATCTTGGAACTGAACGGGTATGATCCCCAATATGCGGCTT
CCGTCTTCCTGGGATGGGCCACGAAAAGGTTCGGCAAGAGGAACACCATCTGGCTGTTTG
GGCCTGCGACTACCGGGAAGACCAACATCGCGGAGGCTATAGCCCACACTGTGCCTTTTT
ACGGGTGCGTAAACTGGACCAATGAGAACTTTCCGTTCAACGACTGCGTCGACAAGATGG
TGATCTGGTGGGAGGAGGGGAAGATGACCGCCAAGGTTGTGGAGTCGGCCAAGGCCATCC
TCGGTGGGAGCAAGGTGCGCGTGGACCAGAAATGCAAGTCCTCGGCCCAGATAGACCCGA
CTCCTGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTCACCCGCCGTC
TGGATCATGACTTTGGGAAGGTCACCAAGCAGGAAGTTAAAGACTTTTTCCGGTGGGCCC
AAGACCACGTGGTAGAGGTGGAGCATGAATTCTACGTCAAAAAGGGTGGAGCTAAGAAAA
GGCCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAAAAAGACTGCCTAGAGTGCTTTCCCG
TGTCAGAATCTCAACCCGTTTCTGTCGTCAAAAAGGCGTATCAGAAACTGTGCTATATTC
ATCATATCATGGGAAAGGTACCAGATGCTTGCACTGCTTGCGACCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCACAAGGACGACAGCAGGGGTCTTGTG
CTTCCTGGGTACAAGTACCTCGGACCCTTCAACGGACTCGACAAAGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAGCCTGTTAAAACGGCTCCGGGAAAAAAGAGGCCGGTA
GAGCACTCTCCTGTGGAGCCAGACTCCTCCTCGGGAACCGGAAAAGCGGGCCAGCAGCCT
GCGAGAAAGAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGCACC
TGGGCTCTGCCCACCTACAACAACCATCTGTACAAGCAGATTTCCAGCCAATCAGGAGCA
TCGAACGACAACCACTACTTTGGCTACAGCACCCCTTGGGGGTATTTTGACTTCAACAGA
TTCCACTGCCACTTCTCCCCACGTGACTGGCAAAGACTCATCAACAACAATTGGGGATTC
CGGCCCAAAAGACTCAACTTCAAGCTCTTTAACATTCAAGTCAAGGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACCGGA
AACAACTTTACCTTCAGCTACACCTTTGAGGACGTTCCTTTCCACAGCAGCTACGCTCAC
AGCCAGAGTTTGGACCGGCTGATGAATCCTCTGATCGACCAGTATCTGTATTACTTGAGC
AAAACAAACGCTCCAAGCGGAACCACCACGATGTCTAGGCTTCAGTTTTCTCAGGCCGGA
GCAAGTGACATTCGGGACCAGTCTAGAAACTGGCTTCCTGGACCCTGTTACCGCCAGCAG
CGAGTATCAAAGACAGCTGCGGACAACAACAACAGTGATTACTCGTGGACTGGAGCTACC
AAGTACCACCTCAATGGAAGAGACTCTCTGGTGAATCCGGGCACAGCCATGGCCAGCCAC
AAGGACGATGAAGAAAAGTATTTTCCTCAGAGCGGGGTTCTCATCTTCGGAAAACAAGAC
TCGGGAAAAACTAATGTGGACATTGAAAAGGTCATGATTACAGACGAAGAGGAAATCAGG
ACCACCAATCCCGTGGCTACGGAGCAGTATGGTTCTGTATCTACCAACCTCCAAAGCGGC
AACACACAAGCAGCTACCTCAGATGTCAACACACAAGGCGTTCTTTCAGGCATGGTCTGG
CAGGACAGAGACGTGTACCTGCAGGGGCCCATCTGGGCAAAGATTCCCCACACGGATGGA
CACTTTCACCCTTCCCCGCTCATGGGAGGATTTGGACTTAAACACCCTCCCCCGCAGATT
CTCATCAAGAATACCCCCGTACCTGCGAATCCTTCGACCACCTTCAGTGCGGCAAAGTTT
GCTTCCTTCATCACACAGTATTCCACGGGGCAGGTCAGCGTGGAGATCGAGTGGGAGCTG
CAGAAAGAGAACAGCAAACGCTGGAACCCCGAGATCCAGTACACTTCCAACTACAACAAG
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTCTTTCTTATCTAGTTTCCATGGCTACGTA
GATAAGTAGCCTGGCGGGTTAATCATTAACTACAAGGAACCCCTAGTGATGGAGTTGGCC
ACTCCCTCTCTGCGAGCGAGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 84
>#3511_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCAGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCGGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCACGTGC
TTGTGGAGACCACCGGGGTGAAGTCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATCAGATCAAAAACCTCAGCCAGGTACATGGAGCTGGTCGGGTGGCTCGTGGATA
AGGGGATTACCTCGGAGAAGCAGTGGATCCAGGAGGACCAGGCCTCATACATCTCCTTCA
ATGCGGCCTCCAACTCGCGGTCCCAGATCAAGGCTGCCTTGGACAATGCGGGAAAAATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACGGTTCCCTTCT
ACGGGTGCGTAAACTGGACCAATGAGAACTTTCCCTTCAATGACTGCGTCGACAAGATGG
TGATCTGGTGGGAGGAGGGAAAAATGACCGCCAAGGTCGTGGAATCGGCCAAAGCCATTC
TCGGCGGAAGCAAGGTGCGCGTGGACCAGAAATGCAAATCCTCGGCCCAGATAGACCCGA
CTCCCGTGATCGTCACCTCCAACACCAACATGTGCGCCGTGATTGACGGAAACTCAACGA
CCTTCGAGCACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCTAAGAAAA
GGCCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATTTGATGCTGTTTCCCTGCAGACAATGCGAGAGAATGA
ATCAGAATTCGAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTGCCAGACGCTTGCACTGCCTGCGATATGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCACAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGCGGAGCCAGATTCCTCCTCCGGAACTGGAAAGTCGGGCCAACAGCCT
GCAAGAAAGAGATTGAATTTTGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCACATGGATGGGCGACAGAGTCATCACCACCAGCACCCGCACC
TGGGCTCTGCCCACCTACAACAACCATCTGTACAAGCAGATTTCCAGCCAGTCTGGAGCC
AGCAACGACAACCACTACTTTGGCTACAGCACCCCCTGGGGGTATTTTGACTTCAACAGA
TTCCACTGCCACTTCTCCCCACGTGACTGGCAAAGACTCATCAACAACAACTGGGGATTC
CGGCCCAAGAGACTCAGCTTCAAGCTCTTTAACATTCAAGTCAAGGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTGCCACAGTATGGATACCTCACCCTGAACAACGGGAGTCAGGCG
GTAGGACGCTCTTCATTTTACTGCCTGGAGTACTTTCCTTCTCAGATGCTGCGTACTGGA
AACAACTTTACCTTCAGCTACACCTTTGAGGACGTTCCTTTCCACAGCAGCTACGCTCAC
AGCCAGAGTTTGGACCGTCTCATGAATCCTCTCATCGACCAGTACCTGTATTACTTGAGC
AGAACAAACACTCCAAGCGGAACCACCACGATGTCCAGGCTTCAGTTTTCTCAGGCCGGA
GCGAGTGACATTCGGGACCAGTCTAGGAACTGGCTTCCTGGACCCTGTTACCGCCAGCAG
CGAGTATCAAAGACAGCTGCGGACAACAACAACAGTGATTACTCGTGGACTGGAGCTACC
AAGTACCACCTCAATGGCCGGGACTCTCTGGTGAATCCAGGACCAGCCATGGCCAGCCAC
AAAGACGATGAAGAAAAGTATTTTCCTCAGAGCGGGGTTCTCATCTTTGGAAAACAAGAC
TCGGGAAAAACTAATGTGGACATTGAAAAGGTTATGATTACAGACGAAGAGGAAATCAGG
ACCACCAATCCCGTGGCTACGGAGCAGTATGGTTCTGTATCTACCAACCTCCAGAGCGGC
AACACACAAGCAGCTACCTCAGATGTCAACACACAAGGCGTTCTTCCTGGCATGGTCTGG
CAGGACAGAGACGTGTACCTGCAGGGACCCATCTGGGCAAAGATTCCACACACGGACGGA
CACTTTCACCCTTCTCCCCTCATGGGCGGATTTGGACTGAAGCACCCTCCCCCGCAGATT
CTCATCAAGAATACCCCGGTACCTGCGAATCCTTCGACCACCTTCAGTGCGGCAAAGTTT
GCTTCCTTCATCACACAGTACTCCACGGGGCAGGTCAGCGTGGAGATAGAGTGGGAGCTG
CAGAAGGAGAACAGCAAACGCTGGAACCCCGAGATCCAGTATACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTAAATTC
GTTTCAGTTGAACTTTGGTCTCTGCGTACTTCTTTCTTATCTAGTTTCCATGGCTACGTA
GATAAGTAGCATGGCGAGTTAATCATTAACTCTAAGGAACCCCTAGTGATGGAGTTGGCC
ACTCCCTCTCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTGGCCAA
SEQ ID NO: 85
>#3013_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGTATTTAAGCCCGAGTGAGCACGCAGGGTCTCCATTTTGAAGCGGGAG
GTTTGAACGCGCAGCCGCCATGCCGGGATTTTACGAGATTGTGATTAAGGTCCCCAGCGA
CCTTGACGAGCATCTGCCCGGCATTTCTGACAGCTTTGTGAACTGGGTGGCCGAGAAGGA
ATGGGAGTTGCCGCCAGATTCTGACATGGATCTGAATCTGATTGAGCAGGCACCCCTGAC
CGTGGCCGAGAAGCTGCAGCGCGACTTTCTGACGGAGTGGCGCCGTGTGAGTAAGGCCCC
GGAGGCTCTTTTCTTTGCGCAATTTGAGAAGGGAGAGAGCTACTTCCACATGCACGTGCT
GGTGGAGACCACCGGGGTGAAATCCATGGTTTTGGGACGTTTCCTGAGTCAGATTCGCGA
AAAACTGATTCAGAGAATTTACCGCGGGATCGAGCCGACTTTGCCAAACTGGTTCGCGGT
CACAAAGACCAGAAATGGCGCCGGAGGCGGGAACAAGGTGGTGGATGAGTGCTACATCCC
CAATTACCTGCTCCCCAAAACCCAGCCTGAGCTCCAGTGGGCGTGGACTAATATGGAACA
GTATTTAAGCGCCTGTTTGAATCTCGCGGAGCGTAAACGGTTGGTGGCGCAGCATCTGAC
GCACGTGTCGCAGACGCAGGAGCAGAATAAAGAGAATCAGAATCCTAATTCTGACGCGCC
GGTGATTAGATCAAAAACCTCAGCCAGGTACATGGAGCTGGTCGGGTGGCTCGTGGATAA
GGGGATCACCTCCGAGAAGCAGTGGATCCAGGAGGACCAGTCTTCATACATCTCCTTCAA
TGCGGCCTCCAACTCGCGGTCCCAGATCAAGGCTGCTCTGGACAATGCGGGGAAGATCAT
GAGCCTGACTAAAACCGCCCCCGACTACCTGGTGGGCCAGCAGCCCGTGGAGGACATTTC
CGAAAATCGGATTTATAAAATCTTGGAACTGAACGGGTACGATCCCCAATACGCGGCTTC
CGTATTCCTGGGATGGGCTACGAAAAAGTTCGGCAAGAGAAACACCATCTGGCTGTTTGG
GCCTGCAACTACCGGGAAGACCAACATCGCGGAGGCCATAGCCCACACGGTTCCCTTCTA
CGGGTGCGTAAACTGGACCAATGAGAACTTTCCCTTCAACGACTGCGTCGACAAGATGGT
GATCTGGTGGGAGGAGGGAAAAATGACCGCCAAGGTCGTGGAGTCGGCCAAAGCCATTCT
CGGCGGAAGCAAGGTGCGCGTGGACCAGAAATGCAAGTCCTCGGCCCAGATAGACCCTAC
TCCTGTGATCGTCACCTCCAACACCAACATGTGCGCCGTGATTGACGGGAACTCAACGAC
CTTCGAGCACCAGCAGCCGTTGCAAGACCGGATGTTCAAATTTGAACTCACCCGCCGTCT
GGATCATGACTTTGGGAAGGTCACCAAGCAGGAAGTCAAAGACTTTTTTCGGTGGGCCCA
AGACCACGTGGTAGAGGTGGAGCATGAATTCTACGTCAAAAAGGGTGGAGCCAAAAAAAG
GCCCGCCCCCAGTGACGCAGATATAAGTGAGCCCAAACGGGCGCGCGAGTCAGTTGCGCA
GCCATCGACGTCAGACGCGGAAGCTTCGATCAACTACGCGGACAGGTACCAAAACAAATG
TTCTCGTCACGTGGGCATGAATCTGATGCTGTTTCCCTGCAGACAATGCGAGAGAATGAA
TCAGAATTCAAATATCTGCTTCACTCACGGACAGAAAGACTGTTTAGACTGCTTTCCCGT
GTCAGAATCTCAACCCGTTTCTGTCGTCAAAAAGTCGTATCAGAAACTTTGCTACATTCA
TCATATCATGGGAAAGGTACCAGACGCTTGCAGTGCTTGCGACCTGGTCAATGTGGATTT
GGATGACTGCATCTCTGAACAATAAATGATTTAAATCAGGTATGGCTGCCGATGGTTATC
TTCCAGATTGGCTCGAGGACACTCTCTCTGAAGGAATAAGACAGTGGTGGAAGCTCAAAC
CTGGCCCACCACCACCAAAGCCCGCAGAGCGGCACAAGGACGACAGTAGGGGTCTTGTGC
TTCCTGGGTACAAGTACCTCGGACCCTTCAACGGACTCGACAAAGGAGAGCCGGTCAACG
AGGCAGACGCCGCGGCCCTCGAACACGACAAGGCCTACGACCGGCAGCTCGACAGCGGAG
ACAACCCGTACCTCAAGTACAACCACGCCGACGCGGAGTTTCAGGAGCGTCTTAAAGAAG
ATACGTCTTTTGGGGGCAACCTCGGACGAGCAGTCTTCCAGGCGAAAAAGAGGGTTCTTG
AACCTCTGGGCCTGGTTGAGGAACCTGTTAAGACGGCTCCGGGAAAAAAGAGGCCGGTAG
AGCATTCTCCTGCGGAGCCAGACTCCTCCTCGGGAACCGGAAAGGCGGGCCAGCAGCCTG
CGAGAAAGAGATTGAATTTCGGTCAGACTGGAGACGCAGACTCCGTACCTGACCCCCAGC
CTCTCGGACAGCCACCAGCAGCCCCCTCTGGTCTGGGAACTAATACGATGGCTACAGGCA
GTGGCGCACCAATGGCAGACAATAACGAGGGCGCCGACGGAGTGGGTAATTCCTCGGGAA
ATTGGCATTGCGATTCCACATGGATGGGCGACAGAGTCATCACCACCAGCACCCGAACCT
GGGCTCTGCCCACCTACAACAACCATCTGTACAAGCAGATTTCCAGCCAATCAGGAGCAT
CGAACGACAACCACTACTTTGGCTACAGCACCCCTTGGGGGTATTTTGACTTCAACAGAT
TCCACTGCCACTTCTCCCCACGTGACTGGCAAAGACTCATCAACAACAACTGGGGATTCC
GACCCAAGAGACTCAACTTCAAGCTCTTTAACATTCAAGTCAAGGAGGTCACGCAGAATG
ACGGTACGACGACGATTGCCAATAACCTTACCAGCACGGTTCAGGTGTTTACTGACTCGG
AGTACCAGCTCCCGTACGTCCTCGGCTCGGCGCATCAAGGATGCCTCCCGCCGTTCCCAG
CAGACGTCTTCATGGTGCCACAGTATGGATACCTCACCCTGAACAACGGGAGTCAGGCGG
TAGGACGCTCTTCATTTTACTGCCTGGAGTACTTTCCTTCTCAGATGCTGCGTACCGGAA
ACAACTTTACCTTCAGCTACACCTTTGAGGACGTTCCTTTCCACAGCAGCTACGCTCACA
GCCAGAGTTTGGACCGTCTCATGAATCCTCTCATCGACCAGTACCTGTATTACTTGAGCA
GAACAAACACTCCAAGCGGAACCACCACGATGTCCAGGCTTCAGTTTTCTCAGGCCGGAG
CGAGTGACATTCGGGACCAGTCTAGGAACTGGCTTCCTGGACCCTGTTACCGCCAGCAGC
GAGTATCAAAGACAGCTGCGGACAACAACAACAGTGATTACTCGTGGACTGGAGCTACCA
AGTACCACCTCAATGGCCGGGACTCGTTGGTTAATCCAGGACCAGCCATGGCCAGCCACA
AAGACGATGAAGAAAAGTATTTTCCTCAGAGCGGGGTTCTCATCTTCGGAAAACAAGACT
CGGGAAAAACTAATGTGGACATTGAAAAGGTCATGATTACAGACGAAGAGGAAATCAGGA
CCACCAATCCCGTGGCTACGGAGCAGTATGGTTCTGTATCTACCAACCTTCAAAGCGGCA
ACACACAAGCAGCTACCTCAGATGTCAACACACAAGGCGTTCTTCCTGGCATGGTCTGGC
AGGACAGAGACGTGTACCTGCAGGGACCCATCTGGGCAAAGATTCCACACACGGACGGAC
ACTTTCACCCTTCTCCCCTCATGGGCGGATTTGGACTGAAGCACCCTCCCCCGCAGATTC
TCATCAAGAATACCCCGGTACCTGCGAATCCTTCTACCACCTTCAGCGCGGCAAAGTTTG
CTTCCTTCATCACACAGTACTCCACGGGGCAGGTCAGCGTGGAGATCGAGTGGGAGCTGC
AGAAGGAGAACAGCAAACGCTGGAACCCCGAGATCCAGTACACTTCCAACTACAACAAAT
CTGTTAATGTGGACTTTACTGTGGACACTAATGGCGTGTATTCAGAGCCTCGCCCCATTG
GCACCAGATACCTGACTCGTAATCTGTAATTGCTTGTTAATCAATAAACCGTTTAATTCG
TTTCAGTTGAACTTCAGTCTCCGCGTACTTCTTTCTTATCTAGTTTCCATGGCTACGTAG
ATAAGTAGCATGGCGGGTTAATCATTAACTTTAAGGAACCCCTAGTGATGGAGTTGGCCA
CTCCCTCTCTGCGCGCTCGCTCGCTCACTGAGGCCGCCCGGCAAAGCCCGGGCGTCGGGC
GACCTTTGGTCGCCCGGCCTCAGTGAGCGAGCGAGCGCGCAGAGAGGGAGTGGCCAA
SEQ ID NO: 86
>#2208_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCAGGGTATTTAAGCCCGAGTGAGCACGCAGGGTCTCCATTTTGAAGCGGGA
GGTTTGAACGCGCAGCCGCCATGCCGGGTTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCACGTGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAGGAGAATCAGAATCCCAATTCTGACGCGC
CGGTGATCAGATCAAAAACCTCAGCCAGGTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAACTCGCGGTCCCAAATCAAGGCTGCTCTGGACAATGCGGGAAAGATCA
TGAGCCTGACTAAAACCGCTCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCTTTCAATGATTGCGTCGACAAGATGG
TGATCTGGTGGGAGGAGGGGAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATTGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCAAGTGACGCAGATATAAGTGAGCCCAAACGGGCGCGCGAGTCAGTTGCGC
AGCCATCGACGTCAGACGCGGAAGCTTCGATCAACTACGCGGACAGGTATCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGCTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGCTGAGCCAGATTCCTCCTCCGGAACTGGAAAGTCGGGCCAACAGCCT
GCAAGAAAAAGATTGAATTTTGGTCAGACTGGAGACGCAGACTCCGTACCAGACCCTCAA
CCTCTCGGAGAACCACCAGCAGCCCCCACAAGTTTGGGATCTACTACAATGGCTTCAGGC
GGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
AGCAACGACAACCACTACTTTGGCTACAGCACCCCTTGGGGGTATTTTGACTTCAACAGA
TTCCACTGCCACTTTTCACCACGTGACTGGCAAAGACTCATCAACAACAATTGGGGATTC
CGGCCCAAAAGACTCAACTTCAAGCTGTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCCCCCGCCGTTCCCA
GCAGACGTCTTCATGGTGCCACAGTATGGATACCTCACCCTGAACAACGGGAGTCAGGCG
GTAGGACGCTCTTCCTTTTACTGCCTGGAGTACTTTCCTTCTCAGATGCTTCGTACCGGA
AACAACTTTACCTTCAGCTACACCTTTGAAGACGTTCCTTTCCACAGCAGCTACGCTCAC
AGTCAAAGTCTGGACCGTCTCATGAATCCTCTCATCGACCAGTACCTGTATTACTTGAGC
AGAACAAACACTCCAAGCGGAACCACTACGCAGTCCAGGCTTCAGTTTTCTCAGGCCGGA
GCGAGTGACATTCGGGACCAGTCTAGGAACTGGCTTCCTGGACCCTGTTACCGCCAGCAG
CGAGTATCAAAGACAGCTGCGGATAACAACAACAGTGAATACTCGTGGACTGGAGCTACC
AAGTACCACCTCAATGGCAGAGACTCTCTGGTGAATCCGGGCCCGGCCATGGCCAGCCAC
AAGGACGATGAAGAAAAGTTTTTTCCTCAAAGCGGGGTTCTCATCTTTGGGAAGCAAGGC
TCAGAGAAAACAAATGTGGACATTGAAAAGGTCATGATTACAGACGAAGAGGAAATCAGG
ACCACCAATCCCGTGGCTACGGAGCAGTATGGTTCTGTATCTACCAACCTCCAGAGCGGC
AACACACAAGCAGCTACCTCAGATGTCAACACACAAGGCGTTCTTCCAGGCATGGTCTGG
CAGGACAGAGACGTGTACCTGCAGGGACCCATTTGGGCCAAGATTCCTCACACCGATGGA
CACTTTCATCCTTCTCCGCTGATGGGAGGTTTTGGACTCAAACACCCACCTCCTCAGATC
ATGATCAAAAACACTCCCGTTCCAGCCAATCCTCCCACAAACTTCAGTTCTGCCAAGTTT
GCTTCCTTCATTACACAGTATTCCACGGGGCAGGTCAGCGTGGAGATCGAGTGGGAGCTG
CAGAAGGAGAACAGCAAGCGCTGGAACCCTGAGATCCAGTACACTTCCAACTATAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCCGCGTACTTCTTTTCTTATCTAGGTTCCATGGCTACGT
AGATAAGTAGCATGGCGGGTTAATCATTAACTACAAGGAACCCCTAGTGATGGAGTTGGC
CACTCCCTCTCTGCGCGCTCGCTCGCTCGCTGAGGCCGGGCGACCAAAGGTCGCCCGACG
CCCGGGCTTTGCCCGGGCGGCCTCAGTGAGCGAGCGAGCGCGCAGAGAGGGAGTGGCCAA
SEQ ID NO: 87
>#129_AAV.FL.linear [organism = Adeno-associated virus][mol_type = genomic DNA]
[isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGGGGTCACGTGAGTGTTTTGCGACATTTTGCGACACCAT
GTGGTCACGCTGGGTATTTAAGCCCGAGTGAGCACGCAGGGTCTCCATTTTGTAGCGGGA
GGTTTGAACGCGCAGCCGCCATGCCGGGATTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCACGTGC
TGGTGCAACCACCGGGGTTTAAATCCATGGTTTTGGGACGTTTCCTGAGTCAAATTCGCG
AAAAACTGATTCAAAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTCAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACCTCGGAGAAGCAGTGGATCCAGGAGGACCAGGCCTCATACATCTCCTTCA
ATGCGGCCTCCAACTCGCGGTCTCAAATCAAGGCTGCTCTGGACAATGCGGGGAAGATCA
TGAGCCTGACTAAAACGGCCCCCGACTACCTGGTGGGCCAGCAGCCCGTGGAGGACATTT
CCAGCAATCGGATCTATAAAATATTGGAACTGAACGGGTACGACCCCCAATATGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCTTTTT
ACGGGTGCGTAAACTGGACCAATGAGAACTTTCCGTTCAACGATTGCGTCGACAAGATGG
TGATTTGGTGGGAGGAGGGGAAGATGACCGCCAAGGTCGTGGAGTCGGCCAAAGCCATTC
TCGGAGGAAGAAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCTTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATCAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAAGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGCGGAGCCAGACTCCTCCTCGGGAACCGGAAAAGCGGGCCAGCAGCCT
GCAAGAAAAAGACTGAATTTCGGTCAGACTGGAGACGCAGACTCCGTACCTGACCCCCAG
CCTCTCGGACAGCCACCAGCAGCCCCCACAAGTTTGGGATCTACTACAATGGCTACAGGC
AGTGGCGCACCAATGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTTTACAAGCAAATCTCCAGCCAATCAGGAGCC
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTGCCACAGTATGGATACCTCACCCTGAACAACGGGAGTCAGGCG
GTAGGACGCTCTTCCTTTTACTGCCTGGAGTACTTTCCTTCTCAGATGCTGCGTACCGGA
AACAACTTTACCTTCAGCTACACTTTTGAGGACGTTCCTTTCCACAGCAGCTACGCTCAC
AGCCAGAGTCTGGATCGTCTGATGAATCCTCTCATCGACCAGTACCTGTATTATCTAAAC
AGAACACAAACARCCAGTGGAACTCCCACGCAGTCTCGGCTACTGTTTAGCCAAGCTGGA
CCCACAAGCATGTCTCTTCAAGCTAAAAACTGGCTGCCTGGACCGTGTTATCGCCAGCAG
CGTTTGTCAAAGCAGGCAAACGACAACAACAACAGCAACTTTCCCTGGACTGGAGCTACC
AAGTACCACCTCAATGGCAGAGACTCTTTGGTGAACCCGGGCCCGGCCATGGCCAGCCAC
AAGGACGATGAAGAAAAGTTTTTCCCCATGCATGGAACCCTAATCTTTGGGAAGCAAGGC
TCAGAGAAAACCAACGCGGAATTGGAAAATGTCATGATTACAGATGAAGAGGAAATCAGG
ACCACCAATCCCGTGGCTACAGAGCAGTATGGTTCTGTATCTACCAACCTCCAGAGAGGC
AACAGACAAGCAGCTACCGCAGATGTCAACACACAAGGCGTTCTTCCAGGCATGGTCTGG
CAGGACAGAGATGTGTACCTTCAGGGGCCCATCTGGGCAAAGATTCCACACACGGACGGA
CATTTTCACCCCTCTCCCCTCATGGGTGGATTCGGACTTAAACACCCTCCTCCACAGATT
CTCATCAAGAACACCCCGGTACCTGCGAATCCTTCGACCACCTTCAGTGCGGCAAAGTTT
GCTTCCTTCATCACACAGTATTCCACGGGACAGGTCAGCGTGGAGATCGAGTGGGAGCTG
CAGAAGGAGAACAGCAAACGCTGGAACCCCGAGATCCAGTACACTTCCAACTACAACAAA
TCTGTTAATGTGGACTTTACTGTGGACACTAATGGTGTGTATTCAGAGGCTCGCCCCATT
GGCACCAGATACCTGACTCGTAATCTGTAATTGCTTGTTAATCAATAAACCGTTTAATTC
GTTTCAGTTGAACTTTGGTCTCCGCGTACTTCTTTCTTATCTAGTTTCCATGGCTACGTA
GATAAGTAGCATGGCGGGTTAATCATTAAATCTAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 88
>#2731_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAAAAGCTGCAGCGTGACTTTCTGACGGAATGGCGCCGTGTGAGTAAGGCCC
CGGAGGCCCTTTTCTTTGTGCAATTTGAGAAGGGAGAGAGCTACTTCCACATGCACGTGC
TCGTGGAAACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACTTGCTCCCCAAAACCCAGCCTGAGCTCCAGTGGGCGTGGACTAATATGGAAC
AGTATTTAAGCGCCTGTTTGAATCTCACGGAGCGTAAACGGTTGGTGGCGCAGCATCTGA
CGCACGTGTCGCAGACGCAGGAGCAGAACAAAGAGAATCAGAATCCCAATTCTGATGCGC
CGGTGATCAGATCAAAAACTTCAGCCAGGTACATGGAGCTGGTCGGGTGGCTCGTGGACA
AGGGGATTACCTCGGAGAAGCAGTGGATCCAGGAGGACCAGGCCTCATACATCTCCTTCA
ATGCGTCCTCCAACTCGCGGTCCCAAATCAAGGCTGCCTTGGACAATGCGGGAAAGATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATATGCGGCTT
CCGTCTTTCTGGGATGGGCCACGAAAAAGTTCGGCAAGAGGAACACCATCTGGCTGTTTG
GGCCTGCAACTACCGGGAAGACCAGGAGGATGTCCTCCAGCTTGGGCAACGTGCCCTTCT
ACGGGTGCGTAAACTGGACCAATGAGAACTTTCCCTTCAACGACTGCGTCGACAAGATGG
TGATCTGGTGGGAGGAGGGAAAGATGACTGCCAAGGTCGTGGAGTCTGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACTAGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAAAAAA
GACCCGCCCCCAGTGACGCAGATATAAGTGAGCCCAAACGGGTGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATTATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACGTGTTAAGACGGCTCCGGGAAAAAAGAGGCCGGTA
GCAGTGGCGCACCAGTGGCAGACAATAACGAGGGTGCCGATGGAGTGGGTAATTCCTCAG
GAAAAAAGATTGAATTTTGGTCAGACTGGAGACGCAGATTGGCATTGCGACCCCCAGCCT
CTCGGACAGCCACCAGCAGCCCCCTCTGGTTTGGGATCTACTACAATGGCTACAGGCAGT
GGCGCACTATTATTATTGGAAAGCTTGAGTGCCGATGGAGTGGGTAATTCCTCAGGAAAT
TGGCATTGCGATTCCCAATGGCTGGGCGACAGAGTCATCACCACCAGCACCCGAACCTGG
GCCCTGCCCACCTACAACAACCACCTCTACAAGCAAATCTCCAGCCAATCAGGAGCCTCA
AACGACAACCACTACTTTGGCTACAGCACCCCTTGGGGGTATTTTGACTTCAACAGATTC
CACTGCCACTTTTCACCACGTGACTGGCAAAGACTCATCAACAACAACTGGGGATTCCGA
CCCAAGAGACTCAACTTCAAGCTCTTTAACATTCAAGTCAAAGAGGTCACGCAGAATGAC
GGTACGACGACGATTGCCAATAACCTTACCAGCACGGTTCAGGTGTTTACTGACTCGGAG
TACCAGCTCCCGTACGTCCTCGGCTCGGCGCATCAAGGATGCCTCCCGCCGTTTCCAGCG
GACGTCTTCATGGTGCCACAGTATGGATACCTCACCCTGAACAACGGGAGTCAGGCAGTA
GGACGCTCTTCATTTTACTGCCTGGAGTACTTTCCTTCTCAGATGCTGCGTACTGGAAAC
AACTTTCAGTTCAGCTACACCTTTGAAGACGTTCCTTTCCACAGCAGCTACGCTCACAGC
CAGAGTCTGGATCGGCTGATGAATCCTCTGATCGACCAGTACCTGTATTATCTGAACAAA
ACACAATCAAATAGTGGAACCACCACGATGTCAAGGCTACTGTTTAGTCAAGCTGGACCC
ACCAGCATGTCTCTTCACGCTAAAAACTGGCTGCCTGGACCTTGCTACAGACAGCAGCGT
CTGTCAAAGCAGGCAAACGACAACAACAACAGCAACTTTCCCTGGACTGCAGCTACAAAG
TATCATCTAAATGGCAGTGACTCGTTGATTAATCCAGGACCAGCTATGGCCAGCCACAAA
GACGATGAAGAAAAGTTTTTTCCTCAGAGCGGGGTTCTCATCTTTGGGAAGCAAGGCTCA
GAGAAAACAAATGTGGACATTGAAAAGGTCATGATTACAGACGAAGAGGAAATCAGGACA
ACCAATCCCGTGGCTACGGAGCAGTATGGATCTGTATCTACCAACCTCCAGAGAGGCAAC
AGACGAGCAGCTACCGCAGATGTCAACACACAAGGCGTTCTTCCAGGCATGGTCTGGCAG
GACAGAGATGTGTACCTTCAGGGGCCCATCTGGGCAAAGATTCCACACACGGACGGACAT
TTTCACCCCTCTCCCCTCATGGGTGGATTCGGACTTAAACACCCTCCTCCACAGATTCTC
ATCAAGAACACCCCGGTACCTGCGAATCCTTCGACCACCTTCAGTGCGGCAAAGTTTGCT
TCCTTCATCACACAGTACTCCACGGGACAGGTCAGCGTGGAGATCGAGTGGGAGCTGCAG
AAGGAAAACAGCAAACGCTGGAATCCCGAAATTCAGTACACTTCCAACTACAACAAGTCT
GTTAATGTGGACTTTACTGTGGACACTAATGGCGTGTATTCAGAGCCTCGCCCCATTGGC
ACCAGATACCTGACTCGTAATCTGTAATTGCTTGTTAATCAATAAACCGTTTAATTCGTT
TCAGTTGAACTTTGGTCTCTGCGTGCTTCTTTCTTATCTAGTTTCCATGGCTACGTAGAT
AAGTAGCATGGCGGGTTAATCATTAACTATAAGGAACCCCTAGTGATGGAGTTGGCCACT
CCCTCTCTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAGAGAGGGAGTGGCCAA
SEQ ID NO: 89
>#1602_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCAGGGTATTTAAGCCCGAGTGAGCACGCAGGGTCTCCATTTTGAAGCGGGA
GGTTTGAACGCGCAGCCGCCATGCCTGGATTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACAGAATGGCGCCGTGTGAGTAAAGCCC
CCGAGGCCCAGATCACAAAGTCAGGAGAGAAGGGAGAGAGCTACTTCCACATGCACGTGC
TGGTGGAAACCACCGGGGTGAAATCCATGGTTTTGGGACGTTTCCTGAGTCAGATTCGCG
AAAAACTGATTCAGAGAATTTACCGCGGGATCGAACCGACTTTGCCATACTGGTTCGCGG
TCACAAAGACCCGAAATGGCGCCGGAGGCGGGAACAAGGTGGTGGATGAGTGCTACATCC
CCAATTACCTGCTCCCCAAAACCCAACCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATCAGATCAAAAACCTCAGCCAGGTACATGGAGCTGGTCGGGTGGCTCGTGGATA
AGGGGATTACCTCGGAGAAGCAGTGGATCCAGGAGGACCAGGCCTCATACATCTCTTTCA
ATGCGGCCTCCAACTCGCGGTCCCATATCAAGGCTGCTTTGGACAATTCTGGAAAGATCA
TGAGCCTTACTAAAATGGCATTTGACCCAGCCTATATCCAGCAGCCCGTGGAGGACATTT
CCAGCAACCGGATCTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCTCACACTGTGCCCTTCT
ACGGGTGCGTAAACTGGACCAATGAGAACTTTCCTTTCAATGATTGCGTCGACAAAATGG
TGATCTGGTGGGAGGAGGGAAAGATGACCGCCAAGGTCGTGGAGTCGGCCAAAGCCATTC
TCGGCGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCAATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGTCCGCAGAGCGGCATAAGGACGACAGCAGGGGTCTTGTG
CTTCCTGGGTACAAGTACCTCGGACCCTTCAASGGACTCGACAAAGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAMCCTCTGGGCCYGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGACTGAATTTCGGTCAGACTGGAGACTCAGACTCAAGCTCCAGGGATATG
AGAAAAGTATTTTCTGCAGCAGCCCCCACAAGTTTGGGATCTACTACAATGGCTTCAGGC
GGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
AGCAACGACAACCACTACTTTGGCTACAGCACCCCTTGGGGGTATTTTGACTTCAACAGA
TTCCACTGCCACTTTTCACCACGTGACTGGCAAAGACTCATCAACAACAATTGGGGATTC
CGGCCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTTAGCTACACTTTTGAAGACGTTCCTTTCCACAGCAGCTACGCTCAC
AGCCAGAGTCTGGATCGGCTGATGAATCCTCTGATCGACCAGTACCTGTATTATCTGAAC
AAGACACAAACAAATAGTGGAACTCTTCAGCAGTCTCGGCTACTGTTTAGCCAAGCTGGA
CCAACCAACATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCAGCTACA
AAGTATCACCTCAATGGCAGAGACTCTCTGGTGAATCCGGGCCCGGCCATGGCAAGCCAC
AAGGACGATGAAGAAAAGTTTTTTCCTCAGAGCGGGGTTCTCATCTTTGGGAAGCAAGGC
TCAGAGAAAACAAATGTGGACATTGAAAAGGTCATGATTACAGACGAAGAGGAAATCAGG
ACAACCAATCCCGTGGCTACGGAGCAGTATGGTTCTGTATCTACCAACCTCCAGAGAGGC
AACAGACAAGCAGCTACCGCAGATGTCAACACACAAGGCGTTCTTCCAGGCATGGTCTGG
CAGGACAGAGATGTGTACCTTCAGGGGCCCATCTGGGCAAAGATTCCACACACGGACGGA
CATTTTCACCCCTCTCCCCTCATGGGTGGATTCGGACTTAAACACCCTCCTCCACAGATT
CTCATCAAGAACACCCCGGTACCTGCGAATCCTTCGACCACCTTCAGTGCTGCCAAGTTT
GCTTCTTTCATCACACAGTATTCCACGGGGCAGGTCAGCGTGGAGATCGAGTGGGAGCTG
CAGAAGGAGAAMAGCAAACGCTGGAATCCCGAGATTCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTGTAATTC
GTTTCAGTTGAACTTTGGTCTCCGCGTACTTCTTTTCTTATCTAGTTTCCATGGCTACGT
AGATAAGTAGCATGGCGGGTTAATCATTAACCACAAGGAACCCCTAGTGATGGAGTTGGC
CACTCCCTCTCTGCGCGCTCGCTCGCTCACTGAGGCCGGGCGACCAAAGGTCGCCCGACG
CCCGGGCTTTGCCCGGGCGGCCTCAGTGAGCGAGCGAGCGCGCAGAGAGGGAGTGGCCAA
SEQ ID NO: 90
>#1010_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGYGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGAGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACAGAGTGGCGCCGCGTGAGTAAGGCCC
CGGAGGCCCTTTTCTTTGTGCAATTTGAGAAGGGAGAGAGCTACTTCCACATGCACGTGC
TCGTGGAAACCACCGGGGTGAAATCCATGGTTTTAGGACGTTTCCTGAGTCAGATTCGCG
AAAAACTGATTCAGAGAATTTACCGCGGGATCGAGCCGACTTTGCCAAACTGGTTCGCGG
TTACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCTGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACCTCGGAGAAGCAGTGGATCCAGGAGGACCAGGCCTCATACATCTCCTTCA
ATGCGGCCTCCAACTCGCGGTCCCAAATCAAGGCTGCCTTGGACAATGCGGGAAAGATCA
TGAGCCTGACTAAAACCGCCCCCGACTACCTGGTGGGCCAGCAGCCCGTGGAGGACATTT
CCAGCAATCGGATTTATAAAATTTTGGAACTAAACGGGTACGATCCCCAATATGCGGCTT
CCGTCTTTCTGGGATGGGCCACGAAAAAGTTTGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCCTTCAACGACTGTGTCGACAAGATGG
TGATCTGGTGGGAGGAGGGAAAGATGACCGCCAAGGTTGTGGAGTCGGCCAAAGCTATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGTGCGCGAGTCAGTTGCGC
AGCCATCGACGTCAGACGCGGAAGCTTCAATCAACTACGCGGACAGGTACCAAAACAAAT
GTTCTCGTCACGTGGGCATGAATCTGATGCTGTTTCCCTGCAGACAATGCGAGAGAATGA
AYCAGAATTCAAATATTTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTTTGAACAATAAATGATTTAAATCAGGTATGGCTGCCGATGGTTAT
CTTCCAGATTGGCTCGGGGAGACCCTCTCTCAAGGAATAAGACAGTGGTGGAAGCTCAAA
CCTGGCCCACCACCACCAAAGCCCGCAGAGCGGCATAAGGRCGACAGCAGGGGTCTTGTG
CTTCCTGGGTACAAGTACCTCGGACCCTTCAACGGACTCGACAAGGGAGAACCGGTCAAC
GAGGCAGACGCCGCGGCCCTCGAACACGACAAGGCCTACGACCGGCAGCTCGACAGCGGA
CACATCCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGATTGAATTTCGGCCAGACTGGAGACGCAGACTCCGTACCTGACCCCCAG
CCTCTCGGACAGCCACCAGCAGCCCCCTCTGGTCTGGGAACTACTACGATGGCTACAGGC
AGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAACTTTTTTCAAATTAAAATAAATGCC
TCGAACGACAATCACTACTTTGGCTACAGCACCCCTTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTCTCATTCAAGTCAAAGAGGTCACGCAGAAT
GACGGTACGACGACGATTGCCAATAACCTTACCAGCACGGTTCAGGTGTTTACTGMCTCG
GAGTACCAGCTCCCGTACGTCCTCGGCTCGGCSCAYCAAGGRTGCCYCCCGCCGTTCCCA
GCGGACGTCTTCATGGTCCCACAGTATGGATACCTCACCCTGAACAACGGGAGTCAGGCG
GKAGGACGCTCTTCATTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAAACACAATCAAATAGTGGAACTCTTCAGCAGTCTCGGCTACTGTTTAGTCAAGCTGGA
CCCACCAGCATGTCTCTTCAAGCTAAAAACTGGCTGCCTGGACCCTGTTACCGCCAGCAG
CGAGTATCAAAGACATCTGCGGATAACAACAACAGTGAATACTCGTGGACTGGAGCTACC
AAGTACCACCTCAATGGCAGAGACTCTCTGGTGAATCCGGGCCCGGCCATGGCAAGCCAC
AAGGACGATGAAGAAAAGTTTTTTCCTCAGAGCGGGGTTCTCATCTTTGGGAAGCAAGGC
TCAGAGAAAACAAATGTGGACATTGAAAAGGTCATGATTACAGACGAAGAAGAAATCCGC
ACCACCAATCCCGTGGCTACGGAGCAGTACGGATATGTGTCAAATAATTTGCAAAACTCA
AATAGACAAGCAGCTACCGCAGATGTCAACACACAAGGCGTTCTTCCAGGCATGGTCTGG
CAGGACAGAGATGTGTACCTTCAGGGGCCCATCTGGGCAAAGATTCCACACACGGACGGA
CATTTTCACCCCTCTCCCCTCATGGGTGGATTCGGACTTAAACACCCTCCTCCACAGATT
CTCATCAAGAACACCCCGGTTCCAGCCAATCCTCCTACAAACTTCAGTTCTGCCAAGTTT
GCTTCTTTCATCACACAGTACTCCACGGGGCAGGTCAGCGTGGAGATCGAGTGGGAGCTG
CAGAAGGAAAACAGCAAACGCTGGAATCCCGAGATTCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTCTTTCTTATCTAGTTTCCATGGCTACGTA
GATAAGTAGCATGGCGGGTTAATCATTAACTATAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 91
>#985_AAV.FL.linear [organism = Adeno-associated virus][mol_type = genomic DNA]
[isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAATGGCGCCGTGTGAGTAAGGCCC
CGGAGGCCCTTTTCTTTGTGCAATTTGAGAAGGGAGAGAGCTACTTCCACATGCACGTGC
TCGTGGAAACCACCGGGGTGAAATCCATGGTTTTGGGACGTTTCCTGAGTCAGATTCGCG
AAAAACTGATTCAGAGAATTTACCGCGGGAGCGAGCCGACTTTGCCAAACTGGTTCGCGG
TGACAAAGACCCGAAATGGCGCCGGAGGCGGGAACGAGGTGGTGGATGAGTGCTACATCC
CCAATTACTTGCTCCCCAAAACCCAGCCTGAGCTCCAGTGGGCGTGGACTAATATGGAAC
AGTATTTAAGCGCCTGTTTGAATCTCACGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATCAGATCAAAAACCTCAGCCAGGTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAACTCGCGGTCCCAAATCAAGGCTGCCTTGGACAATGCGGGGAAGATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATATGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCCTTCAACGACTGTGTCGACAAGATGG
TGATCTGGTGGGAGGAGGGGAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGCC
TGGATCATGACTTTGGGAAGGTCACAAAGCAGGAAGTCAAAGACTTTTTTCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCAGACAGGTACCAAAACAAAT
GTTCTCGTCACGTGGGCATGAATCTGATGCTGTTTCCCTGTAGACAATGCGAAAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCATATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCCCTCTGCAGGAATAAGTCAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAGGCGGGCCAGCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGCAGACAATAACGAGGGCGCCGACGGAGTGGGTAATTCCTCGGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCT
TCAAATGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAAACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGGCTCG
GGGTACGGGCTCCGGTACGTCCTCGGCTCGGCGCATCAAGGATGCCTCCCGCCGTTTCCA
GCGGACGTCTTCATGGTCCCACAGTATGGATACCTCACCCTGAACAACGGGAGTCAGGCA
GTAGGACGCTCTTCCTTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATTGACCAGTACCTGTATTATCTGAAC
AAGACACAATCAAATAGTGGAACTCTTCAGCAGTCAAGGCTTCAGTTTTCTCAGGCCGGA
GCGAGTGACATTCGGGACCAGTCTAGGAACTGGCTTCCTGGACCCTGTTACCGCCAGCAG
CGAGTATCAAAGACATCTGCGGATAACAACAACAGTGAATACTCGTGGACTGGAGCTACC
AAGTACCACCTCAATGGCAGAGACTCTCTGGTGAATCCGGGCCCGGCCATGGCAAGCCAC
AAGGACGATGAAGAAAAGTTTTTTCCTCAGAGCGGGGTTCTCATCTTTGGGAAGCAAGGC
TCAGAGAAAACAAATGTGGACATTGAAAAGGTCATGATTACAGACGAAGAGGAAATCAGG
ACAACCAATCCCGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGACTCAAACACCCGCCTCCTCAGATC
ATGATCAAAAACACTCCCGTTCCAGCCAATCCTCCCACAAACTTCAGTTCTGCCAAGTTT
GCTTCTTTTATCACACAGTATTCCACGGGGCAGGTCAGCGTGGAGATCGAGTGGGAGCTG
CAGAAGGAGAACAGCAAACGCTGGAATCCCGAGATTCAGTACACTTCCAACTACAACAAG
TCTGTTAATGTGGACTTTACTGTGGACACTAATGGCGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTCTTTCTTATCTAGTTTCCATGGCTACGTA
GATAAGTAGCATGGCGGGTTAATCATTAACTACAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 92
>#667_AAV.FL.linear [organism = Adeno-associated virus][mol_type = genomic DNA]
[isolation_source = liver tissue][topology = linear]
TTGGCCACTCCCTCTCTGCGCGCTCGCTCGCTCACTGAGGCCGGGCGACCAAAGGTCGAG
AGAGCTGCCCGCCTCGGCTCTCCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCAGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGAAGGCACCCCTGA
CCGTGGCCGAGAAGCTGCAGCGCGACTTTCTGACGGAGTGGCGCCGTGTGAGTAAGGCTC
CGGAGGCCCTTTTCTTTGTGCAATTTGAGAAGGGAGAGAGCTACTTCCACATGCACGTGC
TTGTGGAGACCACTGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACGCAGACCCTCCATGCCGCCGCAGGCGGGAACACAGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATCACCTCGGAGAAGCAGTGGATCCAGGAGGACCAGGCCTCATACATCTCCTTCA
ATGCGGCCTCCAACTCGCGGTCCCAGATCAAGGCTGCCBTGGACAATGCGGGAAAGATCA
TGAGCCTRACTAAAACCGCCCCCGACTACCTGGTGGGCCAGCAGCCCGTGGAGGACATTT
CCAGCAATCGGATTTATAAAATTTTGGAACTCAACGGGTACGATCCCCAATATGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCCTTCAACGACTGTGTCGACAAGATGG
TGATCTGGTGGGAGGAGGGAAAGATGACCGCCAAGGTCGTGGAGTCCGCCAAAGCCATTC
TCGGAGGAAGCAAGATGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAAAAAA
GACCCGCCCCCAGTGACGCAGATATAAGTGAGCCCAAACGGGCGCGCGAGKCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATTTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAAGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCTGGAAAAAAGAGGCCGGTA
GAGCACTCTCCTGTGGAGCCAGACTCCTCCTCGGGAACCGGAAAAGCGGGCCAGCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTCTGGGAACTAATACGATGGCTACAGGC
AGTGGCGCACCAATGGAAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATTTGCAGCCAATCAGGAGCC
TCAAACGACAACCACTACTTTGGCTACAGCACCCGGTGGGGGGATTTTGACTTCAACAGA
TTCCACTGCCACTTTTCACCACGTGACTGGCAAAGACTCATCAACAACAACTGGGGATTC
CGGCCCAAAAGACTCAACTTCAAGCTCTTTAACATTCAAGTCAAAGAGGTCACGCAGAAT
GACGGTACGAMGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCGGACGTCTTCATGGTTCCACAGTATGGATACCTCACCCTGAACAACGGGAGTCAGGCG
GTAGGACGCTCTTCCTTTTACTGCCTGGAGTACTTTCCTTCTCAGATGCTGCGTACCGGA
AACAACTTTACCTTCAGCTACACCTTTGAAGACGTTCCTTTCCACAGCAGCTACGCTCAC
AGCCAGAGTCTGGATCGGCTGATGAATCCTCTGATCGACCAGTACCTGTATTACTTGAGC
AGAACAAACACTCCAAGTGGAACCACCACGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCGGCTACA
AAGTATCATCTAAATGGCCGGGACTCGTTGGTTAATCCAGGACCAGCTATGGCCAGCCAC
AAGGACGATGAAGAAAAGTTTTTTCCTCAGAGCGGGGTTCTCATCTTTGGGAAGCAAGGC
TCAGAGAAAACAAATGTGGACATTGAAAAGGTCATGATTACAGACGAAGAGGAAATCAGG
ACAACCAATCCCGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCCCTCATGGGTGGATTCGGACTTAAACACCCTCCTCCACAGATC
ATGATCAAAAGCTCTSCCGTTCCAGCCAATCCTCCTACAAACTTCAGTTCTGCCAAGTTT
GCTTCTTTCATCACACAGTACTCCACGGGGCAGGTCAGCGTGGAGATCGAGTGGGAGCTG
CAGAAGGAGAACAGCAAACGCTGGAATCCCGAGATTCAGTACACTTCAAACTACAACAAG
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCCGCGTACTTCTTTCTTATCTAGTTTCCATGGCTACGTA
GATAAGTAGCATGGCGGGTTAATCATTAACTACAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 93
>#2806_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGTTGTCCAGAGCAGCCTTGATTTGGGACCGCGGAAGTTGGAGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGARTGGCGCCGCGTGAGTAAGGCCC
CGGAGGCCCTCTTCTTTGTGCAATTTGAGAAGGGAGAGAGCTACTTCCACATGCACGTGC
TCGTGGAAACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCAGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCTGAGCGTAAACGGTTGGTGGCGCAGCATCTGA
CGCACGTGTCGCAGACGCAGGAGCAGAACAAAGAGAATCAGAATCCCAATTCTGATGCGC
CGGTGATCAGATCAAAAACTTCAGCCAGGTACATGGAGCTGGTCGGGTGGCTCGTGGACA
AGGGGATTACCTCGGAGAAGCAGTGGATCCAGGAGGACCAGGCCTCATACATCTCCTTCA
ATGCGGCCTCCAACTCGCGGTCCCAAATCAAGGCTGCTCTGGACAATGCGGGAAAGATCA
TGAGCCTGACTAAAACCGCCCCAGACTACCTAGTGGGCCAGCAGCCCGTGGAGGACATTT
CCAGCAATCGGATTTATAAAATTTTGGAACTAAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCCTTCAATGACTGCGTCGACAAGATGG
TGATCTGGTGGGAGGAGGGGAAGATGACCGCCAAGGTCGTAGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACAAAGCAGGAAGTCAAAGACTTTTTCCGGTGGGCAA
AGGATCACGTGGTTGAGGTGGAGCATGAATTCTACGTCAAAAAGGGTGGAGCYAAGAAAA
GGCCCGCCCCCAGTGACGCAGATATAAGTGAGCCCAAACGGGCGCGCGAGTCAGTTGCGC
AGCCATCGACGTCAGACGCGGAAGCTTCRATCAACTACGCAGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTGCCAGACGCTTGCACTGCCTGCGACCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACAACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAACACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAGCCTGTTAAGACGGCTCCGGGAAAAAAGAGGCCGGTA
GAGCACTCTCCTGTGGAGCCAGACTCCTCCTCGGGAACCGGAAAAGCGGGCCAACAGCCT
GCGAAAAAAAGACTGAAGTTTGGTCAGACTGGAGACGCAGACTCCGTACCTGACCCCCAG
CCTCTCGGACAGCCACCAGCAGCCCCCTCTGGTCTGGGATCTACTACAATGGCTACAGGC
AGTGGCGCACCAATGGCAGACAATAACGAGGGTGCCGATGGAGTGGGTAATTCCTCGGGA
AATTGGCATTGCGATTCCACATGGATGGGCGACAGAGTCATCACCACCAGCACCCGAACC
TGGGCCCTGCCCACCTACAACAACCACCTCTACAAACAAATTTCCAGCCAATCAGGAGCC
TCGAACGACAATCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAAAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCGGACGTCTTCATGGTCCCACAGTATGGATACCTCACCCTGAACAACGGGAGTCAGGCG
GTAGGACGCTCTTCATTTTACTGCCTGGAGTACTTTCCTTCTCAGATGCTGCGTACCGGA
AACAACTTTACCTTCAGCTACACTTTTGAGGACGTTCCTTTCCACAGCAGCTACGCTCAC
AGCCAGAGTCTGGACCGTCTCATGAATCCTCTCATTGACCAGTACCTGTATTATCTGAAC
AAGACACAATCAAATAGTGAAACAACTTTCAGTTCAGCTACACCTTTGAAGACGTTCCTT
TCCACAGCAGCTACGCTCACAGCCAGAGTCTGGCTGCCTGGACCTTGCTACAGACAGCAG
CGTTTATCAAAGCAGGCAAACGACAACAATAACAGCAACTTTCCCTGGACTGCAGCTACA
AAGTATCATCTAAATGGCCGGGACTCGTTGGTTAATCCAGGACCAGCTATGGCCAGTCAT
AAAGACGATGAAGAAAAGTTTTTCCCTCAGAGCGGGGTTCTCATCTTTGGRAAACAAGGC
TCAGARAAAACCAATGTGGACATTGAAAAGGTCATGATTACAGACGAAGAGGAAATCAGG
ACCACCAATCCCGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATATGGGCCAAGATTCCTCACACCGATGGA
CACTTTCATCCTTCTCCGCTGATGGGAGGTTTTGGACTTAAACACCCTCCTCCACAGATC
ATGATCAAAAACACTCCCGTTCCAGCCAATCCTCCYACAAACTTCAGTTCTGCCAAGTTT
GCTTCTTTCATCACACAGTATTCCACGGGGCAGGTCAGAGTGGAGATCGAGTGGGAGCTG
CAGAAGGAAAACAGCAAACGCTGGAATCCCGAGATCCAGTACACTTCCAACTACAACAAR
TSTGTTAATGTGGACTTTACTGTGGACACTAATGGTGTGTATTCAGAGCYTCGCCCCATT
SGCACCAGATMCCTGACTCGTAATCTGTAATTGCTTGTTAATCAATAAACCGTTTAATTC
GTTTCAGTTGAACTTTGGTCTCTGCGTATTTTTTTCTTATCTAGTTTCCATGGCTACGTA
GATAAGTAGCATGGCGAGTTAATCATTAACTCTAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 94
>#217_AAV.FL.linear [organism = Adeno-associated virus][mol_type = genomic DNA]
[isolation_source = liver tissue][topology = linear]
TTGGCCACTCCCTCTCTGCGCGCTCTGAGACGGAGTTTTGCTTTTGTTGCCCAGGCTGGA
GTGCTATGGCGCGTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTTTGACGGAGTGGCGCCGCGTGAGTAAGGCCC
CGGAGGCCCTTTTCTTTGTGCAATTTGAGAAGGGAGAGAGCTACTTCCACATGCACGTGC
TCGTGGAAACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGATGCGC
CGGTGATCAGATCAAAAACTTCAGCCAGGTACATGGAGCTGGTCGGGTGGCTCGTGGATA
AGGGGATTACCTCGGAGAAGCAGTGGATCCAGGAGGACCAGGCCTCATACATCTCCTTCA
ATGCGGCCTCCAACTCGCGGTCCCAGATCAAGGCTGCCTTGGACAATGCGGGAAAGATTA
TGAGCCTCACTAAAACCGCCCCCGACTACCTGGTGGGCCAGTCTCTCTCTCTGCACAGCC
TTTAGTACAAGTTAAAGTAAAATCTTGGAACTGAACGGGTACGATCCCCAATATGCGGCT
TCCGTCTTTCTGGGATGGGCCACGAAAAAGTTCGGCAAGAGGAACACTATCTGGCTGTTT
GGGCCTGCAACTACCGGGAAGACCAACATCGCGGAGGCCATAGCCCACACTGTGCCCTTC
TACGGGTGCGTAAACTGGACCAATGAGAACTTTCCATTCAACGACTGTGTCGACAAGATG
GTGATCTGGTGGGAGGAGGGGAAAATGACCGCCAAGGTTGTGGAGTCGGCCAAAGCCATT
CTCGGAGGAAGCAAGGTGCGCGTGGACCAGAAATGCAAGTCCTCGGCCCAGATAGACCCG
ACTCCTGTGATTGTCACCTCCAACACCAACATGTGCGCCGTGATTGACGGGAACTCAACG
ACCTTCGAGCACCAGCAGCCGTTGCAAGACCGGATGTTCAAATTTGAACTCACCCGCCGT
CTGGATCATGACTTTGGGAAGGTCACCAAGCAGGAAGTCAAAGACTTTTTCCGGTGGGCA
AAGGATCACGTGGTTGAGGTGGAGCATGAATTCTACGTCAAAAAGGGTGGAGCCAAGAAA
AGACCCGCCCCCAGTGACGCAGATATAAGTGAGCCCAAACGGGCGCGCGAGTCAGTTGCG
CAGCCATCGACGTCAGACGCGGAAGCTTCGATCAACTACGCGGACAGGTACCAAAACAAA
TGTTCTCGTCACGTGGGCATGAATCTGATGCTGTTTCCCTGCAGACAATGCGAGAGAATG
AATCAGAATTCAAATATCTGCTTCACTCACGGACAGAAAGACTGTTTAGAGTGCTTTCCC
GTGTCAGAATCTCAACCCGTTTCTGTCGTCAAAAAGGCGTATCAGAAACTTTGCTACATT
CATCATATCATGGGAAAGGTGCCAGACGCTTGCACTGCCTGCGATCTGGTCAATGTGGAT
TTGGATGACTGCATCTCTGAACAATAAATGATTTAAATCAGGTATGGCTGCCGATGGTTA
TCTTCCAGATTGGCTCGAGGACACTCTCTCTGAAGGAATAAGACAGTGGTGGAAGCTCAA
ACCTGGCCCACCACCACCAAAGCCCGCAGAGCGGCATAAGGACGACAGCAGGGGTCTTGT
GCTTCCTGGGTACAAGTACCTCGGACCCTTCAACGGACTCGACAAGGGAGAGCCGGTCAA
CGAGGCAGACGCCGCGGCCCTCGAGCACGACAAGGCCTACGACCGGCAGCTCGACAGCGG
AGACAACCCGTACCTCAAGTACAACCACGCCGACGCGGAGTTTCAGGAGCGTCTTAAAGA
AGATACGTCTTTTGGGGGCAACCTCGGACGAGCAGTCTTCCAGGCGAAAAAGAGGGTTCT
TGAACCTCTGGGCCTGGTTGAGGAACCTGTTAAGACGGCTCCGGGAAAAAAGAGGCCGGT
AGAGCACTCTCCTGTGGAGCCAGACTCCTCCTCGGGAACCGGAAAAGCGGGCCAGCAGCC
TGCAAGAAAAAGATTGAATTTTGGTCAGACTGGAGACGCAGACTCCGTACCTGACCCCCA
GCCTCTCGGACAGCCACCAGCAGCCCCCTCTGGTTTGGGATCTACTACAATGGCTACAGG
CAGTGGCGCACCAGTGGCAGACAATAACGAGGGTGCCGATGGAGTGGGTAATTCCTCAGG
AAATTGGCATTGCGATTCCCAATGGCTGGGCGACAGAGTCATCACCACCAGCACCCGAAC
CTGGGCCCTGCCCACCTACAACAACCACCTCTACAAGCAAATCTCCAGCCAATCAGGAGC
CTCAAACGACAACCACTACTTTGGCTACAGCACCCCCTGGGGGTATTTTGACTTCAACAG
ATTCCACTGCCACTTTTCACCACGTGACTGGCAAAGACTCATCAACAACAACTGGGGATT
CCGACCCAAGAGACTCAACTTCAAGCTCTTTAACATTCAAGTCAAAGAGGTCACGCAGAA
TGACGGTACGAAGACCCCCGCCAATAACCTTACCAGCACGGTTCAGGTGTTTACTGACTC
GGAGTACCAGCTCCCGTACGTCCTCGGCTCGGCGCACCAAGGATGCCTCCCGCCGTTCCC
AGCGGACGTCTTCATGGTCCCACAGTATGGATACCTCACCCTGAACAACGGGAGTCAGGC
GGTAGGACGCTCTTCCTTTTACTGCCTGGAGTACTTTCCTTCTCAGATGCTGCGTACCGG
AAACAACTTTACCTTCAGCTACACTTTTGAAGACGTTCCTTTCCACAGCAGCTACGCTCA
CAGCCAGAGTCTGGATCGGCTGATGAATCCTCTGATCGACCAGTACCTGTATTATCTGAA
CAAAACACAATCAAATAGTGGAACTAACACGCAGTCAAGGCTTCAGTTTTCTCAGGCCGG
AGCGAGTGACATTCGGGACCAGTCTAGGAACTGGCTGCCTGGACCTTGCTACAGACAGCA
GCGTCTGTCAAAGCAGGCAAACGACAACAACAACAGCAACTTTCCCTGGACAGGAGCTAC
CAAGTACCACCTCAATGGCAGAGACTCTCTGGTGAATCCGGGCCCAGCTATGGCCAGCCA
CAAAGACGATGAAGAAAAGTTTTTCCCCATGCATGGAACCCTGATATTTGGTAAACAAGG
AACAAATGCTAACGACGCAGATTTGGACAATGTCATGATTACAGATGAAGAAGAAATCCG
CACCACCAATCCCGTGGCTACGGAGCAGTACGGATATGTGTCAAATAATTTGCAAAACTC
AAATACTGGTCCAACTACTGGAACTGTCAATCACCAAGGAGCGTTACCTGGTATGGTGTG
GCAGGATCGAGACGTGTACCTGCAGGGACCCATTTGGGCCAAGATTCCTCACACCGATGG
ACACTTTCATCCTTCTCCACTGATGGGAGGTTTTGGGCTCAAACACCCGCCTCCTCAGAT
CATGATCAAAAACACTCCCGTTCCAGCCAATCCTCCTACAAACTTCAGTTCTGCCAAGTT
TGCTTCTTTCATCACACAGTACTCCACGGGGCAGGTCAGCGTGGAGATCGAGTGGGAGCT
GCAGAAGGAGAACAGCAAACGCTGGAATCCCGAAATTCAGTACACTTCCAACTACAACAA
ATCTGTTAATGTGGACTTTACTGTGGACACTAATGGTGTGTATTCAGAGCCTCGCCCCAT
TGGCACCAGATACCTGACTCGTAATCTGTAATTGCTTGTTAATCAATAAACCGTTTAATT
CGTTTCAGTTGAACTTTGGTCTCTGCGTACTTCTTTTCTTATCTAGTTTCCATGGCTACG
TAGATAAGTAGCATGGCGGGTTAATCATTAACTACAAGGAACCCCTAGTGATGGAGTTGG
CCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGAGAGGGAGTGGCCA
A
SEQ ID NO: 95
>#767_AAV.FL.linear [organism = Adeno-associated virus][mol_type = genomic DNA]
[isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCAGGGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGTGTGAGTAAGGCTC
CGGAGGCCCTTTTCTTTGTGCAATTTGAGAAGGGAGAGAGCTACTTCCACATGCACGTGC
TTGTGGAGACCACTGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAGCAAAGAAAATCAGAATCCCAATTCTGACGCGC
CGGTGATTAGATCAAAAACCTCAGCCAGGTACATGGAGCTGGTCGGGTGGCTCGTGGATA
AGGGGATCACCTCGGAGAAGCAGTGGATCCAGGAGGACCAGGCCTCATACATCTCCTTCA
ATGCGGCCTCCAACTCGCGGTCCCAGATCAAGGCTGCCTTGGACAATGCGGGAAAGATCA
TGAGCCTGACTAAAACCGCCCCCGACTACCTGGTGGGCCAGCAGCCCGTGGAGGACATTT
CCAGCAATCGGATTTATAAAATTTTGGAACTCAACGGGTACGATCCCCAATATGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAAGCCATAGCCCACACTGTGCCCTTCT
ACGGGTGCGTAAACTGGACCAATGAGAACTTTCCCTTCAACGACTGTGTCGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAAAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATTTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAAGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAACTCGACAGCGGA
GACAACCCGTACCTCAAGTACAACCACGCCGACGCGGAATTTCAGGAGCGCCTTAAAGAA
GATACGTCTTTTGGGGGCAACCTCGGACGAGCAGTCTTCCAGGCAAAAAAGAGGGTTCTT
GAACCTCTGGGCCTGGTTGAGGAACCTGTTAAGACGGCTCCTGGAAAAAAGAGGCCGGTA
GAGCACTCTCCTGCGGAGCCAGACTCCTCCTCGGGAACCGGAAAAGCGGGCCAGCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTTTGGGATCTACTACAATGGCTACAGGC
AGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATTTCCAGCCAATCAGGAGCC
TCGAACCACTACTTTGGCTACAGCACCCCCTGGGGGTATTTTGACTTCAACAGATTCCAC
TGCCACTTTTCACCACGTGATTGGCAAAGACTCATCAACAACAACTGGGGATTCCGACCC
AAGAGACTCAACTTCAAGCTCTTTAACATTCAAGTCAAAGAGGTCACGCAGAATGACGGT
ACGACGACGATTGCCAATAACCTTACCAGCACGGTTCAGGTGTTTACTGACTCGGAGTAC
CAGCTCCCGTACGTCCTCGGCTCGGCGCATCAAGGATGCCTCCCGCCGTTCCCAGCGGAC
GTCTTCATGGTTCCACAGTATGGATACCTCACCCTGAACAACGGGAGTCAGGCGGTAGGA
CGCTCTTCCTTTTACTGCCTGGAGTACTTTCCTTCTCAGATGCTGCGTACCGGAAACAAC
TTTACCTTCAGCTACACTTTTGAGGACGTTCCTTTCCACAGCAGCTACGCTCACAGCCAG
AGTCTGGATCGGCTGATGAATCCTCTGATCGACCAGTACCTGTATTATCTGAACAAAACA
CAATCTAGTGGAACTCTTCAGCAGTCTCGGTTACTGTTTAGTCAAGCTGGACCCACCAGC
ATGTCTCTTCAAGCTAGGAACTGGCTTCCTGGACCCTGTTACCGCCAGCAGCGAGTATCA
AAGACATCTGCGGATAAAAACAACAGTGAATACTCGTGGACTGCGGCTACAAAGTATCAT
CTAAATGGCCGGGACTCGTTGGTTAATCCAGGACCAGCTATGGCCAGCCACAAAGACGAT
GAAGAAAAGTTTTTCCCCATGCCTGGAACCCTGATATTTGGTAAAGAAGGAACAAATGCT
AACAACGCAGAATTGGAAAATGTCATGATTACAGATGAAGAAGAAATCCGCACCACCAAT
CCCGTGGCTACGGAGCAGTACGGATATGTGTCAAATAATTTGCAAAACTCAAATACTGGT
CCAACTACTGGAACTGTCAATCACCAAGGAGCGTTACCTGGTATGGTGTGGCAGGATCGA
GACGTGTACCTGCAGGGACCCATTTGGGCCAAGATTCCTCACACCGATGGACACTTTCAT
CCTTCTCCACTGATGGGAGGTTTTGGGCTCAAACACCCACCTCCTCAGATCATGATCAAA
AACACTCCCGTTCCAGCCAATCCTCCTACAAACTTCAGTGCGGCAAAATTTGCTTCCTTT
ATCACACAGTATTCCACGGGGCAGGTCAGCGTGGAAATCGAGTGGGAGCTGCAGAAGGAG
AACAGCAAACGCTGGAATCCCGAGATCCAGTACACTTCCAACTACAACAAGTCTGTTAAT
GTGGACTTTACTGTGGACACTAATGGCGTGTATTCAGAGCCTCGCCCCATTGGCACCAGA
TACCTGACTCGTAATCTGTAATTGCTTGTTAATCAATAAACCGTTTAATTCGTTTCAGTT
GAACTTTGGTCTCTGCGTACTTCTTTTCTTATCTAGTTTCCATGGCTACGTAGATAAGTA
GCATGGAGGGTTAATCATTAACTACAAGGAACCCCTAGTGATGGAGTTGGCCACTCCCTC
TCTGCGCGCTCGCTCGCTCACTGAGGCCGGGCGACCAAAGGTCGCCCGACGCCCGGGCTT
TGCCCGGGCGGCCTCAGTGAGCGAGCGAGCGCGCAGAGAGGGAGTGGCCAA
SEQ ID NO: 96
>#1591_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTATACGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCTAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAATGGCGCCGTGTGAGTAAGGCCA
CGGAGGCCCTCTTCTTTGTGCAATTTGAGAAGGGAGAGAGCTACTTCCACATGCACGTGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TTACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTTGTCGGGTGGCTCGTGGATA
AGGGGATTACCTCGGAGAAGCAGTGGATTCAGGAGGACCAGGCCTCATACATCTCCTTCA
ATGCGGCCTCCAACTCGCGGTCGCAAATCAAGGCTGCTCTGGACAATGCGGGAAAGATCA
TGAGCCTGACTAAAACCGCCCCCGACTACCTGGTGGGCCAGCAGCCCGTGGAGGACATTT
CCAGCAATCGGATTTATAAGATCTTGGAACTGAACGGGTACGATCCCCAATACGCGGCTT
CCGTATTTCTGGGATGGGCCACGAAAAAGTTCGGCAAGAGRAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCTTTCAACGACTGTGTTGACAAGATGG
TGATCTGGTGGGAGGAGGGGAAGATGACCGCCAAGGTTGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGCCAGACGCTTGCACAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTGAAAGACTTTTTCCGGTGGGCAA
AGGATCACGTGGTTGAGGTGGAGCATGAATTCTACGTCAAAAAGGGTGGAGCCAAAAAAA
GACCCGCCCCCAGTGACGCAGATATAAGTGAGCCCAAACGGGCGCGCGAGTCAGTTGCGC
AGCCATCGACGTCAGACGCGGAAGCTTCAATCAACTACGCGGACAGGTACCAAAACAAAT
GTTCTCGTCACGTGGGCATGAATCTGATGCTGTTTCCCTGCAGACAATGCGAAAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGCGGAGCCAGACTCCTCCTCGGGAACCGGAAAAGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTCTGGTTTGGGATCTACTACAATGGCTACAGGC
AGTGGCGCACCAGTGGCAGACAATAACGAGGGTGCCGATGGAGTGGGTAATTCCTCAGGA
AATTGGCATTGCGATTCCCAATGGTTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCTTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTGCCACAGTATGGATACCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTCAGCTACACCTTTGARGACGTTCCTTTCCACAGCAGCTACGCTCAC
AGCCAGAGTCTGGATCGGCTGATGAATCCTCTGATTGACCAGTACCTGTATTATCTGAAC
AAGACACAATCAAATAGTGGAACTCTTCAGCAGTCTCGGCTACTGTTTAGTCAAGCTGGA
GCGAGTGACATTCGGGACCAGTCTAGGAACTGGCTTCCTGGACCCTGTTACCGCCAGCAG
CGAGTATCAAAGACATCTGCGGATAACAACAACAGCAACTTTCCCTGGACTGCGGCTACA
AAGTATCATCTAAATGGCCGGGACTCGTTGGTTAATCCAGGACCAGCTATGGCCAGCCAC
AAAGACGATGAAGAAAAGTTTTTCCCCATGCATGGAACCCTGATATTTGGTAAACAAGGA
ACAAATGCTAACGACGCGGATTTGGACAATGTTATGATTACAGATGAAGAAGAAATCCGC
ACCACCAATCCCGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGGCTCAAACACCCGCCTCCTCAGATC
ATGATCAAAAACACTCCCGTTCCAGCCAATCCTCCTACAAACTTCAGTTCTGCCAAGTTT
GCTTCTTTCATCACACAGTATTCCACGGGACAGGTCAGCGTGGAGATCGAGTGGGAGCTG
CAGAAGGAGAACAGCAAACGCTGGAATCCCGAAATTCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTTTGCGTACTTCTTTCTTATCTAGTTTCCATGGCTACGTA
GATAAGTAGCATGGCGGGTTAATCATTAACTACAAGGAACCCCTAGTGATGGAGTTGGCC
ACTCCCTCTCTACGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 97
>#508_AAV.FL.linear [organism = Adeno-associated virus][mol_type = genomic DNA]
[isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGGGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTAGAGGTCACGTGAGTGTTTTGCGACATTTTGCGACACCAT
GTGGTTACGCAGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACAGAATGGCGCCGTGTGAGTAAAGCCC
CCGAGGCCCTTTTCTTTGTGCAATTTGAGAAGGGAGAGAGCTACTTCCACATGCACGTGC
TCGTGGAAACCACGGGGGTGAAATCCATGGTTTTGGGACGTTTCCTGAGTCAGATTCGCG
AAAAACTGATTCAGAGAATTTATCGCGGGATCGAGCCGACTTTGCCAAACTGGTTCGCGG
TCACAAAGACCCGAAATGGCGCCGGAGGCGGGAATAAGGTGGTGGATGAGTGCTACATCC
CCAATTACCTGCTCCCCAAAACCCAACCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATCAGATCAAAAACCTCAGCCAGGTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAACTCGCGGTCCCAGATCAAGGCTGCCTTGGACAATGCGGGAAAGATCA
TGAGCCTGACTAAAACCGCCCCCGACTACCTGGTGGGCCAGCAGCCTGTGGAGGACATTT
CCGGCAATCGGATTTATAAAATCTTGGAACTGAACGGGTACGATCCCCAATACGCGGCTT
CCGTCTTTCTGGGATGGGCCACKAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCKTTCAATGATTGCGTCGACAAAATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAATCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAACATGAACTCACCCGCCGTC
TGGATCATGACTTTGGGAAGGTCACCAAGCAGGAAGTCAAACGCTATTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCAGACAGGTACCAAAACAAAT
GTTCTCGTCACGTGGACATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCTTTCAACGGACTCGACAAGGGAGAGCCGGTCAAC
GAGGCAGACGCCGCGGCCATCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACMTMTGGGCCTGGTTGAGGAACCTGTTAAGACGGCTCCGGGAAAAAAGAGGCCAGTA
GCGCACTCTCCTGCGGAACCAGACTCCTCCTCGGGAACCGGAAARGCGGGCAACCAGCCT
GCAAGAAAGAGATTGAATTTCGGTCAGACTGGAGACGCAGACTCCGTACCYGACCCCCAG
CCTCTCGGACAGCCACCAGCAGCCCCCACAAGTTTGGGATCTACTACAATGGCTACAGGC
AGTGGCGCACCAATGGCAGACAATAACGAGGGTGCCGATGGAGTGGGTAATTCCTCGGGA
AATTGGCATTGCGATTCCACATGGATGGGCGACAGAGTCATCACCACCAGCACCCGAACC
TGGGCTCTGCCCACCTACAACAACCACCTCTACAAGCAGATCTCCAGCCAATCAGGAGCT
TCRAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAAATGCTGCGTACTGGA
AACAACTTTCAGTTCAGCTACACTTTTGAAGACGTGCCTTTCCACAGCAGCTACGCTCAC
AGCCAGAGTTTGGATCGGCTGATGAATCCTCTGATCGACCAGTACCTGTATTATCTAAAC
AGGACACAAACAGCCAGTGGAACTCAGCAGTCTCGGCTACTGTTTAGCCAAGCTGGACCC
ACCAGCATGTCTCTTCAAGCTAAAAACTGGCTGCCTGGACCTTGCTACAGACAGCAGCGT
CTGTCAAAGCAGGCAAACGACAACAACAACAGCAACTACTCGTGGACTGGAGCTACCAAG
TACCACCTCAATGGTAGAGACTCTTTGGTGWACCCGGGCCCTGCTATGGCCAGCCACRAA
GACGATGAAGRAAAGTTTTTCCCCATGCATGGAACCCTGATATTTGGTAAAGAAGGAACA
AATGCTACCAACGCGGATTTGGACAATGTCATGATTACAGACGAAGAGGAAATCAGGACA
ACAAATCCTGTAGCTACGGAGCAGTACGGATATGTGTCAAATAATTTGCAAAACTCAAAC
ACTGCTGCAACTACTGAAACTGTCAATCACCAAGGAGCGTTACCTGGTATGGTGTGGCAG
GACCGAGACGTGTACCTACAGGGACCCATTTGGGCCAAGATTCCTCACACCGATGGACAC
TTTCATCCTTCTCCCCTCATGGGCGGATTCGGACTCAAAAACCCACCTCCTCAGATCATG
ATCAAAAACACTCCCGTTCCAGCCAATCCTCCCACAAACTTCAGTTCTGCCAAGTTTGCT
TCTTTCATCACACAGTATTCCACGGGGCAGGTCAGCGTGGAGATCGAGTGGGAGCTGCAG
AAGGAGAACAGCAAACGCTGGAATCCCGAGATCCAGTACACTTCCAACTACAACAAGTCT
GTTAATGTGGACTTTACTGTGGACACTAATGGTGTGTATTCAGAGCCTCGCCCCATTGGC
ACCAGATACCTGACTCGTAATCTGTAATTGCTTGTTAATCAATAAACCGTTTAATTCGTT
TCAGTTGAACTTTGGTCTCCGCGTCACGACTCCACCCCTAGTTTCCATGACTACGTAGAT
AAGTAGCATGGCGGGTTAATCATTAACTCTAAGGAACCCCTAGTGATGGAGTTGGCCACT
CCCTCTCTGCGCGCTCGCTCGCTCCCTGAGGCCGGGCGACCAAAGGTCGCCCGACGCCCG
GGCTTTGCCCGGGCGGCCTCAGTGAGCGAGCGAGCGCGCAGAGAGGGAGTGGCCAA
SEQ ID NO: 98
>#1704_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCATCACTAGGGGTTCCTGGAGGGGTGGAGTCGTGACGTGAAGTACGTCATAGG
GTTAGGGAGGTCCTGTATTAGAGGTCACGTGAGTGTTTTGCGACATTTTGCGACACCATG
TGGTCACGGTGGGTATTTAAGCCCAAGTGAGCACGCAGGGTCTCCATTTTGAAGCGGGAG
GTTTGAATACGCAGCCGCCATGCCGGGGTTTTACGAGATTGTGATTAAGGTCCCAAGCGA
CCTTGACGAGCATTTGCCCGGCATTTCTGACAGCTTTGTGAACTGGGTGGCCGAGAAGGA
ATGGGAGTTGCCGCCAGATTCTGACATGGATCTGAATCTGATTGAGCAGGCACCCCTGAC
CGTGGCCGAAAAGCTGCAGCGCGACTTTCTGACAGAATGGCGCCGTGTGAGTAAGGCCCC
CGAGGCCCTTTTCTTTGTGCAATTTGAGAAGGGAGAGAGCTATTTCCACATGCACGTGCT
CGTGGAGACCACGGGGGTAAAATCCATGGTTTTAGGACGTTTCCTGAGTCAGATTCGCGA
AAAACTGATTCAGAGAATTTACCGCGGGATCGAGCCGACTTTGCCAAATTGGTTTGCGGT
CACAAAGACTCGAAATGGCGCCGGAGGCGGGAACAAGGTGGTGGATGAGTGCTACATCCC
CAATTACCTGCTCCCCAAAACCCAACCTGAGCTCCAGTGGGCGTGGACTAATATGGAACA
GTATTTAAGCGCCTGTTTGAACCTCGCGGAGCGTAAACGGTTGGTGGCGCAGCATCTGAC
GCACGTGTCGCAGACCCAGGAGCAGAACAAAGAGAATCAGAATCCCAATTCTGACGCGCC
GGTGATTAGATCAAAAACCTCAGCCAGGTACATGGAGCTGGTCGGGTGGCTCGTGGATAA
GGGGATTACCTCTGAGAAGCAGTGGATTCAGGAGGACCAGGCCTCATACATCTCCTTCAA
TGCGGCCTCCAACTCGCGGTCCCAAATCAAGGCTGCTCTGGACAATGCGGGAAAAATCAT
GAGCCTGACTAAAACGGCCCCCGACTACCTGGTGGGCCAGCAGCCCGTGGAGGACATCTC
CGGCAATCGGATTTATAAAATCTTGGAACCGAACGGGTACGATCCCCAATACGCGGCTTC
CGTCTTTCTGGGATGGGCCACGAAAAAGTTCGGCAAGAGGAACACCATCTGGCTGTTTGG
GCCTGCAACTACCGGGAAGACCAACATCGCGGAGGCCATAGCCCACACTGTGCCCTTCTA
CGGGTGCGTAAACTGGACCAATGAAAACTTTCCTTTCAACGATTGCGTCGACAAGATGGT
GATCTGGTGGGAGGAGGGGAAGATGACCGCCAAGGTCGTGGAGTCGGCCAAAGCCATTCT
CGGAGGAAGCAAGGTGCGCGTGGACCAGAAATGCAAGTCCTCGGCCCAGATCGACCCGAC
TCCCGTGATCGTCACCTCCAACACCAACATGTGCGCCGTGATTGACGGGAACTCAACGAC
CTTCGAGCACCAGCAGCCGTTGCAAGGGATGTTCAAATTTGAACTTACCCGCCGTCTGGA
TCATGACTTTGGGAAGGTCACCAAGCAGGAAGTCAAAGACTTTTTCCGGTGGGCAAAGGA
TCACGTGGTTGAGGTGGAGCATGAATTCTACGTCAAAAAGGGTGGAGCCAAGAAAAGACC
CGCCCCCAGTGACGCAGATATAAGTGAGCCCAAACGGGCGCGCGAGTCAGTTGCGCAGCC
ATCGACGTCAGACGCGGAAGCTTCGATCAACTACGCGGACAGGTACCAAAACAAATGTTC
TCGTCACGTGGGCATGAATCTGATGCTGTTTCCCTGCAGACAATGCGAGAGAATGAATCA
GAATTCAAATATCTGCTTTACTCACGGACAGAAAGACTGTTTAGAGTGCTTTCCCGTGTC
AGAATCTCAACCCGTTTCTGTCGTCAAAAAGGCGTATCAGAAACTTTGCTACATTCATCA
TATCATGGGAAAGGTGCCAGACGCTTGCACTGCTTGCGACCTGGTCAATGTGGATTTGGA
TGACTGCATCTCTGAACAATAAATGATTTAAATCAGGTATGGCTGCCGATGGTTATCTTC
CAGATTGGCTCGAGGACACTCTCTCTGAAGGAATAAGACAGTGGTGGAAGCTCAAACCTG
GCCCACCACCACCAAAGCCCGCAGAGCGGCATAAGGACGACAGCAGGGGTCTTGTGCTTC
CTGGGTACAAGTACCTCGGACCCTTCAACGGACTCGACAAAGGAGAGCCGGTCAACGAGG
CAGACGCCGCGGCCCTCGAGCACGACAAGGCCTACGACCGGCAGCTCGACAGCGGAGACA
ACCCGTACCTCAAGTACAACCACGCCGACGCGGAGTTCCAGGAGCGCCTTAAAGAAGATA
CGTCTTTTGGGGGCAACCTCGCAAGAGCAGTTTTCCAGGCAAAAAAGAGGGTTCTTGAAC
CTCTGGGCCTGGTTGAGGAACCTGTTAAGACGGCTCCGGGAAAAAAGAGGCCGGTAGAGC
ACTCTCCTGCGGAGCCAGATTCCTCCTCCGGAACTGGAAAGTCGGGCAACCAGCCTGCAA
GAAAGAGATTGAATTTTGGTCAGACTGGAGACGCAGACTCCGTACCTGACCCCCAGCCTC
TCGGACAGCCACCAGCAGCCCCCTCTGGTTTGGGATCTACTACAATGGCTACAGGCAGTG
GCGCACCAGTGGCAGACAATAACGAGGGTGCCGATGGAGTGGGTAATTCCTCGGGAAATT
GGCATTGCGATTCCCAATGGCTGGGCGACAGAGTCATCACCACCAGCACCCGAACCTGGG
CCCTGCCCACCTACAACAACCACCTCTACAAGCAAATCTCCAGCCAATCAGGAGCCTCGA
ACGACAACCACTACTTTGGCTACAGCACCCCTTGGGGGTATTTTGACTTCAACAGATTCC
ACTGCCACTTCTCGCCACGCGATTGGCAAAGACTCATCAACAACAACTGGGGATTCCGGC
CCAAGAGACTCAACTTCAAGCTCTTTAACATTCAAGTCAAAGAGGTCACGCAGAATGACG
GTACGACGACGATTGCCAATAACCTTACCAGCACGGTTCAGGTGTTTACTGACTCGGAGT
ACCAGCTCCCGTACGTCCTCGGCTCGGCGCATCAAGGATGCCTCCCGCCGTTCCCAGCAG
ACGTCTTCATGGTCCCACAGTATGGATACCTCACCCTGAACAACGGGAGTCAGGCGGTAG
GACGCTCTTCCTTTTACTGCCTGGAGTACTTTCCTTCTCAGATGCTACGTACCGGAAACA
ATTTCACCTTCAGCTACACTTTTGAAGACGTTCCTTTCCACAGCAGCTACGCTCACAGCC
AGAGTCTGGATCGGCTGATGAATCCTCTCATCGACCAGTACCTGTATTATCTGAACAAGA
CACAAACAAATAGTGGAACTCTTCAGCAGTCTCGGCTACTGTTTAGTCAAGCTGGACCCA
CCAGCATGTCTCTTCAAGCTAAAAACTGGCTGCCTGGACCTTGCTACAGACAGCAGCGTC
TGTCAAAGCAGGCAAACGACAACAACAACAGCAATTTTCCCTGGACTGCGGCTACAAAGT
ATCATCTAAATGGCCGGGACTCTCTGGTGAATCCAGGACCAGCTATGGCCAGTCACAAGG
ATGACGAAGAAAAGTTTTTCCCCATGCATGGAACATTGATATTTGGTAAACAAGGAACAA
ATGCCAACGACGCGGATTTGGAAAATGTTATGATTACAGATGAAGAAGAAATCAGGACCA
CCAATCCCGTGGCTACGGAGCAGTACGGATATGTGTCAAATAATTTGCAAAACTCAAACA
CTGGTCCAACTACTGGAACTGTCAATCACCAAGGAGCGTTACCTGGTATGGTGTGGCAGG
ATAGAGACGTATACCTACAGGGTCCCATTTGGGCCAAGATTCCTCACACCGATGGACACT
TTCATCCTTCTCCGCTGATGGGAGGTTTTGGACTCAAACACCCGCCTCCTCAGATCATGA
TCAAAAACACTCCCGTTCCAGCCAATCCTCCCACGAACTTCAGTTCTGCCAAGTTTGCTT
CTTTCATCACACAGTATTCCACGGGGCAGGTCAGCGTGGAGATCGAGTGGGAGCTGCAGA
AGGAGAACAGCAAACGCTGGAATCCCGAGATCCAGTACACTTCCAACTACAACAAGTCTG
TTAATGTGGACTTTACTGTGGACACTAATGGCGTGTATTCAGAGCCTCGCCCCATTGGCA
CCAGATACCTGACTCGTAATCTGTAATTGCTTGTTAATCAATAAACCGTTTAATTCGTTT
CAGTTGAACTTTGGTCTCTGCGTACTTCTTTTCTTATCTAGTTTCCATGGCTACGTAGAT
AAGTAGCATGGAGGGTTAATCATTAACTACAAGGAACCCCTAGTGATGGAGTTGGCCACT
CCCTCTCTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAGAGAGGGAGTGGCCAA
SEQ ID NO: 99
>#2112_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTACGACATTTTGCGACACCAT
GTGGTCACGCTGGGTATTTACGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAAAAGCTGCAGCGCGACTTTCTGACGGAGTGGCGCCGTGTGAGTAAGGCCC
CGGAGGCCCTTTTCTTTGTGCAATTTGAGAAGGGAGAGAGCTACTTCCACATGCACGTGC
TTGTGGAAACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTTGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATCAGATCAAAAACCTCAGCCAGGTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAACTCGCGGTCCCAAATTAAGGCTGCTCTGGACAATGCGGGAAAGATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTCGGCAAGAGAAACACCATCTGGCTGTTTG
GACCTGCGACCACGGGGAAGACCAACATCGCGGAGGCCATAGCCCACACTGTGCCCTTCT
ACGGGTGCGTAAACTGGACCAATGAGAACTTTCCTTTCAATGATTGCGTCGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATYGACGGGAACTCAACGA
CCTTCGAACACCAGCAGCCGTTGCAAGACCGGATGTTCAAATTTGAACTCACCCGCCGTC
TGGATCATGACTTTGGGAAGGTCACAAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCTAGTGACGCAGATATAAGTGAGCCCAAACGGGCGCGCGAGTCAGTTGCGC
AGCCATCGACGTCAGACGCAGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCCAAGAAAACCTATTTTTCGATATACGACAGTGGTGGAAGCTCAAA
CCTGGCCCACCACCACCAAAGCCCGCAGAGCGGCATCAGGACAACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAGCCTGTTAAAACGGCTCCGGGAAAAAAGAGGCCGGTA
GAGCACTCTCCTGCGGAGCCAGACTCCTCCTCGGGAACCGGAAAAGCGGGCAASCAGCCT
GCGRGAAAAAGATTGAATTTTGGTCAGACTGGAGWCGCAGACTCCGTACCTGACCCCCAG
CCTCTCGGACAGCCACCAGCAGCCCCCTCTGGTCTGGGAACTAATACGATGGCTACAGGC
AGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCGGACGTCTTCATGGTGCCACAGTATGGATACCTCACCCTGAACAACGGGAGTCAGGCG
GTAGGACGCTCTTCATTTTACTGCCTGGAGTACTTTCCTTCTCAGATGCTTCGTACTGGA
AACAACTTTCAGTTCAGCTACACCTTTGAAGACGTTCCTTTCCACAGCAGCTACGCTCAC
AGCCAGAGTCTGGATAGGCTGATGAATCCTCTGATTGACCAGTACCTGTATTATCTGAAC
AAAACACAATCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAAAGCAGGCAAACGACAACAACAACAGCAA
CTTTCCCTGGACTGCGGCTACAAAGTATCAACTAAATGGCCGGGACCAGCTATGGCCAGC
CACAAAGACGATGAAGAAAAGTTTTTCCCCATGCATGGAACCCTGATATTTGGTAAACAA
GGAACAAATGCTAACGACGCGGATTTGGACAATGTCATGATTACAGATGAAGAAGAAATC
CGCACCACCAATCCCGTGGCTACGGAGCAGTACGGATATGTGTCAAATAATTTGCAAAAC
TCAAATACTGGTCCAACTACTGGAACTGTCAATCACCAAGGAGCGTTACCTGGTATGGTG
TGGCAGGATCGAGACGTGTACCTGCAGGGACCCATTTGGGCCAAGATTCCTCACACCGAT
GGACACTTTCATCCTTCTCCACTGATGGGAGGTTTTGGGCTCAAACACCCGCCTCCTCAG
ATCATGATCAAAAACACTCCCGTTCCAGCCAATCCTCCCACAAACTTCAGTTCTGCCAAG
TTTGCTTCTTTCATCACACAGTATTCCACGGGGCAGGTCAGCGTGGAGATCGAGTGGGAG
CTGCAGAAGGAGAACAGCAAACGCTGGAATCCCGAGATCCAGTACACTTCCAACTACAAC
AAGTCTGTTAATGTGGACTTTACTGTGGACACTAATGGTGTGTATTCAGAGCCTCGCCCC
ATTGGCACCAGATACCTGACTCGTAATCTGTAATTGCTTGTTAATCAATAAACCGTTTAA
TTCGTTTCAGTTGAACTTTGGTCTCTGTGTACTTCTTTCTTATCTAGTTTCCATGGCTAC
GTAGATAAGTAGCATGGCGGGTTAATCATTAACTACAAGGAACCCCTAGTGATGGAGTTG
GCCACTCCCTCTCTGCGCGCTCGCTCGCTCACTGAGGCCGGGCGACCAAAGGTCGCCCGA
CGCCCGGGCTTTGCCCGGGCGGCCTCAGTGAGCGAGCGAGCGCGCAGAGAGGGAGTGGCC
AA
SEQ ID NO: 100
>#1350_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCTGGGTATTTAAGCCCGAGTGAGCACGCAGGGTCTCCATTTTGAAGCGGGA
GGTTTGAACGCGCAGCCGCCATGCCGGGTTTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCTC
CGGAAGCCCTTTTTTTTGTGCAATTTGAGAAGGGAGAGAGCTACTTCCACATGCACGTGC
TGGTGGAGACCACCGGGGTGAAATCCATGGTCTTAGGACGTTTCCTGAGTCAGATTCGCG
AAAAACTGATTCAGAGAATTTACCGCGGGATCGAGCCGACTTTGCCAAATTGGTTCGCGG
TTACAAAGACCCGAAATGGCGCCGGAGGCGGGAACAAGGTGGTGGATGAGTGCTATATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAAAATCAGAATCCCAATTCTGACGCGC
CGGTGATTAGATCAAAAACCTCAGCCAGGTACATGGAGCTGGTCGGGTGGCTCGTGGATA
AGGGGATTACCTCGGAGAAGCAGTGGATCCAGGAGGACCAGGCTTCATACATCTCCTTTA
ATGCGGCCTCCAACTCGCGGTCCCAAATCAAGGCTGCTCTGGACAATGCGGGGAAGATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATATGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCTTTTT
ACGGGTGCGTAAACTGGACCAATGAGAACTTTCCATTCAACGACTGTGTTGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAGGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGACCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAAACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGACTGAATTTCGGTCAGACTGGAGACGCAGACTCCGTACCTGACCCCCAG
CCTCTCGGACAGCCACCAGCAGCCCCCTCTGGTCTGGGATCTACTACAATGGCTACAGGC
AGTGGCGCACCAATGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAGGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAGACACAGTCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAACAGGCAAACGACAACAACAACAGCAACTTTCCCTGGACTGCGGCTACA
AAGTATCATCTAAATGGCCGGGACTCGTTGGTTAATCCAGGGCCAGCTATGGCCAGCCAC
AAAGACGATGAAGAAAAGTTTTTCCCCATGCATGGAACCCTGATATTTGGTAAACAAGGA
ACAAATGCTAACGACGCGGATTTGGACAATGTCATGATTACAGATGAAGAAGAAATCCGC
ACCACCAATCCCGTGGCTACGGAGCAGTACGGATATGTGTCAAATAATTTGCAGAGCGGC
AACACACAAGCAGCTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGGCTCAAACACCCGCCTCCTCAGATC
ATGATCAAAAACACTCCCGTTCCAGCCAATCCTCCCACAAACTTCAGTTCTGCCAAGTTT
GCTTCTTTCATCACACAGTATTCCACGGGGCAGGTCAGCGTGGAGATCGAGTGGGAGCTG
CAGAAGGAGAACAGCAAACGCTGGAATCCCGAAATTCAGTACACTTCCAACTACAACAAG
TCTGTTAATGTGGACTTTACTGTGGACACTAATGGCGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGTGTACTTCTTTCTTATCTAGTTTCCATGGCTACGTA
GATAAGTAGCATGGCGGGTTAATCATTAACTACAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 101
>#668_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACTCCCGTGATCGCCCGGGCGGCCTCAGTGAGCGAGCGAGCGCGCAGAGAGGGAGTGG
CCAACTCCATCACTAGGGGTTCCTGGAGGGGTGGAGTCGTGACGTGAAGTACGTCATAGG
GTTAGGGAGGTCCTGTATTAGAGGTCACGTGAGTGTTTTGCGACATTTTGCGACACCATG
TGGTCACGCTGGGTATTTAAGCCCGAGTGAGCACGCAGGGTCTCCATTTTGAAGCGGGAG
GTTTGAACACGCAGCCGCCATGCCGGGGTTTTACGAGATTGTGATTAAGGTCCCCAGCGA
CCTTGACGAGCATTTGCCCGGCATTTCTGACAGCTTTGTGAACTGGGTGGCCGAGAAGGA
ATGGGAGTTGCCGCCAGATTCTGACATGGATCTGAATCTGATTGAGCAGGCACCCCTGAC
CGTGGCCGAGAAGCTGCAGCGCGACTTTCTGACGGAGTGGCGCCGTGTGAGTAAGGCCCC
GGAGGCCCTTTTCTTTGTGCAATTTGAGAAGGGAGAGAGCTACTTCCACATGCACGTTCT
GGTGGAGACTACCGGGGTAAAATCCATGGTTTTGGGACGTTTCCTGAGTCAGATTCGCGA
AAAACTGATTCAGAGAATTTACCGCGGGATCGAGCCGACTTTGCCAAACTGGTTCGCGGT
TACAAAGACCCGAAATGGCGCCGGAGGCGGGAACAAGGTGGTGGATGAGTGCTACATCCC
CAATTACCTGCTCCCCAAAACCCAGCCTGAGCTCCAGTGGGCGTGGACTAATATGGAACA
GTATTTAAGCGCCTGTTTGAATCTCGCGGAGCGTAAACGGTTGGTGGCGCAGCATCTGAC
GCACGTGTCGCAGACGCAGGAGCAGAACAAAGAGAATCAAAATCCCAATTCTGACGCGCC
GGTGATTAGATCAAAAACCTCAGCCAGGTACATGGAGCTGGTCGGGTGGCTCGTGGATAA
GGGGATTACGTCGGAGAAGCAGTGGATTCAGGAGGACCAGGCCTCATACATCTCCTTCAA
TGCGGCCTCCAACTCGCGGTCCCAAATCAAGGCTGCTCTGGACAATGCAGGAAAGATCAT
GAGCCTGACTAAAACCGCCCCCGACTACCTGGTGGGCCAGCAGCCCGTAGAGGACATTTC
CAGCAATCGGATTTATAAAATCTTGGAACTGAACGGGTACGATCCCCAATATGCGGCTTC
CGTCTTTCTGGGATGGGCCACGAAAAAGTTCGGCAAGAGGAACACCATCTGGCTGTTTGG
GCCTGCAACTACCGGGAAGACCAACATCGCGGAGGCCATAGCCCACACTGTGCCCTTCTA
CGGGTGCGTAAACTGGACCAATGAGAACTTTCCTTTCAACGACTGTGTCGACAAGATGGT
GATCTGGTGGGAGGAGGGAAAGATGACCGCCAAGGTCGTGGAGTCGGCCAAAGCCATTCT
CGGAGGAAGCAAGGTGCGCGTGGACCAGAAATGCAAGTCCTCGGCCCAGATAGACCCGAC
TCCCGTGATCGTCACCTCCAACACCAACATGTGCGCCGTGATTGACGGAAACTCAACGAC
CTTCGAACACCAGCAGCCGTTGCAAGACCGGATGTTCAAATTTGAACTCACCCGCCGTCT
GGATCATGACTTTGGGAAGGTCACCAAGCAGGAAGTCAAAGACTTTTTCCGGTGGGCAAA
GGATCACGTGGTTGAGGTGGAGCATGAATTCTACGTCAAAAAGGGTGGAGCCAAGAAAAG
ACCCGCCCCCAGTGACGCAGATATAAGTGAGCCCAAACGGGCGCGCGAGTCAGTTGCGCA
GCCATCGACGTCAGACGCGGAAGCTTCGATCAACTACGCGGACAGGTACCAAAACAAATG
TTCTCGTCACGTGGGCAAGAATCTGATGCTGTTTCCCTGCAGACAATGCGAGAGAATGAA
TCAAAATTCAAATATCTGCTTCACTCACGGACAGAAAGACTGTTTAGAGTGCTTTCCCGT
GTCAGAATCTCAACCCGTTTCTGTCGTCAAAAAGGCGTATCAGAAACTTTGCTACATTCA
TCACATAATGGGAAAGGTGCCAGACGCTTGCACTGCCTGCAATCTGGTCAATGTGGATTT
GGATGACTGCATCTCTGAACAATAAATGATTTAAATCAGGTATGGCTGCCGATGGTTATC
TTCCAGATTGGCTCGAGGACACTCTCTCTGAAGGAATAAGACAGTGGTGGAAGCTCAAAC
CTGGCCCACCGCCACCAAAGCCCGCAGAGCGGCATAAGGACGACAACAGGGGTCTTGTGC
TTCCTGGGTACAAGTACCTCGGACCCTTCAACGGACTCGACAAGGGAGAGCCGGTCAACG
AGGCAGACGCCGCGGCCCTCGAGCACGACAAGGCCTACGACCGGCAGCTCGACAGCGGAG
ACAACCCGTACCTCAAGTACAACCACGCCGACGCGGAGTTTCAGGAGCGCCTTAAAGAAG
ATACGTCTTTTGGGGGCAACCTCGGACGAGCAGTCTTCCAGGCGAAAAAGAGGGTTCTTG
AACCTCTGGGCCTGGTTGAGGAACCTGTTAAGACGGCTCCGGGAAAAAAGAGGCCGGTAG
AGCACTCTCCTGTGGAGCCAGACTCCTCCTCGGGAACCGGAAAAGCGGGCCAGCAGCCTG
CAAGAAAAAGATTGAATTTCGGTCAGACTGGAGACGCAGACTCCGTACCTGACCCCCAGC
CTCTCGGACAGCCACCAGCAGCCCCCTCTGGTCTGGGATCTACTACAATGGCTACAGGCA
GTGGCGCACCAATGGCAGACAATAACGAGGGTGCCGATGGAGTGGGTAATTCCTCAGGAA
ATTGGCATTGCGATTCCCAATGGCTGGGCGACAGAGTCATCACCACCAGCACCCGAACCT
GGGCCCTGCCCACCTACAACAACCACCTCTACAAGCAAATCTCCAGCCAATCAGGAGCCT
CAAACGACAACCACTACTTTGGCTACAGCACCCCCTGGGGGTATTTTGACTTCAACAGAT
TCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATG
ACGGTACGACGACGATTGCCAATAACCTTACCAGCACGGTTCAGGTGTTTACTGACTCGG
AGTACCAGCTCCCGTACGTCCTCGGCTCGGCGCACCAAGGATGCCTCCCGCCGTTCCCAG
CGGACGTCTTCATGGTCCCACAGTATGGATACCTCACCCTGAACAACGGGAGTCAGGCGG
TAGGACGCTCTTCCTTTTACTGCCTGGAGTACTTTCCTTCTCAGATGCTGCGTACTGGAA
ACAACTTTCAGTTCAGCTACACCTTTGAAGACGTTCCTTTCCACAGCAGCTACGCTCACA
GCCAGAGTCTGGATCGGCTGATGAATCCTCTGATCGACCAGTACCTGTATTATCTGAACA
AGACACAATCAAATAGTGGAACTCTTCAGCAGTCTCGGCTACTGTTTAGTCAAGCTGGAC
CCACCAGCATGTCTCTTCAAGCTAAAAACTGGCTGCCTGGACCTTGCTACAGACAGCAGC
GTCTGTCAAAGCAGGCAAACGACAACAACAACAGCAACTTTCCCTGGACTGCGGCTACAA
AGTATCATCTAAATGGCCGGGACTCGTTGGTTAATCCGGGCCCGGCTATGGCCAGCCACA
AAGACGATGAAGAAAAGTTTTTCCCCATGCATGGAACCCTGATATTTGGTAAACAAGGAA
CAAATGCTAACGACGCGGATTTGGACAATGTCATGATTACAGATGAAGAAGAAATCCGCA
CCACCAATCCCGTGGCTACGGAGCAGTACGGATATGTGTCAAATAATTTGCAAAACTCAA
ATACTGGTCCAACTACTGGAACTGTCAATCACCAAGGAGCGTTACCTGGTATGGTGTGGC
AGGATCGAGACGTGTACCTGCAGGGACCCATTTGGGCCAAGATTCCTCACACCGATGGAC
ACTTTCATCCTTCTCCACTGATGGGAGGTTTTGGGCTCAAACACCCGCCTCCTCAGATCA
TGATCAAAAACACTCCCGTTCCAGCCAATCCTCCCACAAACTTCAGTTCTGCCAAGTTTG
CTTCTTTCATCACACAGTATTCCACGGGGCAGGTCAGCGTGGAGATCGAGTGGGAGCTGC
AGAAAGAGAACAGCAAACGCTGGAACCCCGAGATCCAGTACACTTCCAACTATAACAAGT
CTGTTAATGTGGACTTTACTGTGGACACTAATGGTGTGTATTCAGAGCCTCGCCCCATTG
GCACCAGATACCTGACTCGTAATCTGTAATTGCTTGTTAATCAATAAACCGTTTAATTCG
TTTCAGTTGAACTTTGGTCTCTGTGTACTTCTTTTCTTATCTAGTTTCCATGGCTACGTA
GATAAGTAGCATGGCGGGTTAATCATTAACTCTAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 102
>#367_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCATGTGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACACAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAACTCGCGGTCCCAAATCAAGGCTGCTCTGGACAATGCGGGAAAGATTA
TGAGCCTGACTAAAACCGCCCCCGACTACCTGGTGGGCCAGCAGCCCGTGGAGGACATTT
CCAACAATCGAATTTATAAAATCTTGGAACTGAACGGGTACGATCCCCAATACGCTGCTT
CCGTCTTTCTGGGATGGGCCACGAAGAAGTTCGGCAAGAGGAACACCATCTGGCTGTTTG
GGCCTGCAACTACCGGAAAGACCAACATCGCGGAGGCCATAGCCCACACTGTGCCCTTCT
ACGGGTGCGTAAACTGGACCAATGAGAACTTTCCCTTCAACGACTGTGTCGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAAAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTCTGGTTTGGGATCTACTACAATGGCTACAGGC
AGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCCTGGGGGTATTTTGACTTCAACAGA
TTCCACTGCCACTTTTCACCACGTGATTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCACCAAGGATGCCTCCCGCCGTTCCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAAACACAATCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCGGCTACA
AAGTATCATCTAAATGGCCGGGACTCGTTGGTTAATCCAGGACCAGCTATGGCCAGCCAC
AAAGACGATGAAGAAAAGTTTTTCCCCATGCATGGAACCCTGATATTTGGTAAACAAGGA
ACAAATGCTAACGACGCAGATTTGGACAATGTCATGATTACAGATGAAGAAGAAATCCGC
ACCACCAATCCCGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGGCTCAAACACCCGCCTCCTCAGATC
ATGATCAAAAACACTCCCGTTCCAGCCAATCCTCCTACAAACTTCAGTTCTGCCAAGTTT
GCTTCTTTCATCACACAGTACTCCACGGGGCAGGTCAGCGTGGAGATCGAGTGGGAGCTG
CAGAAGGAGAACAGCAAACGCTGGAATCCCGAGATTCAGTACACTTCAAACTACAACAAG
TCTGTTAATGTGGACTTTACTGTGGACACTAATGGCGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCCGCGTACTTCTTTTCTTATCTAGTTTCCATGGCTACGT
AGATAAGTAGCATGGCGGGTTAATCATTAACTACAAGGAACCCCTAGTGATGGAGTTGGC
CACTCCCTCTCTGCGCGCTCGCTCGCTCACTGAGGCCGGGCGACCAAAGCTCGCCCGACG
CCCGGGCTTTGCCCGGGCGGCCTCAGTGAGCGAGCGAGCGCGCAGAGAGGGAGTGGCCAA
SEQ ID NO: 103
>#1020_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTAAATTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GTTTTGAACGCGCAGCCGCCATGCCGGGGTTTTACGAGATTGTGATTAAGGTCCCCAGCG
ACCTTGACGAGCATCTGCCGGGCATTTCTGACAGCTTTGTGAACTGGGTGGCCGAGAAGG
AATGGGAGTTGCCGCCAGATTCTGACATGGATTTGAATCTGATTGAGCAGGCACCCCTGA
CCGTGGCCGAGAAGCTGCAGCGCGACTTTCTGACGGAGTGGCGCCGTGTGAGTAAGGCCC
CGGAGGCCCTCTTCTTTGTACAATTTGAGAAGGGAGAGAGCTATTTCCACATGCACGTGC
TCGTGGAAACCACCGGGGTGAAATCCATGGTTTTGGGACGTTTCCTGAGTCAGATTCGTG
AAAAACTGATTCAGAGAATTTACCGCGGGATCGAGCCGACTTTGCCAAACTGGTTCGCGG
TCACAAAGACCAGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTAAATCTCTCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATCAGATCAAAAACCTCAGCCAGGTACATGGAGCTGGTCGGGTGGCTCGTGGACA
AGGGGATTACCTCGGAGAAGCAGTGGATCCAGGAGGACCAGGCCTCATACATCTCCTTCA
ATGCGGCCTCCAACTCGCGGTCCCAAATCAAGGCTGCTCTGGACAATGCGGGAAAGATCA
TGAGCCTCACTAAAACTGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCCTTCAACGACTGTGTCGACAAGATGG
TGATCTGGTGGGAGGAAGGAAAGATGACTGCCAAGGTCGTGGAGTCGGCCAAAGCCATTC
TTGGCGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGATCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAAAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATTTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTACCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTCTGGTCTGGGATCTACTACAATGGCTACAGGC
AGTGGCGCACCAATGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCCTGGGGGTATTTTGACTTCAACAGA
TTCCACTGCCACTTTTCACCACGTGATTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCACCAAGGATGCCTCCCGCCGTTCCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTTCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAAACACAATCAAATAGTGGAACTCTTCAGCAGTCTCGGCTACTGTTTAGTCAAGCTGGA
CCCACCAGCATGTCTCTTCAAGCTAAAAACTGGCTACCTGGACCTTGCTACAGACAGCAG
CGTCTGTCAAAGCAGGCAAACGACAACAACAACAGCAACTTTCCCTGGACTGCAGCTACA
AAGTATCATCTAAATGGCCGGGACTCGTTGGTTAATCCAGGACCAGCTATGGCCAGTCAC
AAGGATGACGAAGAAAAGTTTTTCCCCATGCATGGAACCCTGATATTTGGTAAACAAGGA
ACAAATGCCAACGACGCGGATTTGGAAAATGTCATGATTACAGACGAAGAAGAAATCAGG
ACCACCAATCCCGTGGCTACGGAGCAGTATGGGACTGTGTCAAATAATTTGCAAAACTCA
AACACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGGCTCAAACACCCGCCTCCTCAGATC
ATGATCAAAAACACTCCCGTTCCAGCCAATCCTCCTACAAACTTCAGTTCTGCCAAGTTT
GCTTCTTTCATCACACAGTACTCCACGGGGCAGGTCAGCGTGGAGATCGAGTGGGAGCTG
CAGAAGGAGAACAGCAAACGCTGGAATCCCGAGATTCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTGTAATTC
GTTTCAGTTGAACTTTGGTCTATGCGTACTTCTTTTCTTATCTGGTTTCCATGGCTACGT
AGATAAGTAGCATGGCGGGCTAATCATTAACTCTAAGGAACCCCTAGTGATGGAGTTGGC
CACTCCCTCTCTGCGCGCTCGCTCGCTCACTGAGGCCGGGCGACCAAAGGTCGCCCGACG
CCCGGGCTTTGCCCGGGCGGCCTCAGTGAGCGAGCGAGCGCGCAGAGAGGGAGTGGCCAA
SEQ ID NO: 104
>#1158_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTAGAGGTCACGTGAGTGTTTTGCGACATTTTGCGACACCAT
GTGGTTACGCTGGGTATTTAAGCCCGAGTGAGCACGCAGGGTCTCCATTTTGAAGCGGGA
GGTTTGAACGCGCAGTCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAAAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTATTTCCACATGCACGTGC
TGGTGGAGACCACCGGGGTGAAATCCATGGTTTTGGGACGTTTCCTGAGTCAGATTCGCG
AAAAACTGATTCAGAGAATTTACCGCGGGATTGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACCTCGGAGAAGCAGTGGATCCAGGAGGACCAGGCCTCATACATCTCCTTTA
ATGCGGCCTCCAACTCGCGGTCCCAGATCAAGGCTGCTCTGGACAATGCGGGAAAGATCA
TGAGCCTCACTAAAACGGCCCCCGACTACCTGGTGGGCCAGCAGCCCGTGGAGGACATTT
CCAGCAATAGGATTTATAAAATCTTGGAACTGAACGGGTACGATCCCCAATACGCGGCTT
CCGTCTTTCTGGGATGGGCCACGAAAAAGTTCGGCAAGAGAAACACCATCTGGCTGTTTG
GGCCTGCAACTACCGGGAAGACCAACATCGCGGAAGCCATAGCCCACACTGTGCCCTTCT
ACGGGTGCGTAAACTGGACCAATGAGAACTTTCCCTTCAACGACTGTGTCGACAAGATGG
TGATCTGGTGGGAGGAGGGGAAGATGACCGCCAAGGTCGTGGAGTCGGCCAAAGCCATTC
TCGGTGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TAGATCATGACTTTGGGAAGGTTACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGAGCGCGCGAGTCAGTTGCGC
AGCCATCGACGTCAGACGCGGAAGCTTCAATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCATGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGACTGAATTTCGGTCAGACTGGAGACGCAGACTCCGTACCTGACCCCCAG
CCTCTCGGAAAGCCACCAGCAGCCCCCTCTGGTCTGGGATCTACTACAATGGCTACAGGC
AGTGGCGCACCAATGGCAGACAATAACGAGGGCGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAGACACAATCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCGGCTACA
AAGTATCATCTAAATGGCCGGGACTCGTTGGTTAATCCAGGACCAGCTATGGCCAGCCAC
AAAGACGATGAAGAAAAGTTTTTCCCCATGCATGGAACCCTGATATTTGGTAAACAAGGA
ACAAATGCTAACGACGCGGATTTGGACAATGTCATGATTACAGATGAAGAAGAAATCCGC
ACCACCAATCCCGTAGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGGCTCAAACACCCGCCTCCTCAGATC
ATGATCAAAAACACTCCCGTTCCAGCCAATCCTCCTACAAACTTCAGTTCTTCCAAGTTT
GCTTCTTTCATCACACAGTATTCCACGGGGCAGGTCAGCGTGGAGATCGAGTGGGAGCTG
CAGAAGGAGAACAGCAAACGCTGGAATCCCGAAATTCAGTATACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTGTAATTC
GTTTCAGTTGAACTTTGGTCTTTGCGTACTTCTTTCTTATCTAGTTTCCATGGCTACGTA
GATAAGTAGCATGGAGGGTTAATCATTAACTACAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 105
>#2107_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAAAAGCTGCAGCGTGACTTTCTGACGGAGTGGCGCCGCGTGAGTAAGGCCC
CGGAGGCCCTCTTCTTTGTGCAATTTGAGAAGGGAGAGAGTTACTTCCACATGCACATGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACATCAGCCAGGTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAACTCGCGGTCCCAAATCAAGGCTGCTCTTGACAATGCGGGAAAGATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACCACCGGGAAGACCAACATCGCGGAGGCCATAGCCCACACTGTGCCCTTCT
ACGGGTGCGTAAACTGGACCAATGAGAACTTTCCCTTCAACGACTGCGTCGACAAGATGG
TGATCTGGTGGGAGGAGGGAAAGATGACTGCCAAGGTCGTGGAGTCTGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACATCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAAAAAA
GACCCGCCCCCAGTGACGCAGATATAAGTGAGCCCAAACGGGTGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATTATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTCTGGTTTGGGATCTACTACAATGGCTACAGGC
AGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCTTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAAACACAATCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCAGCTACA
AAGTATCATCTAAATGGCCGGGACTCGTTGGTTAATCCAGGACCAGCTATGGCCAGCCAC
AAAGACGATGAAGAAAAGTTTTTCCCCATGCATGGAACCCTGATATTTGGTAAACAAGGA
ACAAATGCTAACGACGCGGATTTGGACAATGTCATGATTACAGATGAAGAAGAAATCCGC
ACCACCAATCCCGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCGCTGATGGGAGGTTTTGGGCTCAAACACCCGCCTCCTCAGATC
ATGATCAAAAACACTCCAGTTCCAGCCAATCCTCCCACAAACTTCAGTTCTGCCAAGTTT
GCTTCTTTCATCACACAGTATTCCACGGGGCAAGTCAGCGTGGAGATCGAGTGGGAGCTG
CAGAAGGAGAACAGCAAACGCTGGAACCCCGAGATCCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTCCAGTTGAACTTTGGTCTCCGCGTACTTCTTTTCTTATCTAGTTTCCATGGCTACGT
AGATAAGTAGCATGGCGGGTTAATCATTAACTACAAGGAACCCCTAGTGATGGAGTTGGC
CACTCCCTCTCTGCGCGCTCGCTCGCTCACTGAGGCCGGGCGACCAAAGGTCGCCCGACG
CCCGGGCTTTGCCCGGGCGGCCTCAGTGAGCGAGCGAGCGCGCAGAGAGGGAGTGGCCAA
SEQ ID NO: 106
>#1570_AAV.FL.linear [organism = Adeno-associated virus][mol_type = genomic
DNA][isolation_source = liver tissue][topology = linear]
GGACTCGCGGCCGCAAGTGGATAGGTGGAGCGATTTGAGGTCTATGGTGTAAAAGGAAAT
ATCTTCACCACCTTTGGTCGCCCGGCCTCAGTGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAAAAGCTGCAGCGTGACTTTCTGACGGAGTGGCGCCGCGTGAGTAAGGCCC
CGGAGGCCCTCTTCTTTGTGCAATTTGAGAAGGGAGAGAGTTACTTCCACATGCACATGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACATCAGCCAGGTACATGGAGCTGGTCGGGTGGCTCGTGGATA
AGGGGATTACCTCGGAGAAGCAGTGGATCCAGGAGGACCAGGCKTCATACATCTCCTTCA
ATGCGGCCTCCAACTCGCGGTCCCAAATCAAGGCTGCTCTTGACAATGCGGGAAAGATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACCACCGGGAAGACCAACATCGCGGAGGCCATAGCCCACACTGTGCCCTTCT
ACGGGTGCGTAAACTGGACCAATGAGAACTTTCCCTTCAACGACTGCGTCGACAAGATGG
TGATCTGGTGGGAGGAGGGAAAGATGACTGCCAAGGTCGTGGAGTCTGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACATCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAAAAAA
GACCCGCCCCCAGTGACGCAGATATAAGTGAGCCCAAACGGGTGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATTATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTCTGGTTTGGGATCTACTACAATGGCTACAGGC
AGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCTTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAAACACAAGCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCAGCTACA
AAGTATCATCTAAATGGCCGGGACTCGTTGGTTAATCCAGGACCAGCTATGGCCAGCCAC
AAAGACGATGAAGAAGAAAAGTTTTTCCCCATGCATGGAACCCTGATATTTGGTAAACAA
GGAACAAATGCTAACGACGCGGATTTGGACAATGTCATGATTACAGATGAAGAAGAAATC
CGCACCACCAATCCCGTGGCTACGGAGCAGTACGRATATGTGTCAAATAATTTGCAAAAC
TCAAATACTGGTCCAACTACTGGAACTGTCAATCACCAAGGAGCGTTACCTGGTATGGTG
TGGCAGGATCGAGACGTGTACCTGCAGGGACCCATTTGGGCCAAGATTCCTCACACCGAT
GGACACTTTCATCCTTCTCCGCTGATGGGAGGTTTTGGGCTCAAACACCCGCCTCCTCAG
ATCATGATCAAAAACACTCCAGTTCCTTCTCCGCTGATGGGAGGTTTTGGGCTCAAACAC
CCGCCTCCTCAGATCACACAGTATTCCACGGGGCAAGTCAGCGTGGAGATCGAGTGGGAG
CTGCAGAAGGAGAACAGCAAACGCTGGAACCCCGAGATCCAGTACACTTCCAACTACAAC
AAATCTGTTAATGTGGACTTTACTGTGGACACTAATGGTGTGTATTCAGAGCCTCGCCCC
ATTGGCACCAGATACCTGACTCGTAATCTGTAATTGCTTGTTAATCAATAAACCGTTTAA
TTCGTTCCAGTTGAACTTTGGTCTCCGCGTACTTCTTTTCTTATCTAGTTTCCATGGCTA
CGTAGATAAGTAGCATGGCGGGTTAATCATTAACTACAAGGAACCCCTAGTGATGGAGTT
GGCCACTCCCTCTCTGCGCGCTCGCTCGAGGAAAAAACTTTTCTTCATTTTGTGCACTGA
GGCCGTTTTTGCTTTCTTAAGTAATAGGCTTGGAATTTATCCCGGAATACATCTCTTAAA
GTT
SEQ ID NO: 107
>#704_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGGG
CGTCGGGCGACCTTGGCTTGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGAGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTAAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGCGTGAGTAAAGCCC
CGGAGGCCCTCTTCTTTGTGCAATTTGAGAAGGGAGAGAGCTATTTCCACATGCACGTGC
TTGTGGAGACCACCGGAGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACCCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGACA
AGGGGATTACCTCGGAGAAGCAGTGGATCCAGGAGGATCAGGCTTCATACATCTCCTTCA
ATGCGGCCTCCAACTCGCGGTCCCAGATCAAGGCTGCTCTGGACAATGCGGGAAAGATCA
TGAGCCTGACTAAAACTGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTATTTCTGGGATGGGCCACGAAAAAGTTCGGCAAGAGGAACACCATTTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCGTTCAATGACTGCGTCGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGAGGAGCCAAGAAAA
GACCCGCCCCCAGTGACGCAGATATAAGTGAGCCCAAACGGGCGCGCGAGTCAGTTGCGC
AGCCATCAACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATTATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTCTGGTCTGGGATCTACTACAATGGCTACAGGC
AGTGGCGCACCAATGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCGGACGTCTTCATGGTGCCACAGTATGGATACCTCACCCTGAACAACGGGAGTCAGGCA
GTAGGACGCTCTTCATTTTACTGCCTGGAGTACTTTCCTTCTCAGATGCTTCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATTGACCAGTACCTGTATTATCTGAAC
AAAACACAATCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCGGCTACA
AAGTATCATCTAAATGGCCGGGACTCGTTGGTTAATCCAGGACCAGCTATGGCCAGCCAC
AAAGACGATGAAGAAAAGTTTTTCCCCATGCATGGAACCCTGATATTTGGTAAACAAGGA
ACAAATGCTAACGACGCGGATTTGGACAATGTCATGATTACAGATGAAGAAGAAATCCGC
ACCACCAATCCCGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCGCTGATGGGAGGTTTTGGACTCAAACACCCACCTCCTCAGATC
ATGATCAAAAACACTCCAGTTCCAGCCAATCCTCCCACAAACTTCAGTTCTGCCAAGTTT
GCTTCTTTCATCACACAGTACTCCACGGGGCAGGTCAGCGTGGAGATCGAGTGGGAGCTG
CAGAAGGAGAACAGCAAACGCTGGAACCCCGAGATCCAGTACACTTCCAACTACAACAAG
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTCTTTCTTATGTAGTTTCCATGGCTACGTA
GATAAGTAGCATGGCGGGTTAATCATTAACTCTAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 108
>#714_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGTCCAAAGGTCGCC
CGACGGGCGACCTTTGGTCGCCCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCAGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATGTGATTGAGCAGGCACCCCTGA
CCGTGGCCGAGAAGCTGCAGCGCGACTTTCTGACGGAATGGCGCCGTGTGAGTAAGGCCC
CGGAGGCCCTCTTCTTTGTGCAATTTGAGAAGGGAGAGAGCTACTTCCACATGCACGTGC
TTGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACGTCGGAGAAGCAGTGGATCCAGGAGGATCAGGCTTCATACATCTCCTTCA
ATGCGGCCTCCAACTCGCGGTCCCAAATCAAGGCTGCCTTGGACAATGCGGGAAAGATCA
TGAGCCTGACTAAAACTGCCCCCGACTACCTGGTGGGCCAGCAGCCCGTGGAAGACATTT
CCAGCAATCGGATTTATAAAATTTTGGAACTGAACGGGTACGATCCCCAATACGCGGCTT
CCGTCTTTCTGGGATGGGCCACGAAAAAGTTCGGCAAGAGAAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCCTTCAACGACTGTGTTGACAAGATGG
TGATCTGGTGGGAGGAGGGGAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAAAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAAGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCAAAAAAGAGGGTTCTT
GAACCTCTGGGCCTGGTTGAGGAGCCTGTTAAAACGGCTCCGGGAAAAAAGAGGCCGGTA
GAGCACTCTCCTGCGGAGCCAGACTCCTCCTCGGGAATCGGAAAAGCGGGCCAGCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTCTGGGATCTACTACAATGGCTACAGGC
AGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAAAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTGCCACAGTATGGATACCTCACCCTGAACAACGGGAGTCAAGCG
GTAGGACGCTCTTCATTTTACTGCCTGGAGTACTTTCCTTCTCAGATGCTGCGTACCGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAGACACAATCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCGGCTACA
AAGTATCATCTAAATGGCCGGGACTCGTTGGTTAATCCAGGACCAGCTATGGCCAGCCAC
AAAGATGACGAAGAAAAGTTTTTCCCCATGCATGGAACCCTGATATTTGGTAAACAAGGA
ACAAATGCTAACGACGCGGATTTGGACAATGTCATGATTACAGATGAAGAAGAAATCAGG
ACCACCAATCCCGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCACCCTTCTCCACTGATGGGAGGTTTTGGACTCAAACACCCGCCTCCTCAGATC
ATGATCAAAAACACTCCCGTTCCAGCCAATCCTCCCACAAATTTCAGTTCTGCCAAGTTT
GCTTCTTTCATCACACAGTACTCCACGGGGCAGGTCAGCGTGGAAATCGAGTGGGAGCTG
CAGAAGGAGAACAGCAAACGCTGGAATCCCGAAATTCAGTATACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGTGTACTTCTTTTCTTATATAGTTTCCATGGCTACGT
AGATAAGTAGCATGGCGGTTAATCATTAACTCTAAGGAACCCCTAGTGATGGAGTTGGCC
ACTCCCTCTCTGCGCGCTCGCTCGCTCACTGAGGCCGCGCGGGCAAAGCCCGGCCGTCGG
GCGACCTTTGCCCGGGCGACCTTAGTGAGCGAGCGAGCGCGCAGAGAGGGAGTGGCCAA
SEQ ID NO: 109
>#3086_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGCCCGGGCAAAGCCCGGG
CGTCGGGCGACCTTTGGTCGCCCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAATGGCGCCGCGTGAGTAAGGCCC
CGGAGGCCCTCTTCTTTGTGCAATTTGAGAAGGGAGAGAGCTACTTCCACATGCACGTGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCTGAGCGTAAACGGTTGGTGGCGCAGCATCTGA
CGCACGTGTCGCAGACGCAGGAGCAGAACAAAGAGAATCAGAATCTCAATTCTGACGCGC
CGGTTATTAGATCAAAAACCTCAGCCAGGTACATGGAGCTGGTCGGGTGGCTCGTGGATA
AGGGGATTACGTCGGAGAAGCAGTGGATTCAGGAAGACCAGGCTTCATACATATCCTTCA
ATGCGGCCTCCAACTCGCGGTCCCAGATCAAGGCTGCCTTGGACAATGCGGGAAAGATTA
TGAGCCTCACTAAAACTGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACGGTTCCCTTCT
ACGGGTGCGTAAACTGGACCAATGAGAACTTTCCCTTCAATGACTGCGTCGACAAGATGG
TGATCTGGTGGGAGGAGGGGAAGATGACCGCCAAGGTCGTAGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTTACCCGCCGTC
TGGATCATGACTTTGGGAAGGTCACAAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCAATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATATGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACAACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAGCCTGTTAAGACGGCTCCGGGAAAAAAGAGGCCGGTA
GAGCACTCTCCTGTGGAGCCAGACTCCTCCTCGGGAACCGGAAAAGCGGGCCAACAGCCT
GCGAAAAAAAGACTGAATTTTGGTCAGACTGGAGACGCAGACTCCGTACCTGACCCCCAG
CCTCTCGGACAGCCACCAGCAGCCCCCTCTGGTCTGGGATCTACTACAATGGCTACAGGC
AGTGGCGCACCAATGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCTTGGGGGTATTTTGACTTCAACAGA
TTCCACTGCCACTTTTCACCACGTGACTGGCAAAGACTCATCAACAACAACTGGGGATTC
CGACCCAAAAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATTGACCAGTACCTGTATTATCTGAAC
AAGACACAATCAAATAGTGGAACTCTTCAGCAGTCTCGGCTACTGTTTAGCCAAGCTGGA
CCCACCAACATGTCTCTTCAAGCTAAAAACTGGCTGCCTGGACCTTGCTACAGACAGCAG
CGTTTATCAAAGCAGGCAAACGACAACAATAACAGCAACTTTCCCTGGACTGCAGCTACA
AAGTATCATCTAAATGGCCGGGACTCGTTGGTTAATCCAGGACCAGCTATGGCCAGTCAT
AAAGACGATGAAGAAAAGTTTTTCCCCATGCATGGAACCCTGATATTTGGTAAACAAGGA
ACAAATGCCAACGACGCGGATTTGGACAATGTCATGATTACAGATGAAGAAGAAATCCGC
ACCACAAATCCTGTAGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATATGGGCCAAGATTCCTCACACCGATGGA
CACTTTCATCCTTCTCCGCTGATGGGAGGTTTTGGACTCAAACACCCACCTCCTCAGATC
ATGATCAAAAACACTCCCGTTCCAGCCAATCCTCCTACAAACTTCAGTTCTGCCAAGTTT
GCTTCTTTCATCACACAGTATTCCACGGGACAGGTCAGCGTGGAGATCGAGTGGGAGCTG
CAGAAGGAGAACAGCAAACGCTGGAACCCCGAGATCCAGTACACTTCTAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTTTGCGTACTTATTTCTTATCTAGTTTCCATGGCTACGTA
GATAAGTGCCATGGTGGGTTAATCATTAACTCTAAGGAACCCCTAGTGATGGAGTTGGCC
ACTCCCTCTCTGCGCGCTCGCTCGCTCACTGAGGCCGCCCGGGCAAAGCCCGGGCGTCGG
GCGACCTTTGGTCGCCCGGCCTCAGTGAGCGAGATTCAAACAGAGAGGGAGTGGCCAA
SEQ ID NO: 110
>#3142_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGCCCGGGCAAAGCCCGGG
CGTCGGGCGACCTTTGGTCGCCCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGATCACGTGAGTGTTTTGCGACATTTTGCGACACCAT
GTGGTTACGCAGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTAAACTGGGTGGCCGAGAAGG
AATGGGAGTTGCCGCCAGATTCTGACATGGATCTGAATCTGATTGAGCAGGCACCCCTGA
CCGTGGCCGAAAAGCTGCAGCGCGAGTTTCTGACGGAATGGCGCCGTGTGAGTAAAGCCC
CGGAGGCCCTCTTCTTTGTGCAATTTGAGAAGGGAGAGAGCTACTTCCACATGCACGTGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAACCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACCTCCGAGAAGCAGTGGATTCAGGAGGACCAGGCCTCATACATCTCCTTCA
ATGCGGCCTCCAACTCGCGGTCCCAAATCAAGGCTGCTCTGGACAATGCGGGAAAGATCA
TGAGCCTGACTAAAACGGCCCCCGACTACCTGGTGGGTCAGCAGCCCGTGGAGGACATTT
CCGGCAATCGGATTTATAAGATCTTGGAACTGAACGGGTACGATCCCCAATACGCGGCTT
CCGTCTTTCTGGGATGGGCCACGAAAAAGTTCGGCAAGAGAAACACCATCTGGCTGTTTG
GGCCTGCAACTACCGGGAAGACCAACATCGCGGAGGCCATAGCCCACACTGTGCCCTTTT
ACGGGTGCGTAAACTGGACCAATGAGAACTTTCCGTTCAATGACTGCGTCGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAGTCTTCGGCCCAGATCGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCTAAGAAAA
GGCCCGCCCCCAGTGACGCAGATATAAGTGAGCCCAAACGGGCGCGCGAGTCAGTTGCGC
ATCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTTACTCACGGACAGAAAGACTGTTTAGAGTGCTTTCCCG
TGTCAGAATCTCAACCCGTTTCTGTCGTCAAAAAGAAGACTTATCAGAAACTGTGTCCAA
TTCATCACATTTTGGGAAGAGCACCCGAGATTGCGTGTTCGGCCTGCGATCTGGCCAATG
TGGACTTGGATGACTGTGTTTCTGAACAATAAATGATTTAAATCAGGTATGGCTGCCGAT
GGTTATCTTCCAGATTGGCTCGAGGACACTCTCTCTGAAGGAATAAGACAGTGGTGGAAG
CTCAAACCTGGCCCACCACCACCAAAGCCCGCAGAGCGGCATAAGGACGACAGCAGGGGT
CTTGTGCTTCCTGGGTACAAGTACCTCGGACCCTTCAACGGACTCGACAAGGGAGAGCCG
GTCAACGAGGCAGACGCCGCGGCCCTCGAGCACGACAAGGCCTACGACCGGCAGCTCGAC
AGCGGAGACAACCCGTACCTCAAGTACAACCACGCCGACGCGGAGTTTCAGGAGCGTCTT
AAAGAAGATACGTCTTTTGGGGGCAACCTCGGACGAGCAGTCTTCCAGGCGAAAAAGAGG
GTTCTTGAACCTCTGGGCCTGGTTGAGGAACCTGTTAAGACGGCTCCGGGAAAAAAGAGG
CCGGTAGAGCACTCTCCTGCGGAGCCAGACTCCTCCTCGGGAACCGGAAAGGCGGGCAAC
CAGCCTGCAAGAAAAAGACTGAATTTCGGTCAGACTGGAGACGCAGACTCCGTACCTGAC
CCCCAGCCTATCGGACAGCCACCAGCAGCCCCCACAAGTTTGGGATCTACTACAATGGCT
ACAGGCAGTGGCGCACCAATGGCAGACAATAACGAGGGTGCCGATGGAGTGGGTAATTCC
TCAGGAAATTGGCATTGCGATTCCCAATGGCTGGGCGACAGAGTCATTACCACCAGCACC
CGAACCTGGGCCCTGCCCACCTACAACAACCACCTCTACAAGCAGATCTCCAGCCAATCA
GGAGCTTCAAACGACAACCACTACTTTGGCTACAGCACCCCCTGGGGGTATTTTGACTTC
AACAGATTCCACTGCCACTTTTCACCACGTGACTGGCAAAGACTCATCAACAACAATTGG
GGATTCCGGCCCAAAAGACTCAACTTCAAGCTCTTTAACATTCAAGTCAAGGAGGTCACG
CAGAATGACGGTACGACGACGATTGCCAATAACCTTACCAGCACGGTTCAGGTGTTTACT
GACTCGGAGTACCAGCTCCCGTACGTCCTCGGCTCGGCGCATCAAGGATGCCTCCCGCCG
TTTCCAGCGGACGTCTTCATGGTCCCACAGTATGGATACCTCACCCTAAACAACGGGAGT
CAGGCGGTAGGACGCTCTTCCTTTTACTGCCTGGAGTACTTTCCTTCTCAGATGCTGCGT
ACTGGAAACAACTTTCAGTTCAGCTACACCTTTGAAGACGTTCCTTTCCACAGCAGCTAT
GCTCACAGCCAGAGTCTGGATCGGCTGATGAATCCTCTGATCGACCAGTACCTGTATTAT
CTGAACAAGACACAATCAAATAGTGGAACTGTTCAGCAGTCTCGGCTACTGTTTAGCCAA
GCTGGACCCACCAGCATGTCTCTTCAAGCTAAAAACTGGCTGCCTGGACCTTGCTACAGA
CAGCAGCGTCTGTCAAAGCAGGCAAGCGACAACAACAACAGCAACTTTCCCTGGACTGCG
GCTACAAAGTATCATCTAAATGGCCGGGACTCGTTGGTTAATCCAGGACCAGCTATGGCC
AGTCACAAGGATGACGAAGAAAAGTTTTTCCCCATGCATGGAACCCTGATATTTGGTAAA
GAAGGAACAACTTCTAACAACGCGGATTTGGAACATGTCATGATTACAGATGAAGAAGAA
ATCAGGACCACTAATCCAGTGGCTACGGAGCAGTACGGAAATGTGTCAAATAATTTGCAA
AACTCAAATACTGGTCCAACTACTGAAAATGTCAATAACCAAGGAGCGTTACCTGGTATG
GTGTGGCAGGATAGAGACGTGTACCTGCAGGGACCCATTTGGGCCAAGATTCCACACACG
GACGGACACTTTCATCCTTCTCCGCTTATGGGAGGTTTTGGACTCAAACACCCACCTCCT
CAAATCATGATCAAAAACACTCCCGTTCCAGCCAATCCTCCTACAAACTTCAGTGCGGCA
AAGTTTGCTTCTTTCATCACACAGTACTCCACGGGGCAGGTCAGCGTGGAGATCGAGTGG
GAGCTGCAGAAAGAGAACAGCAAACGCTGGAACCCCGAGATCCAGTACACTTCCAATTAC
AACAAATCTGTTAATGTGGACTTTACTGTGGACACTAATGGCGTGTATTCAGAGCCTCGC
CCCATTGGCACCAGATACCTGACTCGTAATCTGTAATTGCTTGTTAATCAATAAACCGTT
TAATTCGTTTCAGTTGAACTTTGGTCTCTGCGTACTTCTTTTATTATCTAGTTTCCATGG
CTACGTAGATAAGTAGCATGGCGGGTTAATCATTAACTCTAAGGAACCCCTAGTGATGGA
GTTGGCCACTCCCTCTCTGCGCGCTCGCTCGCTCACTGAGGCCGCCCGGGCAAAGCCCGG
GCGTCGGGCGACCTTTGGTCGCCCGGCCTCAGTGAGCGAGCGAGCGCGCAGAGAGGGAGT
GGCCAA
SEQ ID NO: 111
>#1024_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCTGGGTATTTAAGCCCGAGTGAGCACGCAGGGTCTCCATTTTGAAGCGGGA
GGTTTGAACGCGCAGCCGCCATGCCGGGGTTTTACGAGATTGTGATTAAGGTCCCCAGCG
ACCTTGACGAGCATCTGCCCGGTATTTCTGACAGCTTTGTGAACTGGGTGGCCGAGAAAG
AATGGGAGTTGCCGCCAGATTCTGACATGGATCTGAATCTGATTGAGCAGGCACCCCTGA
CCGTAGCCGAGAAACTGCAGCGCGACTTTCTGACAGAATGGCGCCGTGTGAGTAAGGCCC
CCGAGGCCCTCTTTTTTGTGCAATTTGAAAAGGGAGAGAGCTACTTCCACATGCACGTGC
TGGTGGAGACCACCGGGGTGAAGTCCATGGTTTTGGGACGTTTCCTGAGTCAGATTCGCG
AAAAACTGATTCAGAGAATTTACCGCGGGATCGAGCCGACTTTGCCAAATTGGTTCGCGG
TCACAAAGACCCGAAATGGCGCCGGAGGCGGGAACAAGGTGGTGGACGAGTGCTACATCC
CCAATTACCTGCTCCCTAAAACCCAGCCTGAGCTCCAGTGGGCGTGGACTAATATGGAAC
AGTATTTAAGCGCCTGTTTGAACCTCGCGGAGCGTAAACGGTTGGTGGCGCAGCATCTGA
CGCACGTGTCGCAGACCCAGGAGCAGAACAAAGAAAATCAGAATCCCAATTCTGACGCGC
CGGTGATCAGATCAAAAACCTCAGCCAGGTACATGGAGCTGGTCGGGTGGCTCGTGGACA
AGGGGATTACCTCCGAGAAACAGTGGATTCAGGAGGACCAGGCTTCATACATCTCCTTCA
ATGCGGCCTCCAACTCGCGGTCTCAAATCAAGGCTGCTCTGGACAATGCGGGAAAGATTA
TGAGCCTCACTAAAACCGCCCCCGACTACCTGGTGGGCCAGCAGCCCGTGGAGGACATTT
CCGGCAATCGGATTTATAAAATCTTGGAACTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACGGTGCCCTTCT
ACGGGTGCGTAAACTGGACCAACGAGAACTTTCCCTTTAACGACTGCGTCGACAAGATGG
TGATTTGGTGGGAGGAGGGGAAGATGACCGCCAAGGTCGTGGAATCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCTAAGAAAA
GGCCCGCCCCCAGTGACGCAGATATAAGTGAGCCCAAACGGGCGCGCGAGTCAGTTGCGC
AGACATCGACGTCAGACGCGGAAGCTTCGATCAACTACGCGGACAGGTACCAAAACAAAT
GTTCTCGTCACGTGGGCATGAATCTGATGCTGTTTCCGTGCAAAACCTGCGAGAGAATGA
ATCAGATTTCAAATGTCTGTTTCACGCACGGTGTCAAAGACTGTGGGGAGTGCTTTCCCG
TGTCAAAATCTCAACCCGTTTCTGTCGTCAAAAAGAAGACTTATCAGAAACTGTGTCCAA
TTCATCACATTTTGGGAAGAGCACCCGAGATTGCGTGTTCGGCCTGCGATATGGCCAATG
TGGACTTGGATGACTGTGTTTCTGAACAATAAATGACTTAAACCAGGTATGGCTGCCGAT
GGTTATCTTCCAGATTGGCTCGAGGACAACCTCAGTGAAGGAATTCGCGAGTGGTGGGCT
TTGAAACCTGGAGCCCCTCAACCCAAGGCAAATCAACAACATCAAGACAACGCTCGGGGT
CTTGTGCTTCCGGGTTACAAATACCTTGGACCCGGCAACGGACTCGACAAGGGGGAGCCG
GTCAACGCAGCAGACGCGGCGGCCCTCGAGCACGACAAGGCATACGACAAGCAGCTCAAG
GCCGGAGACAACCCGTACCTCAAGTACAACCACGCCGACGCGGAGTTTCAGGAGCGTCTT
AAAGAAGATACGTCTTTTGGGGGCAACCTCGGACGAGCAGTCTTCCAGGCGAAAAAGAGG
GTTCTCGAACCTCTGGGCCTGGTTGAGGAACCTGTTAAGACGGCTCCGGGAAAAAAGAGG
CCGGTAGAGCACTCTCCTGCGGAGCCAGATTCCTCCTCCGGAACTGGAAAGTCGGGCCAA
CAGCCTGCAAGAAAAAGATTGAATTTTGGTCAGACTGGAGACGCAGACTCCGTACCTGAC
CCCCAGCCTCTCGGACAGCCACCAGCAGCCCCCTCTGGTCTGGGATCTACTACAATGGCT
ACAGGCAGTGGCGCACCAATGGCAGACAATAACGAGGGTGCCGATGGAGTGGGTAATTCC
TCAGGAAATTGGCATTGCGATTCCCAATGGCTGGGCGACAGAGTCATCACCACCAGCACC
CGAACCTGGGCCCTGCCCACCTACAACAATCACCTCTACAAGCAAATCTCCAGCCAATCA
GGAGCTTCGAACGACAACCACTACTTTGGCTACAGCACCCCTTGGGGGTATTTTGACTTC
AACAGATTCCACTGCCACTTTTCACCACGTGACTGGCAAAGACTCATCAACAACAACTGG
GGATTCCGACCCAAGAGACTCAACTTCAAGCTCTTTAACATTCAAGTCAAAGAGGTCACG
CAGAATGACGGTACGACGACGATTGCCAATAACCTTACCAGCACGGTTCAGGTGTTTACT
GACTCGGAGTACCAGCTCCCGTACGTCCTCGGCTCGGCGCATCAAGGATGCCTCCCGCCG
TTTCCAGCGGACGTCTTCATGGTGCCACAGTATGGATACCTCACCCTAAACAACGGGAGT
CAGGCGGTAGGACGCTCTTCCTTTTACTGCCTGGAGTACTTTCCTTCTCAGATGCTGCGT
ACAGGAAACAACTTTCAGTTCAGCTACACTTTTGAAGACGTGCCTTTCCACAGCAGCTAC
GCTCACAGCCAGAGTCTGGATCGGCTAATGAATCCTCTGATCGACCAGTACCTGTATTAT
CTAAACAGGACACAAACAGCCAGTGGAACTCAGCAGTCTCGGCTACTGTTTAGCCAAGCT
GGACCCACCAGCATGTCTCTTCAAGCTAAAAACTGGCTGCCTGGACCTTGCTACAGACAA
CAGCGTTTGTCAAAGCAGGCAAACGACAACAACAATAGCAACTTTCCCTGGACTGCGGCT
ACAAAGTACCACCTCAATGGCAGAGACTCTCTGGTGAATCCGGGCCCTGCTATGGCCAGT
CACAAAGACGATGAAGAAAAGTTTTTCCCCATGCATGGAACCCTGATATTTGGTAAAGAA
GGAACAAATGCTACCAACGCGGATTTGGACAATGTCATGATTACAGATGAAGAAGAAATC
CGCACCACAAATCCTGTAGCTACGGAGCAGTATGGATATGTGTCAAATAATTTGCAAAAC
TCAAATACTGCTGCAACTACTGAAACTGTCAATCACCAAGGAGCGTTACCTGGTATGGTG
TGGCAGGATAGAGACGTGTACCTGCAGGGACCCATTTGGGCCAAAATTCCTCACACCGAT
GGACACTTTCATCCTTCTCCGCTGATGGGAGGTTTTGGACTCAAACACCCACCTCCTCAG
ATCATGATCAAAAACACTCCCGTTCCAGCCAATCCTCCCACAAACTTTAGTGCGGCAAAG
TTTGCTTCTTTCATCACACAGTATTCCACGGGGCAAGTCAGCGTGGAGATCGAGTGGGAG
CTGCAGAAGGAGAACAGCAAACGCTGGAACCCCGAGATCCAGTACACTTCCAACTACAAC
AAATCTGTTAATGTGGACTTTACTGTGGACACTAATGGTGTGTATTCAGAGCCTCGCCCC
ATTGGCACCAGATACCTGACTCGTAATCTGTAATTGCTTGTTAATTAATAAACCGTTTAA
TTCGTTTCAGTTAAACTTTGGTCTCTGCGTACTTCTTTCTTATCTAGTTTCCATGGCTAC
GTAGATAAGTACCATGGCGGGTTAATCATTAACTCTAAGGAACCCCTAGTGATGGAGTTG
GCCACTCCCTCTCTGCGCGCTCGCTCGCTCACTGAGGCCGCCCGGGCAAAGCCCGGGCGT
CGGGCGACCTTTGGTCGCCCGGCCTCAGTGAGCGAGCGAGCGCGCAGAGAGGGAGTGGCC
AA
SEQ ID NO: 112
>#790_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGCCCGGGCAAAGCCCGGG
CGTCGGGCGACCTTTGGTCGCCCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGTATTTAAGCCCGAGTGAGCACGCAGGGTCTCCATTTTGAAGCGGGAG
GTTTGAACGCGCAGCCGCCATGCCGGGATTTTACGAGATTGTGATTAAGGTCCCCAGCGA
CCTTGACGAGCATCTGCCCGGCATTTCTGACAGCTTTGTGAACTGGGTGGCCGAGAAGGA
ATGGGAGTTGCCGCCAGATTCTGACATGGATCTGAATCTGATTGAGCAGGCACCCCTGAC
CGTGGCCGAGAAGCTGCAGCGCGACTTTCTGACGGAGTGGCGCCGCGTGAGTAAGGCCCC
GGAGGCCCTCTTCTTTGTGCAATTTGAGAAGGGAGAGAGCTACTTCCACATGCACGTGCT
TGTGGAAACTACCGGGGTGAAATCCATGGTTTTGGGACGTTTCCTGAGTCAGATTCGCGA
AAAACTGATTCAGAGAATTTACCGCGGGATCGAGCCGACTTTGCCAAACTGGTTCGCGGT
CACAAAGACCCGAAATGGCGCCGGAGGCGGGAACAAGGTGGTGGATGAGTGCTACATCCC
CAATTACCTGCTCCCCAAAACCCAACCTGAGCTCCAGTGGGCGTGGACTAATATGGAACA
GTATTTAAGCGCCTGTTTGAATCTCGCGGAGCGTAAACGGTTGGTGGCGCAGCATCTGAC
GCACGTGTCGCAGACGCAGGAGCAGAACAAAGAGAATCAGAATCCCAATTCTGACGCGCC
GGTGATCAGATCAAAAACCTCAGCCAGGTACATGGAGCTTGTCGGGTGGCTCGTGGATAA
GGGGATTACGTCGGAGAAGCAGTGGATCCAGGAGGACCAGGCTTCATACATCTCCTTCAA
TGCGGCCTCCAACTCGCGGTCTCAAATCAAGGCTGCTCTGGACAATGCGGGGAAGATTAT
GAGCCTGACTAAAACCGCCCCCGACTACCTGGTGGGCCAGCAGCCCGTGGAGGACATTTC
CAGCAATCGGATTTATAAAATCTTGGAACTGAACGGGTACGATCCCCAATATGCGGCTTC
CGTCTTTCTGGGATGGGCCACGAAAAAGTTCGGCAAGAGGAACACCATCTGGCTGTTTGG
GCCTGCAACTACCGGGAAGACCAACATCGCGGAGGCCATAGCCCACACTGTGCCCTTCTA
CGGGTGCGTAAACTGGACCAATGAGAACTTTCCTTTCAATGATTGCGTCGACAAGATGGT
GATCTGGTGGGAGGAGGGAAAGATGACCGCCAAGGTCGTGGAGTCGGCCAAAGCCATTCT
CGGAGGAAGCAAGGTGCGCGTGGACCAGAAATGCAAGTCCTCGGCCCAGATAGACCCGAC
TCCCGTGATCGTCACTTCAAACACCAACATGTGCGCCGTGATTGACGGGAACTCAACGAC
CTTCGAGCACCAGCAGCCGTTGCAAGACCGGATGTTCAAATTTGAACTCACCCGCCGTCT
GGATCATGACTTTGGGAAGGTCACCAAGCAGGAAGTGAAAGACTTTTTCCGGTGGGCAAA
GGATCACGTGGTTGAGGTGGAGCATGAATTCTACGTCAAAAAGGGTGGAGCCAAGAAAAG
ACCCGCCCCCAGTGACGCAGATATAAGTGAGCCCAAACGGGCGCGCGAGTCAGTTGCACA
GCCATCGACGTCAGACGCGGAAGCTTCGATCAACTACGCGGACAGGTACCAAAACAAATG
TTCTCGTCACGTGGGCATGAATCTGATGCTGTTTCCCTGCAGACAATGCGAGAGAATGAA
TCAGAATTCAAATATCTGCTTCACTCACGGACAGAAAGACTGTTTAGAGTGCTTTCCCGT
GTCAGAATCTCAACCCGTTTCTGTCGTCAAAAAGGCGTATCAGAAACTTTGCTACATTCA
TCATATCATGGGAAAGGTGCCAGACGCTTGCACTGCCTGCGATCTGGTCAATGTGGATTT
GGATGACTGCATCTCTGAACAATAAATGATTTAAATCAGGTATGGCTGCCGATGGTTATC
TTCCAGATTGGCTCGAGGACACTCTCTCTGAAGGAATAAGACAGTGGTGGAAGCTCAAAC
CTGGCCCACCACCACCAAAGCCCGCAGAGCGGCATAAGGACGACAGCAGGGGTCTTGTGC
TTCCTGGGTACAAGTACCTCGGACCCTTCAACGGACTCGACAAGGGAGAGCCGGTCAACG
AGGCAGACGCCGCGGCCCTCGAGCACGACAAGGCCTACGACCGGCAGCTCGACAGCGGAG
ACAACCCGTACCTCAAGTACAACCACGCCGACGCGGAGTTTCAGGAGCGCCTTAAAGAAG
ATACGTCTTTTGGGGGCAACCTCGGACGAGCAGTCTTCCAGGCGAAAAAGAGGGTTCTTG
AACCTCTGGGCCTGGTTGAGGAACCTGTTAAGACGGCTCCGGGAAAAAAGAGGCCGGTAG
AGCACTCTCCTGTGGAGCCAGACTCCTCCTCGGGAACCGGAAAAGCGGGCAACCAGCCTG
CAAGAAAAAGATTGAATTTTGGTCAGACTGGAGACGCAGACTCCGTACCTGACCCCCAGC
CTCTCGGACAACCACCAGCAGCCCCCTCTGGTCTGGGATCTACTACAATGGCTACAGGCA
GTGGCGCACCAGTGGCAGACAATAACGAGGGTGCCGATGGAGTGGGTAATTCCTCAGGAA
ATTGGCATTGCGATTCCCAATGGCTGGGCGACAGAGTCATCACCACCAGCACCCGAACCT
GGGCTCTGCCCACCTACAACAACCACCTCTACAAGCAAATCTCCAGCCAATCAGGAGCTT
CAAACGACAACCACTATTTTGGCTACAGCACCCCCTGGGGGTATTTTGACTTCAACAGAT
TCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATG
ACGGTACGACGACGATTGCCAATAACCTTACCAGCACGGTTCAGGTGTTTACTGACTCGG
AGTACCAGCTCCCGTACGTCCTCGGCTCGGCGCATCAAGGATGCCTCCCGCCGTTTCCAG
CGGACGTCTTCATGGTCCCACAGTATGGATACCTCACCCTGAACAACGGGAGTCAGGCGG
TAGGACGCTCTTCCTTTTACTGCCTGGAGTACTTTCCTTCTCAGATGCTGCGTACTGGAA
ACAACTTTCAGTTTAGCTACACCTTTGAAGACGTTCCTTTCCACAGCAGCTACGCTCACA
GCCAGAGTCTGGATCGGCTGATGAATCCTCTGATTGACCAGTACCTGTATTATCTAAACA
AGACACAATCAAATAGTGGAACTCTTCAACAGTCTCGGCTACTGTTTAGTCAAGCTGGAC
CCACCAGCATGTCTCTTCAAGCTAAAAACTGGCTGCCTGGACCTTGCTACAGACAGCAGC
GTCTGTCAAAGCAGGCAAACGACAACAACAACAGCAACTTTCCCTGGACTGCGGCTACAA
AGTATCATCTAAATGGCCGGGACTCGTTGGTTAATCCAGGACCAGCTATGGCCAGCCACA
AAGACGATGAAGAAAAGTTTTTCCCCATGCATGGAACCCTGATATTTGGTAAACAAGGAA
CAAATGCTAACGACGCGGATTTGGACAATGTCATGATTACAGATGAAGAAGAAATCCGCA
CCACCAATCCCGTGGCTACGGAGCAGTACGGATATGTGTCAAATAATTTGCAAAACTCAA
ATACTGGTCCAACTACTGGAACTGTCAATCACCAAGGAGCGTTACCTGGTATGGTGTGGC
AGGATCGAGACGTGTACCTGCAGGGACCCATTTGGGCCAAGATTCCTCACACCGATGGAC
ACTTTCATCCTTCTCCACTGATGGGAGGTTTTGGGCTCAAACACCCGCCTCCTCAGATCA
TGATCAAAAACACTCCCGTTCCAGCCAATCCTCCTACAAACTTCAGTTCTGCCAAGTTTG
CTTCTTTCATCACACAGTACTCCACGGGGCAGGTCAGCGTGGAGATCGAGTGGGAGCTGC
AGAAGGAGAACAGCAAACGCTGGAACCCCGAGATTCAGTACACTTCCAACTACAACAAAT
CTGTTAATGTGGACTTTACTGTGGACACTAATGGTGTGTATTCAGAGCCTCGCCCCATTG
GCACCAGATACCTGACTCGTAATCTGTAATTGCTTGTTAATCAATAAACCGTGTAATTCG
TTTCAGTTGAACTTTGGTCTCTGCGTACTTTTTTCTTATCTAGTTTCCATGGCTACGTAG
ATAAGTAGCATGGCGGGGTAATCATTAACTCTAAGGAACCCCTAGTGATGGAGTTGGCCA
CTCCCTCTCTGCGCGCTCGCTCGCTCACTGAGGCCGGGCGACCAAAGGTCGGGCGTCGCC
CGGGCTTTGCCCGGGCGGCCTCAGTGAGCGAGCGAGCGCGCAGAGAGGGAGTGGCCAA
SEQ ID NO: 113
>#976_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATTTGAATCTGATTGAGCAGGCACCCCTGA
CCGTGGCCGAGAAGCTGCAGCGCGACTTTCTGACGGAATGGCGCCGTGTGAGTAAGGCCC
CGGAGGCCCTCTTCTTTGTGCAATTTGAGAAGGGAGAGAGCTACTTCCACATGCACGTGC
TGGTGGAAACCACCGGGGTGAAATCTATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCATGTATCGCAGACGCAGGAGCAGAACAAAGAGAATCAGAATCCCAATTCTGACGCGC
CGGTGATCAGATCAAAAACCTCAGCCAGGTACATGGAGCTTGTCGGGTGGCTCGTGGATA
AGGGGATTACCTCGGAGAAGCAGTGGATCCAGGAGGACCAGGCCTCATACATCTCCTTCA
ATGCGGCCTCCAACTCGCGGTCGCAGATCAAGGCTGCTCTGGACAATGCGGGAAAGATCA
TGAGCCTGACTAAAACGGCCCCCGACTACCTGGTGGGCCAGCAACCCGTGGAGGACATTT
CCAGCAATCGGATTTATAAAATCTTGGAACTGAACGGGTATGATCCCCAATACGCGGCTT
CCGTCTTTCTGGGATGGGCTACGAAAAAGTTCGGCAAGAGGAACACCATCTGGCTGTTTG
GGCCTGCAACTACCGGGAAGACCAACATCGCGGAGGCTATAGCCCACACTGTGCCCTTTT
ACGGGTGCGTGAACTGGACCAATGAGAACTTTCCTTTCAACGATTGCGTCGACAAGATGG
TGATCTGGTGGGAGGAGGGGAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGTGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAGGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTCTGGTCTGGGATCTACTACAATGGCTACAGGC
AGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCTCTGCCCACCTACAACAACCACCTCTACAAGCAAATCTCCAGCCAATCAGGAGCC
AGCAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGGCCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTCCCACAGTATGGATATCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTTAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAGACACAATCAAATAGTGGAACTCTTCAGCAGTCTCGGCTACTGTTTAGCCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCGGCTACA
AAGTATCATCTAAATGGCCGGGACTCGTTGGTTAATCCAGGACCAGCTATGGCCAGCCAC
AAAGACGATGAAGAAAAGTTTTTCCCCATGCATGGAACCCTGATATTTGGTAAACAAGGA
ACAAATGCTAACGACGCGGATTTGGACAATGTCATGATTACAGATGAAGAAGAAATCCGC
ACCACCAATCCAGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGACTCAAACACCCACCTCCTCAGATC
ATGATCAAAAACACTCCCGTTCCAGCCAATCCTCCTACAAACTTCAGTTCTGCCAAGTTT
GCTTCTTTCATCACACAGTATTCCACGGGGCAGGTCAGCGTGGAGATCGAGTGGGAGCTG
CAGAAGGAGAACAGCAAACGCTGGAATCCCGAGATTCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGTGTGCTTCTTTCTTATCTAGTTTCCATGGCTACGTA
GATAAGTACCATGGCGGGTTAATCATTAACTCTAAGGAACCCCTAGTGATGGAGTTGGCC
ACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGAGAGGGAGTGGCCAA
SEQ ID NO: 114
>#1286_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTATAGGTCACGTGAGTGTTTTGCGACATTTTGCGACACCAT
GTGGTTACGCTGGGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGTGTGAGTAAGGCCC
CGGAGGCTCTTTTCTTTGTGCAATTTGAGAAGGGAGAGAGCTACTTCCACATGCACGTGC
TTGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTCGCGG
TCACAAAGACCCGAAATGGCGCCGGAGGCGGGAATAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAACTCGCGGTCCCAGATCAAGGCTGCCTTGGACAATGCGGGAAAGATCA
TGAGCCTGACTAAAACCGCCCCCGACTACCTGGTGGGCCAGCAGCCCGTGGAGGACATTT
CCAGCAATCGGATTTATAAAATCTTGGAACTGAACGGATACGATCCCCATTACGCGGCTT
CCGTCTTTCTGGGATGGGCCACTAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCGTTCAATGATTGCGTCGACAAGATGG
TGATTTGGTGGGAGGAGGGAAAGATGACCGCCAAGGTCGTGGAGTCGGCCAAAGCCATTC
TCGGCGGAAGCAAGGTGCGCGTGGACCAAAAGTGCAAGTCTTCGGCCCAGATCGACCCGA
CTCCCGTGATAGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTGTGCTACATTC
ATCACATCATGGGAAAGGTGCCAGACGCTTGCACTGCTTGCGACCTGGTCAATGTGGACT
TGGATGACTGTGTTTCTGAACAATAAATGACTTAAACCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTCTGGTTTGGGATCTACTACAATGGCTACAGGC
AGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCTCTGCCCACCTACAACAACCACCTCTACAAGCAAATCTCCAGCCAATCAGGAGCC
AGCAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGGCCCAAGAGACTCAACTTCAAGCTCTTTAACATTCAAGTCAAAGAGGTCACGCAGAAT
GACGGTACGACAACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTCATGGTCCCACAGTATGGATACCTCACCCTGAACAACGGGAGTCAGGCA
GTAGGACGCTCTTCATTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAAACACAATCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCGGCTACA
AAGTATCATCTAAATGGCCGGGACTCGTTGGTTAATCCAGGACCAGCTATGGCCAGCCAC
AAAGACGATGAAGAAAAGTTTTTCCCCATGCATGGAACCCTGATATTTGGTAAACAAGGA
ACAAATGCTAACGACGCGGATTTGGACAATGTTATGATTACAGATGAAGAAGAAATCCGC
ACCACCAATCCCGTGGCTACGGAGCAGTACGGATATGTGTCAAATAATTTGCAAAACTCA
AACACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACGGACGGA
CATTTTCATCCTTCTCCACTGATGGGAGGTTTTGGGCTCAAACACCCGCCTCCTCAGATC
ATGATCAAAAACACTCCCGTTCCAGCCAATCCTCCTACAAACTTCAGTTCTGCCAAGTTT
GCTTCTTTCATCACACAGTATTCCACGGGGCAGGTCAGCGTGGAGATCGAGTGGGAGCTA
CAGAAGGAGAACAGCAAACGCTGGAATCCCGAAATTCAGTATACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTCTTTTCTTATCTAGTTTCCATGGCTACGT
AGATAAGTAGCATGGAGGGTTAATCATTAACTACAAGGAACCCCTAGTGATGGAGTTGGC
CACTCCCTCTCTGCGCGCTCGCTCGCTCACTGAGGCCGGGCGACCAAAGGTCGCCCGACG
CCCGGGCTTTGCCCGGGCGGCCTCAGTGAGCGAGCGAGCGCGCAGAGAGGGAGTGGCCAA
SEQ ID NO: 115
>#163_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAATTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTGAAGCGGGA
GGTTTGAACGCGCAGCCGCCATGCCGGGATTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGTGTGAGTAAGGCCC
CGGAGGCCCTTTTCTTTGTGCAATTTGAGAAGGGAGAGAGCTACTTCCACATGCACGTGC
TTGTGGAGACCACCGGGGTGAAATCCATGGTTTTGGGACGTTTCCTGAGTCAGATTCGCG
AAAAACTGATTCAGAGAATTTACCGCGGAATCGAGCCGACTTTGCCAAACTGGTTCGCGG
TCACAAAGACCAGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACTCAGGAGCAGAACAAGGAGAATCAGAATCCCAATTCTGACGCGC
CGGTGATCAGATCAAAAACCTCAGCCAGGTACATGGAGCTGGTCGGGTGGCTCGTGGATA
AGGGGATTACGTCGGAGAAGCAGTGGATCCAGGAGGACCAGGCTTCATACATCTCCTTCA
ATGCGGCCTCCAACTCGCGGTCCCAAATCAAGGCTGCTCTGGACAATGCGGGAAAGATCA
TGAGCCTGACTAAAACCGCTCCCGACTACCTGGTGGGCCAGCAGCCCGTGGAGGACATTT
CCAGCAATCGGATTTATAAAATCTTGGAACTGAACGGGTACGACCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCCATAGCCCACACTGTGCCCTTCT
ACGGGTGCGTAAACTGGACCAATGAGAACTTTCCTTTCAATGATTGCGTCGACAAGATGG
TGATCTGGTGGGAGGAGGGGAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCATGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGAAGCGGAAGCTTCGATCAACTACGCGGACAGGTACCAAAACAAAT
GTTCTCGTCACGTGGGCATGAATCTGATGCTGTTTCCCTGCAGACAATGCGAAAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTGCCAGACGCTTGCACTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTCTGGTTTGGGATCTACTACAATGGCTACAGGC
AGTGGCGCACCAGTGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCT
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAGAAACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATTGACCAGTACCTGTATTATCTGAAC
AAGACACAATCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCGGCTACA
AAGTATCATCTAAATGGCCGGGACTCGTTGGTTAATCCAGGACCAGCTATGGCCAGCCAC
AAAGACGATGAAGAAAAGTTTTTCCCCATGCATGGAACCCTGATATTTGGTAAACAAGGA
ACAAATGCTAACGACGCGGATTTGGACAATGTCATGATTACAGATGAAGAAGAAATCCGC
ACCACCAATCCCGTGGCTACGGAGCAGTACGGATATGTCTCAAATAATTTGCAAAACTCA
AATACTGGTCCAACTACTGGAACTGTAAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGACTCAAACACCCGCCTCCTCAGATC
ATGATCAAAAACACTCCCGTTCCAGCCAATCCTCCCACAAACTTCAGTTCTGCCAAGTTT
GCTTCTTTCATCACACAGTATTCCACGGGGCAGGTCAGCGTGGAGATCGAGTGGGAGCTG
CAGAAGGAGAACAGCAAACGCTGGAATCCCGAGATTCAGTACACTTCCAACTACAACAAA
TCTGTTAATGTGGACTTTACTGTGGACACTAATGGTGTGTATTCAGAGCCTCGCCCCATT
GGCACCAGATACCTGACTCGCAATCTGTAATTGCTTGTTAATCAATAAACCGTTTGATTC
GTTTCAGTTGAACTTTGGTCTCCGCGTACTTCTTTCTTATCTAGTTTCCATGGCTACGTA
GATAAGTAGCATGGCGGGTTAATCATTAACTACAAGGAACCCCTAGTGATGGAGTTGGCC
ACTCCCTCTCTGCGCGCTCGCTCGCTCACTGAGGCCGCCCGGGCAAACCTCTCTGCGCGC
TCGCTCTTTGCCCGGGCGGCCTCAGTGAGCGAGCGAGCGCGCAGAGAGGGAGTGGCCAA
SEQ ID NO: 116
>#685_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTTACGCAGGGTATTTAAGCCCGAGTGAGCACGCAGGGTCTCCATTTTAAAGCGGGA
GGTTTGAACACGCAGCCGCCATGCCGGGGTTTTACGAGATTGTGATTAAGGTCCCCAGCG
ACCTTGACGAGCATTTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGA
CCGTGGCCGAGAAGCTGCAGCGCGACTTTCTGACGGAGTGGCGCCGTGTGAGTAAGGCCC
CGGAGGCTCTTTTCTTTGTGCAATTTGAGAAGGGAGAGAGCTACTTCCACATGCACGTGC
TGGTGGAGACCACCGGGGTGAAATCCATGGTTTTGGGACGTTTCCTGAGTCAGATTCGCG
AAAAACTGATTCAGAGAATTTACCGCGGGATCGAGCCGACTTTGCCAAACTGGTTTGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGACA
AGGGGATTACCTCGGAGAAGCAGTGGATCCAGGAGGACCAGGCTTCATACATCTCCTTCA
ATGCGGCCTCCAACTCGCGGTCCCAGATCAAGGCTGCTCTGGACAATGCGGGAAAGATCA
TGAGCCTAACTAAAACCGCCCCCGACTACCTGGTGGGCCAGCAGCCCGTGGAGGACATTT
CCAGCAATCGGATTTATAAAATCTTGGAACTGAACGGGTACGATCCCCAATACGCGGCTT
CCGTCTTTCTGGGATGGGCTACGAAAAAGTTCGGCAAGAGGAACACCATCTGGCTGTTTG
GGCCTGCAACTACCGGGAAGACCAACATTGCGGAGGCTATAGCCCACACTGTGCCCTTCT
ACGGGTGCGTAAACTGGACCAATGAGAACTTTCCATTCAATGATTGCGTCGACAAGATGG
TGATCTGGTGGGAGGAGGGAAAGATGACCGCCAAGGTCGTGGAGTCGGCCAAAGCCATTC
TCGGAGGAAGCAAGGTGCGCGTGGACCAGAAATGCAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGAAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCAAGTGACGCAGATATAAGTGAGCCCAAACGGGTGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTATTTCCCTGCAGACAATGCGAGAGAATGA
ATCAGAATTCAAATATTTGCTTCACTCACGGACAGAAAGACTGTTTAGAGTGCTTTCCCG
TGTCAGAATCTCAACCCGTTTCTGTCGTCAAAAAGGCATATCAGAAACTGTGCTACATTC
ATCATATCATGGGAAAGGTGCCAGACGCTTGCACTGCCTGCGATCTGGTCAATGTGGATT
TGGATGACTGTATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTCAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAAACGGCTCCGGGAAAAAAGAGGCCGGTA
GAGCACTCTCCTGCGGAGCCAGACTCCTCCTCGGGAACCGGAAAAGCGGGCCAGCAGCCT
GCAAGAAAAAGATTGAATTTCGGTCAGACTGGAGACGCAGACTCCGTACCTGACCCCCAG
CCTCTCGGACAGCCACCAGCAGCCCCCTCTGGTTTGGGATCTACTACAATGGCTACAGGC
AGTGGCGCACCAATGGCAGACAATAACGAGGGTGCCGATGGAGTGGGTAATTCCTCAGGA
AATTGGCATTGCGATTCCCAATGGCTGGGCAACAGAGTCATCACCACCAGCACCCGAACC
TGGGCCCTGCCCACCTACAACAACCACCTCTACAAGCAAATTTCCAGCCAATCAGGAGCC
TCAAACGACAACCACTACTTTGGCTACAGCACCCCTTGGGGGTATTTTGACTTCAACAGA
TTCCACTGCCACTTTTCACCACGTGACTGGCAAAGACTCATCAACAACAACTGGGGATTC
CGGCCCAAAAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTCTTTATGGTCCCACAGTATGGATACCTCACCCTGAACAACGGGAGTCAGGCG
GTAGGACGCTCTTCCTTTTACTGCCTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTAAAC
AAGACACAATCAAATAGTGGAACTCTTCAGCAGTCTCGGCTACTGTTTAGCCAAGCTGGA
CCCACCAGCATGTCTCTTCAAGCTAAAAACTGGCTGCCTGGACCTTGCTACAGACAGCAG
CGTCTGTCAAAGCAGGCAAACGACAACAACAACAGCAACTTTCCCTGGACTGCGGCTACA
AAGTATCATCTAAATGGCCGGGACTCGTTGGTTAATCCAGGACCAGCTATGGCCAGCCAC
AAAGACGATGAAGAAAAGTTTTTCCCCATGCATGGAACCCTGATATTTGGTAAACAAGGA
ACAAATGCCAACGACGCGGATTTGGACAATGTCATGATTACAGATGAAGAAGAAATCCGC
ACCACCAATCCCGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGGCTCAAACACCCGCCTCCTCAGATC
ATGATCAAAAACACTCCCGTTCCAGCCAATCCTCCAACAAACTTCAGTTCTGCCAAGTTT
GCTTCTTTCATCACACAGTATTCCACGGGACAGGTCAGCGTGGAGATTGAGTGGGAGCTG
CAGAAGGAGAACAGCAAACGCTGGAATCCCGAGATTCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCCGCGTACTTCTTTTCTTATCTAGTTTCCATGGCTACGT
AGATAAGTAGCATGGTGGGTTTATCATTAACTATAAGGAACCCCTAGTGATGGAGTTGGC
CACTCCCTCTCTGCGCGCTCGCTCGCTCACTGAGGCCGCCCGGGCAAAGCCCGGGCGTCG
GGCGACCTTTGCCCGGGCGGCCTCAGTGAGCGAGCGAGCGCGCAGAGAGGGAGTGGCCAA
SEQ ID NO: 117
>#442_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGGTCACGTGAGTGTTTTGCGACATTTTGCGACACCAT
GTGGTCACGCTGGGTATTTAAGCCCGAGTGAGCACGCAGGGTCTCCATTTTAAAGCGGGA
GGTTTGAACGGGCAGCCGCCATGCCGGGGTTTTACGAGATTGTGATTAAGGTCCCCAGCG
ACCTTGACGAGCATCTGCCCGGCATTTCTGACAGCTTTGTGCACTGGGTGGCCGAGAAGG
AATGGGAGTTGCCGCCAGATTCTGACATGAATCTGAATCTGATTGAGCAGGCACCCCTGA
CCGTGGCCGAGAAGCTGCAGCGCGACTTTCTGACGGAGTGGCGCCGCGTGAGTAAGGCCC
CGGAGGCCCTCTTCTTTGTGCAATTTGAGAAGGGAGAGAGCTACTTCCACATGCACGTGC
TGGTGGAGACCACCGGGGTGAAATCCATGGTTTTGGGACGTTTCCTGAGTCAGATTCGCG
AAAAACTGATTCAGAGAGTTTACCGCGGGATCGAGCCGACTTTGCCAAATTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAATCTCGCGGAGCGTAAACGGTTGGTGGCGCAGCATCTGA
CGCACGTGTCGCAGACGCAGGAGCAGAACAAAGAGAATCAGAATCCCAATTCTGACGCAC
CGGTGATCAGATCAAAAACCTCAGCCAGGTACATGGAGCTGGTCGGGTGGCTCGTGGACA
AGGGGATTACCTCGGAGAAGCAGTGGATCCAGGAGGACCAGGCTTCATACATCTCCTTCA
ATGCGGCCTCCAACTCGCGGTCCCAAATCAAGGCTGCTCTGGACAATGCGGGAAAGATCA
TGAGCCTGACTAAAACCGCCCCAGACTACCTAGTGGGCCAGCAGCCCGTGGAGGACATTT
CCAGCAATCGGATTTATAAAATCTTGGAATTGAACGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAGGCTATAGCCCACACTGTGCCCTTCT
ACGGGTGCGTAAACTGGACCAATGAGAACTTTCCCTTCAACGACTGTGTCGACAAGATGG
TGATCTGGTGGGAGGAGGGGAAGATGACCGCCAAGGTCGTGGAGTCGGCCAAAGCCATTC
TCGGAGGAAGCAAGGTGCGCGTGGACCAGAAATGTAAGTCCTCGGCCCAGATAGACCCGA
CTCCCGTGATCGTCACCTCCAACACCAACATGTGCGCCGTGATTGACGGGAACTCAACGA
CCTTCGAACACCAGCAGCCGTTGCAAGACCGGATGTTCAAATTTGAACTCACCCGCCGTC
TGGATCATGACTTTGGGAAGGTCACCAAGCAGGAAGTCAAAGACTTTTTCCGGTGGGCAA
AGGATCACGTGGTTGAGGTGGAGCATGAATTCTACGTCAAAAAGGGTGGAGCCAAGAAAA
GACCCGCCCCCAGTGACGCAGATATAAGTGAGCCCAAACGGGCGCGCGAGTCAGTTGCGC
AGCCATCGACGTCAGACGCGGAAGCTTCGATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAAGTGCCAGACGCTTGCAGTGCCTGCGATCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTTAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGCCTTAAAGAA
GATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAAGCGGGCCAGCAGCCT
GCAAGAAAGAGATTGAATTTCGGTCAGACTGGAGACGCAGACTCCGTACCTGACCCCCAG
CCTCTCGGACAACCACCAGCAGCCCCCTCTGGTCTGGGATCTACTACAATGGCTACAGGC
AGTGGCGCACCAATGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCC
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGGCCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCA
GCAGACGTTTTCATGGTCCCACAGTATGGATACCTCACCCTGAACAACGGGAGTCAGGCG
GTAGGACGCTCTTCCTTTTACTGCTTGGAGTACTTTCCTTCTCAGATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATCGACCAGTACCTGTATTATCTGAAC
AAGACACAATCAAATAGTGGAACACTTCAGCAGTCTCGGCTACTGTTTAGCCAAGCTGGA
CCCACCAGCATGTCTCTTCAAGCTAAAAATTGGCTGCCTGGACCTTGCTACAGACAGCAG
CGTCTGTCAAAGCAGGCAAACGACAACAACAACAGCAACTTTCCCTGGACTGCGGCTACA
AAGTATCATCTCAATGGCCGGGACTCGTTGGTTAATCCAGGACCAGCTATGGCCAGCCAC
AAAGATGATGAAGAAAAGTTTTTCCCCATGCATGGAACCCTGATATTTGGTAAACAAGGA
ACTGGAAGAGACAACGCGGATTTGGACAATGTCATGATTACAGATGAAGAAGAAATCCGC
ACCACCAATCCCGTGGCTACGGAGCAGTACGGATATGTGTCAAATAATTTGCAAAACTCA
AACACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGACTCAAACACCCACCTCCTCAGATC
ATGATTAAAAACACTCCCGTTCCAGCCAATCCTCCCACAAACTTCAGTTCTGCCAAGTTT
GCTTCTTTCATCACACAGTATTCCACGGGGCAGGTCAGCGTGGAGATCGAGTGGGAGCTG
CAGAAGGAGAACAGCAAACGCTGGAACCCCGAGATCCAGTACACTTCCAACTACAACAAA
TCTGTTAATGTGGACTTTACTGTGGACACTAATGGTGTGTATTCAGAGCCTCGCCCCATT
GGCACCAGATACCTGACTCGTAATCTGTAATTGCTTGTTAATCAATAAACCGTTTAATTC
GTTTCAGTTGAACTTTGGTCTCTGCGTACTTCTTTCTTATCTAGTTTCCATGGCTACGTA
GATAAGTAGCATGGCGGGTTAATCATTAACTACAAGGAACCCCTAGTGATGGAGTTGGCC
ACTCCCTCTCTGCGCGCTCGCTCGCTCACTGAGGCCGGGCGACCAAAGCCCGGGCGTCGG
GCGACCTTTGGTCGCCCGGCCTCAGTGAGCGAGCGAGCGCGCAGAGAGGGAGTGGCCAA
SEQ ID NO: 118
>#2320_AAV.FL.linear [organism = Adeno-associated virus][mol_type = genomic
DNA][isolation_source = liver tissue][topology = linear]
TTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTG
GCCAACTCCATCACTAGGGGTTCCTGGAGGGGTGGAGTCGTGACGTGAAGTACGTCATAG
GGTTAGGGAGGTCCTGTATTAGAGATCACGTGAGTGTTTTGCGACATTTTGCGACACCAT
GTGGTCACGCCGGTTAGTTAAGCTCGAGTGAGCACGCAGGGTCTCCATTTTGAAGCGGGA
AGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGG
AATGGGAGTTGCCGCCAGATTCTGACATGAATCTGAATCTGATTGAGCAGGCACCCCTGA
CCGTGGCCGAGAAGCTGCAGCGCGACTTTCTGACAGAATGGCGCCGCGTGAGTAAGGCCC
CGGAGGCCCTCTTCTTTGTGCAATTTGAGAAGGGAGAAAGCTATTTCCACATGCACGTGC
TGGTGGAAACCACCGGGGTGAAATCCATGGTTTTGGGACGTTTCCTGAGTCAGATTCGCG
AAAAACTGATTCAGAGAATTTACCGCGGGATCGAGCCGACTTTGCCAAATTGGTTCGCGG
TCACAAAGACCCGAAATGGCGCCGGAGGCGGGAACAAGGTGGTGGATGAGTGCTACATCC
CCAATTACCTGCTCCCCAAAACCCAGCCTGAGCTCCAGTGGGCGTGGACTAATATGGAAC
AGTATTTAAGCGCCTGTTTGAACCTCGCGGAGCGTAAACGGTTGGTGGCGCAGCATCTGA
CGCACGTGTCGCAGACCCAGGAGCAGAACAAAGAGAATCAGAATCCCAATTCTGACGCGC
CGGTGATTAGATCAAAAACCTCAGCCAGGTACATGGAGCTGGTCGGGTGGCTCGTGGATA
AGGGGATTACCTCGGAGAAGCAGTGGATCCAGGAGGACCAGGCTTCATACATCTCCTTCA
ATGCGGCCTCCAATTCGCGGTCCCAAATCAAGGCTGCTCTGGACAATGCGGGAAAGATCA
TGAGCCTGACTAAAACCGCCCCCGACTACCTGGTGGGCCAGCAGCCCGTGGAGGACATTT
CCGGCAATCGAATTTATAAAATCTTGGAACTGAATGGGTACGATCCCCAATACGCGGCTT
CCGTCTTTCTGGGATGGGCCACGAAAAAGTTCGGCAAGAGGAACACCATCTGGCTGTTTG
GGCCTGCAACTACCGGGAAGACCAACATCGCGGAAGCCATAGCCCACACGGTGCCCTTCT
ACGGGTGCGTAAACTGGACCAATGAGAACTTTCCCTTCAACGACTGTGTCGACAAGATGG
TGATCTGGTGGGAGGAGGGGAAGATGACCGCCAAGGTCGTGGAGTCGGCCAAAGCCATTC
TCGGAGGAAGCAAGGTGCGCGTGGACCAGAAATGTAAGTCCTCGGCCCAGATAGACCCGA
CTCCCGTGATCGTCACCTCCAACACCAACATGTGCGCCGTGATTGACGGGAACTCAACGA
CCTTCGAGCACCAGCAGCCGTTGCAAGACCGGATGTTCAAATTTGAACTTACCCGCCGTC
TGGATCATGACTTTGGGAAGGTCACCAAGCAGGAAGTGAAAGACTTTTTCCGGTGGGCAA
AGGATCACGTGGTTGAGGTGGAGCATGAATTCTACGTCAAAAAGGGTGGAGCTAAGAAAA
GGCCCGCCCCCAGTGACGCAGATATAAGTGAGCCCAAACGGGCGCGCGAGTCAGTTGCGC
AGCCATCGACGTCAGACGCGGAAGCTTCAATCAACTACGCGGACAGGTACCAAAACAAAT
GTTCTCGTCACGTGGGCATGAATCTGATGCTGTTTCCCTGCAGACAATGCGAGAGAATGA
ATCAGAATTCAAATATCTGCTTCACTCACGGACAGAAAGACTGTTTAGAGTGCTTTCCCG
TGTCAGAATCTCAACCCGTTTCTGTCGTCAAAAAGGCGTATCAGAAACTTTGCTACATTC
ATCATATCATGGGAAAGGTACCAGACGCTTGCACTGCTTGCGACCTGGTCAATGTGGATT
TGGATGACTGCATCTCTGAACAATAAATGATTTAAATCAGGTATGGCTGCCGATGGTTAT
CTTCCAGATTGGCTCGAGGACACTCTCTCTGAAGGAATAAGACAGTGGTGGAAGCTYAAA
CCTGGCCCACCACCACCAAAGCCCGCAGAGCGGCATAAGGACGACAGCAGGGGTCTTGTG
CTTCCTGGGTACAAGTACCTCGGACCCTTCAACGGACTCGACAAGGGAGAGCCGGTCAAC
GAGGCAGACGCCGCGGCCCTCGAGCACGACAAGGCCTACGACCGGCAGCTCGACAGCGGA
GACAACCCGTACCTCAAGTACAACCACGCCGACGCGGAGTTTCAGGAGCGTCTTAAAGAA
GATACGTCTTTTGGGGGCAACCTCGGACGAGCAGTCTTCCAGGCGAAAAAGAGGGTTCTT
GAAACTCTGGGCCTGGTTGAGGAACATGTTAAGACGGCTCCGGGTAAAAAGAGGGCGGTA
GAGCATGCTCATGTGGAGCCAGACTCCTCCTCGGGAACCGGAAAGGCGGGCCAGCAGCCT
GCAAGAAAAAGATTGAATTTTGGTCAGACTGGAGACGCAGACTCCGTACCTGACCCCCAG
CCTCTCGGACAGCCACCAGCAGCCCCCTYTGGTYTGGGATCTACTACAATGGCTTCAGGC
AGTGGCGCACCAATGGCAGACAATAACGAGGGTGCCGATGGAGTGGGTAATTCCTCAGGA
AATTGGCATTGCGATTCCCAATGGCTGGGCGACAGAGTCATCACCACCAGCACCCGAACC
TGGGCCCTGCCCACCTACAACAACCACCTCTACAAGCAAATCTCCAGCCAATCAGGAGCT
TCAAACGACAACCACTACTTTGGCTACAGCACCCCCTGGGGGTATTTTGACTTCAACAGA
TTCCACTGCCACTTTTCACCACGTGACTGGCAAAGACTCATCAACAACAACTGGGGATTC
CGACCCAAAAGACTCAACTTCAAGCTCTTTAACATTCAAGTCAAGGAGGTCACGCAGAAT
GACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTTCCA
GCGGACGTCTTCATGGTCCCACAGTATGGATACCTCACCCTGAACAACGGGAGTCAGGCG
GTAGGACGCTCTTCCTTTTACTGCCTGGAGTACTTTCCTTCTCAAATGCTGCGTACTGGA
AACAACTTTCAGTTCAGCTACACCTTTGAAGACGTTCCTTTCCACAGCAGCTACGCTCAC
AGCCAGAGTCTGGATCGGCTGATGAATCCTCTGATTGACCAGTACCTGTATTATCTGAAC
AAGACACAATCAAATAGTGGAACTCTTCAGCAGTCTCGGCTACTGTTTAGTCAAGCTGGA
CCCACCAGCATGTCTCTTCAAGCTAAAAACTGGCTGCCTGGACCTTGCTACAGACAGCAG
CGTCTGTCAAAGCAGGCAACGGATAACAACAACAGTGAATTTCCCTGGACTGCGGCTACA
AAGTATCATCTCAATGGCCGGGACTCGTTGGTTAATCCAGGACCAGCTATGGCCAGCCAC
AAAGACGATGAAGAAAAGTTTTTCCCCATGCATGGAACCCTGATATTTGGTAAACAAGGA
ACTAAATGCAACGACGCGGATTTGGACAATGTTATGATTACAGATGAAGAAGAAATCCGC
ACCACCAATCCCGTGGCTACGGAGCAGTACGGATATGTGTCAAATAATTTGCAAAACTCA
AATACTGGTCCAACTACTGGAACTGTCAATCACCAAGGAGCGTTACCTGGTATGGTGTGG
CAGGATCGAGACGTGTACCTGCAGGGACCCATTTGGGCCAAGATTCCTCACACCGATGGA
CACTTTCATCCTTCTCCACTGATGGGAGGTTTTGGACTCAAACACCCACCTCCTCAGATC
ATGATCAAAAACACTCCCGTTCCAGCCAATCCTCCCACAAACTTTAGTTCTGCCAAGTTT
GCTTCTTTCATCACACAGTATTCCACGGGGCAAGTCAGCGTGGAGATCGAGTGGGAGCTG
CAGAAGGAGAACAGCAAACGCTGGAACCCCGAGATCCAGTACACTTCCAACTACAACAAG
TCTGTTAATGTGGACTTTACTGTGGACACTAATGGCGTGTATTCAGAGCCTCGCCCCATT
GGCACCAGATACCTGACTCGTAATCTGTAATTGCTTGTTAATCAATAAACCGTGTAATTC
GTTTCAGTTGAACTTTGGTCTCCGCGTACTTCTTTTTCTTATCTAGTTTCCATGGCTACG
TAGATAAGTAGCATGGCGGGTGAATCATTAACTACAAGGAACCCCTAGTGATGGAGTTGG
CCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGAGAGGGAGTGGCCA
A
SEQ ID NO: 119
>#1055_AAV.FL.linear_Capsid sequence_ [organism = Adeno-associated virus]
[mol_type = genomic DNA][isolation_source = liver tissue][topology = linear]
GGTATGGCTGCCGATGGTTATCTTCCAGATTGGCTCGAGGACACTCTCTCTGAAGGAATA
AGACAGTGGTGGAAGCTCAAACCTGGCCCACCACCACCAAAGCCCGCAGAGCGGCATAAG
GACGACAGCAGGGGTCTTGTGCTTCCTGGGTACAAGTACCTCGGACCCTTCAACGGACTC
GACAAGGGAGAGCCGGTCAACGAGGCAGACGCCGCGGCCCTCGAGCACGACAAAGCCTAC
GACCGGCAGCTCGACAGCGGAGACAACCCGTACCTCAAGTACAACCACGCCGACGCGGAG
TTTCAGGAGCGCCTTAAAGAAGATACGTCTTTTGGGGGCAACCTCGGACGAGCAGTCTTC
CAGGCGAAAAAGAGGGTTCTTGAACCTCTGGGCCTGGTTGAGGAACCTGTTAAGACGGCT
CCGGGAAAAAAGAGGCCGGTAGAGCACTCTCCTGTGGAGCCAGACTCCTCCTCGGGAACC
GGAAAGGCGGGCCAGCAGCCTGCAAGAAAAAGATTGAATTTTGGTCAGACTGGACCTTAT
GCTTCCGTACCTGACCCCCAGCCTCTCGGACAGCCACCAGCAGCCCCCTCTGGTCTGGGA
ACTAATACGATGGCTACAGGCAGTGGCGCACCAATGGCAGACAATAACGAGGGCGCCGAC
GGAGTGGGTAATTCCTCGGGAAATTGGCATTGCGATTCCACATGGATGGGCGACAGAGTC
ATCACCACCAGCACCCGCACCTGGGCTCTGCCCACCTACAACAACCATCTGTACAAGCAG
ATTTCCAGCCAGTCTGGAGCCAGCAACGACAACCACTACTTTGGCTACAGCACCCCCTGG
GGGTATTTTGACTTCAACAGATTCCACTGCCACTTTTCGCCACGCGACTGGCAGAGACTC
ATCAACAACAACTGGGGATTCCGGCCCAAAAGACTCAACTTCAAGCTGTTTAACATTCAA
GTCAAGGAGGTCACGCAGAATGACGGTACGACGACGATTGCCAATAACCTTACCAGCACG
GTTCAGGTGTTTACTGACTCGGAGTACCAGCTCCCGTACGTCCTCGGCTCGGCGCATCAA
GGATGCCTCCCGCCGTTCCCAGCAGACGTCTTCATGGTGCCACAGTATGGATACCTCACC
CTGAACAACGGGAGTCAGGCGGTCGGACGCTCTTCCTTTTACTGCCTGGAGTACTTTCCT
TCTCAGATGCTTCGTACAGGAAACAACTTTACCTTCAGCTACACCTTTGAAGACGTTCCT
TTCCACAGCAGCTACGCTCACAGCCAGAGTCTGGACCGTCTCATGAATCCTCTCATCGAC
CAGTACCTGTATTACTTGAGCAGAACAAACACGCCGAGCGGAACCACCACGCAGTCCAGG
CTTCAGTTTTCTCAGGCCGGAGCGAGTGACATTCGGGACCAGTCTAGGAACTGGCTTCCT
GGACCCTGTTACCGCCAGCAGCGAGTATCAAAGACAGCTGCGGATAACAACAACAGTGAA
TACTCGTGGACTGGAGCTACCAAGTACCACCTCAATGGCAGAGACTCTTTGGTGAATCCG
GGCCCGGCCATGGCCAGCCACAAGGACGATGAAGAAAAGTTTTTTCCTCAAAGCGGGGTT
CTCATCTTTGGGAAGCAAGGCTCAGAGAAAACAAATGTGGACATTGAAAAGGTCATGATT
ACAGACGAAGAGGAAATCAGGACCACCAATCCCGTGGCTACGGAGCAGTATGGTGCTGTA
TCTACCAACCTCCAGAGCGGCAACACACAAGCAGCTACCTCAGATGTCAACACACAAGGC
GTTCTTCCAGGCATGGTCTGGCAGGACAGAGACGTGTACCTGCAGGGGCCCATCTGGGCA
AAGATTCCACACACGGACGGACATTTTCACCCCTCTCCCCTCATGGGCGGATTTGGACTG
AAGCACCCTCCTCCACAGATTCTCATTAAGAATACCCCAGTACCTGCGAATCCTTCTACC
ACTTTCAGCGCGGCAAAGTTTGCTTCCTTCATCACACAGTATTCCACGGGGCAGGTCAGC
GTGGAGATCGAATGGGAGCTGCAAAAGGAGAACAGCAAACGCTGGAATCCCGAAATTCAG
TACACTTCAAACTACAACAAATCTGTTAATGTGGACTTTACTGTGGACACTAATGGTGTG
TATTCAGAGCCTCGCCCCATTGGCACCAGATACCTGACTCGTAATCTGTAATTGCTTGTT
AATCAATAAACCGTGTAATTCGTTTCAGTTGAACTTTGGTCTCTGCGTACTTCTTTCTTA
TCTAGTTTCCATGGCTACGTAGATAAGTAGCATGGCGGGTTAATCATTAACACAAGGAAC
CCCTAGTGATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGAGCGAGCGC
GCAGAGAGGGAGTGGCCAA
SEQ ID NO: 120
>#M258_AAV.FL.linear_Capsid sequence_ [organism = Adeno-associated virus]
[mol_type = genomic DNA][isolation_source = liver tissue][topology = linear]
TTTAAATCAGGTATGGCTGCCGATGGTTATCTTCCAGATTGGCTCGAGGACACTCTCTCT
GAAGGAATAAGACAGTGGTGGAAGCTCAAACCTGGCCCACCACCGCCAAAGCCCGCAGAG
CGGCATAAGGACGACAGCAGGGGTCTTGTGCTTCCTGGGTACAAGTACCTCGGACCCTTC
AACGGACTCGACAAGGGAGAGCCGGTCAACGAGGCAGACGCCGCGGCCCTCGAGCACGAC
AAGGCCTACGACCGGCAGCTCGACAGCGGAGACAACCCGTACCTCAAGTACAACCACGCC
GACGCGGAGTTTCAGGAGCGCCTTAAAGAAGATACGTCTTTTGGGGGCAACCTCGGACGA
GCAGTCTTCCAGGCAAAAAAGAGGGTTCTTGAACCTCTGGGCCTGGTTGAGGAGCCTGTT
AAGACGGCTCCGGGAAAAAAGAGGCCGGTAGAGCACTCTCCTGCGGAGCCAGACTCCTCC
TCGGGAACCGGAAAAGCGGGCAACCAGCCTGCAAGAAAAAGATTGAATTTCGGTCAGACT
GGAGACGCAGACTCCGTACCTGACCCCCAGCCTCTCGGAGAACCACCAGCAGCCCCCACA
AGTTTGGGATCTACTACAATGGCTTCAGGCGGTGGCGCACCAGTGGCAGACAATAACGAG
GGTGCCGATGGAGTGGGTAATTCCTCGGGGAAATTGGCATTGCGATTCCCAATGGCTGGG
CGACAGAGTCATTACCACCAGCACCCGAACCTGGGCCCTGCCCACCTACAACAACCATCT
GTACAAGCAAATTTCCAGCCAATCAGGAGCCAGCAACGACAACCACTAATCTTGGCTCAC
TGCAACCCCTGGGGGTATTTTGACTTCAACAGATTCCACTGCCACTTTTCACCACGTGAC
TGGCAAAGACTCATCAACAACAACTGGGGATTCCGGCCCAAAAGACTCAACTTCAAGCTC
TTTAACATTCAAGTCAAAGAGGTCACGCAGAATGACGGTACGACGACGATTGCCAATAAC
CTTACCAGCACGGTTCAGGTGTTTACTGACTCGGAGTACCAGATCCCGTACGTCCTCGGC
TCGGCGCATCAAGGATGCCTCCCGCCGTTCCCAGCAGACGTCTTCATGGTGCCACAGTAT
GGATACCTCACCCTGAACAACGGGAGTCAGGCGGTAGGACGCTCTTCCTTTTACTGCTTG
GAGTACTTTCCTTCTCAGATGCTGCGTACTGGAAACAACTTCCAATTCAGCTACACCTTT
GAAGACGTTCCTTTCCACAGCAGCTACGCTCACAGCCAGAGTCTGGATCGGCTGATGAAT
CCTCTGATCGACCAGTACCTGTATTATCTGAACAAGACACAATCAGCCAGTGGAACTGTT
CAGCAGTCTCGGCTACTGTTTAGCCAAGCTGGACCCACCAGCATGTCTCTTCAAGCTAAA
AACTGGCTGCCTGGACCTTGCTACAGACAGCAGCGTCTGTCAAAGCAGGCAAACGAAAAC
AACAACAGCAACTTTCCCTGGACTGCGGCTACAAAGTATCATCTCAATGGCCGGGACTCG
TTGGTTAATCCAGGACCAGCTATGGCCAGTCACAAGGATGACGAAGAAAAGTTTTTCCCC
ATGCATGGAACCCTGATATTTGGTAAACAAGGAACTGGAAGAGACAACGCGGATTTGGAC
AATGTCATGATTACAGATGAAGAAGAAATCCGCACCACCAATCCCGTGGCTACGGAGCAG
TACGGATATGTGTCAAATAATTTGCAAAACTCAAACACTGCTCCGAGTACTGGAACTGTC
AATCACCAAGGAGCGTTACCTGGTATGGTGTGGCAGGATAGAGACGTGTACCTGCAGGGA
CCCATTTGGGCCAAGATTCCTCACACCGATGGACACTTTCATCCTTCTCCGCTGATGGGA
GGTTTTGGACTCAAACACCCACCTCCTCAGATCATGATCAAAAACACTCCCGTTCCAGCC
AATCCTCCTACAAACTTCAGTGCGGCAAAATTTGCTTCCTTTATCACACAGTATTCCACG
GGACAGGTCAGCGTGGAGATCGAGTGGGAGCTGCAGAAGGAGAACAGCAAACGCTGGAAC
CCCGAGATCCAGTACACTTCCAACTACAACAAGTCTGTTAATGTGGACTTTACTGTGGAC
ACTAATGGTGTGTATTCAGAGCCTCGCCCCATTGGCACCAGATACCTGACTCGTAATCTG
TAATTGCTTGTTAATCAATAAACCGTTTAATTCGTTTCAGTTGAACTTTGGTCTCTGCGT
ACTTCTTTTCTTATCTAGTTTCCATGGCTACGTAGATAAGTAGCATGGCGGGTTAATCAT
TAACTCTAAGGAACCCCTAGTGATGGAGTTGGCCACTCCCTCTCTGCGCGCTCGCYMSCY
SMCTSWGSCYSKSAAA
SEQ ID NO: 122
>>consensus AAV2
atggctgccgatggttatcttccagattggctcgaggacactctctctgaaggaataagacagtggtggaagct
caaacctggcccaccaccaccaaagcccgcagagcggcataaggacgacagcaggggtcttgtgcttcctgggt
acaagtacctcggacccttcaacggactcgacaagggagagccggtcaacgaggcagacgccgcggccctcgag
cacgacaaggcctacgaccggcagctcgacagcggagacaacccgtacctcaagtacaaccacgccgacgcgga
gtttcaggagcgccttaaagaagatacgtcttttgggggcaacctcggacgagcagtcttccaggcgaaaaaga
gggttcttgaacctctgggcctggttgaggaacctgttaagacggctccgggaaaaaagaggccggtagagcac
tctcctgcggagccagactcctcctcgggaaccggaaaagcgggccaccagcctgcaagaaaaagattgaattt
cggtcagactggagacgcagactccgtacctgacccccagcctctcggacagccaccagcagccccctctggtc
tgggaactaatacgatggctacaggcagtggcgcaccaatggcagacaataacgagggcgccgacggagtgggt
aattcctcgggaaattggcattgcgattccacatggatgggcgacagagtcatcaccaccagcacccgaacctg
ggctctgcccacctacaacaaccatctgtacaagcagatttccagccaatcaggagcctcaaacgacaaccact
actttggctacagcaccccctgggggtattttgacttcaacagattccactgccacttttcaccacgtgactgg
caaagactcatcaacaacaactggggattccgacccaagagactcaacttcaagctctttaacattcaagtcaa
agaggtcacgcagaatgacggtacgacgacgattgccaataaccttaccagcacggttcaggtgtttactgact
cggagtaccagctcccgtacgtcctcggctcggcgcatcaaggatgcctcccgccgttcccagcagacgtcttc
atggtgccacagtatggatacctcaccctgaacaacgggagtcaggcagtaggacgctcttcattttactgcct
ggagtactttccttctcagatgctgcgtaccggaaacaactttaccttcagctacacctttgaggacgttcctt
tccacagcagctacgctcacagccagagtttggaccgtctcatgaatcctctcatcgaccagtacctgtattac
ttgagcagaacaaacactccaagcggaaccaccacgcagtccaggcttcagttttctcaggccggagcgagtga
cattcgggaccagtctaggaactggcttcctggaccctgttaccgccagcagcgagtatcaaagacagctgcgg
ataacaacaacagtgaatactcgtggactggagctaccaagtaccacctcaatggcagagactctctggtgaat
ccgggcccggccatggccagccacaaggacgatgaagaaaagttttttcctcagagcggggttctcatctttgg
gaagcaaggctcagagaaaacaaatgtggacattgaaaaggtcatgattacagacgaagaggaaatcaggacca
ccaatcccgtggctacggagcagtatggttctgtatctaccaacctccagagcggcaacacacaagcagctacc
tcagatgtcaacacacaaggcgttcttccaggcatggtctggcaggacagagacgtgtacctgcaggggcccat
ctgggcaaagattccacacacggacggacattttcacccctctcccctcatgggcggatttggacttaaacacc
ctcctccgcagattctcatcaagaacaccccggtacctgcgaatccttcgaccaccttcagtgcggcaaagttt
gcttccttcatcacacagtactccacggggcaggtcagcgtggagatcgagtgggagctgcagaaggagaacag
caaacgctggaatcccgagatccagtacacttccaactacaacaaatctgttaatgtggactttactgtggaca
ctaatggtgtgtattcagagcctcgccccattggcaccagatacctgactcgtaatctgtaa

AAV Comprising a Capsid Protein of the Present Invention:

A further object of the present invention relates to an adeno-associated virus (AAV) that comprises at least one capsid protein of the present invention.

In some embodiments, the AAV is a wildtype AAV. In some embodiments, the AAV comprises a genome sequence as set forth in SEQ ID NO: 62-120.

A further object of the present invention relates to an AAV wherein the amino acid sequence of VP1 capsid protein is encoded by the nucleotides 2203-4410 of in SEQ ID NO: 85 (i.e. capsid #3013), SEQ ID NO: 68 (i.e. capsid #2087), SEQ ID NO: 79 (i.e. capsid #1449), SEQ ID NO: 69 (i.e. capsid #2206), SEQ ID NO: 71 (i.e. capsid #1534), SEQ ID NO: 115 (i.e. capsid #163), SEQ ID NO: 82 (i.e. capsid #1343), SEQ ID NO: 112 (i.e. capsid #790), SEQ ID NO:65 (i.e. capsid #1017), SEQ ID NO: 70 (i.e. capsid #877), SEQ ID NO: 67 (i.e. capsid #1273), SEQ ID NO: 62 (i.e. capsid #2497); SEQ ID NO: 119 (i.e. capsid #1055); SEQ ID NO: 73 (i.e. capsid #1919), SEQ ID NO:63, SEQ ID NO:64; SEQ ID NO:66, SEQ ID NO: 72, SEQ ID NO: 74, SEQ ID NO: 75, SEQ ID NO: 76, SEQ ID NO: 77, SEQ ID NO: 78, SEQ ID NO:80, SEQ ID NO:81, SEQ ID NO:83, SEQ ID NO:84, SEQ ID NO:86, SEQ ID NO:87; SEQ ID NO:88; SEQ ID NO:89; SEQ ID NO:90; SEQ ID NO: 91; SEQ ID NO:92; SEQ ID NO:93; SEQ ID NO:94; SEQ ID NO:95; SEQ ID NO:96; SEQ ID NO:97; SEQ ID NO:98, SEQ ID NO: 99; SEQ ID NO:100; SEQ ID NO:101; SEQ ID NO:102; SEQ ID NO:103, SEQ ID NO:104; SEQ ID NO:105, SEQ ID NO: 106; SEQ ID NO:107, SEQ ID NO:108, SEQ ID NO:109, SEQ ID NO: 110; SEQ ID NO:111, SEQ ID NO:113; SEQ ID NO:114; SEQ ID NO:116, SEQ ID NO: 117; SEQ ID NO:118 or SEQ ID NO:120; or, the amino acid sequence of VP2 capsid protein is encoded by nucleotides 2614-4410 of in SEQ ID NO: 85 (i.e. capsid #3013), SEQ ID NO: 68 (i.e. capsid #2087), SEQ ID NO: 79 (i.e. capsid #1449), SEQ ID NO: 69 (i.e. capsid #2206), SEQ ID NO: 71 (i.e. capsid #1534), SEQ ID NO: 115 (i.e. capsid #163), SEQ ID NO: 82 (i.e. capsid #1343), SEQ ID NO: 112 (i.e. capsid #790), SEQ ID NO:65 (i.e. capsid #1017), SEQ ID NO: 70 (i.e. capsid #877), SEQ ID NO: 67 (i.e. capsid #1273), SEQ ID NO: 62 (i.e. capsid #2497); SEQ ID NO: 119 (i.e. capsid #1055); SEQ ID NO: 73 (i.e. capsid #1919), SEQ ID NO:63, SEQ ID NO:64; SEQ ID NO:66, SEQ ID NO: 72, SEQ ID NO: 74, SEQ ID NO: 75, SEQ ID NO: 76, SEQ ID NO: 77, SEQ ID NO: 78, SEQ ID NO:80, SEQ ID NO:81, SEQ ID NO:83, SEQ ID NO:84, SEQ ID NO:86, SEQ ID NO:87; SEQ ID NO:88; SEQ ID NO:89; SEQ ID NO:90; SEQ ID NO: 91; SEQ ID NO:92; SEQ ID NO:93; SEQ ID NO:94; SEQ ID NO:95; SEQ ID NO:96; SEQ ID NO:97; SEQ ID NO:98, SEQ ID NO: 99; SEQ ID NO:100; SEQ ID NO:101; SEQ ID NO:102; SEQ ID NO:103, SEQ ID NO:104; SEQ ID NO:105, SEQ ID NO: 106; SEQ ID NO:107, SEQ ID NO:108, SEQ ID NO:109, SEQ ID NO: 110; SEQ ID NO:111, SEQ ID NO:113; SEQ ID NO:114; SEQ ID NO:116, SEQ ID NO: 117; SEQ ID NO:118 or SEQ ID NO:120 and the amino acid sequence of VP3 capsid protein is encoded by nucleotides 2809-4410 of in SEQ ID NO: 85 (i.e. capsid #3013), SEQ ID NO: 68 (i.e. capsid #2087), SEQ ID NO: 79 (i.e. capsid #1449), SEQ ID NO: 69 (i.e. capsid #2206), SEQ ID NO: 71 (i.e. capsid #1534), SEQ ID NO: 115 (i.e. capsid #163), SEQ ID NO: 82 (i.e. capsid #1343), SEQ ID NO: 112 (i.e. capsid #790), SEQ ID NO:65 (i.e. capsid #1017), SEQ ID NO: 70 (i.e. capsid #877), SEQ ID NO: 67 (i.e. capsid #1273), SEQ ID NO: 62 (i.e. capsid #2497); SEQ ID NO: 119 (i.e. capsid #1055); SEQ ID NO: 73 (i.e. capsid #1919), SEQ ID NO:63, SEQ ID NO:64; SEQ ID NO:66, SEQ ID NO: 72, SEQ ID NO: 74, SEQ ID NO: 75, SEQ ID NO: 76, SEQ ID NO: 77, SEQ ID NO: 78, SEQ ID NO:80, SEQ ID NO:81, SEQ ID NO:83, SEQ ID NO:84, SEQ ID NO:86, SEQ ID NO:87; SEQ ID NO:88; SEQ ID NO:89; SEQ ID NO:90; SEQ ID NO: 91; SEQ ID NO:92; SEQ ID NO:93; SEQ ID NO:94; SEQ ID NO:95; SEQ ID NO:96; SEQ ID NO:97; SEQ ID NO:98, SEQ ID NO: 99; SEQ ID NO:100; SEQ ID NO:101; SEQ ID NO:102; SEQ ID NO:103, SEQ ID NO:104; SEQ ID NO:105, SEQ ID NO: 106; SEQ ID NO:107, SEQ ID NO:108, SEQ ID NO:109, SEQ ID NO: 110; SEQ ID NO:111, SEQ ID NO:113; SEQ ID NO:114; SEQ ID NO:116, SEQ ID NO: 117; SEQ ID NO:118 or SEQ ID NO:120.

In some embodiments, the AAV of the present invention is a rAAV that comprises a heterologous nucleic acid comprising a transgene.

In some embodiments, the transgene encodes for a therapeutic protein. The term “therapeutic protein” refers to a protein which is defective or missing from the subject in question, thus resulting in a disease state or disorder in the subject, or to a protein which confers a benefit to the subject in question, such as an antiviral, antibacterial or antitumor function. A therapeutic protein can also be one which modifies any one of a wide variety of biological functions, such as endocrine, immunological and metabolic functions. Typically, the transgene may encode for a polypeptide involved in immune response, hematopoiesis, inflammation, cell growth and proliferation, cell lineage differentiation, or stress response.

In some embodiments, the transgene encodes for a site-specific endonuclease that provides for site-specific knock-down of gene function, e.g., where the endonuclease knocks out an allele associated with a disease. For example, where a dominant allele encodes a defective copy of a gene that, a site-specific endonuclease can be targeted to the defective allele and knock out the defective allele. In some embodiments, the rAAV of the present invention comprises a polynucleotide that encodes a site-specific endonuclease; and a polynucleotide that encodes a functional copy of a defective allele, where the functional copy encodes a functional protein. Site-specific endonucleases that are suitable for use include, e.g., zinc finger nucleases (ZFNs); transcription activator-like effector nucleases (TALENs), and CRISPR-associated endonuclease. As used herein, the term “CRISPR-associated endonuclease” has its general meaning in the art and refers to clustered regularly interspaced short palindromic repeats associated which are the segments of prokaryotic DNA containing short repetitions of base sequences. In particular, the CRISPR-associated endonuclease is Cas9 or a derivative thereof. The Cas9 nuclease can have a nucleotide sequence identical to the wild type Streptococcus pyrogenes sequence. Alternatively, the wild type Streptococcus pyrogenes Cas9 sequence can be modified. For instance, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of PX330 or PX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). For example the Cas9 nuclease can be mutated in the conserved FiNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. In some embodiments, the rAAV of the present invention comprises one or more guide RNA. As used herein, the term “one or more guide RNA” refers to the RNAs that guide the insertion or deletion of residues. In the context of the invention, the guide RNA is used for recruiting Cas9 to specific genomic loci. In some embodiments, the guide RNA can be a sequence complementary to a coding or a non-coding sequence. In some embodiments, the subject is administered with a combination of at least one vectors comprising one polynucleotide encoding for a Cas9 endonuclease and at least one vector comprising the guide RNA.

In some embodiments, the transgene encodes for an interfering RNA (RNAi), in particular a siRNA. A “small interfering” or “short interfering RNA” or siRNA is a RNA duplex of nucleotides that is targeted to a gene interest (a “target gene”). An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides.

In some embodiments, the transgene encodes for an antisense oligonucleotide. As used herein, the term “antisense oligonucleotide” is understood to refer to a nucleotide sequence which is substantially complementary to a target nucleotide sequence in a pre-mRNA molecule, hnRNA (heterogenous nuclear RNA) or mRNA molecule. The degree of complementarity (or substantial complementarity) of the antisense sequence is preferably such that a molecule comprising the antisense sequence can form a stable hybrid with the target nucleotide sequence in the RNA molecule under physiological conditions.

Other transgenes of interest include, without limitation, synthetic long non-coding RNAs (SINEUPs; Carrieri et al., 2012, Nature 491: 454-7; Zucchelli et al., 2015, RNA Biol 12(8): 771-9; Indrieri et al., 2016, Sci Rep 6: 27315) and artificial microRNAs. Other specific transgene of interest useful in the practice of the present invention are described below.

In some embodiments, the transgene is operably linked to a constitutive promoter. In some embodiments the transgene is operably linked to an inducible promoter. In some embodiments, the transgene is operably linked to a tissue-specific or cell type-specific regulatory element.

Numerous methods are known in the art for production of rAAV vectors, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a transgene (such as a therapeutic transgene) flanked by at least one AAV ITR sequences; and 5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors. The rAAV particles can be produced using methods known in the art. See, e.g., U.S. Pat. Nos. 6,566,118; 6,989,264; and 6,995,006. In practicing the invention, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or HeLa cells. AAV vectors are purified and formulated using standard techniques known in the art.

In some embodiments, rAAV particles may be produced by a triple transfection method, such as the exemplary triple transfection method provided infra. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified.

In some embodiments, rAAV particles may be produced by a producer cell line method, such as the exemplary producer cell line method provided infra (see also (referenced in Martin et al., (2013) Human Gene Therapy Methods 24:253-269). Briefly, a cell line (e.g., a HeLa cell line) may be stably transfected with a plasmid containing a rep gene, a capsid gene, and a promoter-transgene sequence. Cell lines may be screened to select a lead clone for rAAV production, which may then be expanded to a production bioreactor and infected with an adenovirus (e.g., a wild-type adenovirus) as helper to initiate rAAV production. Virus may subsequently be harvested, adenovirus may be inactivated (e.g., by heat) and/or removed, and the rAAV particles may be purified.

In some embodiments, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein; (ii) a rAAV pro-vector comprising a transgene flanked by at least one AAV ITR, and (iii) an AAV helper function; and (b) recovering the rAAV particles produced by the host cell. rAAV vector particles of the invention may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In some embodiments, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like. rAAV particles may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. In some embodiments, the method comprises all the steps in the order as described below. Methods to purify rAAV particles are found, for example, in Xiao et al., (1998) Journal of Virology 72:2224-2232; U.S. Pat. Nos. 6,989,264 and 8,137,948; and WO 2010/148143.

Therapeutic Uses and Pharmaceutical Compositions:

The rAAV of the present invention are particularly suitable for delivering the transgene to a cell of interest. Accordingly, a further object of the present invention relates to a method of delivering a transgene to a cell comprising contacting the cell with an amount of a rAAV of the present invention.

In particular, the rAAV of the present invention are particularly suitable for gene therapy. Gene therapy consists in introducing in tissue of interest a functional copy of a transgene (e.g. a gene) that is deficient therein (gene replacement therapy), or by delivering to the tissue a transgene which will have a beneficial effect on the disease to be treated (symptomatic therapy). In some embodiments, the rAAV of the present invention is particularly suitable for delivering a transgene in liver.

Thus a further object of the present invention relates to a method for delivering a transgene to a tissue in a subject, the method comprising administering to the subject: an effective amount of a rAAV of the present invention.

More specifically, the present invention provides a method of therapy in a patient in need thereof, comprising administering to the patient a therapeutically effective amount of a rAAV of the present invention.

A further object of the present invention relates to a pharmaceutical composition comprising the rAAV of the present invention with pharmaceutically acceptable excipients, and optionally sustained-release matrices, such as biodegradable polymers. The pharmaceutical composition of the present invention thus comprises a pharmaceutically acceptable excipient, carrier, buffer, stabiliser or other materials well known to those skilled in the art. A pharmaceutically acceptable carrier or excipient refers to a non-toxic solid, semi-solid or liquid filler, diluent, encapsulating material or formulation auxiliary of any type. Such materials should be non-toxic and should not interfere with the efficacy of the active ingredient (i.e. the rAAV of the present invention of the invention). The precise nature of the carrier or other material may be determined by the skilled person according to the route of administration, i.e. here subretinal injection. The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. For injection, the active ingredient will be in the form of an aqueous solution, which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required. For delayed release, the rAAV of the present invention may be included in a pharmaceutical composition, which is formulated for slow release, such as in microcapsules formed from biocompatible polymers or in liposomal carrier systems according to methods known in the art. Typically, the pharmaceutical composition of the present invention is supplied in a prefilled syringe. A “ready-to-use syringe” or “prefilled syringe” is a syringe which is supplied in a filled state, i.e. the pharmaceutical composition to be administered is already present in the syringe and ready for administration. Prefilled syringes have many benefits compared to separately provided syringe and vial, such as improved convenience, affordability, accuracy, sterility, and safety. The use of prefilled syringes results in greater dose precision, in a reduction of the potential for needle sticks injuries that can occur while drawing medication from vials, in pre-measured dosage reducing dosing errors due to the need to reconstituting and/or drawing medication into a syringe, and in less overfilling of the syringe helping to reduce costs by minimising drug waste. In some embodiments the pH of the liquid pharmaceutical composition of the present invention is in the range of 5.0 to 7.0, 5.1 to 6.9, 5.2 to 6.8, 5.3 to 6.7 or 5.4 to 6.6.

The invention will be further illustrated by the following figures and examples. However, these examples and figures should not be interpreted in any way as limiting the scope of the present invention.

### FIGURES

FIG. 1. AAV Full-length sequences in 57 human liver tissues. A) Location of the two open reading frames encoding replication proteins (Rep78, Rep68, Rep52 and Rep40), structural proteins (VP1, VP2 and VP3) and AAP protein coding by AAV genome. Promoters (p5, p19 and p40) are indicated with arrows. B) Amino acid variations compared to the reference AAV2 are indicated. The triangles indicate genome location of specific AAV2/13 (top) or AAV2 (middle) variants in the series of 57 human liver AAV isolates. Common variants shared by both genotypes are shown (bottom). Grey and black colors refer to silent and missense AAV variants, respectively; numbers correspond to wild-type AAV2 nucleotide sequence coordinates (NC_001401).

FIG. 2. All new capsids detarget the liver in this mouse model

Luciferase activity of controls and new capsids capsids. Each column represents the average of activity in at least 3 mice expressed as fold change versus AAV2. Standard deviations are displayed. On the right, the phylogenetic tree of the VP1 amino acid sequences was constructed using the Neighbor-Joining method. The evolutionary distances were computed using the Poisson correction method and are in the units of the number of amino acid substitutions per site.

FIG. 3. The new capsids efficiently target the muscles

A-C). Luciferase activity of controls and new capsids. Each column represents the average of activity in at least 3 mice expressed as fold change versus AAV2. Standard deviations are displayed. Statistical analysis on fold change was performed using one-way ANOVA. Dunnett's multiple comparison test was used to compare the mean of each capsid with the mean of the controls (* versus PBS, # versus AAV2 and $ versus AAV*). *, #, $=p<0.05; **, ##, $$=p<0.01; ***, ###, $$$=p<0.001.

### EXAMPLE 1: ADENO-ASSOCIATED VIRUS IN THE LIVER: NATURAL HISTORY

Results:

Identification of Two Major AAV Genotypes in the Liver

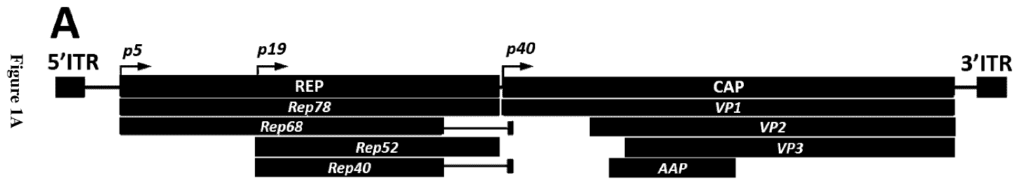

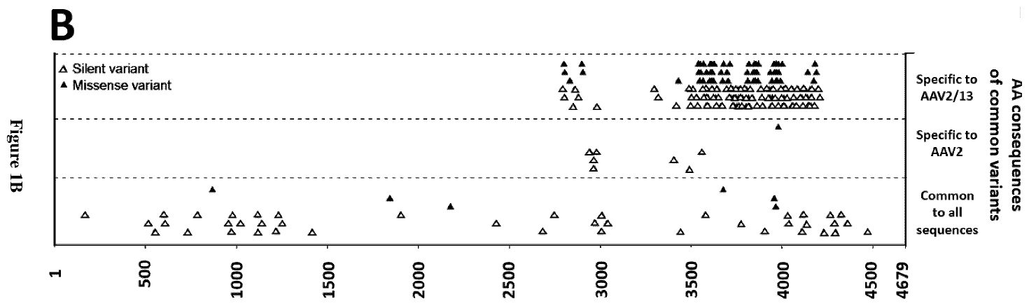

We screened frozen liver tissues from 1,319 patients for the presence of AAV viral DNA with 6 Taqman probes distributed along the genome that collectively recognize all known AAV genotypes 1 to 13. We identified AAV DNA in 18% of non-tumor liver tissues (n=233), 82 of which were further analyzed by viral capture with 120-mer oligonucleotide probes covering the entire genome of all known AAV genotypes (350 probes/genotype). After sequencing, a complete coverage of the viral genome was obtained in 57 samples, which allowed to reconstruct full-length AAV sequences (FIG. 1A). These sequences revealed two major AAV subtypes. The first subtype (n=25) is highly similar to AAV2 reference sequence (NC_001401) also corresponding to VP1 Clade B genotype isolated in human^{14, 43} (data not shown). The second subtype (n=32), here named AAV2/13, showed hybrid sequences including various parts of the AAV13 capsid and c-ter in the context of an AAV2 5′part (FIG. 1B). VP1 sequences of this subtype corresponded to various pieces of AAV13 and were previously termed Clade C (data not shown).^{14, 43} We identified several variants shared by both AAV subtypes, but different from the AAV2 reference NC_001401, corresponding mostly to positions common in AAV2, AAV3 or AAV13, silent at the protein level (n=42, 87.5%) and distributed along the viral genome (FIG. 1B). In contrast, several nucleotide variants leading to amino acid substitutions in AAV2/13 sequences were located in the hypervariable regions (HVRs) 5, 6 7 and 10 and originated from AAV13 sequence (FIG. 1B). By screening the overall series of 1,319 samples with two probes specific of AAV2/13 subtype and located in the CAP2 region (data not shown), we determined the AAV genotype in 143 samples and identified an equal number of the two AAV subtypes (47.6% AAV2 and 52.4% AAV2/13 genotypes).

AAV Infection and Episomal Form

In the 233 liver samples positive for AAV DNA, quantification of AAV followed a bimodal distribution with 97% of the tissues showing a low number of copy/cell (ranging from 4.6e-05 to 0.04) and only 8 patients with higher quantity of AAV ranging from 0.07 to 0.18 copy/cell (data not shown). Relationship between AAV detection, clinical and histological features of the patients showed that AAV was significantly enriched in female (p<0.001), young patients (p=0.016) and occurred more frequently in a background of non-fibrotic liver (p<0.001; data not shown).

In 64/233 (27.5%) of the tissues positive for AAV, all the genomic AAV regions were amplified suggesting the presence of the entire viral genome. In order to experimentally search for episomal AAV form, we designed a DNAse/TaqMan based assay (data not shown) which allowed to detected episomal AAV in 60 patients, corresponding to 26% of AAV positive samples and 4.5% of all patients. Using in silico analyses of the AAV capture sequencing, among the 57 cases with a complete reconstructed AAV genomic sequence, we identified 14 cases with 3′ITR-5′ITR junctions without clonal insertion. The 3′ITR-5′ITR junctions showed various sequences presenting a double-D ITR structure, in flip or flop configuration, with a 125 bp deletion confirmed by Sanger sequencing (data not shown).

AAV Transcription is Associated with Episomal Form

Then, we screened for AAV RNA expression in 101 non-tumor liver tissues positive for AAV by qRT-PCR. AAV transcript was identified in 64% of the tested liver tissues. We showed that either AAV REP or CAP expression were enriched in liver tissues with episomal form (p<0.001) and both transcripts were more frequently associated in presence of episomal than not-episomal AAV form (p=0.022), defining a population of patients with an “episomal-expressed AAV” (data not shown). A higher AAV copies per cell was identified in liver tissues with episomal-expressed AAV, supporting the hypothesis of a viral active infection in these liver samples (data not shown). Episomal AAV were also more frequent in female (p<0.001) and non-cirrhotic patients (p<0.001; data not shown). Analysis of AAV positivity in function of age showed a peak of frequency at 25% in the 30-40 years class. AAV episomal form was more frequent in young patients (<40 years old) reaching the highest frequency level in the twenties (data not shown). These results suggest that AAV active infection is more frequent in the second and third decade during life, while inactive not-episomal forms subsist after the primary infection.

Discussion:

In this study, we provided a comprehensive description at large scale of wild type AAV infection in the liver and of its oncogenic consequences in human. Despite the large number of studies available on recombinant AAV vectors (rAAV), the natural history of wild type AAV infection is still poorly understood. Here, we provided an extensive characterization of the different AAV viral forms in the largest series of tumor and non-tumor liver tissues to better understand the AAV infection in human.

The prevalence of AAV was observed in 21% of patients in non-tumor and/or tumor liver in agreement with the seroprevalence of antibody against AAV identified in 40 to 80% of the general population.¹⁰⁻¹² Our result showed that half of the patients demonstrate persistent AAV DNA in the liver during life, mainly in the population of young and female patients without liver fibrosis. However, since most of our liver tissues were sampled from patients with liver diseases, the exact prevalence of AAV DNA in the liver of healthy individuals remains to be evaluated.

Two major AAV genotypes, AAV2 and hybrids AAV2/13, were identified in our cohort. The classification of AAV viruses is currently based on their serological response and on the amino acid sequence of the VP1 region defined by Gao and colleagues.¹⁴ However, this classification in viral clades does not include all the AAV serotypes and the most recently isolated viral clones from non-human primates.^{16, 46} Moreover, most of the sequences isolated from human tissues were restricted to VP1 region, in particular only one full-length AAV sequence from clade C was publicly available.⁴³ Here, we provided the whole AAV genome sequences of 57 human clones classified with half of them belonging to the reference AAV2 sequence, whereas the others were hybrids between AAV2 in the 5′part and AAV13 in the 3′ corresponding to the previous clade C of the VP1 classification. Our work dramatically increased the number human AAV full-length sequences publicly available enlightening the genotypes and genomic variants associated with an efficient natural AAV infection of the liver.

In conclusion, these findings are important to understand wild type AAV biology. Our data are particularly relevant considering the large usage of AAV vector in liver-targeted gene therapy.

### EXAMPLE 2: PRODUCTION, IN VITRO AND IN VIVO TESTING OF NATURAL VARIANTS IDENTIFIED

Material and Methods

Plasmid Construction for New Serotypes

To construct the plasmid containing AAV2 Rep sequence and the new Cap genes, the capsid sequences were synthesized (GENEWIZ). The fragment was inserted in the plasmid pAAV2 which contains AAV2 Rep and AAV2 Cap in order to replace the AAV2 Cap with the corresponding new Cap sequence.

AAV Production

HEK293T cells were grown in suspension in 50 mL of serum-free medium. The cells were transfected with 3 plasmids: i) a transgene plasmid, containing AAV2 ITRs flanking an expression cassette ii) the helper plasmid pXX6, containing adenoviral sequences necessary for AAV production, and iii) a plasmid containing AAV Rep and Cap genes, defining the serotype of AAV. Two days after transfection, the cells were lysed to release the AAV particles.

The viral lysate was purified by affinity chromatography. Viral genomes were quantified by a TaqMan real-time PCR assay using primers and probes corresponding to the ITRs of the AAV vector genome (Rohr et al., J. Virol. Methods, 2002, 106, 81-88).

In Vivo Studies

All mouse studies were performed according to the French and European legislation on animal care and experimentation (2010/63/EU) and approved by the local institutional ethical committee (protocol no. 2016-002C). AAV vectors were administered intravenously via the tail vein to 6 weeks old male C57B16/J mice. PBS-injected littermates were used as controls. 15 days after vector injections, tissues were harvested and homogenized in DNAse/RNAse free water using Fastprep tubes (6.5 m/s; 60 seconds).

Luciferase Activity

Luciferase assay was used to measure the expression of the reporter gene used as transgene. Tissue lysates were centrifuged at 10000 rpm for 10 min, the supernatant was diluted in lysis buffer in a white opaque 96-well plate. Luciferase activity was measured using EnSpire (PerkinElmer) through sequential injections of assay buffer containing ATP and luciferine.

Protein quantification was performed on the samples using BCA assay in order to normalize the RLU (relative luminescence unit) on the quantity protein. The final results were expressed as RLU/mg of protein and normalized as fold change versus AAV2 control.

Results:

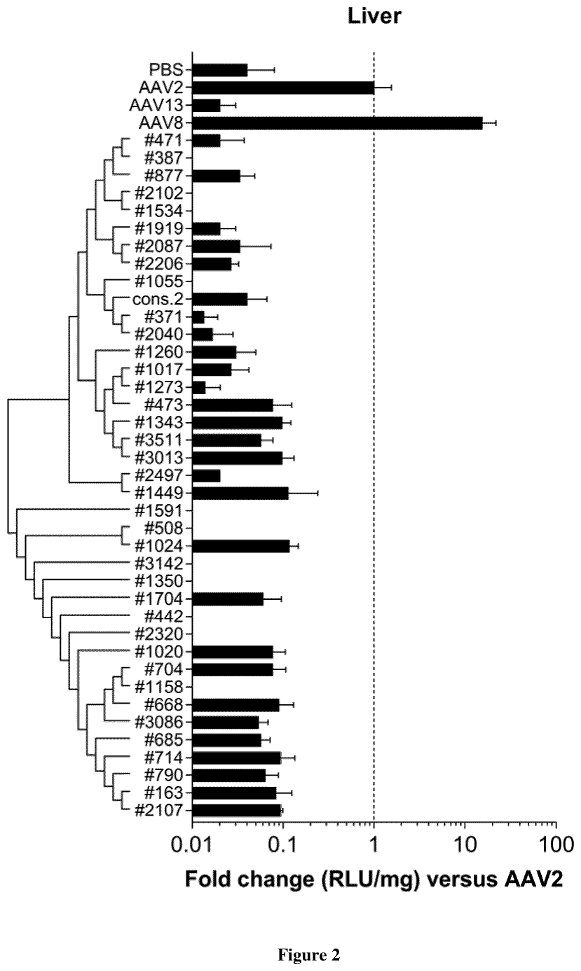

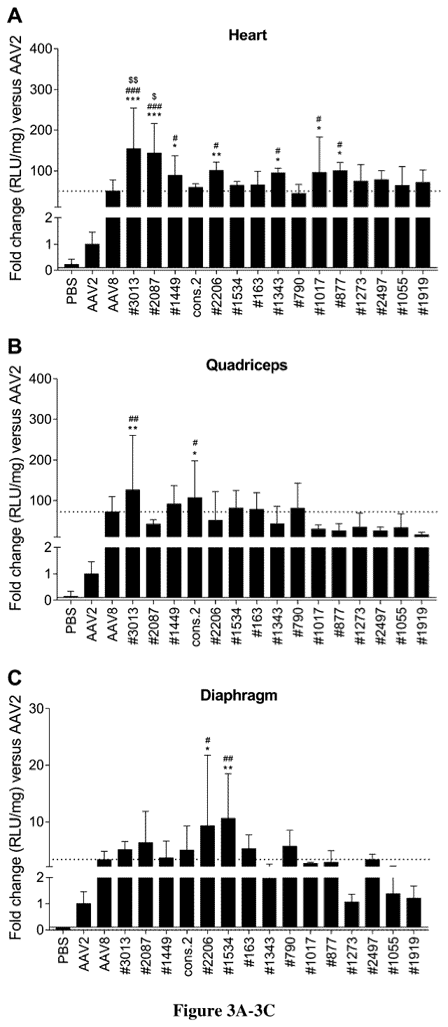

Recombinant AAV vectors were produced by cloning the Cap genes identified in EXAMPLE 1 in a plasmid suitable for vector production. A transgene expression cassette flanked by AAV2 ITRs and expressing a luciferase reporter gene was encapsidated in the so derived AAV vectors. Triple transfection of HEK293 cells was used to produce the vectors followed by immunoaffinity column purification. The capsid sequences that were not efficiently produced as rAAV vectors were excluded. The vectors were tested in wild-type C57Bl6/J mice through intravenous injection of the different vectors at the dose of 1×10¹¹ vg/mice. Fifteen days post-injection, animals were sacrificed and the levels of expression of the transgene were measured in isolated tissues. All new capsids detargeted the liver (FIG. 2) and displayed a marked tropism for the tested tissues: heart, quadriceps and diaphragm. The average fold change of transgene expression in the three tissues tested was used to rank the capsids according to their efficiency. The capsids represented in FIG. 3A-3C are the top scoring in muscle targeting.

### REFERENCES

Throughout this application, various references describe the state of the art to which this invention pertains. The disclosures of these references are hereby incorporated by reference into the present disclosure.
1. Rose J A, Berns K I, Hoggan M D, et al. Evidence for a single-stranded adenovirus-associated virus genome: formation of a DNA density hybrid on release of viral DNA. Proc Natl Acad Sci USA 1969; 64:863-9.
2. Atchison R W, Casto B C, Hammon W M. Adenovirus-Associated Defective Virus Particles. Science 1965; 149:754-6.
3. Balakrishnan B, Jayandharan G R. Basic biology of adeno-associated virus (AAV) vectors used in gene therapy. Curr Gene Ther 2014; 14:86-100.
4. Sonntag F, Schmidt K, Kleinschmidt J A. A viral assembly factor promotes AAV2 capsid formation in the nucleolus. Proc Natl Acad Sci USA 2010; 107:10220-5.
5. Yang C C, Xiao X, Zhu X, et al. Cellular recombination pathways and viral terminal repeat hairpin structures are sufficient for adeno-associated virus integration in vivo and in vitro. J Virol 1997; 71:9231-47.
6. Nakai H, Wu X, Fuess S, et al. Large-scale molecular characterization of adeno-associated virus vector integration in mouse liver. J Virol 2005; 79:3606-14.
7. Huser D, Gogol-Doring A, Chen W, et al. Adeno-associated virus type 2 wild-type and vector-mediated genomic integration profiles of human diploid fibroblasts analyzed by third-generation PacBio DNA sequencing. J Virol 2014; 88:11253-63.
8. Schultz B R, Chamberlain J S. Recombinant adeno-associated virus transduction and integration. Mol Ther 2008; 16:1189-99.
9. Schnepp B C, Jensen R L, Chen C L, et al. Characterization of adeno-associated virus genomes isolated from human tissues. J Virol 2005; 79:14793-803.
10. Boutin S, Monteilhet V, Veron P, et al. Prevalence of serum IgG and neutralizing factors against adeno-associated virus (AAV) types 1, 2, 5, 6, 8, and 9 in the healthy population: implications for gene therapy using AAV vectors. Hum Gene Ther 2010; 21:704-12.
11. Calcedo R, Vandenberghe L H, Gao G, et al. Worldwide epidemiology of neutralizing antibodies to adeno-associated viruses. J Infect Dis 2009; 199:381-90.
12. Erles K, Sebokova P, Schlehofer J R. Update on the prevalence of serum antibodies (IgG and IgM) to adeno-associated virus (AAV). J Med Virol 1999; 59:406-11.
13. Gao G P, Alvira M R, Wang L, et al. Novel adeno-associated viruses from rhesus monkeys as vectors for human gene therapy. Proc Natl Acad Sci USA 2002; 99:11854-9.
14. Gao G, Vandenberghe L H, Alvira M R, et al. Clades of Adeno-associated viruses are widely disseminated in human tissues. J Virol 2004; 78:6381-8.
15. Mori S, Wang L, Takeuchi T, et al. Two novel adeno-associated viruses from cynomolgus monkey: pseudotyping characterization of capsid protein. Virology 2004; 330:375-83.
16. Schmidt M, Voutetakis A, Afione S, et al. Adeno-associated virus type 12 (AAV12): a novel AAV serotype with sialic acid- and heparan sulfate proteoglycan-independent transduction activity. J Virol 2008; 82:1399-406.
17. Chandler R J, Sands M S, Venditti C P. Recombinant Adeno-Associated Viral Integration and Genotoxicity: Insights from Animal Models. Hum Gene Ther 2017; 28:314-322.
18. Hermonat P L, Muzyczka N. Use of adeno-associated virus as a mammalian DNA cloning vector: transduction of neomycin resistance into mammalian tissue culture cells. Proc Natl Acad Sci USA 1984; 81:6466-70.
19. Berns K I, Muzyczka N. AAV: An Overview of Unanswered Questions. Hum Gene Ther 2017; 28:308-313.
20. Donsante A, Miller D G, Li Y, et al. AAV vector integration sites in mouse hepatocellular carcinoma. Science 2007; 317:477.
21. Chandler R J, LaFave M C, Varshney G K, et al. Vector design influences hepatic genotoxicity after adeno-associated virus gene therapy. J Clin Invest 2015; 125:870-80.
22. Wang P R, Xu M, Toffanin S, et al. Induction of hepatocellular carcinoma by in vivo gene targeting. Proc Natl Acad Sci USA 2012; 109:11264-9.
23. Hermonat P L, Plott R T, Santin A D, et al. Adeno-associated virus Rep78 inhibits oncogenic transformation of primary human keratinocytes by a human papillomavirus type 16-ras chimeric. Gynecol Oncol 1997; 66:487-94.
24. Liu T, Cong M, Wang P, et al. Adeno-associated virus Rep78 protein inhibits Hepatitis B virus replication through regulation of the HBV core promoter. Biochem Biophys Res Commun 2009; 385:106-11.
25. Kokorina N A, Santin A D, Li C, et al. Involvement of protein-DNA interaction in adeno-associated virus Rep78-mediated inhibition of HIV-1. J Hum Virol 1998; 1:441-50.
26. Nault J C, Datta S, Imbeaud S, et al. Recurrent AAV2-related insertional mutagenesis in human hepatocellular carcinomas. Nat Genet 2015; 47:1187-93.
27. Zhao L H, Liu X, Yan H X, et al. Genomic and oncogenic preference of HBV integration in hepatocellular carcinoma. Nat Commun 2016; 7:12992.
28. Sung W K, Zheng H, Li S, et al. Genome-wide survey of recurrent HBV integration in hepatocellular carcinoma. Nat Genet 2012; 44:765-9.
29. Llovet J M, Zucman-Rossi J, Pikarsky E, et al. Hepatocellular carcinoma. Nat Rev Dis Primers 2016; 2:16018.
30. Haberman R P, McCown T J, Samulski R J. Novel transcriptional regulatory signals in the adeno-associated virus terminal repeat A/D junction element. J Virol 2000; 74:8732-9.
31. Li H, Durbin R. Fast and accurate long-read alignment with Burrows-Wheeler transform. Bioinformatics 2010; 26:589-95.
32. Li H, Handsaker B, Wysoker A, et al. The Sequence Alignment/Map format and SAMtools. Bioinformatics 2009; 25:2078-9.
33. Quinlan A R, Hall I M. BEDTools: a flexible suite of utilities for comparing genomic features. Bioinformatics 2010; 26:841-2.
34. Bayard Q, Meunier L, Peneau C, et al. Cyclin A2/E1 activation defines a hepatocellular carcinoma subclass with a rearrangement signature of replication stress. Nat Commun 2018; 9:5235.
35. Kim D, Pertea G, Trapnell C, et al. TopHat2: accurate alignment of transcriptomes in the presence of insertions, deletions and gene fusions. Genome Biol 2013; 14:R36.
36. Trapnell C, Roberts A, Goff L, et al. Differential gene and transcript expression analysis of RNA-seq experiments with TopHat and Cufflinks. Nat Protoc 2012; 7:562-78.
37. Sloutskin A, Danino Y M, Orenstein Y, et al. ElemeNT: a computational tool for detecting core promoter elements. Transcription 2015; 6:41-50.
38. Nishikawa T, Ota T, Isogai T. Prediction whether a human cDNA sequence contains initiation codon by combining statistical information and similarity with protein sequences. Bioinformatics 2000; 16:960-7.
39. Liu H, Han H, Li J, et al. An in-silico method for prediction of polyadenylation signals in human sequences. Genome Inform 2003; 14:84-93.
40. Benaglia T, Chauveau D, Hunter D R, et al. mixtools: An R Package for Analyzing Mixture Models. Journal of Statistical Software 2009; 32:1-29.
41. Werle-Lapostolle B, Bowden S, Locarnini S, et al. Persistence of cccDNA during the natural history of chronic hepatitis B and decline during adefovir dipivoxil therapy. Gastroenterology 2004; 126:1750-8.
42. Mroske C, Rivera H, Ul-Hasan T, et al. A capillary electrophoresis sequencing method for the identification of mutations in the inverted terminal repeats of adeno-associated virus. Hum Gene Ther Methods 2012; 23:128-36.
43. Chen C L, Jensen R L, Schnepp B C, et al. Molecular characterization of adeno-associated viruses infecting children. J Virol 2005; 79:14781-92.
44. Cancer Genome Atlas Research Network. Electronic address wbe, Cancer Genome Atlas Research N. Comprehensive and Integrative Genomic Characterization of Hepatocellular Carcinoma. Cell 2017; 169:1327-1341 e23.
45. Fujimoto A, Furuta M, Totoki Y, et al. Whole-genome mutational landscape and characterization of noncoding and structural mutations in liver cancer. Nat Genet 2016; 48:500-9.
46. Schmidt M, Govindasamy L, Afione S, et al. Molecular characterization of the heparin-dependent transduction domain on the capsid of a novel adeno-associated virus isolate, AAV(VR-942). J Virol 2008; 82:8911-6.
47. Schnepp B C, Jensen R L, Clark K R, et al. Infectious molecular clones of adeno-associated virus isolated directly from human tissues. J Virol 2009; 83:1456-64.
48. Logan G J, Dane A P, Hallwirth C V, et al. Identification of liver-specific enhancer-promoter activity in the 3′ untranslated region of the wild-type AAV2 genome. Nat Genet 2017; 49:1267-1273.
49. Park K J, Lee J, Park J H, et al. Adeno-Associated Virus 2-Mediated Hepatocellular Carcinoma is Very Rare in Korean Patients. Ann Lab Med 2016; 36:469-74.
50. Nault J C, Couchy G, Balabaud C, et al. Molecular Classification of Hepatocellular Adenoma Associates With Risk Factors, Bleeding, and Malignant Transformation. Gastroenterology 2017; 152:880-894 e6.

1-45. (canceled)

46. A variant VP1 capsid protein that consists of the amino acid sequence as set forth in SEQ ID NO: 2 comprising at least one mutation selected from Table 1; or a derived variant VP2 or variant VP3 capsid protein.

47. A polynucleotide that encodes for a variant capsid protein according to claim 46.

48. An adeno-associated virus (AAV) that comprises at least one capsid protein according to claim 46, in particular a rAAV further comprising a heterologous nucleic acid comprising a transgene.

49. A method of delivering a transgene to a cell comprising contacting the cell with an amount of a rAAV comprising at least one capsid protein according to claim 46 and a heterologous nucleic acid comprising a transgene.

50. A method of therapy in a patient in need thereof, comprising administering to the patient a therapeutically effective amount of a rAAV comprising at least one capsid protein according to claim 46 and a heterologous nucleic acid comprising a transgene.

51. A pharmaceutical composition comprising a rAAV comprising at least one capsid protein according to claim 46 and a heterologous nucleic acid comprising a transgene, with pharmaceutically acceptable excipients, and optionally sustained-release matrices, such as biodegradable polymers.

52. A variant VP1 capsid protein that has an amino acid sequence selected from the group consisting of SEQ ID NO: 26 (capsid #3013), SEQ ID NO: 9 (capsid #2087), SEQ ID NO: 20 (capsid #1449), SEQ ID NO: 10 (capsid #2206), SEQ ID NO: 12 (capsid #1534), SEQ ID NO: 56 (capsid #163), SEQ ID NO: 23 (capsid #1343), SEQ ID NO: 53 (capsid #790), SEQ ID NO: 6 (capsid #1017), SEQ ID NO: 11 (capsid #877), SEQ ID NO: 8 (capsid #1273), SEQ ID NO: 3 (capsid #2497), SEQ ID NO: 60 (capsid #1055), SEQ ID NO: 14 (capsid #1919), SEQ ID NO: 4, SEQ ID NO: 5, SEQ ID NO: 7, SEQ ID NO: 13, SEQ ID NO: 15 to SEQ ID NO: 19, SEQ ID NO: 21, SEQ ID NO: 22, SEQ ID NO: 24, SEQ ID NO: 25, SEQ ID NO: 27 to SEQ ID NO: 55, SEQ ID NO: 57 to SEQ ID NO: 59, or SEQ ID NO: 61; or a derived variant VP2 or variant VP3 capsid protein.

53. A variant VP1 capsid protein that consists of the amino acid sequence as set forth in SEQ ID NO:2 comprising at least one mutation selected from the group consisting of V151A, Q164N; T200S; N201T; M211V; T233Q; M235L; T410Q; S446N; R447K; N449Q; T450S; P451N; T455L; T456Q; Q461L; A467P; S468T; D469S; I470M; R471S; D472L; S474A; R475K; V488L; T491Q; S492A; A493N; E499N; Y500F; S501P; G504A; Q536M; S537H; V539T; S547T; E548N; K549A; T550N; N551D; V552A; I554L; E555D; K556N; S578Y; T581N; R585N; R585S; G586S; R588T; Q589G; A590P; A591T; A593S; A593G; D594T; T597H; V600A; L647M; S658P; T660N; and A663S; or a derived variant VP2 or variant VP3 capsid protein.
